(19) United States
(12) Patent Application Publication
(10) Pub. No.: US 2020/0291089 A1
(43) Pub. Date: Sep. 17, 2020

(54) MULTIFUNCTIONAL MOLECULES COMPRISING A TRIMERIC LIGAND AND USES THEREOF

(71) Applicant: ELSTAR THERAPEUTICS, INC., Cambridge, MA (US)

(72) Inventors: Andreas Loew, Boston, MA (US); Brian Edward Vash, Cambridge, MA (US); Stephanie J. Maiocco, Arlington, MA (US)

(21) Appl. No.: 16/486,734

(22) PCT Filed: Feb. 16, 2018
(86) PCT No.: PCT/US2018/000031

§ 371 Date: Aug. 16, 2019

Related U.S. Application Data
(60) Provisional application No. 62/501,620, filed on May 4, 2017.
(60) Provisional application No. 62/491,626, filed on Apr. 28, 2017.
(60) Provisional application No. 62/459,846, filed on Feb. 16, 2017.

(51) Int. Cl. C07K 14/705; C07K 14/725

(52) U.S. Cl. CPC C07K 14/70575; C07K 2319/30; C07K 14/7051

(57) ABSTRACT

Disclosed herein are novel multifunctional, e.g., bifunctional, or trifunctional, molecules (e.g., fusion polypeptides or nucleic acids) that include a trimeric ligand, and optionally, an immunoglobulin constant domain, as well as methods of making and using the multifunctional molecules, e.g., for treating cancer.

### RELATED APPLICATION

This application claims priority to U.S. Ser. No. 62/459,846 filed Feb. 16, 2017 and U.S. Ser. No. 62/501,620 filed May 4, 2017, the content of each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Feb. 16, 2018, is named E2070-7004WO_SL.txt and is 522,047 bytes in size.

### BACKGROUND

Multifunctional molecules comprising a trimeric ligand, e.g., one, two or three tumor necrosis factor superfamily (TNFSF) or TNFSF-like members, and, optionally, a dimerization module, e.g., an immunoglobulin constant region, are disclosed.

### SUMMARY OF THE INVENTION

The present application discloses, at least in part, a novel multifunctional molecule (also interchangeably referred to herein as “multispecific molecule”) comprising a trimeric ligand, e.g., one, two or three trimeric ligands. In embodiments, the trimeric ligand includes three monomer molecules, e.g., wherein two of the monomer molecules are coupled, e.g., covalently linked, to one another, and the third monomer molecule is non-covalently associated to the other two monomer molecules. In some embodiments, the multifunctional molecule comprises two, three or more trimeric ligands that are the same or different. In some embodiments, the trimeric ligand in the multifunctional molecule is a homotrimer, e.g., is composed of the same monomer molecules, or a heterotrimer, e.g., is composed of two or three different monomer molecules. In some embodiments, the trimeric ligand is a member of the tumor necrosis factor superfamily (TNFSF) or TNFSF-like members, or a combination of TNFSF- and TNFSF-like monomers.

In some embodiments, the multifunctional molecule comprises a trimeric ligand (e.g., one or more (e.g., one, two or three) trimeric ligands), wherein said trimeric ligand comprises three monomer molecules, (e.g., three monomer molecules of the tumor necrosis factor superfamily (TNFSF) or TNSF-like member, or a combination thereof), wherein two of the monomer molecules are coupled, e.g., covalently linked, to one another, and the third monomer molecule is non-covalently associated to the other two monomer molecules.

In some embodiments, the multifunctional molecule further comprises a dimerization module (e.g., an immunoglobulin constant domain (e.g., an Fc or a dimerization module comprising a non-immunoglobulin dimerization domain, e.g., a TCRα constant domain and a TCRβ constant domain)).

In some embodiments, the multifunctional molecule further comprises one or more other binding specificities or functionalities chosen from one, two or more of: a targeting moiety, e.g., a tumor targeting moiety; an immune cell engager (e.g., chosen from one, two, three, or all of an NK cell engager, a B cell engager, a dendritic cell engager, or a macrophage cell engager); and/or a cytokine molecule.

In some embodiments, the multifunctional molecule comprises one trimeric ligand, two trimeric ligands, or three trimeric ligands.

In an embodiment, the multifunctional molecule comprises any of the configurations depicted in FIGS. 11A-11C, e.g., multispecific molecules attached to a dimerization module, e.g., an immunoglobulin constant domain. In embodiments, A, B, C and D, are coupled to, e.g., covalently linked, to a heterodimeric Fc domain, see FIG. 11A. In other embodiments, A, B, C and D, are coupled to, e.g., covalently linked, to a homodimeric Fc domain, see FIG. 11B. In other embodiments, A, B, C and D, covalently linked to a heterodimeric heavy and light variable region constant domains (e.g., a Fab CH1 and a Fab CL), see e.g., FIG. 11C. In one embodiment, the multifunctional molecule comprises a dimerization module comprising an immunoglobulin heavy chain constant region (e.g., an Fc region), e.g., a heterodimeric heavy chain constant region (e.g., a knob-in-hole heterodimer) as illustrated below:

In some embodiments, the multifunctional molecule comprises any of the configurations depicted in FIGS. 11A-11C or any of the configurations depicted in FIGS. 11A-11C wherein the dimerization domain does not contain a disulfide bond. In some embodiments, A, B, C and D, are coupled to, e.g., covalently linked, to a heterodimeric or a homodimeric dimerization domain (e.g., an immunoglobulin constant domain (e.g., an Fc or a dimerization module comprising a non-immunoglobulin dimerization domain, e.g., a TCRα constant domain and a TCRβ constant domain)). In some embodiments, A, B, C and D, are coupled to, e.g., covalently linked, to a heterodimeric Fab heavy and light constant domains (e.g., a CH1 domain and a CL domain).

In some embodiments, the multifunctional molecule includes the following configuration:
A, B-[dimerization module]-C, -D
   wherein:

1) the dimerization module comprises an immunoglobulin constant domain, e.g., a heavy chain constant domain (e.g., a homodimeric or heterodimeric heavy chain constant region, e.g., an Fc region), or a constant domain of an immunoglobulin Fab region; or a dimerization module comprising a non-immunoglobulin dimerization domain, e.g., a TCRa constant domain and a TCRβ constant domain);

2) A, B, C, and D are independently absent; a trimeric ligand; a targeting moiety, e.g., a tumor targeting moiety; an immune cell engager; or a cytokine molecule,

Provided that:

i) at least one, two or three of A, B, C, and D comprises a trimeric ligand;

ii) at least one of A, B, C, and D comprises a trimeric ligand, and any of the remaining A, B, C, and D comprises one, two or more of a targeting moiety, an immune cell engager, or a cytokine molecule;

iii) at least two of A, B, C, and D comprise a trimeric ligand, and any of the remaining A, B, C, and D comprises one, or two of a targeting moiety, an immune cell engager, or a cytokine molecule; or

iv) at least three of A, B, C, and D comprise a trimeric ligand, and any of the remaining A, B, C, and D is absent.

In some embodiments, 1) A, B, C, and D are independently chosen from being absent; a trimeric ligand; a targeting moiety, e.g., a tumor targeting moiety; an immune cell engager; or a cytokine molecule, or a combination thereof, wherein:

i) A is or comprises a trimeric ligand, and at least one, two or three of B, C, and D is or comprises a second trimeric ligand, a targeting moiety, an immune cell engager, a cytokine molecule, or is absent;

ii) A is or comprises a trimeric ligand; B is or comprises a targeting moiety; and C and D are absent;

iii) A is or comprises a trimeric ligand; B is or comprises a targeting moiety; and C is or comprises a second targeting moiety and D is absent, or C is absent and D comprises a second targeting moiety;

iv) A is or comprises a trimeric ligand; B is absent; and C is or comprises a targeting moiety; and D is absent or comprises a second targeting moiety;

v) A is or comprises a trimeric ligand; B is or comprises a targeting moiety; and C is or comprises an immune cell engager and D is absent, or C is absent and D is or comprises an immune cell engager;

vi) A is or comprises a trimeric ligand; B is or comprises a targeting moiety; and C is or comprises a cytokine molecule and D is absent, or C is absent and D is or comprises a cytokine molecule;

vii) A is or comprises a trimeric ligand; B is or comprises a targeting moiety; and C is or comprises an immune cell engager and D is or comprises a cytokine molecule, or C is or comprises a cytokine molecule and D is or comprises an immune cell engager;

viii) B is or comprises a trimeric ligand, and at least one, two or three of A, C, and D is or comprises a second trimeric ligand, a targeting moiety, an immune cell engager, a cytokine molecule, or is absent;

ix) B is or comprises a trimeric ligand; A is or comprises a targeting moiety; and C and D are absent;

x) B is or comprises a trimeric ligand; A is or comprises a targeting moiety; and C is or comprises a second targeting moiety and D is absent, or C is absent and D comprises a second targeting moiety;

xi) B is or comprises a trimeric ligand; A is absent; and C is or comprises a targeting moiety; and D is absent or comprises a second targeting moiety;

xii) B is or comprises a trimeric ligand; A is or comprises a targeting moiety; and C is or comprises an immune cell engager and D is absent, or C is absent and D is or comprises an immune cell engager;

xiii) B is or comprises a trimeric ligand; A is or comprises a targeting moiety; and C is or comprises a cytokine molecule and D is absent, or C is absent and D is or comprises a cytokine molecule;

xiv) B is or comprises a trimeric ligand; A is or comprises a targeting moiety; and C is or comprises an immune cell engager and D is absent, or C is absent and D is or comprises an immune cell engager;

xv) B is or comprises a trimeric ligand; A is or comprises a targeting moiety; and C is or comprises an immune cell engager and D is or comprises a cytokine molecule, or C is or comprises a cytokine molecule and D is or comprises an immune cell engager;

xvi) C is or comprises a trimeric ligand, and at least one of A or B is or comprises a second trimeric ligand or is absent, or at least one of A or B is or comprises a targeting moiety, an immune cell engager, a cytokine molecule, or is absent;

xvii) C is or comprises a trimeric ligand; A or B is or comprises a targeting moiety; and D is absent;

xviii) C is or comprises a trimeric ligand; A is or comprises a targeting moiety; and B is or comprises a second targeting moiety and D is absent, or B is absent and D comprises a second targeting moiety;

xix) C is or comprises a trimeric ligand; A is absent; and B is or comprises a targeting moiety; and D is absent;

xx) C is or comprises a trimeric ligand; A is or comprises a targeting moiety; and B is or comprises an immune cell engager and D is absent;

xxi) C is or comprises a trimeric ligand; A is or comprises a targeting moiety; and B is or comprises a cytokine molecule and D is absent, or B is absent and D is or comprises a cytokine molecule;

xxii) C is or comprises a trimeric ligand; A is or comprises a targeting moiety; and B is or comprises an immune cell engager, or B is or comprises a cytokine molecule;

xxiii) D is or comprises a trimeric ligand, and at least one, two or three of A or B is or comprises a targeting moiety, an immune cell engager, a cytokine molecule, or is absent;

xxiv) D is or comprises a trimeric ligand; A or B is or comprises a targeting moiety; and C is absent;

xxiv) D is or comprises a trimeric ligand; A is or comprises a targeting moiety; and B is or comprises a second targeting moiety and C is absent, or B is absent and C are absent;

xxv) D is or comprises a trimeric ligand; A is absent; and B is or comprises a targeting moiety; and C is absent;

xxvi) D is or comprises a trimeric ligand; A is or comprises a targeting moiety; and B is or comprises an immune cell engager and C is absent;

xxvii) D is or comprises a trimeric ligand; A is or comprises a targeting moiety; and B is or comprises a cytokine molecule and C is absent, or B is absent and C is or comprises a cytokine molecule;

xxviii) D is or comprises a trimeric ligand; A is or comprises a targeting moiety; and B is or comprises an immune cell engager, or B is or comprises a cytokine molecule; or

xxix) A, B, C or D is or comprises one, two, or three trimeric ligands.

In some embodiments, the multifunctional molecule comprises 1a) a first polypeptide comprising: a first monomer molecule of a trimeric ligand, e.g., homo- or heterotrimeric ligand, coupled to, e.g., covalently linked, e.g., via a linker, to a second monomer molecule of the trimeric ligand, wherein said first or second monomer molecule is connected, e.g., via a linker, to a first Fab heavy chain constant domain (e.g., a first CH1 domain, (e.g., to the N-terminus of the first Fab heavy chain constant domain)), and wherein said first Fab heavy chain constant domain is connected, e.g., via a linker, to a first member of a dimerization module, e.g., a homo- or heterodimerization module (e.g., a first Fc region (e.g., the N-terminal of the first Fc region, e.g., a knob or hole Fc region as described herein; or a first non-immunoglobulin dimerization domain, e.g., a TCRa constant domain or a TCRβ constant domain); and

2a) a second polypeptide comprising: a third monomer molecule of the trimeric ligand connected, e.g., via a linker, to a first Fab light chain constant domain (e.g., a first CL domain) (e.g., to the N-terminus of the first Fab light chain constant domain)).

In some embodiments, the multifunctional molecule further comprises 1b) a first polypeptide comprising: a first monomer molecule of a trimeric ligand, e.g., homo- or heterotrimeric ligand, coupled to, e.g., covalently linked, e.g., via a linker, to a first Fab heavy chain constant domain (e.g., to a first CH1 domain (e.g., to the N-terminus of the first Fab heavy chain constant domain)), wherein said first Fab heavy chain constant domain is connected, e.g., via a linker, to a first member of a dimerization module, e.g., a homo- or heterodimerization module (e.g., a first Fc region (e.g., the N-terminal of the first Fc region, e.g., a knob or hole Fc region as described herein; or a first non-immunoglobulin dimerization domain, e.g., a TCRa constant domain or a TCRβ constant domain); and 2b) a second polypeptide comprising: a second monomer molecule of the trimeric ligand connected, e.g., via a linker, to a third monomer molecule of the trimeric ligand, wherein, said second or third monomer molecule is connected, e.g., via a linker, to a first Fab light chain constant domain (e.g., to a first CL domain)

In some embodiments, said first Fab light chain constant region (e.g., said first CL domain) is covalently associated with said first Fab heavy chain constant region (e.g., said first CH1 domain). In some embodiments, said first Fab light chain constant region (e.g., said first CL domain) is linked to said first Fab heavy chain constant region (e.g., said first CH1 domain) by a disulfide bond.

In some embodiments, said first Fab light chain constant region (e.g., said first CL domain) is non-covalently associated with said first Fab heavy chain constant region (e.g., said first CH1 domain). In some embodiments, said first Fab light chain constant region (e.g., said first CL domain) is not linked to said first Fab heavy chain constant region (e.g., said first CH1 domain) by a disulfide bond. In some embodiments, said Fab heavy chain constant region (e.g., said first CH1 domain) is of IgG1 isotype and has a non-cysteine amino acid (e.g., a serine) at position 220 according to EU-index numbering system. In some embodiments, said first Fab light chain constant region (e.g., said first CL domain) is of kappa isotype and either a) lacks a C-terminal cysteine residue at position 214 according to Kabat numbering system (e.g., as compared to a parent CL kappa, e.g., a WT CL kappa); or b) has a non-cysteine amino acid residue substituted for a C-terminal cysteine at position 214 according to Kabat numbering system (e.g., as compared to a parent CL kappa, e.g., a WT CL kappa). In some embodiments, said first Fab light chain constant region (e.g., said first CL domain) is of lambda isotype and has a non-cysteine amino acid residue substituted for the cysteine at position 214 according to Kabat numbering system (e.g., as compared to a parent CL lambda, e.g., a WT CL lambda).

In some embodiments, the multispecific molecule further comprises 3) a third polypeptide comprising a second member of the dimerization module, e.g., a homo- or heterodimerization module (e.g., a second Fc region, e.g., the corresponding hole or knob Fc region as described herein; or a second non-immunoglobulin dimerization domain, a non-Fc region, e.g., a TCRα constant domain or TCRβ constant domain).

In some embodiments, said first and second member of a dimerization module, e.g., a homo- or heterodimerization module (e.g., a first and second Fc region; or a first and second half of a non-immunoglobulin dimerization domain, e.g., a TCRα constant domain or TCRβ constant domain), are covalently associated. In some embodiments, said first and second member of a dimerization module, e.g., a homo- or heterodimerization module (e.g., a first and second Fc region; or a first and second half of a non-immunoglobulin dimerization domain, e.g., a TCRα constant domain or TCRβ constant domain), are linked by a disulfide bond. In some embodiments, said first and second member of a dimerization module, e.g., a homo- or heterodimerization module (e.g., a first and second Fc region; or a first and second half of a non-immunoglobulin dimerization domain, a non-Fc region, e.g., a TCRα constant domain or TCRβ constant domain), are non-covalently associated. In some embodiments, said first and second member of a dimerization module, e.g., a homo- or heterodimerization module (e.g., a first and second Fc region; or a first and second half of a non-immunoglobulin dimerization domain, e.g., a TCRα constant domain or TCRβ constant domain), are not linked by a disulfide bond.

In some embodiments, said first member comprises a first Fc region and said second member comprises a second Fc region. In some embodiments, said first member comprises a first Fc region and said second member comprises a second Fc region, wherein each cysteine in the hinge region of said first and second Fc regions has been substituted with a non-cysteine amino acid (e.g., a serine) (e.g., as compared to a parent Fc region, e.g., a WT Fc region). In some embodiments, said first and second Fc regions are of IgG1 isotype and each Fc region has a non-cysteine amino acid (e.g., a serine) at position 226 and 229 according to EU-index numbering system. In some embodiments, said first member comprises a TCRα constant domain (or a functional fragment thereof, e.g., a fragment capable of forming stable association with a TCRβ constant domain); and said second member comprises a TCRβ constant domain (or a functional fragment thereof, e.g., a fragment capable of forming stable association with a TCRα constant domain). In some embodiments, said first member further comprises an immunoglobulin CH2 domain (e.g., an IgG1, IgG2, or IgG4 CH2 domain) connected to (optionally via a linker) the TCRα constant domain; and said second member further comprises an immunoglobulin CH2 domain (e.g., an IgG1, IgG2, or IgG4 CH2 domain) connected to (optionally via a linker) the TCRβ constant domain. In some embodiments, neither the first member nor the second member contains an immunoglobulin CH3 domain (e.g., any portion of a CH3 domain). In some embodiments, neither the first member nor the second member contains any portion of an immunoglobulin CH3 domain capable of stable self-association (i.e., the first member does not contain any portion of a CH3 domain capable of stable association with the CH3 domain of the second member).

In some embodiments, said first member comprises a TCRα variable domain connected to the TCRα constant domain, and the second member comprises a TCRβ variable domain connected to the TCRβ constant domain. In some embodiments, neither the first nor the second member contains more than 50, 25, 10, or 5 amino acids of an immunoglobulin CH2 domain and/or more than 50, 25, 10, or 5 amino acids of an immunoglobulin CH3 domain. In some embodiments, neither the first nor the second polypeptide chain of the heterodimerization domain contains an immunoglobulin CH2 and/or CH3 domain (e.g., any portion of a CH2 and/or CH3 domain).

In some embodiments, the TCRα constant domain comprises or consists of the amino acid sequence of SEQ ID NO: 118 and/or the TCRβ constant domain comprises or consists of the amino acid sequence of SEQ ID NO: 120. In some embodiments, the TCRα domain has 1 or more (e.g., 1, 2, 3, 4, 5, 5, 6, 7, 8, 9, 10, or more) amino acid substitutions; and/or the TCRβ domain has 1 or more (e.g., 1, 2, 3, 4, 5, 5, 6, 7, 8, 9, 10, or more) amino acid substitutions. In some embodiments, the TCRα domain has no more than 10 (e.g., 10, 9, 8, 7, 6, 5, 4, 3, 2, or 1) amino acid substitutions; and/or the TCRβ domain has no more than 10 (e.g., 10, 9, 8, 7, 6, 5, 4, 3, 2, or 1) amino acid substitutions. In some embodiments, the TCRα constant domain comprises or consists of the amino acid sequence of SEQ ID NO: 119 and/or the TCRβ constant domain comprises or consists of the amino acid sequence of SEQ ID NO: 121. In some embodiments, the TCRα domain has 1 or more (e.g., 1, 2, 3, 4, 5, 5, 6, 7, 8, 9, 10, or more) amino acid substitutions; and/or the TCRβ domain has 1 or more (e.g., 1, 2, 3, 4, 5, 5, 6, 7, 8, 9, 10, or more) amino acid substitutions. In some embodiments, the TCRα domain has no more than 10 (e.g., 10, 9, 8, 7, 6, 5, 4, 3, 2, or 1) amino acid substitutions; and/or the TCRβ domain has no more than 10 (e.g., 10, 9, 8, 7, 6, 5, 4, 3, 2, or 1) amino acid substitutions.

In some embodiments, the multifunctional molecule further comprises: 4) a first binding specificity or functionality chosen from a targeting moiety (e.g., a tumor targeting moiety); an immune cell engager; or a cytokine molecule, wherein the first binding specificity or functionality is coupled, e.g., covalently linked, to the third polypeptide, e.g., to the N-terminus of the third polypeptide.

In some embodiments, the first binding specificity or functionality comprises an antibody molecule (e.g., a scFv or Fab) against a first target antigen, e.g., a first tumor antigen (e.g., mesothelin)). In some embodiments, the multispecific molecule comprises: 5) a second binding specificity or functionality chosen from a targeting moiety (e.g., a tumor targeting moiety); an immune cell engager or a cytokine molecule, wherein the second binding specificity or functionality is connected to, e.g., optionally via a linker, to the first member of the dimerization module, e.g., to the C-terminus of the first polypeptide.

In some embodiments, the second binding specificity or functionality comprises an antibody molecule (e.g., a scFv or a Fab) against a second target antigen, e.g., a second tumor antigen (e.g., PD-L1)).

In some embodiments, the multifunctional molecule further comprises: 6) a third binding specificity or functionality chosen from a targeting moiety; an immune cell engager or a cytokine molecule, wherein the third binding specificity or functionality is connected to, e.g., optionally via a linker, to the second member of the dimerization module, e.g., to the C-terminus of the third polypeptide.

In some embodiments, the third binding specificity or functionality comprises an antibody molecule (e.g., a scFv or a Fab) against a third target antigen.

In some embodiments, the multifunctional molecule comprises: 7) a first polypeptide comprising: a first binding specificity or functionality chosen from a targeting moiety (e.g., a tumor targeting moiety); an immune cell engager; or a cytokine molecule, wherein the first binding specificity or functionality is connected to, e.g., optionally via a linker, to a first member of a dimerization module, e.g., a homo- or heterodimerization module (e.g., a first Fc region (e.g., the N-terminal of the first Fc region, e.g., a knob or hole Fc region as described herein), or a dimerization module comprising a non-immunoglobulin dimerization domain, e.g., a TCRα constant domain and a TCRβ constant domain).

In some embodiments, the first binding specificity or functionality comprises an antibody molecule (e.g., a scFv or a Fab) against a first target antigen, e.g., a first tumor antigen (e.g., mesothelin), wherein the antibody molecule comprises a first Fab heavy chain region (e.g., a first VH or VH-CH1) connected, e.g., via a linker, to the first member of the dimerization module, e.g., to the N-terminus of the first polypeptide; and 8) (optionally) wherein if the first binding specificity is a Fab, a second polypeptide comprising a first light chain.

In some embodiments, the multifunctional molecule further comprises: 9) a third polypeptide comprising a second member of the dimerization module, e.g., a homo- or heterodimerization module (e.g., a second Fc region, e.g., the corresponding hole or knob Fc region as described herein, or a dimerization module comprising a non-immunoglobulin dimerization domain, e.g., a TCRα constant domain and a TCRβ constant domain)).

In some embodiments, the multifunctional molecule further comprises: 10) a second binding specificity or functionality chosen from a targeting moiety (e.g., a tumor targeting moiety); an immune cell engager or a cytokine molecule connected, e.g., via a linker, to the second member of the dimerization module, e.g., to the N-terminus of the third polypeptide. In some embodiments, the second binding specificity or functionality comprises an antibody molecule (e.g., a scFv or a Fab) against a second target antigen, e.g., a second tumor antigen (e.g., PDL1), wherein the antibody molecule comprises a second Fab heavy chain region (e.g., a second VH or VH-CH1) connected, e.g., via a linker, to the second member of the dimerization module, e.g., to the N-terminus of the third polypeptide; and 11) (optionally) wherein if the second binding specificity is a Fab, a fourth polypeptide comprising a second light chain.

In some embodiments, the multifunctional molecule further comprises: 12a) a first monomer molecule of a trimeric ligand, e.g., homo- or heterotrimeric ligand, coupled to, e.g., covalently linked, e.g., via a linker, to a second monomer molecule of the trimeric ligand, wherein said first or second monomer molecule is connected, e.g., via a linker, to the second member of the dimerization module, e.g., the C-terminus of the third polypeptide; and 13a) a third monomer molecule of the trimeric ligand is coupled to, e.g., covalently linked, e.g., via a linker, to the first member of the dimerization module, e.g., the C-terminus of the first polypeptide.

In some embodiments, the multifunctional molecule further comprises: 12b) a first monomer molecule of a trimeric ligand, e.g., homo- or heterotrimeric ligand, coupled to, e.g., covalently linked, e.g., via a linker, to the second member of the dimerization module, e.g., the C-terminus of the third polypeptide; and 13b) a second monomer molecule of the trimeric ligand coupled, e.g., covalently linked, e.g., via a linker, to a third monomer molecule of the trimeric ligand, wherein the third monomer is coupled to, e.g., covalently linked, e.g., via a linker, to the first member of the dimerization module, e.g., the C-terminus of the first polypeptide.

In some embodiments, said first and second member of a dimerization module, e.g., a homo- or heterodimerization module (e.g., a first and second Fc region; or a first and second half of a non-immunoglobulin dimerization domain, a non-Fc region, e.g., a TCRα constant domain or TCRβ constant domain), are covalently associated. In some embodiments, said first and second member of a dimerization module, e.g., a homo- or heterodimerization module (e.g., a first and second Fc region; or a first and second half of a non-immunoglobulin dimerization domain, a non-Fc region, e.g., a TCRα constant domain or TCRβ constant domain), are linked by a disulfide bond.

In some embodiments, said first and second member of a dimerization module, e.g., a homo- or heterodimerization module (e.g., a first and second Fc region; or a first and second half of a non-immunoglobulin dimerization domain, a non-Fc region, e.g., a TCRα constant domain or TCRβ constant domain), are non-covalently associated. In some embodiments, said first and second member of a dimerization module, e.g., a homo- or heterodimerization module (e.g., a first and second Fc region; or a first and second half of a non-immunoglobulin dimerization domain, a non-Fc region, e.g., a TCRα constant domain or TCRβ constant domain), are not linked by a disulfide bond.

In some embodiments, said first member comprises a first Fc region and said second member comprises a second Fc region. In some embodiments, said first member comprises a first Fc region and said second member comprises a second Fc region, wherein each cysteine in the hinge region of said first and second Fc regions has been substituted with a non-cysteine amino acid (e.g., a serine) (e.g., as compared to a parent Fc region, e.g., a WT Fe region). In some embodiments, said first and second Fc regions are of IgG1 isotype and each Fc region has a non-cysteine amino acid (e.g., a serine) at position 226 and 229 according to EU-index numbering system.

In some embodiments, said first member comprises a TCRα constant domain (or a functional fragment thereof, e.g., a fragment capable of forming stable association with a TCR constant domain); and said second member comprises a TCRβ constant domain (or a functional fragment thereof, e.g., a fragment capable of forming stable association with a TCRα constant domain).

In some embodiments, said first member further comprises an immunoglobulin CH2 domain (e.g., an IgG1, IgG2, or IgG4 CH2 domain) connected to (optionally via a linker) the TCRα constant domain; and said second member further comprises an immunoglobulin CH2 domain (e.g., an IgG1, IgG2, or IgG4 CH2 domain) connected to (optionally via a linker) the TCRβ constant domain. In some embodiments, neither the first member nor the second member contains an immunoglobulin CH3 domain (e.g., any portion of a CH3 domain). In some embodiments, neither the first member nor the second member contains any portion of an immunoglobulin CH3 domain capable of stable self-association (i.e., the first member does not contain any portion of a CH3 domain capable of stable association with the CH3 domain of the second member).

In some embodiments, said first member comprises a TCRα variable domain connected to the TCRα constant domain, and the second member comprises a TCRβ variable domain connected to the TCRβ constant domain. In some embodiments, neither the first nor the second member contains more than 50, 25, 10, or 5 amino acids of an immunoglobulin CH2 domain and/or more than 50, 25, 10, or 5 amino acids of an immunoglobulin CH3 domain. In some embodiments, neither the first nor the second polypeptide chain of the heterodimerization domain contains an immunoglobulin CH2 and/or CH3 domain (e.g., any portion of a CH2 and/or CH3 domain).

In some embodiments, the TCRα constant domain comprises or consists of the amino acid sequence of SEQ ID NO: 118 and/or the TCRβ constant domain comprises or consists of the amino acid sequence of SEQ ID NO: 120. In some embodiments, the TCRα domain has 1 or more (e.g., 1, 2, 3, 4, 5, 5, 6, 7, 8, 9, 10, or more) amino acid substitutions; and/or the TCR domain has 1 or more (e.g., 1, 2, 3, 4, 5, 5, 6, 7, 8, 9, 10, or more) amino acid substitutions. In some embodiments, the TCRα domain has no more than 10 (e.g., 10, 9, 8, 7, 6, 5, 4, 3, 2, or 1) amino acid substitutions; and/or the TCRβ domain has no more than 10 (e.g., 10, 9, 8, 7, 6, 5, 4, 3, 2, or 1) amino acid substitutions. In some embodiments, the TCRα constant domain comprises or consists of the amino acid sequence of SEQ ID NO: 119 and/or the TCR constant domain comprises or consists of the amino acid sequence of SEQ ID NO: 121. In some embodiments, the TCRα domain has 1 or more (e.g., 1, 2, 3, 4, 5, 5, 6, 7, 8, 9, 10, or more) amino acid substitutions; and/or the TCRβ domain has 1 or more (e.g., 1, 2, 3, 4, 5, 5, 6, 7, 8, 9, 10, or more) amino acid substitutions. In some embodiments, the TCRα domain has no more than 10 (e.g., 10, 9, 8, 7, 6, 5, 4, 3, 2, or 1) amino acid substitutions; and/or the TCRβ domain has no more than 10 (e.g., 10, 9, 8, 7, 6, 5, 4, 3, 2, or 1) amino acid substitutions.

In some embodiments, the multifunctional molecule comprises one trimeric ligand, e.g., a homo- or heterotrimeric ligand as depicted in FIGS. 1A-3D, 13A-13D, 14A-14D, 15A-15D, 22A-22C, and 23A-23C.

In some embodiments, the multifunctional molecule comprises two or three trimeric ligands that are the same or different trimeric ligands, e.g., the same or different homotrimers or heterotrimer ligand, or a combination of homotrimer and heterotrimer ligands.

In some embodiments, the multifunctional molecule comprises two trimeric ligands, e.g., the same homotrimeric ligand coupled to a homodimeric immunoglobulin Fc region and Fab light chain constant region-Fab heavy chain constant region (e.g., CL-CH1) as depicted in FIGS. 4A, 4D, 16A, and 16D, or the same homotrimeric ligand coupled to a heterodimeric immunoglobulin Fc region and Fab light chain constant region-Fab heavy chain constant region (e.g., CL-CH1) as depicted in FIGS. 4B, 4E, 16B, and 16E.

In some embodiments, the multifunctional molecule comprises two different homotrimeric ligands coupled to a heterodimeric immunoglobulin Fc region and Fab light chain constant region-Fab heavy chain constant region (e.g., CL-CH1) as depicted in FIGS. 4C, 4F, 16C, and 16F.

In some embodiments, the multifunctional molecule comprises two heterotrimeric ligands, e.g., the same heterotrimeric ligand coupled to a homodimeric immunoglobulin Fc region and Fab light chain constant region-Fab heavy chain constant region (e.g., CL-CH1) as depicted in FIGS. 5A, 5D, 17A, and 17D, or the same heterotrimeric ligand coupled to a heterodimeric immunoglobulin Fc region and Fab light chain constant region-Fab heavy chain constant region (e.g., CL-CH1) as depicted in FIGS. 5B, 5E, 17B, and 17E.

In some embodiments, the multifunctional molecule comprises two different heterotrimeric ligands coupled to a heterodimeric immunoglobulin Fe region and Fab light chain constant region-Fab heavy chain constant region (e.g., CL-CH1) as depicted in FIGS. 5C, 5F, 17C, and 17F.

In some embodiment, the multifunctional molecule comprises three trimeric ligands that are the same or different trimeric ligands, e.g., the same or different homotrimer or heterotrimer ligands, or a combination of homotrimer and heterotrimer ligands, e.g., as depicted in FIGS. 6A-7H, and 18A-19H.

In some embodiments, the multifunctional molecule comprises three trimeric ligands, e.g., the same three homotrimeric ligand coupled to a heterodimeric immunoglobulin Fc region and Fab light chain constant region-Fab heavy chain constant region (e.g., CL-CH1) as depicted in FIGS. 6A, 6B, 18A, and 18B, or two of the same homotrimeric ligands and a third different homotrimeric ligand coupled to a heterodimeric immunoglobulin Fc region and Fab light chain constant region-Fab heavy chain constant region (e.g., CL-CH1) as depicted in FIGS. 6C, 6D, 18C, and 18D.

In some embodiments, the multifunctional molecule comprises three different homotrimeric ligands coupled to a heterodimeric immunoglobulin Fc region and Fab light chain constant region-Fab heavy chain constant region (e.g., CL-CH1) as depicted in FIGS. 6E-6H and 18E-18H.

In some embodiments, the multifunctional molecule comprises three different heterotrimeric ligands coupled to a heterodimeric immunoglobulin Fc region and Fab light chain constant region-Fab heavy chain constant region (e.g., CL-CH1) as depicted in FIGS. 7A-7H and 19A-19H.

In some embodiments, the multifunctional molecule comprises a homodimeric or a heterodimeric heavy chain constant region (e.g., the Fc region, e.g., a first Fc region and a second Fc region). In some embodiments, the said first and second Fc regions are non-covalently associated (e.g., the first and second Fc region are not linked by a disulfide bond). In some embodiments, each cysteine in the hinge region of said first and second Fc regions has been substituted with a non-cysteine amino acid (e.g., serine) (e.g., as compared to a parent Fc region, e.g., a WT Fc region). In some embodiments, said first and second Fc regions are of IgG1 isotype and each Fc region has a non-cysteine amino acid (e.g., has a serine) at position 226 and 229 according to EU-index numbering system. In some embodiments, one or both of the first and second Fc regions are altered, e.g., mutated, to increase dimerization, e.g., relative to a non-engineered interface, e.g., a naturally-occurring interface. In some embodiments, the trimeric ligand (e.g., a homotrimer or a heterotrimer ligand) is coupled, e.g., covalently coupled or fused, optionally via a linker, to the N- or C-terminus of the heavy chain constant region, e.g., the Fc region.

In some embodiments, the trimeric ligand (e.g., a homotrimer or a heterotrimer ligand) is coupled, e.g., via a linker, to the C-terminus of an Fc region (e.g., the C-terminus of a homo- or heterodimeric Fc region comprising a first and second polypeptide chain).

In some embodiments, one of the heavy chain constant region contains two ligand monomer molecules (e.g., two non-covalently or covalently coupled ligand monomer molecules), and the other heavy chain constant region comprises a single ligand monomer molecule, e.g., as depicted in FIGS. 1A-1B and 13A-B).

In some embodiments, the trimeric ligand (e.g., a homotrimer or a heterotrimer ligand) is coupled, e.g., via a linker, to the N-terminus of an Fc region (e.g., the N-terminus of a homo- or heterodimeric Fc region comprising a first and second polypeptide chain).

In some embodiments, one of the immunoglobulin constant region contains two ligand monomer molecules (e.g., two non-covalently or covalently coupled ligand monomer molecules), and the other immunoglobulin constant region comprises a single ligand monomer molecule, e.g., as depicted in FIGS. 1C-1D and 13C-13D.

In some embodiments, the trimeric ligand is coupled, e.g., covalently coupled or fused, optionally via a linker, to a Fab constant domain (e.g., a CH1 or CL domain).

In some embodiments, the trimer ligand is coupled, e.g., covalently coupled or fused, to the N-terminus of a Fab heavy chain constant region (e.g., a CH1) and/or a Fab light chain constant region (e.g., a CL).

In some embodiments, the trimeric ligand (e.g., a homotrimer or a heterotrimer ligand) is coupled, e.g., via a linker, to the N-terminus of a Fab constant domain, e.g., a CH1 and CL.

In some embodiments, the CH1 domain comprises two trimer monomer molecules coupled, e.g., via a linker, to the N-terminus of the CH1 domain, and the CL domain comprises a single trimer monomer molecule coupled, e.g., via a linker to the N-terminus of the CL domain (e.g., as depicted in FIGS. 2A-2B and 14A-14B).

In some embodiments, the CH1 domain comprises one monomer molecule of the trimer coupled, e.g., via a linker, to the N-terminus of the CH1 domain, and the CL domain comprises two monomer molecules of the trimer coupled, e.g., via a linker to the N-terminus of the CL domain (e.g., as depicted in FIGS. 2C-2D).

In some embodiments, the CH1 domain comprises two trimer monomer molecules coupled, e.g., via a linker, to the N-terminus of the CH1 domain, and the CL domain comprises a single trimer monomer molecule coupled, e.g., via a linker to the N-terminus of the CL domain (e.g., as depicted in FIGS. 3A-3B).

In some embodiments, the CH1 domain comprises one trimer monomer molecule coupled, e.g., via a linker, to the N-terminus of the CH1 domain, and the CL domain comprises two trimer monomer molecule coupled, e.g., via a linker to the N-terminus of the CL domain (e.g., as depicted in FIGS. 3C-3D).

In some embodiments, the trimeric ligand is chosen from a TNFSF family member or a TNF-like family member, or a combination thereof, e.g., as described herein in Tables 1 and 2.

In some embodiments, the trimeric ligand is a homotrimer, e.g., of the same TNFSF family member or the same TNF-like family member.

In some embodiments, the trimeric ligand is a heterotrimer, e.g., it comprises a combination of monomer molecules from two or three trimeric ligands, e.g., two or three TNFSF or TNF-like family members.

In some embodiments, the trimeric ligand comprises the amino acid sequence of a monomer molecule chosen from a TNFSF family member or a TNF-like family member, e.g., as described herein in Tables 1 and 2, or an amino acid sequence substantially identical thereto, e.g., at least 85%, 90%, 95%, 99% or more identical to an amino acid described in Tables 1 and 2.

In some embodiments, the trimeric ligand comprises any of the TNFSF amino acid sequences for TNSF1, TNSF2, TNSF3, TNSF4, TNSF5, TNSF6, TNSF7, TNSF8, TNSF9, TNSF10, TNSF1, TNSF11, TNSF12, TNSF13, TNSF13B, TNSF14, TNSF15, TNF18 or EDA, corresponding to SEQ ID NOs: 1-17 and 218, in Table 1, or an amino acid sequence substantially identical thereto, e.g., at least 85%, 90%, 95%, 99% or more identical to the amino acid sequence of any of SEQ ID NOs: 1-17 and 218, as a homotrimer, or a heterotrimer comprising any combination of two or three of the aforesaid monomer molecules.

In some embodiments, the trimeric ligand comprises any of the TNF-like amino acid sequences for Complement C1Q (e.g., subcomponents A, B and C), C1QL1, C1QL2, C1QL3, Caprin-2 C1q domain, cerebellin-1 C1q domain or adiponectin, corresponding to SEQ ID NOs: 239-247, in Table 2, or an amino acid sequence substantially identical thereto, e.g., at least 85%, 90%, 95%, 99% or more identical to the amino acid sequence of any of SEQ ID NOs: 239-247, as a homotrimer (e.g., comprises of homomonomer molecules), or a heterotrimer (e.g., comprises of heteromonomer molecules) comprising any combination of two or three of the aforesaid monomers.

In some embodiments, the trimeric ligand comprises a combination of TNFSF monomer molecules and TNF-like monomer molecules, wherein the trimer ligand is a heterotrimer comprising any combination of two or three monomer molecules comprising the amino acid sequence of any of SEQ ID NOs: 1-17, 218, and 239-247.

In some embodiments, the immunoglobulin constant region comprises an Fc region, said Fc region comprising a paired amino acid substitution at a position chosen from one or more of 347, 349, 350, 351, 366, 368, 370, 392, 394, 395, 397, 398, 399, 405, 407, or 409, e.g., of the Fc region of human IgG1. In some embodiments, the Fc region comprises a paired amino acid substitution chosen from: T366S, L368A, or Y407V (e.g., corresponding to a cavity or hole), and T366W (e.g., corresponding to a protuberance or knob).

In some embodiments, the immunoglobulin constant region comprises a constant domain of a Fab region.

In some embodiments, the multifunctional molecule comprises the amino acid sequence of SEQ ID NO: 18 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 18 (without the signal peptide SEQ ID NO: 74), and optionally, the other heavy chain sequence from the N-terminus to the C-terminus is comprised the amino acid sequence of SEQ ID NO: 19 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 19 (without the signal peptide SEQ ID NO: 74).

In some embodiments, the multifunctional molecule comprises the amino acid sequence of SEQ ID NO: 20 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 20 (without the signal peptide SEQ ID NO: 74), and optionally, the other heavy chain is comprised of the amino acid sequence of SEQ ID NO: 21 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 21 (without the signal peptide SEQ ID NO: 74).

In some embodiments, the light chain that pairs with SEQ ID NO: 21 (without the signal peptide SEQ ID NO: 74) is comprised of the amino acid sequence of SEQ ID NO: 22 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 22 (without the signal peptide SEQ ID NO: 74).

In some embodiments, the multifunctional molecule comprises the amino acid sequence of SEQ ID NO: 23 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 23 (without the signal peptide SEQ ID NO: 74); and optionally, the other heavy chain sequence from the N-terminus to the C-terminus is comprised the amino acid sequence of SEQ ID NO: 24 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 24 (without the signal peptide SEQ ID NO: 74).

In some embodiments, the multifunctional molecule comprises the amino acid sequence of SEQ ID NO: 20 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 20 (without the signal peptide SEQ ID NO: 74); and optionally, the other heavy chain is comprised of the amino acid sequence of SEQ ID NO: 25 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 25 (without the signal peptide SEQ ID NO: 74).

In some embodiments, the light chain that pairs with SEQ ID NO: 25 (without the signal peptide SEQ ID NO: 74) is comprised of the amino acid sequence of SEQ ID NO: 26 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 26 (without the signal peptide SEQ ID NO: 74).

In some embodiments, the multifunctional molecule comprises the amino acid sequence of SEQ ID NO: 27 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 27 (without the signal peptide SEQ ID NO: 74); and optionally, the other heavy chain sequence from the N-terminus to the C-terminus is comprised of the amino acid sequence of SEQ ID NO: 28 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 28 (without the signal peptide SEQ ID NO: 74).

In some embodiments, the multifunctional molecule comprises the amino acid sequence of SEQ ID NO: 29 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 29 (without the signal peptide SEQ ID NO: 74).

In some embodiments, the light chain that pairs with SEQ ID NO: 29 (without the signal peptide SEQ ID NO: 74) is comprised of the amino acid sequence of SEQ ID NO: 30 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 30 (without the signal peptide SEQ ID NO: 74).

In some embodiments, the multifunctional molecule comprises the amino acid sequence of SEQ ID NO: 31 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 31 (without the signal peptide SEQ ID NO: 74); and optionally, the other heavy chain sequence from the N-terminus to the C-terminus is comprised the amino acid sequence of SEQ ID NO: 32 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 32 (without the signal peptide SEQ ID NO: 74).

In some embodiments, the multifunctional molecule comprises the amino acid sequence of SEQ ID NO: 33 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 33 (without the signal peptide SEQ ID NO: 74).

In some embodiments, the light chain that pairs with SEQ ID NO: 25 (without the signal peptide SEQ ID NO: 74) is comprised of the amino acid sequence of SEQ ID NO: 34 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 34 (without the signal peptide SEQ ID NO: 74).

In some embodiments, the multifunctional molecule comprises the amino acid sequence of SEQ ID NO: 35 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 35 (without the signal peptide SEQ ID NO: 74); and optionally, wherein the other heavy chain sequence from the N-terminus to the C-terminus is comprised of the amino acid sequence of SEQ ID NO: 36 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 36 (without the signal peptide SEQ ID NO: 74).

In some embodiments, the multifunctional molecule comprises the amino acid sequence of SEQ ID NO: 37 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 37 (without the signal peptide SEQ ID NO: 74).

In some embodiments, the light chain that pairs with SEQ ID NO: 25 (without the signal peptide SEQ ID NO: 74) is comprised of the amino acid sequence of SEQ ID NO: 38 (without the signal peptide SEQ ID NO: 74), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 38 (without the signal peptide SEQ ID NO: 74).

In one aspect, disclosed herein is a multifunctional molecule, comprising a trimeric ligand (e.g., one or more (e.g., one, two or three) trimeric ligands), wherein said trimeric ligand comprises a first monomer molecule, a second monomer molecule, and a third monomer molecule (e.g., three monomer molecules of the tumor necrosis factor superfamily (TNFSF) or TNSF-like member, or a combination thereof), wherein the first and second monomer molecules are coupled, e.g., covalently linked, to one another, and the third monomer molecule is non-covalently associated to the first and second monomer molecules, wherein the first and second monomer molecules are coupled, e.g., covalently linked, to a first dimerization molecule, and the third monomer molecule is coupled, e.g., covalently linked, to a second dimerization molecule, wherein the first dimerization molecule is non-covalently associated with the second dimerization molecule.

In some embodiments, the first dimerization molecule is not linked to the second dimerization molecule by a disulfide bond. In some embodiments, the first dimerization molecule comprises a Fab heavy chain constant region (e.g., a CH1 domain), and the second dimerization molecule comprises a Fab light chain constant region (e.g., a CL domain). In some embodiments, the first dimerization molecule comprises a Fab light chain constant region (e.g., a CL domain), and the second dimerization molecule comprises a Fab heavy chain constant region (e.g., a CH1 domain). In some embodiments, the first and second monomer molecules are coupled, e.g., covalently linked, to the N-terminus of the first dimerization molecule, and the third monomer molecule is coupled, e.g., covalently linked, to the N-terminus of the second dimerization molecule.

In some embodiments, the Fab heavy chain constant region (e.g., the CH1 domain) is of IgG1 isotype and lacks a cysteine residue at position 220 according to EU-index numbering system (e.g., as compared to a parent CH1, e.g., a WT CH1), optionally wherein the Fab heavy chain constant region comprises a deletion at position 220 according to EU-index numbering system (e.g., as compared to a parent CH1, e.g., a WT CH1). In some embodiments, the Fab heavy chain constant region (e.g., the CH1 domain) is of IgG1 isotype and has a non-cysteine amino acid residue (e.g., a serine) at position 220 according to EU-index numbering system, optionally wherein the Fab heavy chain constant region comprises a C220S substitution according to EU-index numbering system (e.g., as compared to a parent CH1, e.g., a WT CH1), optionally wherein the Fab heavy chain constant region comprises the amino acid sequence of SEQ ID NO: 192.

In some embodiments, the Fab light chain constant region (e.g., the CL domain) is of kappa isotype and lacks a cysteine residue at position 214 according to Kabat numbering system (e.g., as compared to a parent CL kappa, e.g., a WT CL kappa), optionally wherein the Fab light chain constant region comprises a deletion at position 214 according to Kabat numbering system (e.g., as compared to a parent CL kappa, e.g., a WT CL kappa). In some embodiments, the Fab light chain constant region (e.g., the CL domain) is of kappa isotype and has a non-cysteine amino acid residue (e.g., a serine) at position 214 according to Kabat numbering system, optionally wherein the Fab light chain constant region comprises a C214S substitution according to Kabat numbering system (e.g., as compared to a parent CL kappa, e.g., a WT CL kappa), optionally wherein the Fab light chain constant region comprises the amino acid sequence of SEQ ID NO: 191.

In some embodiments, the Fab light chain constant region (e.g., the CL domain) is of lambda isotype and lacks a cysteine residue at position 214 according to Kabat numbering system (e.g., as compared to a parent CL lambda, e.g., a WT CL lambda), optionally wherein the Fab light chain constant region comprises a deletion at position 214 according to Kabat numbering system (e.g., as compared to a parent CL lambda, e.g., a WT CL lambda). In some embodiments, the Fab light chain constant region (e.g., the CL domain) is of lambda isotype and has a non-cysteine amino acid residue (e.g., a serine) at position 214 according to Kabat numbering system, optionally wherein the Fab light chain constant region comprises a C214S substitution according to Kabat numbering system (e.g., as compared to a parent CL lambda, e.g., a WT CL lambda).

In some embodiments, the first dimerization molecule comprises an amino acid sequence chosen from SEQ ID NO: 181, 197, 186, 192, 187, 188, 193, or 194, and the second dimerization molecule comprises the amino acid sequence of SEQ ID NO: 183 or 191. In some embodiments, the first dimerization molecule comprises the amino acid sequence of SEQ ID NO: 183 or 191, and the second dimerization molecule comprises an amino acid sequence chosen from SEQ ID NO: 181, 197, 186, 192, 187, 188, 193, or 194.

In some embodiments, the first dimerization molecule comprises a first heavy chain constant region (e.g., a first hinge region, a first CH2 region, a first CH3 region, or a first Fc region), and the second dimerization molecule comprises a second heavy chain constant region (e.g., a second hinge region, a second CH2 region, a second CH3 region, or a second Fc region). In some embodiments, the first and second monomer molecules are coupled, e.g., covalently linked, to the C-terminus of the first dimerization molecule, and the third monomer molecule is coupled, e.g., covalently linked, to the C-terminus of the second dimerization molecule. In some embodiments, one or more cysteine residues (e.g., all cysteine residues) in the first and/or second heavy chain constant region (e.g., the first and/or second hinge region) have been substituted with a non-cysteine amino acid residue (e.g., a serine) (e.g., as compared to a parent heavy chain constant region, e.g., a WT hinge region). In some embodiments, the first and second heavy chain constant regions are of IgG1 isotype, wherein the first and/or second heavy chain constant region (e.g., the first and/or second hinge regions) has a non-cysteine amino acid residue (e.g., a serine) at positions 226 and 229 according to EU-index numbering system. In some embodiments, the first heavy chain constant region comprises a C226S substitution and a C229S substitution. In some embodiments, the second heavy chain constant region comprises a C226S substitution and a C229S substitution. In some embodiments, the first and second heavy chain constant regions comprise the amino acid sequences of SEQ ID NOs: 184 and 185, respectively. In some embodiments, the first and second heavy chain constant regions comprise the amino acid sequences of SEQ ID NOs: 185 and 184, respectively. In some embodiments, the first and second heavy chain constant regions comprise the amino acid sequences of SEQ ID NOs: 184 and 43, respectively. In some embodiments, the first and second heavy chain constant regions comprise the amino acid sequences of SEQ ID NOs: 43 and 184, respectively. In some embodiments, the first and second heavy chain constant regions comprise the amino acid sequences of SEQ ID NOs: 77 and 185, respectively. In some embodiments, the first and second heavy chain constant regions comprise the amino acid sequences of SEQ ID NOs: 185 and 77, respectively.

In some embodiments, the first dimerization molecule comprises a TCRα constant domain (e.g., a naturally existing sequence or a functional variant thereof, e.g., a functional variant capable of forming stable association with a TCRβ constant domain), and the second dimerization molecule comprises a TCRβ constant domain (e.g., a naturally existing sequence or a functional variant thereof, e.g., a functional variant capable of forming stable association with a TCRα constant domain). In some embodiments, the first dimerization molecule comprises a TCRβ constant domain (e.g., a naturally existing sequence or a functional variant thereof, e.g., a functional variant capable of forming stable association with a TCRα constant domain), and the second dimerization molecule comprises a TCRα constant domain (e.g., a naturally existing sequence or a functional variant thereof, e.g., a functional variant capable of forming stable association with a TCRβ constant domain).

In some embodiments, the first dimerization molecule comprises an immunoglobulin CH2 domain (e.g., an IgG1, IgG2, or IgG4 CH2 domain) connected to (optionally via a linker) a TCRα constant domain (e.g., a naturally existing sequence or a functional variant thereof, e.g., a functional variant capable of forming stable association with a TCRβ constant domain), and the second dimerization molecule comprises an immunoglobulin CH2 domain (e.g., an IgG1, IgG2, or IgG4 CH2 domain) connected to (optionally via a linker) a TCR constant domain (e.g., a naturally existing sequence or a functional variant thereof, e.g., a functional variant capable of forming stable association with a TCRα constant domain). In some embodiments, the first dimerization molecule comprises an immunoglobulin CH2 domain (e.g., an IgG1, IgG2, or IgG4 CH2 domain) connected to (optionally via a linker) a TCRβ constant domain (e.g., a naturally existing sequence or a functional variant thereof, e.g., a functional variant capable of forming stable association with a TCRα constant domain), and the second dimerization molecule comprises an immunoglobulin CH2 domain (e.g., an IgG1, IgG2, or IgG4 CH2 domain) connected to (optionally via a linker) a TCRα constant domain (e.g., a naturally existing sequence or a functional variant thereof, e.g., a functional variant capable of forming stable association with a TCRβ constant domain). In some embodiments, the TCRα constant domain comprises or consists of the amino acid sequence of SEQ ID NO: 118 (or a sequence having 1 or more (e.g., 1, 2, 3, 4, 5, 5, 6, 7, 8, 9, 10, or more) amino acid substitutions relative to the amino acid sequence of SEQ ID NO: 118, or a sequence having no more than 10 (e.g., 10, 9, 8, 7, 6, 5, 4, 3, 2, or 1) amino acid substitutions relative to the amino acid sequence of SEQ ID NO: 118). In some embodiments, the TCRβ constant domain comprises or consists of the amino acid sequence of SEQ ID NO: 120 (or a sequence having 1 or more (e.g., 1, 2, 3, 4, 5, 5, 6, 7, 8, 9, 10, or more) amino acid substitutions relative to the amino acid sequence of SEQ ID NO: 120, or a sequence having no more than 10 (e.g., 10, 9, 8, 7, 6, 5, 4, 3, 2, or 1) amino acid substitutions relative to the amino acid sequence of SEQ ID NO: 120). In some embodiments, the TCRα constant domain comprises or consists of the amino acid sequence of SEQ ID NO: 119 (or a sequence having 1 or more (e.g., 1, 2, 3, 4, 5, 5, 6, 7, 8, 9, 10, or more) amino acid substitutions relative to the amino acid sequence of SEQ ID NO: 119, or a sequence having no more than 10 (e.g., 10, 9, 8, 7, 6, 5, 4, 3, 2, or 1) amino acid substitutions relative to the amino acid sequence of SEQ ID NO: 119). In some embodiments, the TCR constant domain comprises or consists of the amino acid sequence of SEQ ID NO: 121 (or a sequence having 1 or more (e.g., 1, 2, 3, 4, 5, 5, 6, 7, 8, 9, 10, or more) amino acid substitutions relative to the amino acid sequence of SEQ ID NO: 121, or a sequence having no more than 10 (e.g., 10, 9, 8, 7, 6, 5, 4, 3, 2, or 1) amino acid substitutions relative to the amino acid sequence of SEQ ID NO: 121).

In some embodiments, the first dimerization molecule comprises a TCRα variable domain connected to a TCRα constant domain (e.g., a naturally existing sequence or a functional variant thereof, e.g., a functional variant capable of forming stable association with a TCR constant domain), and the second dimerization molecule comprises a TCRβ variable domain connected to a TCR constant domain (e.g., a naturally existing sequence or a functional variant thereof, e.g., a functional variant capable of forming stable association with a TCRα constant domain). In some embodiments, the first dimerization molecule comprises a TCR variable domain connected to a TCRβ constant domain (e.g., a naturally existing sequence or a functional variant thereof, e.g., a functional variant capable of forming stable association with a TCRα constant domain), and the second dimerization molecule comprises a TCRα variable domain connected to a TCRα constant domain (e.g., a naturally existing sequence or a functional variant thereof, e.g., a functional variant capable of forming stable association with a TCR constant domain).

In some embodiments, neither the first nor the second dimerization molecule comprises an immunoglobulin CH3 domain (e.g., any portion of a CH3 domain). In some embodiments, neither the first nor the second dimerization molecule comprises any portion of an immunoglobulin CH3 domain capable of stable self-association (i.e., the first dimerization molecule does not comprise any portion of a CH3 domain capable of stable association with the CH3 domain of the second dimerization molecule).

In some embodiments, neither the first nor the second dimerization molecule comprises more than 50, 25, 10, or 5 amino acids of an immunoglobulin CH2 domain and/or more than 50, 25, 10, or 5 amino acids of an immunoglobulin CH3 domain. In some embodiments, neither the first nor the second dimerization molecule comprises an immunoglobulin CH2 and/or CH3 domain (e.g., any portion of a CH2 and/or CH3 domain).

In some embodiments, the trimeric ligand is a heterotrimer. In some embodiments, the first monomer molecule, the second monomer molecule, and the third monomer molecule are not identical. In some embodiments, the first and second monomer molecules are the same (or sharing no less than 70%, 75%, 80%, 85%, or 90% sequence identity), and the third monomer molecule is different from the first and second monomer molecules (e.g., sharing no more than 40%, 45%, 50%, 55%, or 60% sequence identity with the first or second monomer molecule). In some embodiments, the first and third monomer molecules are the same (or sharing no less than 70%, 75%, 80%, 85%, or 90% sequence identity), and the second monomer molecule is different from the first and third monomer molecules (e.g., sharing no more than 40%, 45%, 50%, 55%, or 60% sequence identity with the first or second monomer molecule). In some embodiments, the second and third monomer molecules are the same (or sharing no less than 70%, 75%, 80%, 85%, or 90% sequence identity), and the first monomer molecule is different from the second and third monomer molecules (e.g., sharing no more than 40%, 45%, 50%, 55%, or 60% sequence identity with the first or second monomer molecule).

In some embodiments, the first, second, and third monomer molecules are independently chosen from BAFF (e.g., a naturally existing sequence or a functional variant thereof) or APRIL (e.g., a naturally existing sequence or a functional variant thereof), wherein at least one of the three monomer molecules is BAFF (e.g., a naturally existing sequence or a functional variant thereof), and at least one of the three monomer molecules is APRIL (e.g., a naturally existing sequence or a functional variant thereof). In some embodiments, the first, second, and third monomer molecules independently comprise the amino acid sequence of SEQ ID NO: 14 (or a sequence having at least 70%, 75%, 80%, 85%, 90%, or 95% sequence identity thereof) or the amino acid sequence of SEQ ID NO: 13 or 180 (or a sequence having at least 70%, 75%, 80%, 85%, 90%, or 95% sequence identity). In some embodiments, the first monomer molecule is BAFF (e.g., a naturally existing sequence or a functional variant thereof) (e.g., a sequence comprising the amino acid sequence of SEQ ID NO: 14, or a sequence having at least 70%, 75%, 80%, 85%, 90%, or 95% sequence identity thereof), the second monomer molecule is BAFF (e.g., a naturally existing sequence or a functional variant thereof) (e.g., a sequence comprising the amino acid sequence of SEQ ID NO: 14, or a sequence having at least 70%, 75%, 80%, 85%, 90%, or 95% sequence identity thereof), and the third monomer molecule is APRIL (e.g., a naturally existing sequence or a functional variant thereof) (e.g., a sequence comprising the amino acid sequence of SEQ ID NO: 13 or 180, or a sequence having at least 70%, 75%, 80%, 85%, 90%, or 95% sequence identity thereof). In some embodiments, the first monomer molecule is APRIL (e.g., a naturally existing sequence or a functional variant thereof) (e.g., a sequence comprising the amino acid sequence of SEQ ID NO: 13 or 180, or a sequence having at least 70%, 75%, 80%, 85%, 90%, or 95% sequence identity thereof), the second monomer molecule is APRIL (e.g., a naturally existing sequence or a functional variant thereof) (e.g., a sequence comprising the amino acid sequence of SEQ ID NO: 13 or 180, or a sequence having at least 70%, 75%, 80%, 85%, 90%, or 95% sequence identity thereof), and the third monomer molecule is BAFF (e.g., a naturally existing sequence or a functional variant thereof) (e.g., a sequence comprising the amino acid sequence of SEQ ID NO: 14, or a sequence having at least 70%, 75%, 80%, 85%, 90%, or 95% sequence identity thereof).

In some embodiments, the first, second, and third monomer molecules are independently chosen from lymphotoxin-alpha (or a functional variant thereof) or lymphotoxin-beta (or a functional variant thereof), wherein at least one of the three monomer molecules is lymphotoxin-alpha (or a functional variant thereof), and at least one of the three monomer molecules is lymphotoxin-beta (or a functional variant thereof). In some embodiments, the first, second, and third monomer molecules independently comprise the amino acid sequence of SEQ ID NO: 1 (or a sequence having at least 70%, 75%, 80%, 85%, 90%, or 95% sequence identity thereof) or SEQ ID NO: 3 (or a sequence having at least 70%, 75%, 80%, 85%, 90%, or 95% sequence identity). In some embodiments, the first monomer molecule is lymphotoxin-alpha (or a functional variant thereof) (e.g., a sequence comprising the amino acid sequence of SEQ ID NO: 1, or a sequence having at least 70%, 75%, 80%, 85%, 90%, or 95% sequence identity thereof), the second monomer molecule is lymphotoxin-alpha (or a functional variant thereof) (e.g., a sequence comprising the amino acid sequence of SEQ ID NO: 1, or a sequence having at least 70%, 75%, 80%, 85%, 90%, or 95% sequence identity thereof), and the third monomer molecule is lymphotoxin-beta (or a functional variant thereof) (e.g., a sequence comprising the amino acid sequence of SEQ ID NO: 3, or a sequence having at least 70%, 75%, 80%, 85%, 90%, or 95% sequence identity thereof). In some embodiments, the first monomer molecule is lymphotoxin-beta (or a functional variant thereof) (e.g., a sequence comprising the amino acid sequence of SEQ ID NO: 3, or a sequence having at least 70%, 75%, 80%, 85%, 90%, or 95% sequence identity thereof), the second monomer molecule is lymphotoxin-beta (or a functional variant thereof) (e.g., a sequence comprising the amino acid sequence of SEQ ID NO: 3, or a sequence having at least 70%, 75%, 80%, 85%, 90%, or 95% sequence identity thereof), and the third monomer molecule is lymphotoxin-alpha (or a functional variant thereof) (e.g., a sequence comprising the amino acid sequence of SEQ ID NO: 1, or a sequence having at least 70%, 75%, 80%, 85%, 90%, or 95% sequence identity thereof).

In one aspect, disclosed herein is a multifunctional molecule, comprising a trimeric ligand (e.g., one or more (e.g., one, two or three) trimeric ligands), wherein said trimeric ligand comprises a first monomer molecule, a second monomer molecule, and a third monomer molecule (e.g., three monomer molecules of the tumor necrosis factor superfamily (TNFSF) or TNSF-like member, or a combination thereof), wherein the first and second monomer molecules are coupled, e.g., covalently linked, to one another, and the third monomer molecule is non-covalently associated to the first and second monomer molecules, wherein the multifunctional molecule further comprises a TCR constant domain.

In some embodiments, one or both of: (a) the first and second monomer molecules, and (b) the third monomer molecule, are coupled, e.g., covalently linked, e.g., via a linker, to the TCR constant domain.

In some embodiments, the multifunctional molecule comprises a TCRα constant domain (e.g., a naturally existing sequence or a functional variant thereof, e.g., a functional variant capable of forming stable association with a TCRβ constant domain) and a TCRβ constant domain (e.g., a naturally existing sequence or a functional variant thereof, e.g., a functional variant capable of forming stable association with a TCRα constant domain). In some embodiments, the first and second monomer molecules are coupled, e.g., covalently linked, e.g., via a linker, to the TCRα constant domain (e.g., a naturally existing sequence or a functional variant thereof, e.g., a functional variant capable of forming stable association with a TCRβ constant domain) (e.g., to the C-terminus of the TCRα constant domain), and the third monomer molecule is coupled, e.g., covalently linked, e.g., via a linker, to the TCRβ constant domain (e.g., a naturally existing sequence or a functional variant thereof, e.g., a functional variant capable of forming stable association with a TCRα constant domain) (e.g., to the C-terminus of the TCRβ constant domain). In some embodiments, the first and second monomer molecules are coupled, e.g., covalently linked, e.g., via a linker, to the TCRβ constant domain (e.g., a naturally existing sequence or a functional variant thereof, e.g., a functional variant capable of forming stable association with a TCRα constant domain) (e.g., to the C-terminus of the TCRβ constant domain), and the third monomer molecule is coupled, e.g., covalently linked, e.g., via a linker, to the TCRα constant domain (e.g., a naturally existing sequence or a functional variant thereof, e.g., a functional variant capable of forming stable association with a TCRβ constant domain) (e.g., to the C-terminus of the TCRα constant domain). In some embodiments, the multifunctional molecule comprises a configuration depicted in FIG. 23A, 23B, or 23C.

In some embodiments, the multifunctional molecule comprises (i) a first polypeptide comprising, from N- to C-terminus, a first VL domain and a first CL domain, (ii) a second polypeptide comprising, from N- to C-terminus, a first VH domain, a first CH1 domain, a first CH2 domain, the TCRα constant domain (e.g., a naturally existing sequence or a functional variant thereof, e.g., a functional variant capable of forming stable association with a TCRβ constant domain), the first monomer molecule, and the second monomer molecule, (iii) a third polypeptide comprising, from N- to C-terminus, a second VL domain and a second CL domain, and (iv) a fourth polypeptide comprising, from N- to C-terminus, a second VH domain, a second CH1 domain, a second CH2 domain, the TCRβ constant domain (e.g., a naturally existing sequence or a functional variant thereof, e.g., a functional variant capable of forming stable association with a TCRα constant domain), and the third monomer molecule.

In some embodiments, the multifunctional molecule comprises (i) a first polypeptide comprising, from N- to C-terminus, a first VL domain and a first CL domain, (ii) a second polypeptide comprising, from N- to C-terminus, a first VH domain, a first CH1 domain, a first CH2 domain, the TCRα constant domain (e.g., a naturally existing sequence or a functional variant thereof, e.g., a functional variant capable of forming stable association with a TCRβ constant domain), and the third monomer molecule, (iii) a third polypeptide comprising, from N to C-terminus, a second VL domain and a second CL domain, and (iv) a fourth polypeptide comprising, from N- to C-terminus, a second VH domain, a second CH1 domain, a second CH2 domain, the TCRβ constant domain (e.g., a naturally existing sequence or a functional variant thereof, e.g., a functional variant capable of forming stable association with a TCRα constant domain), the first monomer molecule, and the second monomer molecule.

In some embodiments, the multifunctional molecule comprises (i) a first polypeptide comprising, from N- to C-terminus, a first scFv domain, a first CH2 domain, the TCRα constant domain (e.g., a naturally existing sequence or a functional variant thereof, e.g., a functional variant capable of forming stable association with a TCRβ constant domain), the first monomer molecule, and the second monomer molecule, and (ii) a second polypeptide comprising, from N to C-terminus, a second scFv domain, a second CH2 domain, the TCRβ constant domain (e.g., a naturally existing sequence or a functional variant thereof, e.g., a functional variant capable of forming stable association with a TCRα constant domain), and the third monomer molecule.

In some embodiments, the multifunctional molecule comprises (i) a first polypeptide comprising, from N- to C-terminus, a first scFv domain, a first CH2 domain, the TCRα constant domain (e.g., a naturally existing sequence or a functional variant thereof, e.g., a functional variant capable of forming stable association with a TCR constant domain), and the third monomer molecule, and (ii) a second polypeptide comprising, from N- to C-terminus, a second scFv domain, a second CH2 domain, the TCRβ constant domain (e.g., a naturally existing sequence or a functional variant thereof, e.g., a functional variant capable of forming stable association with a TCRα constant domain), the first monomer molecule, and the second monomer molecule.

In some embodiments, the first and second monomer molecules are coupled, e.g., covalently linked, e.g., via a linker, to a Fab heavy chain constant region (e.g., a CH1 domain, e.g., to the N-terminus of the CH1 domain), and the third monomer molecule is coupled, e.g., covalently linked, e.g., via a linker, to a Fab light chain constant region (e.g., a CL domain, e.g., to the N-terminus of the CL domain). In some embodiments, the first and second monomer molecules are coupled, e.g., covalently linked, e.g., via a linker, to a Fab light chain constant region (e.g., a CL domain, e.g., to the N-terminus of the CL domain), and the third monomer molecule is coupled, e.g., covalently linked, e.g., via a linker, to a Fab heavy chain constant region (e.g., a CH1 domain, e.g., to the N-terminus of the CH1 domain). In some embodiments, the CH1 domain is further linked, e.g., covalently linked, to a CH2 domain, which is further linked, e.g., covalently linked, to the TCRα constant domain (e.g., a naturally existing sequence or a functional variant thereof, e.g., a functional variant capable of forming stable association with a TCRβ constant domain) or the TCRβ constant domain (e.g., a naturally existing sequence or a functional variant thereof, e.g., a functional variant capable of forming stable association with a TCRα constant domain). In some embodiments, the multifunctional molecule comprises a configuration depicted in FIG. 22A, 22B, or 22C.

In some embodiments, the multifunctional molecule comprises (i) a first polypeptide comprising, from N- to C-terminus, a first CH2 domain and a first TCR constant domain, (ii) a second polypeptide comprising, from N- to C-terminus, the first and second monomer molecules, a first CH1 domain, a second CH2 domain, and a second TCR constant domain, and (iii) a third polypeptide comprising, from N- to C-terminus, the third monomer molecule and a second CH2 domain.

In some embodiments, the multifunctional molecule comprises (i) a first polypeptide comprising, from N- to C-terminus, a first CH2 domain and a first TCR constant domain, (ii) a second polypeptide comprising, from N- to C-terminus, the third monomer molecule, a first CH1 domain, a second CH2 domain, and a second TCR constant domain, and (iii) a third polypeptide comprising, from N- to C-terminus, the first and second monomer molecules, and a second CH2 domain.

In some embodiments, the multifunctional molecule comprises (i) a first polypeptide comprising, from N- to C-terminus, a VL domain and a CL domain, (ii) a second polypeptide comprising, from N- to C-terminus, a VH domain, a first CH1 domain, a first CH2 domain, and a first TCR constant domain, (iii) a third polypeptide comprising, from N- to C-terminus, the first and second monomer molecules, a second CH1 domain, a second CH2 domain, and a second TCR constant domain, and (iv) a fourth polypeptide comprising, from N- to C-terminus, the third monomer molecule and a second CH2 domain.

In some embodiments, the multifunctional molecule comprises (i) a first polypeptide comprising, from N- to C-terminus, a VL domain and a CL domain, (ii) a second polypeptide comprising, from N- to C-terminus, a VH domain, a first CH1 domain, a first CH2 domain, and a first TCR constant domain, (iii) a third polypeptide comprising, from N- to C-terminus, the third monomer molecule, a second CH1 domain, a second CH2 domain, and a second TCR constant domain, and (iv) a fourth polypeptide comprising, from N- to C-terminus, the first and second monomer molecules, and a second CH2 domain.

In some embodiments, the multifunctional molecule comprises (i) a first polypeptide comprising, from N- to C-terminus, an scFv domain, a first CH2 domain, and a first TCR constant domain, (ii) a second polypeptide comprising, from N- to C-terminus, the first and second monomer molecules, a first CH1 domain, a second CH2 domain, and a second TCR constant domain, and (iii) a third polypeptide comprising, from N- to C-terminus, the third monomer molecule and a second CH2 domain.

In some embodiments, the multifunctional molecule comprises (i) a first polypeptide comprising, from N- to C-terminus, an scFv domain, a first CH2 domain, and a first TCR constant domain, (ii) a second polypeptide comprising, from N- to C-terminus, the third monomer molecule, a first CH1 domain, a second CH2 domain, and a second TCR constant domain, and (iii) a third polypeptide comprising, from N- to C-terminus, the first and second monomer molecules and a second CH2 domain.

In some embodiments, the first TCR constant domain is the TCRα constant domain (e.g., a naturally existing sequence or a functional variant thereof, e.g., a functional variant capable of forming stable association with a TCR constant domain) and the second TCR constant domain is the TCRβ constant domain (e.g., a naturally existing sequence or a functional variant thereof, e.g., a functional variant capable of forming stable association with a TCRα constant domain). In some embodiments, the first TCR constant domain is the TCR constant domain (e.g., a naturally existing sequence or a functional variant thereof, e.g., a functional variant capable of forming stable association with a TCRα constant domain) and the second TCR constant domain is the TCRα constant domain (e.g., a naturally existing sequence or a functional variant thereof, e.g., a functional variant capable of forming stable association with a TCR constant domain).

In some embodiments, the multifunctional molecule comprises an immunoglobulin CH2 domain (e.g., an IgG1, IgG2, or IgG4 CH2 domain) connected to (optionally via a linker) the TCRα constant domain (e.g., a naturally existing sequence or a functional variant thereof, e.g., a functional variant capable of forming stable association with a TCR constant domain), and an immunoglobulin CH2 domain (e.g., an IgG1, IgG2, or IgG4 CH2 domain) connected to (optionally via a linker) the TCRβ constant domain (e.g., a naturally existing sequence or a functional variant thereof, e.g., a functional variant capable of forming stable association with a TCRα constant domain). In some embodiments, the multifunctional molecule comprises an immunoglobulin CH2 domain (e.g., an IgG1, IgG2, or IgG4 CH2 domain) connected to (optionally via a linker) the TCR constant domain (e.g., a naturally existing sequence or a functional variant thereof, e.g., a functional variant capable of forming stable association with a TCRα constant domain), and an immunoglobulin CH2 domain (e.g., an IgG1, IgG2, or IgG4 CH2 domain) connected to (optionally via a linker) the TCRα constant domain (e.g., a naturally existing sequence or a functional variant thereof, e.g., a functional variant capable of forming stable association with a TCR constant domain).

In some embodiments, the TCRα constant domain comprises or consists of the amino acid sequence of SEQ ID NO: 118 (or a sequence having 1 or more (e.g., 1, 2, 3, 4, 5, 5, 6, 7, 8, 9, 10, or more) amino acid substitutions relative to the amino acid sequence of SEQ ID NO: 118, or a sequence having no more than 10 (e.g., 10, 9, 8, 7, 6, 5, 4, 3, 2, or 1) amino acid substitutions relative to the amino acid sequence of SEQ ID NO: 118). In some embodiments, the TCR constant domain comprises or consists of the amino acid sequence of SEQ ID NO: 120 (or a sequence having 1 or more (e.g., 1, 2, 3, 4, 5, 5, 6, 7, 8, 9, 10, or more) amino acid substitutions relative to the amino acid sequence of SEQ ID NO: 120, or a sequence having no more than 10 (e.g., 10, 9, 8, 7, 6, 5, 4, 3, 2, or 1) amino acid substitutions relative to the amino acid sequence of SEQ ID NO: 120).

In some embodiments, the TCRα constant domain comprises or consists of the amino acid sequence of SEQ ID NO: 119 (or a sequence having 1 or more (e.g., 1, 2, 3, 4, 5, 5, 6, 7, 8, 9, 10, or more) amino acid substitutions relative to the amino acid sequence of SEQ ID NO: 119, or a sequence having no more than 10 (e.g., 10, 9, 8, 7, 6, 5, 4, 3, 2, or 1) amino acid substitutions relative to the amino acid sequence of SEQ ID NO: 119). In some embodiments, the TCRβ constant domain comprises or consists of the amino acid sequence of SEQ ID NO: 121 (or a sequence having 1 or more (e.g., 1, 2, 3, 4, 5, 5, 6, 7, 8, 9, 10, or more) amino acid substitutions relative to the amino acid sequence of SEQ ID NO: 121, or a sequence having no more than 10 (e.g., 10, 9, 8, 7, 6, 5, 4, 3, 2, or 1) amino acid substitutions relative to the amino acid sequence of SEQ ID NO: 121).

In some embodiments, the first dimerization molecule comprises a TCRα variable domain connected to a TCRα constant domain (e.g., a naturally existing sequence or a functional variant thereof, e.g., a functional variant capable of forming stable association with a TCRβ constant domain), and the second dimerization molecule comprises a TCR variable domain connected to a TCRβ constant domain (e.g., a naturally existing sequence or a functional variant thereof, e.g., a functional variant capable of forming stable association with a TCRα constant domain). In some embodiments, the first dimerization molecule comprises a TCR variable domain connected to a TCR constant domain (e.g., a naturally existing sequence or a functional variant thereof, e.g., a functional variant capable of forming stable association with a TCRα constant domain), and the second dimerization molecule comprises a TCRα variable domain connected to a TCRα constant domain (e.g., a naturally existing sequence or a functional variant thereof, e.g., a functional variant capable of forming stable association with a TCRβ constant domain).

In some embodiments, the multifunctional molecule does not comprise an immunoglobulin CH3 domain (e.g., any portion of a CH3 domain). In some embodiments, the multifunctional molecule does not comprise any portion of an immunoglobulin CH3 domain capable of stable self-association.

In some embodiments, the multifunctional molecule does not comprise more than 50, 25, 10, or 5 amino acids of an immunoglobulin CH2 domain and/or more than 50, 25, 10, or 5 amino acids of an immunoglobulin CH3 domain. In some embodiments, the multifunctional molecule does not comprise an immunoglobulin CH2 and/or CH3 domain (e.g., any portion of a CH2 and/or CH3 domain).

In some embodiments, the trimeric ligand is a heterotrimer. In some embodiments, the first monomer molecule, the second monomer molecule, and the third monomer molecule are not identical. In some embodiments, the first and second monomer molecules are the same (or sharing no less than 70%, 75%, 80%, 85%, or 90% sequence identity), and the third monomer molecule is different from the first and second monomer molecules (e.g., sharing no more than 40%, 45%, 50%, 55%, or 60% sequence identity with the first or second monomer molecule). In some embodiments, the first and third monomer molecules are the same (or sharing no less than 70%, 75%, 80%, 85%, or 90% sequence identity), and the second monomer molecule is different from the first and third monomer molecules (e.g., sharing no more than 40%, 45%, 50%, 55%, or 60% sequence identity with the first or second monomer molecule). In some embodiments, the second and third monomer molecules are the same (or sharing no less than 70%, 75%, 80%, 85%, or 90% sequence identity), and the first monomer molecule is different from the second and third monomer molecules (e.g., sharing no more than 40%, 45%, 50%, 55%, or 60% sequence identity with the first or second monomer molecule).

In some embodiments, the first, second, and third monomer molecules are independently chosen from BAFF (e.g., a naturally existing sequence or a functional variant thereof) or APRIL (e.g., a naturally existing sequence or a functional variant thereof), wherein at least one of the three monomer molecules is BAFF (e.g., a naturally existing sequence or a functional variant thereof), and at least one of the three monomer molecules is APRIL (e.g., a naturally existing sequence or a functional variant thereof). In some embodiments, the first, second, and third monomer molecules independently comprise the amino acid sequence of SEQ ID NO: 14 (or a sequence having at least 70%, 75%, 80%, 85%, 90%, or 95% sequence identity thereof) or the amino acid sequence of SEQ ID NO: 13 or 180 (or a sequence having at least 70%, 75%, 80%, 85%, 90%, or 95% sequence identity). In some embodiments, the first monomer molecule is BAFF (e.g., a naturally existing sequence or a functional variant thereof) (e.g., a sequence comprising the amino acid sequence of SEQ ID NO: 14, or a sequence having at least 70%, 75%, 80%, 85%, 90%, or 95% sequence identity thereof), the second monomer molecule is BAFF (e.g., a naturally existing sequence or a functional variant thereof) (e.g., a sequence comprising the amino acid sequence of SEQ ID NO: 14, or a sequence having at least 70%, 75%, 80%, 85%, 90%, or 95% sequence identity thereof), and the third monomer molecule is APRIL (e.g., a naturally existing sequence or a functional variant thereof) (e.g., a sequence comprising the amino acid sequence of SEQ ID NO: 13 or 180, or a sequence having at least 70%, 75%, 80%, 85%, 90%, or 95% sequence identity thereof). In some embodiments, the first monomer molecule is APRIL (e.g., a naturally existing sequence or a functional variant thereof) (e.g., a sequence comprising the amino acid sequence of SEQ ID NO: 13 or 180, or a sequence having at least 70%, 75%, 80%, 85%, 90%, or 95% sequence identity thereof), the second monomer molecule is APRIL (e.g., a naturally existing sequence or a functional variant thereof) (e.g., a sequence comprising the amino acid sequence of SEQ ID NO: 13 or 180, or a sequence having at least 70%, 75%, 80%, 85%, 90%, or 95% sequence identity thereof), and the third monomer molecule is BAFF (e.g., a naturally existing sequence or a functional variant thereof) (e.g., a sequence comprising the amino acid sequence of SEQ ID NO: 14, or a sequence having at least 70%, 75%, 80%, 85%, 90%, or 95% sequence identity thereof).

In some embodiments, the first, second, and third monomer molecules are independently chosen from lymphotoxin-alpha (or a functional variant thereof) or lymphotoxin-beta (or a functional variant thereof), wherein at least one of the three monomer molecules is lymphotoxin-alpha (or a functional variant thereof), and at least one of the three monomer molecules is lymphotoxin-beta (or a functional variant thereof). In some embodiments, the first, second, and third monomer molecules independently comprise the amino acid sequence of SEQ ID NO: 1 (or a sequence having at least 70%, 75%, 80%, 85%, 90%, or 95% sequence identity thereof) or SEQ ID NO: 3 (or a sequence having at least 70%, 75%, 80%, 85%, 90%, or 95% sequence identity). In some embodiments, the first monomer molecule is lymphotoxin-alpha (or a functional variant thereof) (e.g., a sequence comprising the amino acid sequence of SEQ ID NO: 1, or a sequence having at least 70%, 75%, 80%, 85%, 90%, or 95% sequence identity thereof), the second monomer molecule is lymphotoxin-alpha (or a functional variant thereof) (e.g., a sequence comprising the amino acid sequence of SEQ ID NO: 1, or a sequence having at least 70%, 75%, 80%, 85%, 90%, or 95% sequence identity thereof), and the third monomer molecule is lymphotoxin-beta (or a functional variant thereof) (e.g., a sequence comprising the amino acid sequence of SEQ ID NO: 3, or a sequence having at least 70%, 75%, 80%, 85%, 90%, or 95% sequence identity thereof). In some embodiments, the first monomer molecule is lymphotoxin-beta (or a functional variant thereof) (e.g., a sequence comprising the amino acid sequence of SEQ ID NO: 3, or a sequence having at least 70%, 75%, 80%, 85%, 90%, or 95% sequence identity thereof), the second monomer molecule is lymphotoxin-beta (or a functional variant thereof) (e.g., a sequence comprising the amino acid sequence of SEQ ID NO: 3, or a sequence having at least 70%, 75%, 80%, 85%, 90%, or 95% sequence identity thereof), and the third monomer molecule is lymphotoxin-alpha (or a functional variant thereof) (e.g., a sequence comprising the amino acid sequence of SEQ ID NO: 1, or a sequence having at least 70%, 75%, 80%, 85%, 90%, or 95% sequence identity thereof).

In one aspect, disclosed herein is a multifunctional molecule, comprising a trimeric ligand (e.g., one or more (e.g., one, two or three) trimeric ligands), wherein said trimeric ligand comprises a first monomer molecule, a second monomer molecule, and a third monomer molecule (e.g., three monomer molecules of the tumor necrosis factor superfamily (TNFSF) or TNSF-like member, or a combination thereof), wherein the first and second monomer molecules are coupled, e.g., covalently linked, to one another, and the third monomer molecule is non-covalently associated to the first and second monomer molecules, wherein the trimeric ligand is a heterotrimer.

In some embodiments, the first monomer molecule, the second monomer molecule, and the third monomer molecule are not identical. In some embodiments, the first and second monomer molecules are the same (or sharing no less than 70%, 75%, 80%, 85%, or 90% sequence identity), and the third monomer molecule is different from the first and second monomer molecules (e.g., sharing no more than 40%, 45%, 50%, 55%, or 60% sequence identity with the first or second monomer molecule). In some embodiments, the first and third monomer molecules are the same (or sharing no less than 70%, 75%, 80%, 85%, or 90% sequence identity), and the second monomer molecule is different from the first and third monomer molecules (e.g., sharing no more than 40%, 45%, 50%, 55%, or 60% sequence identity with the first or second monomer molecule). In some embodiments, the second and third monomer molecules are the same (or sharing no less than 70%, 75%, 80%, 85%, or 90% sequence identity), and the first monomer molecule is different from the second and third monomer molecules (e.g., sharing no more than 40%, 45%, 50%, 55%, or 60% sequence identity with the first or second monomer molecule).

In some embodiments, the first, second, and third monomer molecules are independently chosen from BAFF (e.g., a naturally existing sequence or a functional variant thereof) or APRIL (e.g., a naturally existing sequence or a functional variant thereof), wherein at least one of the three monomer molecules is BAFF (e.g., a naturally existing sequence or a functional variant thereof), and at least one of the three monomer molecules is APRIL (e.g., a naturally existing sequence or a functional variant thereof). In some embodiments, the first, second, and third monomer molecules independently comprise the amino acid sequence of SEQ ID NO: 14 (or a sequence having at least 70%, 75%, 80%, 85%, 90%, or 95% sequence identity thereof) or the amino acid sequence of SEQ ID NO: 13 or 180 (or a sequence having at least 70%, 75%, 80%, 85%, 90%, or 95% sequence identity). In some embodiments, the first monomer molecule is BAFF (e.g., a naturally existing sequence or a functional variant thereof) (e.g., a sequence comprising the amino acid sequence of SEQ ID NO: 14, or a sequence having at least 70%, 75%, 80%, 85%, 90%, or 95% sequence identity thereof), the second monomer molecule is BAFF (e.g., a naturally existing sequence or a functional variant thereof) (e.g., a sequence comprising the amino acid sequence of SEQ ID NO: 14, or a sequence having at least 70%, 75%, 80%, 85%, 90%, or 95% sequence identity thereof), and the third monomer molecule is APRIL (e.g., a naturally existing sequence or a functional variant thereof) (e.g., a sequence comprising the amino acid sequence of SEQ ID NO: 13 or 180, or a sequence having at least 70%, 75%, 80%, 85%, 90%, or 95% sequence identity thereof). In some embodiments, the first monomer molecule is APRIL (e.g., a naturally existing sequence or a functional variant thereof) (e.g., a sequence comprising the amino acid sequence of SEQ ID NO: 13 or 180, or a sequence having at least 70%, 75%, 80%, 85%, 90%, or 95% sequence identity thereof), the second monomer molecule is APRIL (e.g., a naturally existing sequence or a functional variant thereof) (e.g., a sequence comprising the amino acid sequence of SEQ ID NO: 13 or 180, or a sequence having at least 70%, 75%, 80%, 85%, 90%, or 95% sequence identity thereof), and the third monomer molecule is BAFF (e.g., a naturally existing sequence or a functional variant thereof) (e.g., a sequence comprising the amino acid sequence of SEQ ID NO: 14, or a sequence having at least 70%, 75%, 80%, 85%, 90%, or 95% sequence identity thereof).

In some embodiments, the first, second, and third monomer molecules are independently chosen from lymphotoxin-alpha (or a functional variant thereof) or lymphotoxin-beta (or a functional variant thereof), wherein at least one of the three monomer molecules is lymphotoxin-alpha (or a functional variant thereof), and at least one of the three monomer molecules is lymphotoxin-beta (or a functional variant thereof). In some embodiments, the first, second, and third monomer molecules independently comprise the amino acid sequence of SEQ ID NO: 1 (or a sequence having at least 70%, 75%, 80%, 85%, 90%, or 95% sequence identity thereof) or SEQ ID NO: 3 (or a sequence having at least 70%, 75%, 80%, 85%, 90%, or 95% sequence identity). In some embodiments, the first monomer molecule is lymphotoxin-alpha (or a functional variant thereof) (e.g., a sequence comprising the amino acid sequence of SEQ ID NO: 1, or a sequence having at least 70%, 75%, 80%, 85%, 90%, or 95% sequence identity thereof), the second monomer molecule is lymphotoxin-alpha (or a functional variant thereof) (e.g., a sequence comprising the amino acid sequence of SEQ ID NO: 1, or a sequence having at least 70%, 75%, 80%, 85%, 90%, or 95% sequence identity thereof), and the third monomer molecule is lymphotoxin-beta (or a functional variant thereof) (e.g., a sequence comprising the amino acid sequence of SEQ ID NO: 3, or a sequence having at least 70%, 75%, 80%, 85%, 90%, or 95% sequence identity thereof). In some embodiments, the first monomer molecule is lymphotoxin-beta (or a functional variant thereof) (e.g., a sequence comprising the amino acid sequence of SEQ ID NO: 3, or a sequence having at least 70%, 75%, 80%, 85%, 90%, or 95% sequence identity thereof), the second monomer molecule is lymphotoxin-beta (or a functional variant thereof) (e.g., a sequence comprising the amino acid sequence of SEQ ID NO: 3, or a sequence having at least 70%, 75%, 80%, 85%, 90%, or 95% sequence identity thereof), and the third monomer molecule is lymphotoxin-alpha (or a functional variant thereof) (e.g., a sequence comprising the amino acid sequence of SEQ ID NO: 1, or a sequence having at least 70%, 75%, 80%, 85%, 90%, or 95% sequence identity thereof).

In some embodiments of the aforementioned aspects and embodiments, the first, second, or third monomer of the trimeric ligand is chosen from a TNFSF family member or a TNF-like family member, or a combination thereof, e.g., as described herein in Tables 1 and 2, e.g., BAFF, APRIL, lymphotoxin-alpha, lymphotoxin-beta, CD40L, or GITRL. In some embodiments, the trimeric ligand is a homotrimer, e.g., of the same TNFSF family member or the same TNF-like family member. In some embodiments, the trimeric ligand is a heterotrimer, e.g., it comprises a combination of monomer molecules from two or three trimeric ligands, e.g., two or three TNFSF or TNF-like family members. In some embodiments, the trimeric ligand comprises the amino acid sequence of a monomer molecule chosen from a TNFSF family member or a TNF-like family member, e.g., as described herein in Tables 1 and 2, or an amino acid sequence substantially identical thereto, e.g., at least 85%, 90%, 95%, 99% or more identical to an amino acid sequence described in Tables 1 and 2. In some embodiments, the trimeric ligand comprises any of the TNFSF amino acid sequences for TNSF1, TNSF2, TNSF3, TNSF4, TNSF5, TNSF6, TNSF7, TNSF8, TNSF9, TNSF10, TNSF1, TNSF11, TNSF12, TNSF13, TNSF13B, TNSF14, TNSF15, TNF18 or EDA, e.g., corresponding to SEQ ID NOs: 1-17 and 218, in Table 1, or an amino acid sequence substantially identical thereto, e.g., at least 85%, 90%, 95%, 99% or more identical to the amino acid sequence of any of SEQ ID NOs: 1-17 and 218, as a homotrimer, or a heterotrimer comprising any combination of two or three of the aforesaid monomer molecules. In some embodiments, the trimeric ligand comprises any of the TNF-like amino acid sequences for Complement C1Q (e.g., subcomponents A, B and C), C1QL1, C1QL2, C1QL3, Caprin-2 C1q domain, cerebellin-1 C1q domain or adiponectin, e.g., corresponding to SEQ ID NOs: 239-247, in Table 2, or an amino acid sequence substantially identical thereto, e.g., at least 85%, 90%, 95%, 99% or more identical to the amino acid sequence of any of SEQ ID NOs: 239-247, as a homotrimer (e.g., comprises of homomonomer molecules), or a heterotrimer (e.g., comprises of heteromonomer molecules) comprising any combination of two or three of the aforesaid monomers. In some embodiments, the trimeric ligand comprises a combination of TNFSF monomer molecules and TNF-like monomer molecules, wherein the trimer ligand is a heterotrimer comprising any combination of two or three monomer molecules comprising the amino acid sequence of any of SEQ ID NOs: 1-17, 218, and 239-247. In some embodiments, the trimeric ligand comprises a TNFSF family member chosen from CD40L, GITRL, FasL, 4-1BBL, BAFF, APRIL, OX40L, TNF alpha, LIGHT, lymphotoxin-alpha, or lymphotoxin-beta (e.g., a naturally existing sequence or a functional variant thereof) (e.g., SEQ ID NO: 5, 198, 17, 6, 9, 14, 13, 180, 4, 2, 15, 1, 3, or a sequence having at least 70%, 75%, 80%, 85%, 90%, or 95% sequence identity thereof).

In some embodiments of the aforementioned aspects and embodiments, the multifunctional molecule comprises one trimeric ligand, two trimeric ligands, or three trimeric ligands.

In some embodiments of the aforementioned aspects and embodiments, the multifunctional molecule further comprises one or more other binding specificities or functionalities chosen from one, two or more of: a targeting moiety, e.g., a tumor targeting moiety; an immune cell engager (e.g., chosen from one, two, three, or all of an NK cell engager, a B cell engager, a dendritic cell engager, or a macrophage cell engager); and/or a cytokine molecule.

In some embodiments, the multifunctional molecule comprises the amino acid sequence of SEQ ID NO: 94 (without the signal peptide SEQ ID NO: 74) (or a sequence having at least 70%, 75%, 80%, 85%, 90%, or 95% sequence identity thereof), and the amino acid sequence of SEQ ID NO: 95 (without the signal peptide SEQ ID NO: 74) (or a sequence having at least 70%, 75%, 80%, 85%, 90%, or 95% sequence identity thereof). In some embodiments, the multifunctional molecule comprises the amino acid sequence of SEQ ID NO: 96 (without the signal peptide SEQ ID NO: 74) (or a sequence having at least 70%, 75%, 80%, 85%, 90%, or 95% sequence identity thereof), the amino acid sequence of SEQ ID NO: 97 (without the signal peptide SEQ ID NO: 74) (or a sequence having at least 70%, 75%, 80%, 85%, 90%, or 95% sequence identity thereof), and the amino acid sequence of SEQ ID NO: 98 (without the signal peptide SEQ ID NO: 74) (or a sequence having at least 70%, 75%, 80%, 85%, 90%, or 95% sequence identity thereof). In some embodiments, the multifunctional molecule comprises the amino acid sequence of SEQ ID NO: 99 (without the signal peptide SEQ ID NO: 74) (or a sequence having at least 70%, 75%, 80%, 85%, 90%, or 95% sequence identity thereof), and the amino acid sequence of SEQ ID NO: 100 (without the signal peptide SEQ ID NO: 74) (or a sequence having at least 70%, 75%, 80%, 85%, 90%, or 95% sequence identity thereof). In some embodiments, the multifunctional molecule comprises the amino acid sequence of SEQ ID NO: 101 (without the signal peptide SEQ ID NO: 74) (or a sequence having at least 70%, 75%, 80%, 85%, 90%, or 95% sequence identity thereof), and the amino acid sequence of SEQ ID NO: 102 (without the signal peptide SEQ ID NO: 74) (or a sequence having at least 70%, 75%, 80%, 85%, 90%, or 95% sequence identity thereof). In some embodiments, the multifunctional molecule comprises the amino acid sequence of SEQ ID NO: 103 (without the signal peptide SEQ ID NO: 74) (or a sequence having at least 70%, 75%, 80%, 85%, 90%, or 95% sequence identity thereof), and the amino acid sequence of SEQ ID NO: 104 (without the signal peptide SEQ ID NO: 74) (or a sequence having at least 70%, 75%, 80%, 85%, 90%, or 95% sequence identity thereof). In some embodiments, the multifunctional molecule comprises the amino acid sequence of SEQ ID NO: 105 (without the signal peptide SEQ ID NO: 74) (or a sequence having at least 70%, 75%, 80%, 85%, 90%, or 95% sequence identity thereof), and the amino acid sequence of SEQ ID NO: 106 (without the signal peptide SEQ ID NO: 74) (or a sequence having at least 70%, 75%, 80%, 85%, 90%, or 95% sequence identity thereof). In some embodiments, the multifunctional molecule comprises the amino acid sequence of SEQ ID NO: 107 (without the signal peptide SEQ ID NO: 74) (or a sequence having at least 70%, 75%, 80%, 85%, 90%, or 95% sequence identity thereof), and the amino acid sequence of SEQ ID NO: 108 (without the signal peptide SEQ ID NO: 74) (or a sequence having at least 70%, 75%, 80%, 85%, 90%, or 95% sequence identity thereof). In some embodiments, the multifunctional molecule comprises the amino acid sequence of SEQ ID NO: 158 (without the signal peptide SEQ ID NO: 74) (or a sequence having at least 70%, 75%, 80%, 85%, 90%, or 95% sequence identity thereof), and the amino acid sequence of SEQ ID NO: 159 (without the signal peptide SEQ ID NO: 74) (or a sequence having at least 70%, 75%, 80%, 85%, 90%, or 95% sequence identity thereof). In some embodiments, the multifunctional molecule comprises the amino acid sequence of SEQ ID NO: 160 (without the signal peptide SEQ ID NO: 74) (or a sequence having at least 70%, 75%, 80%, 85%, 90%, or 95% sequence identity thereof), and the amino acid sequence of SEQ ID NO: 161 (without the signal peptide SEQ ID NO: 74) (or a sequence having at least 70%, 75%, 80%, 85%, 90%, or 95% sequence identity thereof). In some embodiments, the multifunctional molecule comprises the amino acid sequence of SEQ ID NO: 162 (without the signal peptide SEQ ID NO: 74) (or a sequence having at least 70%, 75%, 80%, 85%, 90%, or 95% sequence identity thereof), the amino acid sequence of SEQ ID NO: 163 (without the signal peptide SEQ ID NO: 74) (or a sequence having at least 70%, 75%, 80%, 85%, 90%, or 95% sequence identity thereof), and the amino acid sequence of SEQ ID NO: 164 (without the signal peptide SEQ ID NO: 74) (or a sequence having at least 70%, 75%, 80%, 85%, 90%, or 95% sequence identity thereof). In some embodiments, the multifunctional molecule comprises the amino acid sequence of SEQ ID NO: 165 (without the signal peptide SEQ ID NO: 74) (or a sequence having at least 70%, 75%, 80%, 85%, 90%, or 95% sequence identity thereof), the amino acid sequence of SEQ ID NO: 166 (without the signal peptide SEQ ID NO: 74) (or a sequence having at least 70%, 75%, 80%, 85%, 90%, or 95% sequence identity thereof), and the amino acid sequence of SEQ ID NO: 164 (without the signal peptide SEQ ID NO: 74) (or a sequence having at least 70%, 75%, 80%, 85%, 90%, or 95% sequence identity thereof). In some embodiments, the multifunctional molecule comprises the amino acid sequence of SEQ ID NO: 167 (without the signal peptide SEQ ID NO: 74) (or a sequence having at least 70%, 75%, 80%, 85%, 90%, or 95% sequence identity thereof), and the amino acid sequence of SEQ ID NO: 168 (without the signal peptide SEQ ID NO: 74) (or a sequence having at least 70%, 75%, 80%, 85%, 90%, or 95% sequence identity thereof). In some embodiments, the multifunctional molecule comprises the amino acid sequence of SEQ ID NO: 169 (without the signal peptide SEQ ID NO: 74) (or a sequence having at least 70%, 75%, 80%, 85%, 90%, or 95% sequence identity thereof), and the amino acid sequence of SEQ ID NO: 170 (without the signal peptide SEQ ID NO: 74) (or a sequence having at least 70%, 75%, 80%, 85%, 90%, or 95% sequence identity thereof). In some embodiments, the multifunctional molecule comprises the amino acid sequence of SEQ ID NO: 171 (without the signal peptide SEQ ID NO: 74) (or a sequence having at least 70%, 75%, 80%, 85%, 90%, or 95% sequence identity thereof), and the amino acid sequence of SEQ ID NO: 172 (without the signal peptide SEQ ID NO: 74) (or a sequence having at least 70%, 75%, 80%, 85%, 90%, or 95% sequence identity thereof). In some embodiments, the multifunctional molecule comprises the amino acid sequence of SEQ ID NO: 173 (without the signal peptide SEQ ID NO: 74) (or a sequence having at least 70%, 75%, 80%, 85%, 90%, or 95% sequence identity thereof), and the amino acid sequence of SEQ ID NO: 174 (without the signal peptide SEQ ID NO: 74) (or a sequence having at least 70%, 75%, 80%, 85%, 90%, or 95% sequence identity thereof). In some embodiments, the multifunctional molecule comprises the amino acid sequence of SEQ ID NO: 175 (without the signal peptide SEQ ID NO: 74) (or a sequence having at least 70%, 75%, 80%, 85%, 90%, or 95% sequence identity thereof), and the amino acid sequence of SEQ ID NO: 176 (without the signal peptide SEQ ID NO: 74) (or a sequence having at least 70%, 75%, 80%, 85%, 90%, or 95% sequence identity thereof). In some embodiments, the multifunctional molecule comprises the amino acid sequence of SEQ ID NO: 177 (without the signal peptide SEQ ID NO: 74) (or a sequence having at least 70%, 75%, 80%, 85%, 90%, or 95% sequence identity thereof), and the amino acid sequence of SEQ ID NO: 178 (without the signal peptide SEQ ID NO: 74) (or a sequence having at least 70%, 75%, 80%, 85%, 90%, or 95% sequence identity thereof).

In another aspect, the present disclosure provides, isolated nucleic acid molecules, comprising the nucleotide sequence encoding any of the multifunctional molecules described herein, or a nucleotide sequence substantially identical thereto (e.g., at least 85%, 95%, 99.9% or more identical thereto).

In another aspect the present disclosure provides, vectors, e.g., expression vectors, comprising one or more of the nucleic acid molecule described herein.

In another aspect the present disclosure provides, host cells comprising a nucleic acid molecule or a vector described herein.

In another aspect the present disclosure provides, methods of making, e.g., producing, a multifunctional molecule described herein, comprising culturing a host cell described herein, under suitable conditions, e.g., conditions suitable for gene expression and/or heterodimerization.

In another aspect the present disclosure provides, pharmaceutical compositions comprising a multifunctional molecule described herein and a pharmaceutically acceptable carrier, excipient, or stabilizer.

In another aspect the present disclosure provides, methods of treating a cancer, comprising administering to a subject in need thereof a multifunctional molecule described herein, wherein the multifunctional antibody is administered in an amount effective to treat the cancer.

In some embodiments, the cancer is a solid tumor cancer, or a metastatic lesion. In some embodiments, the solid tumor cancer is one or more of pancreatic (e.g., pancreatic adenocarcinoma), breast, colorectal, lung (e.g., small or non-small cell lung cancer), skin, ovarian, or liver cancer. In some embodiments, the cancer is a hematological cancer.

In some embodiments, the method further comprises administering a second therapeutic treatment. In some embodiments, the second therapeutic treatment comprises a therapeutic agent (e.g., a chemotherapeutic agent, a biologic agent, hormonal therapy), radiation, or surgery. In some embodiments, the therapeutic agent is selected from: a chemotherapeutic agent, or a biologic agent.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIGS. 1A-1D depict exemplary formats and configurations of multispecific molecules attached to a heterodimeric Fc domain.

FIG. 1A shows a homotrimer ligand coupled, e.g., fused, through a linker to the C-terminus of a heterodimeric Fc domain wherein one of the immunoglobulin constant regions contains two ligand monomer molecules attached through a linker and the other immunoglobulin constant region contains a single ligand monomer molecule.

FIG. 1B shows a heterotrimer ligand coupled, e.g., fused, through a linker to the C-terminus of a heterodimeric Fc domain wherein one of the immunoglobulin constant regions contains two ligand monomer molecules attached through a linker and the other immunoglobulin constant region contains a single ligand monomer molecule from a different family member.

FIG. 1C shows a homotrimer ligand coupled, e.g., fused, through a linker to the N-terminus of a heterodimeric Fc domain wherein one of the immunoglobulin constant regions contains two ligand monomer molecules attached through a linker and the other immunoglobulin constant region contains a single ligand monomer molecule.

FIG. 1D shows a heterotrimer ligand coupled, e.g., fused, through a linker to the N-terminus of a heterodimeric Fc domain wherein one of the immunoglobulin constant regions contains two ligand monomer molecules attached through a linker and the other immunoglobulin constant region contains a single ligand monomer molecule from a different family member.

FIGS. 2A-2D depict exemplary formats and configurations of multispecific molecules comprising homotrimers attached to the constant domain of an immunoglobulin Fab region.

FIGS. 2A-2B show a homotrimer ligand coupled, e.g., fused, through a linker to the N-terminus of the constant domain of an immunoglobulin Fab region (e.g., CH1 and CL) wherein the CH1 domain contains two trimer monomer molecules attached through a linker and the CL domain contains a single trimer monomer molecule.

FIGS. 2C-2D show a homotrimer ligand coupled, e.g., fused, through a linker to the N-terminus of the constant domain of an immunoglobulin Fab region (e.g., CH1 and CL) wherein the CH1 domain contains a trimer monomer molecule attached through a linker and the CL domain contains two trimer monomer molecules.

FIGS. 3A-3D depict exemplary formats and configurations of multispecific molecules comprising heterotrimers attached to the constant domain of an immunoglobulin Fab region.

FIGS. 3A-3B show a heterotrimer ligand coupled, e.g., fused, through a linker the constant domain of an immunoglobulin Fab region wherein the CH1 domain contains two trimer monomer molecules attached through a linker and the CL domain contains a single trimer monomer molecule from a different family member.

FIGS. 3C-3D show a heterotrimer ligand coupled, e.g., fused, through a linker to the constant domain of an immunoglobulin Fab region wherein the CH1 domain contains a single trimer monomer molecule and the CL domain contains two trimer monomer molecules attached through a linker from a different family member.

FIGS. 4A-4F depict exemplary formats and configurations of multispecific molecules comprising two homotrimer ligands, which are identical or dissimilar, fused through a linker to the constant domains of immunoglobulin Fab regions whose Fc regions may be homodimers or heterodimers.

FIGS. 5A-5F depict exemplary formats and configurations of multispecific molecules comprising two heterotrimer ligands composed of 2 monomer molecules of one ligand member and 1 monomer molecule of another ligand member, which are identical or dissimilar, fused through a linker to the constant domains of immunoglobulin Fab regions whose Fc regions may be homodimers or heterodimers.

FIGS. 6A-6H depict exemplary formats and configurations of multispecific molecules comprising three homotrimer ligands, which are identical or dissimilar, fused through a linker to the Fab regions and the Fc region of an immunoglobulin heterodimer.

FIGS. 7A-7H depict exemplary formats and configurations of multispecific molecules comprising three heterotrimer ligands, which are identical or dissimilar, fused through a linker to the Fab regions and the Fc region of an immunoglobulin heterodimer.

FIGS. 8A-8B depict bar graphs showing the results of functional assays of Fc-CD40L.

FIG. 9 depicts an exemplary multispecific molecule that includes a trimer ligand (1-3), a Fab VH (8), a Fab VL (9), a heterodimeric Fc (6-7) and a single chain Fv (10).

FIG. 10 depicts an exemplary multispecific molecule that includes a first Fab VH (first heavy variable and constant region 1-2, respectively), a first Fab VL (first light variable and constant region 3-4, respectively), a second Fab VH (second heavy variable and constant region 5-6, respectively), a second Fab VL (second light variable and constant region 7-8, respectively), a heterodimeric Fc (9-10) and a trimer ligand (11-13).

FIGS. 11A-11C depict exemplary formats and configurations of multispecific molecules attached to a dimerization module, e.g., an immunoglobulin constant domain. FIG. 1A depicts moieties A, B, C and D, covalently linked to a heterodimeric Fc domain. FIG. 1B depicts moieties A, B, C and D, covalently linked to a homodimeric Fc domain. FIG. 1C depicts moieties A, B, C and D, covalently linked to a heterodimeric heavy and light variable region constant domains (e.g., a Fab CH₁ and a Fab CL).

FIG. 12 depicts an exemplary multispecific molecule that includes a trimer ligand (1-3), a Fab VH (8), a Fab VL (9), a heterodimeric Fc (6-7) and a single chain Fv (10) with the alternate trimer linkage from FIG. 9.

FIGS. 13A-13D depict exemplary formats and configurations of multispecific molecules attached to a heterodimeric Fc domain with no hinge region disulfide bonds between the two Fc chains.

FIGS. 14A-14D depict exemplary formats and configurations of multispecific molecules comprising homotrimers attached to the constant domain of an immunoglobulin Fab region with no disulfide bond between the Fab constant domains.

FIGS. 15A-15D depict exemplary formats and configurations of multispecific molecules comprising heterotrimers attached to the constant domain of an immunoglobulin Fab region with no disulfide bond between the Fab constant domains.

FIGS. 16A-16F depict exemplary formats and configurations of multispecific molecules comprising two homotrimer ligands, which are identical or dissimilar, fused through a linker to the constant domains of immunoglobulin Fab regions with no disulfide bond between the Fab constant domains whose Fc regions may be homodimers or heterodimers.

FIGS. 17A-17F depict exemplary formats and configurations of multispecific molecules comprising two heterotrimer ligands composed of 2 monomer molecules of one ligand member and 1 monomer molecule of another ligand member, which are identical or dissimilar, fused through a linker to the constant domains of immunoglobulin Fab regions with no disulfide bond between the Fab constant domains whose Fc regions may be homodimers or heterodimers.

FIGS. 18A-18H depict exemplary formats and configurations of multispecific molecules comprising three homotrimer ligands, which are identical or dissimilar, fused through a linker to the Fab regions and the Fc region of an immunoglobulin heterodimer with no inter chain disulfides present in the molecule.

FIGS. 19A-19H depict exemplary formats and configurations of multispecific molecules comprising three heterotrimer ligands, which are identical or dissimilar, fused through a linker to the Fab regions and the Fc region of an immunoglobulin heterodimer with no inter chain disulfides present in the molecule.

FIGS. 20A-20B depict exemplary formats and configuration of multispecific molecules comprising homotrimers attached to the constant domain of an immunoglobulin Fab region with no disulfide bond between the Fab constant domains fused to one Fc region of a Fc heterodimer and a Fab or scFv fused to the other.

FIGS. 21A-21B depict exemplary formats and configurations of multispecific molecules attached to the C-terminus of a heterodimeric Fc domain with no hinge region disulfide bonds between the two Fc chains with either a Fab or an scFv fused to the N-terminus.

FIGS. 22A-22C depict exemplary formats and configurations of multispecific molecules comprising homotrimers attached to the constant domain of an immunoglobulin Fab region with no disulfide bond between the Fab constant domains fused to the N-terminus of a CH2-TCRα/CH2-TCRβ heterodimer.

FIGS. 23A-23C depict exemplary formats and configurations of multispecific molecules attached to the C-terminus of a heterodimeric CH2-TCRα/CH2-TCRβ domain with no hinge region disulfide bonds between the CH2 domains with either a Fab or an scFv fused to the N-terminus.

FIG. 24 shows the final gel of Fc (ΔCys)-GITRL.

FIG. 25 shows the analytical size exclusion chromatogram of Fc (ΔCys)-GITRL.

FIG. 26 shows the final gel of Fc-GITRL.

FIG. 27 shows the analytical size exclusion chromatogram of Fc-GITRL.

FIG. 28 shows the final gel of GITRL-Fc (ΔCys).

FIG. 29 shows the analytical size exclusion chromatogram of GITRL-Fc (ΔCys).

FIG. 30 shows the final gel of GITRL-Fc.

FIG. 31 shows the analytical size exclusion chromatogram of GITRL-Fc.

FIG. 32 shows the final gel of Fc-FASL.

FIG. 33 shows the analytical size exclusion chromatogram of Fc-FASL.

FIG. 34 shows the final gel of Fc (ΔCys)-FASL.

FIG. 35 shows the analytical size exclusion chromatogram of Fc (ΔCys)-FASE

FIG. 36 shows the final gel of Fc (ΔCys)-41BBL.

FIG. 37 shows the final gel of Fc-41BBL.

FIG. 38 shows the analytical size exclusion chromatogram of Fc-41BBL.

FIG. 39 shows the final gel of Fc-BAFF/APRIL.

FIG. 40 shows the analytical size exclusion chromatogram of Fc-BAFF/APRIL.

FIG. 41 shows the final gel of Fc (ΔCys)-BAFF/APRIL.

FIG. 42 shows the analytical size exclusion chromatogram of Fc (ΔCys)-BAFF/APRIL.

### DETAILED DESCRIPTION OF THE INVENTION

Disclosed herein are multifunctional molecules (also interchangeably referred to herein as “multispecific molecules”) that comprise a trimeric ligand, e.g., one, two or three trimeric ligands. In some embodiments, the multifunctional molecules have at least one, two, three or more activities, e.g., binding activities and/or other functional activities.

As used herein, a “trimeric ligand” refers to a molecule comprising three members, also referred to herein as “monomer molecules.” In embodiments, the three members are three covalently or non-covalently associated polypeptides. In some embodiments, the trimeric ligand comprises three non-covalently associated monomer molecules. In one embodiment, the trimeric ligand comprises two covalently associated monomer molecules, e.g., joined by a linker, e.g., a peptide linker (e.g., the two covalently associated monomers and the linker forming a fusion protein) and one non-covalently associated monomer molecule. In yet other embodiments, the trimeric ligand comprises three covalently associated monomer molecules, e.g., joined by a linker, e.g., a peptide linker (e.g., the three covalently associated monomers and the linker forming a fusion protein). In embodiments wherein two monomer molecules are covalently linked monomer, the linked monomers can be reading from left to right have the following configuration: Amino terminal to carboxy terminal, or carboxy terminal to amino terminal. In embodiments, the trimeric ligand interacts, e.g., binds to a target molecule, e.g., a receptor.

In some embodiments, the trimeric ligand includes three monomer molecules, e.g., wherein two of the monomer molecules are coupled, e.g., covalently linked, to one another, and the third monomer molecule is non-covalently associated to the other two monomer molecules. In some embodiments, the multifunctional molecule comprises two, three or more trimeric ligands that are the same or different. In some embodiments, the trimeric ligand in the multifunctional molecule is a homotrimer, e.g., is composed of the same monomer molecules, or a heterotrimer, e.g., is composed of two or three different monomer molecules. In some embodiments, the trimeric ligand is a member of the tumor necrosis factor superfamily (TNFSF) or TNFSF-like members, or a combination of TNFSF- and TNFSF-like monomers. The multifunctional molecules can further include a dimerization module.

As used herein, a “dimerization module” comprises at least two members, a first and second member, e.g., at least two polypeptides, that are associated, e.g., covalently or non-covalently, with one another. In some embodiments, the first and second members provide for an interface that allows for dimerization. In embodiments where the first and second members are different, e.g., the affinity of the first member for the second member is sufficiently greater than its affinity for another first member, such that under preselected conditions, e.g., in aqueous buffer, e.g., at pH 7, in saline, e.g., at pH 7, or under physiological conditions, at least 75%, 80, 90, 95, 98, 99, 99.5, or 99.9% of the multispecific molecules have a first member complexed, or interfaced with, the second member. In some embodiments, the interface between the first and second member is altered, e.g., modified to provide greater affinity between an altered dimer having the altered interface, e.g., relative to a non-engineered interface, e.g., a naturally-occurring interface.

In one embodiment, the dimerization module comprises at least two non-covalently associated first and second members, e.g., first and second polypeptides. In other embodiments, the dimerization module comprises at least two covalently associated first and second members, e.g., polypeptides. In some embodiments, the first and second members, e.g., polypeptides are the same, e.g., thus providing a homodimer upon association of the first and second members. In other embodiments, the first and second members, e.g., polypeptides, are different, e.g., thus providing a heterodimer.

In some embodiments, the dimerization module comprises an immunoglobulin constant domain. In some embodiments, the dimerization module comprises a heavy chain constant region or a constant domain of an immunoglobulin variable region.

In some embodiments, each trimeric ligand is coupled, e.g., covalently linked or fused, to a heavy chain constant region (e.g., an Fc region), e.g., a homodimeric or heterodimeric heavy chain constant region. The trimeric ligand can be coupled to the heavy chain, e.g., at the N- or C-terminus of the heavy chain constant region.

In other embodiments, the trimeric ligand is coupled, e.g., covalently linked or fused, to a constant domain of a Fab region. In some embodiments, the trimeric ligand is coupled, e.g., covalently coupled or fused, to the N-terminus of the heavy chain variable constant region (e.g., a Fab CH₁) and/or to the N-terminus of the light chain variable constant region (e.g., a Fab CL).

Without wishing to be bound by theory, the use of a dimerization module, e.g., an immunoglobulin constant domain (e.g., a homodimeric or heterodimeric Fc region) or a constant domain of an immunoglobulin variable region, can allow for the construction of a multifunctional molecule comprising one or more homotrimer or heterotrimer ligands, optionally, in conjunction with, one, two, or three other binding specificities or functionalities.

In some embodiments, the multifunctional molecule further comprises one or more other binding specificities or functionalities. In embodiments, the other binding specificity or functionality is chosen from one, two or more of: a targeting moiety, e.g., a tumor targeting moiety; an immune cell engager (e.g., chosen from one, two, three, or all of an NK cell engager, a B cell engager, a dendritic cell engager, or a macrophage cell engager); and/or a cytokine molecule, e.g., as described herein.

Thus, disclosed herein are, inter alia, novel multifunctional, e.g., bifunctional, trifunctional, or tetrafunctional, molecules (e.g., fusion polypeptides or nucleic acids) that include a trimeric ligand, e.g., one or more (e.g., one, two or three) trimeric ligands, and an immunoglobulin constant domain, as well as methods of making and using the multifunctional molecules, e.g., for treating a disorder, e.g., cancer.

Additional terms are defined below.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

“Antibody molecule” as used herein refers to a protein, e.g., an immunoglobulin chain or fragment thereof, comprising at least one immunoglobulin variable domain sequence. An antibody molecule encompasses antibodies (e.g., full-length antibodies) and antibody fragments. In an embodiment, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs).

As used herein, the term “molecule” as used in, e.g., an antibody molecule, a cytokine molecule, a receptor molecule, a monomer molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule remains. In an embodiment, the molecule comprises at least 70%, 80%, 85%, 90%, 95%, 99% or higher sequence identity to a naturally-occurring molecule.

The term “functional variant” refers to polypeptides that have a substantially identical amino acid sequence to the naturally-occurring sequence, or are encoded by a substantially identical nucleotide sequence, and are capable of having one or more activities of the naturally-occurring sequence.

“Derived from” as used herein in reference the relationship of a first sequence, to a second sequence (e.g., in the context of nucleic acid sequence or protein sequences) imposes no process limitations and refers only to structural similarity. In embodiments a derived sequence will differ from the reference sequence by levels of homology or sequence identity described elsewhere herein.

As used herein, a “CH2 domain” refers to an immunoglobulin CH2 domain, e.g., an IgG1, an IgG2, an IgG3, an IgG4 CH2 domain, or any fragment thereof.

As used herein, a “CH3 domain” refers to an immunoglobulin CH3 domain, e.g., an IgG1, an IgG2, an IgG3, an IgG4 CH3 domain, or any fragment thereof.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Multispecific antibody molecule” as that term is used herein, refers to an antibody molecule having specificity for two non-identical epitopes, e.g., having a first variable region specific for a first epitope and a second variable region specific for a second epitope, wherein the first and second epitopes are non-identical. Multispecific antibody molecules include bispecific antibody molecules.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site,” or “binding portion” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” as that term is used herein, refers to a cell that is involved in an immune response, e.g., in the promotion of an immune effector response. Examples of immune effector cells include, but are not limited to, T cells, e.g., alpha/beta T cells and gamma/delta T cells, B cells, natural killer (NK) cells, natural killer T (NK T) cells, and mast cells.

The term “effector function” or “effector response” refers to a specialized function of a cell. Effector function of a T cell, for example, may be cytolytic activity or helper activity including the secretion of cytokines.

In some embodiments, the multifunctional molecule includes a tumor-targeting moiety. A “tumor-targeting moiety,” as used herein, refers to a binding agent that recognizes or associates with, e.g., binds to, a target in a cancer cell. The tumor-targeting moiety can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the cancer antigen (e.g., the tumor and/or the stromal antigen). In embodiments, the tumor-targeting moiety specifically binds to the target tumor, e.g., binds preferentially to the target tumor. For example, when the tumor-targeting moiety is an antibody molecule, it binds to the cancer antigen (e.g., the tumor antigen and/or the stromal antigen) with a dissociation constant of less than about 10 nM, and more typically, 10-100 pM.

In some embodiments, the multifunctional molecule includes an immune cell engager. “An immune cell engager” refers to one or more binding specificities that bind and/or activate an immune cell, e.g., a cell involved in an immune response. In embodiments, the immune cell is chosen from an NK cell, a B cell, a dendritic cell, and/or the macrophage cell. The immune cell engager can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the immune cell antigen (e.g., the NK cell antigen, the B cell antigen, the dendritic cell antigen, and/or the macrophage cell antigen). In embodiments, the immune cell engager specifically binds to the target immune cell, e.g., binds preferentially to the target immune cell. For example, when the immune cell engager is an antibody molecule, it binds to the immune cell antigen (e.g., the NK cell antigen, the B cell antigen, the dendritic cell antigen, and/or the macrophage cell antigen) with a dissociation constant of less than about 10 nM, and more typically, 10-100 pM.

In some embodiments, the multifunctional molecule includes a cytokine molecule. As used herein, a “cytokine molecule” refers to full length, a fragment or a variant of a cytokine; a cytokine further comprising a receptor domain, e.g., a cytokine receptor dimerizing domain; or an agonist of a cytokine receptor, e.g., an antibody molecule (e.g., an agonistic antibody) to a cytokine receptor, that elicits at least one activity of a naturally-occurring cytokine. In some embodiments the cytokine molecule is chosen from interleukin-2 (IL-2), interleukin-12 (IL-12), interleukin-15 (IL-15), interleukin-18 (IL-18), interleukin-21 (IL-21), or interferon gamma, or a fragment or variant thereof, or a combination of any of the aforesaid cytokines. The cytokine molecule can be a monomer molecule or a dimer. In embodiments, the cytokine molecule can further include a cytokine receptor dimerizing domain. In other embodiments, the cytokine molecule is an agonist of a cytokine receptor, e.g., an antibody molecule (e.g., an agonistic antibody) to a cytokine receptor chosen from an IL-2, IL-15Ra or IL-21R.

The compositions and methods of the present invention encompass polypeptides and nucleic acids having the sequences specified, or sequences substantially identical or similar thereto, e.g., sequences at least 80%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% or 99% identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant.

The term “functional variant” refers to a polypeptide that has a substantially identical amino acid sequence to a reference amino acid sequence, or is encoded by a substantially Identical nucleotide sequence, and is capable of having one or more activities of the reference amino acid sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

Various aspects of the invention are described in further detail below. Additional definitions are set out throughout the specification.

### Antibody Molecules

In one embodiment, the antibody molecule binds to an antigen, e.g., an immune effector cell, a tumor antigen or a stromal antigen. In some embodiments, the antigen is, e.g., a mammalian, e.g., a human, antigen. In other embodiments, the antibody molecule binds to an immune cell antigen, e.g., a mammalian, e.g., a human, immune cell antigen. For example, the antibody molecule binds specifically to an epitope, e.g., linear or conformational epitope, on the cancer antigen or the immune cell antigen.

In an embodiment, an antibody molecule is a monospecific antibody molecule and binds a single epitope. E.g., a monospecific antibody molecule having a plurality of immunoglobulin variable domain sequences, each of which binds the same epitope.

In an embodiment an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a multispecific antibody molecule comprises a third, fourth or fifth immunoglobulin variable domain. In an embodiment, a multispecific antibody molecule is a bispecific antibody molecule, a trispecific antibody molecule, or a tetraspecific antibody molecule.

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv or a Fab, or fragment thereof, have binding specificity for a first epitope and a scFv or a Fab, or fragment thereof, have binding specificity for a second epitope.

In an embodiment, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In an embodiment an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The a preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg er al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the invention. Antibody molecules generated in a non-human organism, e.g., a rat or mouse, and then modified, e.g., in the variable framework or constant region, to decrease antigenicity in a human are within the invention.

An “effectively human” protein is a protein that does substantially not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### Multifunctional or Multispecific Antibody Molecules

In embodiments, multispecific antibody molecules can comprise more than one antigen-binding site, where different sites are specific for different antigens. In embodiments, multispecific antibody molecules can bind more than one (e.g., two or more) epitopes on the same antigen. In embodiments, multispecific antibody molecules comprise an antigen-binding site specific for a target cell (e.g., cancer cell) and a different antigen-binding site specific for an immune effector cell. In one embodiment, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, κλ-body, orthogonal Fab. See Spiess et al. Mol. Immunol. 67(2015):95-106. Exemplary BslgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fc regions. See Spiess et al. Mol. Immunol. 67(2015):95-106. Strategies that have been used to avoid heavy chain pairing of homodimers in BsIgG include knobs-in-holes, duobody, azymetric, charge pair, HA-TF, SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). See Id. Examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HER3. Examples of DVD-Ig include ABT-981 (AbbVie), which binds IL-1α and IL-1β; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody. See Id. For example, the BiTE format comprises tandem scFvs, where the component scFvs bind to CD3 on T cells and a surface antigen on cancer cells

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In embodiments, the dock-and-lock (DNL) method can be used to generate bispecific antibody molecules with higher valency. Also, fusions to albumin binding proteins or human serum albumin can be extend the serum half-life of antibody fragments. See Id.

### CDR-Grafted Scaffolds

In embodiments, the antibody molecule is a CDR-grafted scaffold domain. In embodiments, the scaffold domain is based on a fibronectin domain, e.g., fibronectin type III domain. The overall fold of the fibronectin type III (Fn3) domain is closely related to that of the smallest functional antibody fragment, the variable domain of the antibody heavy chain. There are three loops at the end of Fn3; the positions of BC, DE and FG loops approximately correspond to those of CDR1, 2 and 3 of the VH domain of an antibody. Fn3 does not have disulfide bonds; and therefore Fn3 is stable under reducing conditions, unlike antibodies and their fragments (see, e.g., WO 98/56915; WO 01/64942; WO 00/34784). An Fn3 domain can be modified (e.g., using CDRs or hypervariable loops described herein) or varied, e.g., to select domains that bind to an antigen/marker/cell described herein.

In embodiments, a scaffold domain, e.g., a folded domain, is based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomer moleculeic DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). See, e.g., US 20040009530 and U.S. Pat. No. 7,501,121, incorporated herein by reference.

In embodiments, a scaffold domain is evaluated and chosen, e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In embodiments, the scaffold domain is a small, stable protein domain, e.g., a protein of less than 100, 70, 50, 40 or 30 amino acids. The domain may include one or more disulfide bonds or may chelate a metal, e.g., zinc.

Exemplary structures of the multifunctional molecules defined herein are described below. Exemplary structures are further described in: Weidle U et al. (2013) The Intriguing Options of Multispecific Antibody Formats for Treatment of Cancer. Cancer Genomics & Proteomics 10: 1-18 (2013); and Spiess C et al. (2015) Alternative molecular formats and therapeutic applications for bispecific antibodies. Molecular Immunology 67: 95-106; the full contents of each of which is incorporated by reference herein).

### Non-Immunoglobulin Heterodimerization Domains

Non-immunoglobulin heterodimerization domains described herein include, e.g., TCRα constant domain and TCRβ constant domain. In some embodiments, the TCRα constant domain comprises a naturally existing sequence. In some embodiments, the TCRα constant domain comprises a functional variant of a naturally existing sequence, e.g., a functional variant capable of forming stable association with a TCRβ constant domain. In some embodiments, the TCRβ constant domain comprises a naturally existing sequence. In some embodiments, the TCR constant domain comprises a functional variant of a naturally existing sequence, e.g., a functional variant capable of forming stable association with a TCRα constant domain.

### TCRα Constant Domain

In some embodiments, the TCRα domain comprises the WT human TCRα constant domain having the following amino acid sequence (human WT full length TCRα constant domain):

Uniprot Reference: P01848
SEQ ID NO: 118
PNIQNPDPAVYQLRDSKSSDKSVCLFTDFDSQTNVSQSKDSDVYITDKT
VLDMRSMDFKSNSAVAWSNKSDFACANAFNNSIIPEDTFFPSPESSCDV
KLVEKSFETDTNLNFQNLSVIGFRILLLKVAGFNLLMTLRLWSS.

In some embodiments, the TCRα domain comprises a fragment of SEQ ID NO: 118.

In some embodiments, the TCRα domain comprises or consists of amino acids 1-85 of SEQ ID NO: 118.

In some embodiments, the TCRα domain comprises or consists of the following amino acid sequence:

SEQ ID NO: 119
PDIQNPDPAVYQLRDSKSSDKSVCLFTDFDSQTNVSQSKDSDVYITDKC
VLDMRSMDFKSNSAVAWSNKSDFACANAFNNSIIPEDTFFPSPE.

In some embodiments, the TCRα domain comprises or consists of at least 5, 10, 20, 30, 40, 50, 60, 70, 80, 90, or 100 contiguous amino acids of SEQ ID NO: 118.

In some embodiments, the TCRα domain comprises or consists of at least 5, 10, 20, 30, 40, 50, 60, 70, or 80 contiguous amino acids of SEQ ID NO: 119.

In some embodiments, the TCRα domain comprises or consists of at least 5, 10, 20, 30, 40, 50, 60, 70, 80, 90, or 100 contiguous amino acids of SEQ ID NO: 118, with no more than 10, 9, 8, 7, 6, 5, 4, 3, 2, or 1 amino acid substitutions. In some embodiments, the TCRα domain comprises or consists of at least 5, 10, 20, 30, 40, 50, 60, 70, or 80 contiguous amino acids of SEQ ID NO: 119, with no more than 10, 9, 8, 7, 6, 5, 4, 3, 2, or 1 amino acid substitutions.

In some embodiments, the TCRα domain comprises or consists of at least 5, 10, 20, 30, 40, 50, 60, 70, 80, 90, or 100 contiguous amino acids of SEQ ID NO: 118, with 1, 2, 3, 4, 5, 5, 6, 7, 8, 9, 10, or more amino acid substitutions. In some embodiments, the TCRα domain comprises or consists of at least 5, 10, 20, 30, 40, 50, 60, 70, 80, 90, or 100 contiguous amino acids of SEQ ID NO: 119, with 1, 2, 3, 4, 5, 5, 6, 7, 8, 9, 10, or more amino acid substitutions.

In some embodiments, the TCRβ domain comprises the WT human TCRβ constant domain having the following amino acid sequence (human WT full length TCRβ constant domain):

Uniprot Reference: P01850
SEQ ID NO: 120
EDLNKVFPPEVAVFEPSEAEISHTQKATLVCLATGFFPDHVELSWWVNG
KEVHSGVSTDPQPLKEQPALNDSRYCLSSRLRVSATFWQNPRNHFRCQV
QFYGLSENDEWTQDRAKPVTQIVSAEAWGRADCGFTSVSYQQGVLSATI
LYEILLGKATLYAVLVSALVLMAMVKRKDF.

In some embodiments, the TCRβ domain comprises a fragment of SEQ ID NO: 120.

In some embodiments, the TCRβ domain comprises or consists of amino acids 1-130 of SEQ ID NO: 120.

In some embodiments, the TCRβ constant domain comprises or consists of the following amino acid sequence:

SEQ ID NO: 121
EDLNKVFPPEVALFEPSEAEISHTQKATLVCLATGFYPDHVELSWWVNG
KEVHSGVCTDPQPLKEQPALNDSRYALSSRLRVSATFWQDPRNHFRCQV
QFYGLSEADEWTQARAKPVTQIVSAEAWGRAD.

In some embodiments, the TCRβ domain comprises or consists of at least 5, 10, 20, 30, 40, 50, 60, 70, 80, 90, 100, 110, 120, 130, 140, 150, 160, or 170 contiguous amino acids of SEQ ID NO: 120.

In some embodiments, the TCRβ domain comprises or consists of at least 5, 10, 20, 30, 40, 50, 60, 70, 80, 90, 100, 110, 120, or 130 contiguous amino acids of SEQ ID NO: 121.

In some embodiments, the TCRβ domain comprises or consists of at least 5, 10, 20, 30, 40, 50, 60, 70, 80, 90, 100, 110, 120, 130, 140, 150, 160, or 170 contiguous amino acids of SEQ ID NO: 120, with no more than 10, 9, 8, 7, 6, 5, 4, 3, 2, or 1 amino acid substitutions. In some embodiments, the TCRβ domain comprises or consists of at least 5, 10, 20, 30, 40, 50, 60, 70, 80, 90, 100, 110, 120, or 130 contiguous amino acids of SEQ ID NO: 121, with no more than 10, 9, 8, 7, 6, 5, 4, 3, 2, or 1 amino acid substitutions.

In some embodiments, the TCRβ domain comprises or consists of at least 5, 10, 20, 30, 40, 50, 60, 70, 80, 90, 100, 110, 120, 130, 140, 150, 160, or 170 contiguous amino acids of SEQ ID NO: 120, with 1, 2, 3, 4, 5, 5, 6, 7, 8, 9, 10, or more amino acid substitutions. In some embodiments, the TCR domain comprises or consists of at least 5, 10, 20, 30, 40, 50, 60, 70, 80, 90, 100, 110, 120, or 130 contiguous amino acids of SEQ ID NO: 121, with 1, 2, 3, 4, 5, 5, 6, 7, 8, 9, 10, or more amino acid substitutions.

In some embodiments, the multispecific molecules disclosed herein include a portion immunoglobulin constant region (e.g., an Fc region) (e.g., CH2 domain of an Fc). Exemplary Fc regions can be chosen from the heavy chain constant regions of IgG1, IgG2, IgG3 or IgG4; more particularly, the CH2 heavy chain constant region of human IgG1, IgG2, IgG3, or IgG4.

In some embodiments, the immunoglobulin chain constant region (e.g., CH2 of the Fc region) is altered, e.g., mutated, to increase or decrease one or more of: Fc receptor binding, antibody glycosylation, the number of cysteine residues, effector cell function, or complement function.

In some embodiments, a linker is present between the CH2 and TCRα and TCRβ domain.

In some embodiments, the TCRα and/or β constant domain is altered, e.g., mutated, to increase or decrease dimerization. For example, dimerization of the chain is enhanced by introducing a cysteine residue in the TCRα and TCRβ domains creating an engineered disulfide, such that a greater ratio of heteromultimer to homomultimer forms, e.g., relative to a non-engineered interface.

In other embodiments, the multispecific molecule includes a half-life extender, e.g., a human serum albumin or an antibody molecule to human serum albumin.

### Trimer Ligands

A trimeric ligand can comprise comprising three members, also referred to herein as “monomer molecules.” In embodiments, the three members are three covalently or non-covalently associated polypeptides. In some embodiments, the trimeric ligand comprises three non-covalently associated monomer molecules. In one embodiment, the trimeric ligand comprises two covalently associated monomer molecules, e.g., joined by a linker, e.g., a peptide linker (e.g., the two covalently associated monomers and the linker forming a fusion protein) and one non-covalently associated monomer molecule. In yet other embodiments, the trimeric ligand comprises three covalently associated monomer molecules, e.g., joined by a linker, e.g., a peptide linker (e.g., the three covalently associated monomers and the linker forming a fusion protein). In embodiments wherein two monomer molecules are covalently linked monomer, the linked monomers can be reading from left to right have the following configuration: Amino terminal to carboxy terminal, or carboxy terminal to amino terminal. In embodiments, the trimeric ligand interacts, e.g., binds to a target molecule, e.g., a receptor.

In embodiments, the multispecific molecule comprises a plurality of, e.g., two or three, trimeric ligands, independently each of the trimeric ligand(s), can comprise any combinations of homomonomer(s) and heteromonomer(s). (A monomer is a homomonomer if it is identical with, differs by less than 10 amino acid residues from, or is from the same TNFSF or TNF-like monomer as, at least one other monomer of the trimeric ligand; a monomer is a heteromonomer if it differs from, differs by 10 or more amino acid residues from, or is from a different TNFSF or TNF-like monomer, as another monomer in the trimeric ligand.

In an embodiment, the trimeric ligand comprises a plurality of, e.g., two or three, trimeric ligands, independently each of the trimeric ligand(s), can comprise:

(i) three homomonomers (with any coupling configuration, e.g., none of the three monomers covalently linked, two of the three monomers covalently linked, or all for the three of the monomers covalently linked);

(ii) two homomonomers and one heteromonomer (with any coupling configuration, e.g., none of the three monomers covalently linked, two of the three monomers covalently linked, or all for the three of the monomers covalently linked, wherein when the heteromonomer is covalently linked to another monomer, the heteromonomer a can be covalently linked to either a homomonomer or a heteromonomer); or

(iii), a heteromonomer¹, heteromonomer², and heteromonomer³ (with any coupling configuration, e.g., none of the three monomers are covalently linked, two of the three monomers are covalently linked, or all of the three of the monomers are covalently linked, and, e.g., wherein when two monomers are covalently linked, heteromonomer¹ is linked to heteromonomer², heteromonomer¹ is linked to heteromonomer³, or heteromonomer² is linked to heteromonomer³); (wherein for covalently linked monomers, the linked monomers can be reading from left to right, amino terminal to carboxy terminal, or carboxy terminal to amino terminal).

### TNFSF and TNF-Like Family Members

In some embodiments, the trimeric ligand is a member of the TNF superfamily, also referred to herein as a “TNFSF family member.” The tumor necrosis factor (TNF) superfamily refers to a superfamily of cytokines identified as part of the TNF family on the basis of, e.g., sequence, function and/or structural similarities, e.g., reviewed in Sun M, Fink P J (2007) J Immunol. 179 (7): 4307-12; Peitsch M C, Jongeneel C V (1993) Int. Immunol. 5 (2): 233-8; Farrah T, Smith C A (July 1992) Nature 358 (6381): 26; Bazan J F (1993). Curr. Biol. 3 (9): 603-6. TNFSF family members typically form homotrimeric or heterotrimeric complexes.

In embodiments, the trimeric ligand is chosen from a TNFSF family member or a TNF-like family member, or a combination thereof, e.g., as described herein in Tables 1 and 2. In some embodiments, the trimeric ligand is a homotrimer, e.g., it comprises three monomer molecules from the same trimeric ligand, e.g., the same TNFSF family member or the same TNF-like family member. In other embodiments, the trimeric ligand is a heterotrimer, e.g., it comprises a combination of monomer molecules from two or three trimeric ligands, e.g., two or three TNFSF or TNF-like family members.

In embodiments, the trimeric ligand comprises the amino acid sequence of a monomer molecule chosen from a TNFSF family member or a TNF-like family member, e.g., as described herein in Tables 1 and 2, or an amino acid sequence substantially identical thereto, e.g., at least 85%, 90%, 95%, 99% or more identical to an amino acid described in Tables 1 and 2. For example, the trimeric ligand can comprise any of the TNFSF amino acid sequences for TNSF1, TNSF2, TNSF3, TNSF4, TNSF5, TNSF6, TNSF7, TNSF8, TNSF9, TNSF10, TNSF1, TNSF11, TNSF12, TNSF13, TNSF13B, TNSF14, TNSF15, TNF18 or EDA, corresponding to SEQ ID NOs: 1-17 and 218, in Table 1, or an amino acid sequence substantially identical thereto, e.g., at least 85%, 90%, 95%, 99% or more identical to the amino acid sequence of any of SEQ ID NOs: 1-17 and 218, as a homotrimer, or a heterotrimer comprising any combination of two or three of the aforesaid monomer molecules. In some embodiments, the trimeric ligand can comprise any of the TNF-like amino acid sequences for Complement C1Q (e.g., subcomponents A, B and C), C1QL1, C1QL2, C1QL3, Caprin-2 Clq domain, cerebellin-1 C1q domain or adiponectin, corresponding to SEQ ID NOs: 239-247, in Table 2, or an amino acid sequence substantially identical thereto, e.g., at least 85%, 90%, 95%, 99% or more identical to the amino acid sequence of any of SEQ ID NOs: 239-247, as a homotrimer (e.g., comprises of homomonomer molecules), or a heterotrimer (e.g., comprises of heteromonomer molecules) comprising any combination of two or three of the aforesaid monomers. In other embodiments, the trimeric ligand comprises a combination of TNFSF monomer molecules and TNF-like monomer molecules, wherein the trimer ligand is a heterotrimer comprising any combination of two or three monomer molecules comprising the amino acid sequence of any of SEQ ID NOs: 1-17, 218, and 239-247.

In some embodiments, the tumor necrosis factor superfamily member is chosen from one or more of the sequences in Table 1.

TABLE 1

| Family | | Uniprot | |
|---|---|---|---|
| Member | Common Names | Accession | Monomer molecule Sequence |
| TNFSF1 | lymphotoxin-alpha, | P01374 | SEQ ID NO: 1 |
| | LTα, TNFβ | | KPAAHLIGDPSKQNSLLWRANTDRAFLQDGFSLS |
| | | | NNSLLVPTSGIYFVYSQVVFSGKAYSPKATSSPL |
| | | | YLAHEVQLFSSQYPFHVPLLSSQKMVYPGLQEPW |
| | | | LHSMYHGAAFQLTQGDQLSTHTDGIPHLVLSPST |
| | | | VFFGAFAL |
| TNFSF2 | tumor necrosis | P01375 | SEQ ID NO: 2 |
| | factor, TNFα | | RTPSDKPVAHVVANPQAEGQLQWLNRRANALLAN |
| | | | GVELRDNQLVVPSEGLYLIYSQVLFKGQGCPSTH |
| | | | VLLTHTISRIAVSYQTKVNLLSAIKSPCQRETPE |
| | | | GAEAKPWYEPIYLGGVFQLEKGDRLSAEINRPDY |
| | | | LDFAESGQVYFGIIAL |
| TNFSF3 | lymphotoxin-beta | Q06643 | SEQ ID NO: 3 |
| | | | LPAAHLIGAPLKGQGLGWETTKEQAFLTSGTQFS |
| | | | DAEGLALPQDGLYYLYCLVGYRGRAPPGGGDPQG |
| | | | RSVTLRSSLYRAGGAYGPGTPELLLEGAETVTPV |
| | | | LDPARRQGYGPLWYTSVGFGGLVQLRRGERVYVN |
| | | | ISHPDMVDFARGKTFFGAVMVG |
| TNFSF4 | OX40L, OX40 | P23510 | SEQ ID NO: 4 |
| | ligand, CD252 | | RIQSIKVQFTEYKKEKGFILTSQKEDEIMKVQNN |
| | | | SVIINCDGFYLISLKGYFSQEVNISLHYQKDEEP |
| | | | LFQLKKVRSVNSLMVASLTYKDKVYLNVTTDNTS |
| | | | LDDFHVNGGELILIHQNPGEFCVL |
| TNFSF5 | CD40L, CD40 | P29965 | SEQ ID NO: 5 |
| | ligand, CD154 | | GDQNPQIAAHVISEASSKTTSVLQWAEKGYYTMS |
| | | | NNLVTLENGKQLTVKRQGLYYIYAQVTFCSNREA |
| | | | SSQAPFIASLCLKSPGRFERILLRAANTHSSAKP |
| | | | CGQQSIHLGGVFELQPGASVFVNVTDPSQVSHGT |
| | | | GFTSFGLLKL |
| | CD40L C194A (the | | SEQ ID NO: 198 |
| | position where a | | GDQNPQIAAHVISEASSKTTSVLQWAEKGYYTMS |
| | cysteine residue | | NNLVTLENGKQLTVKRQGLYYIYAQVTFCSNREA |
| | is mutated to an | | SSQAPFIASLALKSPGRFERILLRAANTHSSAKP |
| | alanine residue | | CGQQSIHLGGVFELQPGASVFVNVTDPSQVSHGT |
| | is underlined) | | GFTSFGLLKL |
| TNFSF6 | FasL, Fas antigen | P48023 | SEQ ID NO: 6 |
| | ligand, CD95L, | | LRKVAHLTGKSNSRSMPLEWEDTYGIVLLSGVKY |
| | CD95 ligand, | | KKGGLVINETGLYFVYSKVYFRGQSCNNLPLSHK |
| | CD178 | | VYMRNSKYPQDLVMMEGKMMSYCTTGQMWARSSY |
| | | | LGAVFNLTSADHLYVNVSELSLVNFEESQTFFGL |
| | | | YKL |
| TNFSF7 | CD70, CD27L, | P32970 | SEQ ID NO: 7 |
| | CD27 ligand | | QRFAQAQQQLPLESLGWDVAELQLNHTGPQQDPR |
| | | | LYWQGGPALGRSFLHGPELDKGQLRIHRDGIYMV |
| | | | HIQVTLAICSSTTASRHHPTTLAVGICSPASRSI |
| | | | SLLRLSFHQGCTIASQRLTPLARGDTLCTNLTGT |
| | | | LLPSRNTDETFFGVQWVRP |
| TNFSF8 | CD30L, CD30 | P32971 | SEQ ID NO: 8 |
| | ligand, CD153 | | QRTDSIPNSPDNVPLKGGNCSEDLLCILKRAPFK |
| | | | KSWAYLQVAKHLNKTKLSWNKDGILHGVRYQDGN |
| | | | LVIQFPGLYFIICQLQFLVQCPNNSVDLKLELLI |
| | | | NKHIKKQALVTVCESGMQTKHVYQNLSQFLLDYL |
| | | | QVNTTISVNVDTFQYIDTSTFPLENVLSIFLYSN |
| | | | SD |
| TNFSF9 | 4-1BB ligand, 4- | P41273 | SEQ ID NO: 9 |
| | 1BBL | | DPAGLLDLRQGMFAQLVAQNVLLIDGPLSWYSDP |
| | | | GLAGVSLTGGLSYKEDTKELVVAKAGVYYVFFQM |
| | | | ELRRVVAGEGSGSVSLALHLMPLRSAAGAAALAL |
| | | | TVDLPPASSEARNSAFGFQGRLLHLSAGQRLGVH |
| | | | HTEARARHAWQLTQGATVLGLFRVTPEIPA |
| TNFSF10 | TRAIL, TNF- | P50591 | SEQ ID NO: 10 |
| | related apoptosis- | | PQRVAAHITGTRGRSNTLSSPNSKNEKALGRKIN |
| | inducing ligand, | | SWESSRSGHSFLSNLHLRNGELVIHEKGFYYIYS |
| | Apo-2 ligand, Apo- | | QTYFRFQEEIKENTKNDKQMVQYIYKYTSYPDPI |
| | 2L, CD253 | | LLMKSARNSCWSKDAEYGLYSIYQGGIFELKEND |
| | | | RIFVSVTNEHLIDMDHEASFFGAFLVG |
| TNFSF11 | RANKL, Receptor | O14788 | SEQ ID NO: 11 |
| | activator of nuclear | | AQPFAHLTINATDIPSGSHKVSLSSWYHDRGWAK |
| | factor kappa-B | | ISNMTFSNGKLIVNQDGFYYLYANICFRHHETSG |
| | ligand, Osteoclast | | DLATEYLQLMVYVTKTSIKIPSSHTLMKGGSTKY |
| | differentiation | | WSGNSEFHFYSINVGGFFKLRSGEEISIEVSNPS |
| | factor, ODF, | | LLDPDQDATYFGAFKVRDID |
| | Osteoprotegerin | | |
| | ligand, OPGL, | | |
| | TNF-related | | |
| | activation-induced | | |
| | cytokine, | | |
| | TRANCE, CD254 | | |
| TNFSF12 | TWEAK, TNF- | O43508 | SEQ ID NO: 12 |
| | related weak | | RAIAAHYEVHPRPGQDGAQAGVDGTVSGWEEARI |
| | inducer of | | NSSSPLRYNRQIGEFIVTRAGLYYLYCQVHFDEG |
| | apoptosis, APO3 | | KAVYLKLDLLVDGVLALRCLEEFSATAASSLGPQ |
| | ligand, APO3L | | LRLCQVSGLLALRPGSSLRIRTLPWAHLKAAPFL |
| | | | TYFGLFQVH |
| TNFSF13 | APRIL (A | O75888 | SEQ ID NO: 13 |
| | proliferation- | | HSVLHLVPINATSKDDSDVTEVMWQPALRRGRGL |
| | inducing ligand, | | QAQGYGVRIQDAGVYLLYSQVLFQDVTFTMGQVV |
| | TNF- and APOL- | | SREGQGRQETLFRCIRSMPSHPDRAYNSCYSAGV |
| | related leukocyte | | FHLHQGDILSVIIPRARAKLNLSPHGTFLGFVKL |
| | expressed ligand 2, | | |
| | TALL-2, TNF- | | |
| | related death ligand | | |
| | 1, TRDL-1, CD256 | | |
| | APRIL | | SEQ ID NO: 180 |
| | | | HSVLHLVPINAASKDDSDVTEVMWQPALRRGRGL |
| | | | QAQGYGVRIQDAGVYLLYSQVLFQDVTFTMGQVV |
| | | | SREGQGRQETLFRCIRSMPSHPDRAYNSCYSAGV |
| | | | FHLHQGDILSVIIPRARAKLNLSPHGTFLGFVKL |
| TNFSF13B | BAFF, B-cell- | Q9Y275 | SEQ ID NO: 14 |
| | activating factor, | | ETVTQDCLQLIADSETPTIQKGSYTFVPWLLSFK |
| | B lymphocyte | | RGSALEEKENKILVKETGYFFIYGQVLYTDKTYA |
| | stimulator, BlyS, | | MGHLIQRKKVHVFGDELSLVTLFRCIQNMPETLP |
| | Dendritic cell- | | NNSCYSAGIAKLEEGDELQLAIPRENAQISLDGD |
| | derived TNF-like | | VTFFGALKLL |
| | molecule, TNF- and | | |
| | APOL-related | | |
| | leukocyte expressed | | |
| | ligand 1, TALL-1, | | |
| | CD257 | | |
| TNFSF14 | LIGHT, HVEM-L, | O43557 | SEQ ID NO: 15 |
| | Herpes virus entry | | NPAAHLTGANSSLTGSGGPLLWETQLGLAFLRGL |
| | mediator ligand, | | SYHDGALVVTKAGYYYIYSKVQLGGVGCPLGLAS |
| | CD258 | | TITHGLYKRTPRYPEELELLVSQQSPCGRATSSS |
| | | | RVWWDSSFLGGVVHLEAGEKVVVRVLDERLVRLR |
| | | | DGTRSYFGAFMV |
| TNFSF15 | TNF ligand-related | O95150 | SEQ ID NO: 16 |
| | molecule 1, TL1, | | DGDKPRAHLTVVRQTPTQHFKNQFPALHWEHELG |
| | Vascular | | LAFTKNRMNYTNKFLLIPESGDYFIYSQVTFRGM |
| | endothelial cell | | TSECSEIRQAGRPNKPDSITVVITKVTDSYPEPT |
| | growth inhibitor, | | QLLMGTKSVCEVGSNWFQPIYLGAMFSLQEGDKL |
| | VEGI | | MVNVSDISLVDYTKEDKTFFGAFLL |
| TNFSF18 | GITRL | Q9UNG2 | SEQ ID NO: 17 |
| | (Glucocorticoid- | | QLETAKEPCMAKFGPLPSKWQMASSEPPCVNKVS |
| | induced TNF- | | DWKLEILQNGLYLIYGQVAPNANYNDVAPFEVRL |
| | related ligand, | | YKNKDMIQTLTNKSKIQNVGGTYELHVGDTIDLI |
| | Activation- | | FNSEHQVLKNNTYWGIILLANPQFIS |
| | inducible TNF- | | |
| | related ligand, | | |
| | AITRL, TL6 | | |
| EDA | EDA1, XLHED, | Q92838 | SEQ ID NO: 218 |
| | HED, XHED, ED1- | | QPAVVHLQGQGSAIQVKNDLSGGVLNDWSRITMN |
| | A1, ED1-A2, EDA- | | PKVFKLHPRSGELEVLVDGTYFIYSQVEVYYINF |
| | A1, EDA-A2 | | TDFASYEVVVDEKPFLQCTRSIETGKTNYNTCYT |
| | | | AGVCLLKARQKIAVKMVHADISINMSKHTTFFGA |
| | | | IRLGEAPA |

In other embodiments, the trimer ligand is a tumor necrosis factor superfamily member chosen from one or more of the TNF-like Superfamily members including the amino acid sequence in Table 2, or an amino acid sequence substantially identical thereto, e.g., at least 80%, 85%, 90%, 95%, 99% or more identical to any of the amino acid sequences in Table 2. In one embodiment, the trimer ligand is a homotrimer, e.g, it comprises three ligand monomer molecules having the same amino acid sequence. In another embodiment, the trimer ligand is a heterotrimer, e.g., it comprises three ligand monomer molecules having different amino acid sequences. For example, in some embodiments, 2 ligand monomer molecule are identical and 1 is different, or each ligand monomer molecule is different (e.g., having different ligand monomer molecules of the TNF-like Superfamily members or a combination of TNF Superfamily members and TNF-like Superfamily members).

TABLE 2
TNF-like Superfamily members

| | Uniprot | |
|---|---|---|
| Names | Accession | Monomer molecule Sequence |
| Complement C1Q. | P02745 | SEQ ID NO: 239 (subcomponent A) |
| Comprised of three | (A) | QPRPAFSAIRRNPPMGGNVVIFDTVITNQEEPYQNHS |
| independent chains | P02746 | GRFVCTVPGYYYFTFQVLSQWEICLSIVSSSRGQVRR |
| (subcomponents A, B and | (B) | SLGFCDTTNKGLFQVVSGGMVLQLQQGDQVWVEKDPK |
| C) which come together | P02747 | KGHIYQGSEADSVFSGFLIFPS |
| to make a trimer with a | (C) | SEQ ID NO: 240 (subcomponent B) |
| TNF-like fold. | | TQKIAFSATRTINVPLRRDQTIRFDHVITNMNNNYEP |
| | | RSGKFTCKVPGLYYFTYHASSRGNLCVNLMRGRERAQ |
| | | KVVTFCDYAYNTFQVTTGGMVLKLEQGENVFLQATDK |
| | | NSLLGMEGANSIFSGFLLFPD |
| | | SEQ ID NO: 241 (subcomponent C) |
| | | KFQSVFTVTRQTHQPPAPNSLIRFNAVLTNPQGDYDT |
| | | STGKFTCKVPGLYYFVYHASHTANLCVLLYRSGVKVV |
| | | TFCGHTSKTNQVNSGGVLLRLQVGEEVWLAVNDYYDM |
| | | VGIQGSDSVFSGFLLFPD |
| C1QL1, Complement | O75973 | SEQ ID NO: 242 |
| component 1 Q | | PRVAFYAGLKNPHEGYEVLKFDDVVTNLGNNYDAASG |
| subcomponent-like 1, C1q | | KFTCNIPGTYFFTYHVLMRGGDGTSMWADLCKNGQVR |
| and tumor necrosis factor- | | ASAIAQDADQNYDYASNSVILHLDAGDEVFIKLDGGK |
| related protein 14, | | AHGGNSNKYSTFSGFIIYSD |
| C1q/TNF-related protein | | |
| 14 | | |
| C1QL2, Complement | Q7Z5L3 | SEQ ID NO: 243 |
| component 2 Q | | PKIAFYVGLKSPHEGYEVLKFDDVVTNLGNHYDPTTG |
| subcomponent-like 2, C1q | | KFSCQVRGIYFFTYHILMRGGDGTSMWADLCKNGQVR |
| and tumor necrosis factor- | | ASAIAQDADQNYDYASNSVVLHLDSGDEVYVKLDGGK |
| related protein 10, | | AHGGNNNKYSTFSGFLLYPD |
| C1q/TNF-related protein | | |
| 10 | | |
| C1QL3, Complement | Q5VWW1 | SEQ ID NO: 244 |
| component 3 Q | | PKIAFYAGLKRQHEGYEVLKFDDVVTNLGNHYDPTTG |
| subcomponent-like 3, C1q | | KFTCSIPGIYFFTYHVLMRGGDGTSMWADLCKNNQVR |
| and tumor necrosis factor- | | ASAIAQDADQNYDYASNSVVLHLEPGDEVYIKLDGGK |
| related protein 13, | | AHGGNNNKYSTFSGFIIYAD |
| C1q/TNF-related protein | | |
| 13 | | |
| Caprin-2 C1q Domain | Q6IMN6 | SEQ ID NO: 245 |
| | | MRVAFSAARTSNLAPGTLDQPIVFDLLLNNLGETFDL |
| | | QLGRFNCPVNGTYVFIFHMLKLAVNVPLYVNLMKNEE |
| | | VLVSAYANDGAPDHETASNHAILQLFQGDQIWLRLHR |
| | | GAIYGSSWKYSTFSGYLLYQD |
| Cerebellin-1 C1q Domain | P23435 | SEQ ID NO: 246 |
| | | GSAKVAFSAIRSTNHEPSEMSNRTMIIYFDQVLVNIG |
| | | NNFDSERSTFIAPRKGIYSFNFHVVKVYNRQTIQVSL |
| | | MLNGWPVISAFAGDQDVTREAASNGVLIQMEKGDRAY |
| | | LKLERGNLMGGWKYSTFSGFLVFPL |
| Adiponectin | Q15848 | SEQ ID NO: 247 |
| | | PGEGAYVYRSAFSVGLETYVTIPNMPIRFTKIFYNQQ |
| | | NHYDGSTGKFHCNIPGLYYFAYHITVYMKDVKVSLFK |
| | | KDKAMLFTYDQYQENNVDQASGSVLLHLEVGDQVWLQ |
| | | VYGEGERNGLYADNDNDSTFTGFLLYHDTN |

### Multifunctional Molecules Comprising TNFSF Ligands

In some embodiments, the TNFSF fusion molecule includes at least two, or at least three or at least four non-contiguous polypeptide chains. Exemplary TNFSF fusion molecules having at least two, or at least three or at least four non-contiguous polypeptide chains, wherein each chain can include a TNFSF member monomer molecule or linker joined dimer. In one embodiment, the TNFSF fusion molecule contains two immunoglobulin Fc domains which together form a complete TNFSF homotrimer or heterotrimer. In another embodiment, the TNFSF fusion molecule contains one immunoglobulin Fc domain, a complete heavy constant chain and a complete light constant chain in which the heavy and light chain form a complete TNFSF homotrimer or heterotrimer. In another embodiment, the TNFSF fusion molecule contains two complete heavy constant chains and two complete light constant chains in which each heavy and light chain form a complete TNFSF homotrimer or heterotrimer, which may be the same of different family members. In another embodiment, the TNFSF fusion molecule contains two complete heavy constant chains which form a complete TNFSF homotrimer or heterotrimer and two complete light constant chains in which each heavy and light chain form a complete TNFSF homotrimer or heterotrimer, which may be the same of different family members.

### Exemplary Multifunctional Molecules

In one embodiment, the multifunctional molecule is a TNFSF fusion molecule that is comprised of a heterodimeric, knob-in-a-hole (KiH) human immunoglobulin IgG1 Fc domain with a C-terminal CD40L wherein the protein sequence from the N-terminus to the C-terminus of one of the heavy chains is comprised of an Ig kappa signal peptide (optional), an immunoglobulin constant heavy domain containing the T366W mutation, a 3×4GS linker (SEQ ID NO: 84), a CD40L monomer molecule containing a C194A mutation, a 4GS linker (SEQ ID NO: 80) and a second CD40L containing a C194A mutation. In some embodiments, the multifunctional molecule comprising the amino acid sequence of:

(SEQ ID NO: 18)
METDTLLLWVLLLWVPGSTGDKTHTCPPCPAPELLGGPSVFLFPPKPKD
TLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQ
VYTLPPCREEMTKNQVSLWCLVKGFYPSDIAVEWESNGQPENNYKTTPP
VLDSDGSFFLYSKLTVDKSRWQQGNVFSCSVMHEALHNHYTQKSLSLSP
GKGGGGSGGGGSGGGGSGDQNPQIAAHVISEASSKTTSVLQWAEKGYYT
MSNNLVTLENGKQLTVKRQGLYYIYAQVTFCSNREASSQAPFIASLALK
SPGRFERILLRAANTHSSAKPCGQQSIHLGGVFELQPGASVFVNVTDPS
QVSHGTGFTSFGLLKLGGGGSGDQNPQIAAHVISEASSKTTSVLQWAEK
GYYTMSNNLVTLENGKQLTVKRQGLYYIYAQVTFCSNREASSQAPFIAS
LALKSPGRFERILLRAANTHSSAKPCGQQSIHLGGVFELQPGASVFVNV
TDPSQVSHGTGFTSFGLLKL

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 18 (with or without the signal peptide METDTLLLWVLLLWVPGSTG (SEQ ID NO: 74)).

In embodiments, the other heavy chain sequence from the N-terminus to the C-terminus is comprised of an Ig kappa signal peptide (optional), an immunoglobulin constant heavy domain containing the T366S, L368A and Y408V mutations; a 3×4GS linker (SEQ ID NO: 84) and a CD40L monomer molecule containing a C194A mutation:

(SEQ ID NO: 19)
METDTLLLWVLLLWVPGSTGDKTHTCPPCPAPELLGGPSVFLFPPKPKD
TLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQ
VCTLPPSREEMTKNQVSLSCAVKGFYPSDIAVEWESNGQPENNYKTTPP
VLDSDGSFFLVSKLTVDKSRWQQGNVFSCSVMHEALHNHYTQKSLSLSP
GKGGGGSGGGGSGGGGSGDQNPQIAAHVISEASSKTTSVLQWAEKGYYT
MSNNLVTLENGKQLTVKRQGLYYIYAQVTFCSNREASSQAPFIASLALK
SPGRFERILLRAANTHSSAKPCGQQSIHLGGVFELQPGASVFVNVTDPS
QVSHGTGFTSFGLLKL

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 19 (with or without the signal peptide METDTLLLWVLLLWVPGSTG (SEQ ID NO: 74)).

In another embodiment, the multifunctional molecule is a TNFSF fusion molecule that is comprised a heterodimeric KiH immunoglobulin IgG1 with a single CD40L in the Fab position of the molecule wherein the protein sequence for one heavy chain is comprised of an Ig kappa signal peptide (optional) and an immunoglobulin constant heavy domain containing the T366W mutation:

(SEQ ID NO: 20)
METDTLLLWVLLLWVPGSTGDKTHTCPPCPAPELLGGPSVFLFPPKPKD
TLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQ
VYTLPPCREEMTKNQVSLWCLVKGFYPSDIAVEWESNGQPENNYKTTPP
VLDSDGSFFLYSKLTVDKSRWQQGNVFSCSVMHEALHNHYTQKSLSLSP
GK

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 20 (with or without the signal peptide METDTLLLWVLLLWVPGSTG (SEQ ID NO: 74)).

In embodiments, the other heavy chain is comprised of an Ig kappa signal peptide (optional), a CD40L monomer molecule containing a C194A mutation, a 4GS linker (SEQ ID NO: 80), a second CD40L containing a C194A mutation, a 3×4GS linker (SEQ ID NO: 84) and a complete IgG1 heavy constant region composed of CH1, CH2 and CH3 with the T366S, L368A and Y408V mutations:

(SEQ ID NO: 21)
METDTLLLWVLLLWVPGSTGGDQNPQIAAHVISEASSKTTSVLQWAEKG
YYTMSNNLVTLENGKQLTVKRQGLYYIYAQVTFCSNREASSQAPFIASL
ALKSPGRFERILLRAANTHSSAKPCGQQSIHLGGVFELQPGASVFVNVT
DPSQVSHGTGFTSFGLLKLGGGGSGDQNPQIAAHVISEASSKTTSVLQW
AEKGYYTMSNNLVTLENGKQLTVKRQGLYYIYAQVTFCSNREASSQAPF
IASLALKSPGRFERILLRAANTHSSAKPCGQQSIHLGGVFELQPGASVF
VNVTDPSQVSHGTGFTSFGLLKLGGGGSGGGGSGGGGSASTKGPSVFPL
APSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVS
NKALPAPIEKTISKAKGQPREPQVCTLPPSREEMTKNQVSLSCAVKGFY
PSDIAVEWESNGQPENNYKTTPPVLDSDGSFFLVSKLTVDKSRWQQGNV
FSCSVMHEALHNHYTQKSLSLSPGK

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 21 (with or without the signal peptide METDTLLLWVLLLWVPGSTG (SEQ ID NO: 74)).

In embodiments, the light chain that pairs with SEQ ID NO: 21 (with or without the signal peptide METDTLLLWVLLLWVPGSTG (SEQ ID NO: 74)) is comprised of an Ig kappa signal peptide (optional), a CD40L monomer molecule containing a C194A mutation, a 3×4GS linker (SEQ ID NO: 84) and an Ig kappa light chain constant domain:

(SEQ ID NO: 22)
METDTLLLWVLLLWVPGSTGGDQNPQIAAHVISEASSKTTSVLQWAEKG
YYTMSNNLVTLENGKQLTVKRQGLYYIYAQVTFCSNREASSQAPFIASL
ALKSPGRFERILLRAANTHSSAKPCGQQSIHLGGVFELQPGASVFVNVT
DPSQVSHGTGFTSFGLLKLGGGGSGGGGSGGGGSRTVAAPSVFIFPPSD
EQLKSGTASVVCLLNNFYPREAKVQWKVDNALQSGNSQESVTEQDSKDS
TYSLSSTLTLSKADYEKHKVYACEVTHQGLSSPVTKSFNRGEC

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 22 (with or without the signal peptide METDTLLLWVLLLWVPGSTG (SEQ ID NO: 74)).

In another embodiment, the multifunctional molecule is a TNFSF fusion molecule that is comprised of a heterodimeric, KiH human immunoglobulin IgG1 Fc domain with a C-terminal GITRL wherein the protein sequence from the N-terminus to the C-terminus of one of the heavy chains is comprised of an Ig kappa signal peptide (optional), an immunoglobulin constant heavy domain containing the T366W mutation, a 4×4GS linker (SEQ ID NO: 190), a GITRL monomer molecule, a glycine residue and a second GITRL monomer molecule:

(SEQ ID NO: 23)
METDTLLLWVLLLWVPGSTGDKTHTCPPCPAPELLGGPSVFLFPPKPKD
TLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQ
VYTLPPCREEMTKNQVSLWCLVKGFYPSDIAVEWESNGQPENNYKTTPP
VLDSDGSFFLYSKLTVDKSRWQQGNVFSCSVMHEALHNHYTQKSLSLSP
GKGGGGSGGGGSGGGGSGGGGSQLETAKEPCMAKFGPLPSKWQMASSEP
PCVNKVSDWKLEILQNGLYLIYGQVAPNANYNDVAPFEVRLYKNKDMIQ
TLTNKSKIQNVGGTYELHVGDTIDLIFNSEHQVLKNNTYWGIILLANGQ
LETAKEPCMAKFGPLPSKWQMASSEPPCVNKVSDWKLEILQNGLYLIYG
QVAPNANYNDVAPFEVRLYKNKDMIQTLTNKSKIQNVGGTYELHVGDTI
DLIFNSEHQVLKNNTYWGIILLANPQFIS

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 23 (with or without the signal peptide METDTLLLWVLLLWVPGSTG (SEQ ID NO: 74)).

In embodiments, the other heavy chain sequence from the N-terminus to the C-terminus is comprised of an Ig kappa signal peptide (optional), an immunoglobulin constant heavy domain containing the T366S, L368A and Y408V mutations; a 4×4GS linker (SEQ ID NO: 190) and a GITRL monomer molecule:

(SEQ ID NO: 24)
METDTLLLWVLLLWVPGSTGDKTHTCPPCPAPELLGGPSVFLFPPKPKD
TLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQ
VCTLPPSREEMTKNQVSLSCAVKGFYPSDIAVEWESNGQPENNYKTTPP
VLDSDGSFFLVSKLTVDKSRWQQGNVFSCSVMHEALHNHYTQKSLSLSP
GKGGGGSGGGGSGGGGSGGGGSQLETAKEPCMAKFGPLPSKWQMASSEP
PCVNKVSDWKLEILQNGLYLIYGQVAPNANYNDVAPFEVRLYKNKDMIQ
TLTNKSKIQNVGGTYELHVGDTIDLIFNSEHQVLKNNTYWGIILLANPQ
FIS

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 24 (with or without the signal peptide METDTLLLWVLLLWVPGSTG (SEQ ID NO: 74)).

In another embodiment, the multifunctional molecule is a TNFSF fusion molecule that is comprised a heterodimeric KiH immunoglobulin IgG1 with a single GITRL in the Fab position of the molecule wherein the protein sequence for one heavy chain is comprised of an Ig kappa signal peptide (optional) and an immunoglobulin constant heavy domain containing the T366W mutation (SEQ ID NO: 20) (with or without the signal peptide METDTLLLWVLLLWVPGSTG (SEQ ID NO: 74).

In embodiments, the other heavy chain is comprised of an Ig kappa signal peptide (optional), a GITRL monomer molecule, a glycine residue, a second GITRL monomer molecule, a 3×4GS linker (SEQ ID NO: 84) and a complete IgG1 heavy constant region composed of CH1, CH2 and CH3 with the T366S, L368A and Y408V mutations:

(SEQ ID NO: 25)
METDTLLLWVLLLWVPGSTGQLETAKEPCMAKFGPLPSKWQMASSEPPC
VNKVSDWKLEILQNGLYLIYGQVAPNANYNDVAPFEVRLYKNKDMIQTL
TNKSKIQNVGGTYELHVGDTIDLIFNSEHQVLKNNTYWGIILLANGQLE
TAKEPCMAKFGPLPSKWQMASSEPPCVNKVSDWKLEILQNGLYLIYGQV
APNANYNDVAPFEVRLYKNKDMIQTLTNKSKIQNVGGTYELHVGDTIDL
IFNSEHQVLKNNTYWGIILLANPQFISGGGGSGGGGSGGGGSASTKGPS
VFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDK
THTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYK
CKVSNKALPAPIEKTISKAKGQPREPQVCTLPPSREEMTKNQVSLSCAV
KGFYPSDIAVEWESNGQPENNYKTTPPVLDSDGSFFLVSKLTVDKSRWQ
QGNVFSCSVMHEALHNHYTQKSLSLSPGK

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 25 (with or without the signal peptide METDTLLLWVLLLWVPGSTG (SEQ ID NO: 74)).

In other embodiments, the light chain that pairs with SEQ ID NO: 25 (with or without the signal peptide METDTLLLWVLLLWVPGSTG (SEQ ID NO: 74)) is comprised of an Ig kappa signal peptide (optional), a GITRL monomer molecule, a 3×4GS linker (SEQ ID NO: 84) and an Ig kappa light chain constant domain:

(SEQ ID NO: 26)
METDTLLLWVLLLWVPGSTGQLETAKEPCMAKFGPLPSKWQMASSEPPCV
NKVSDWKLEILQNGLYLIYGQVAPNANYNDVAPFEVRLYKNKDMIQTLTN
KSKIQNVGGTYELHVGDTIDLIFNSEHQVLKNNTYWGIILLANPQFISGG
GGSGGGGSGGGGSRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 26 (with or without the signal peptide METDTLLLWVLLLWVPGSTG (SEQ ID NO: 74)).

In another embodiment, the multifunctional molecule is a TNFSF fusion molecule that is comprised of a heterodimeric, KiH human immunoglobulin IgG1 Fc domain with a C-terminal TNFα wherein the protein sequence from the N-terminus to the C-terminus of one of the heavy chains is comprised of an Ig kappa signal peptide (optional), an immunoglobulin constant heavy domain containing the T366W mutation, a 4×4GS linker (SEQ ID NO: 190), a TNFα monomer molecule, a GGSGG linker (SEQ ID NO: 81) and a second TNFα monomer molecule:

(SEQ ID NO: 27)
METDTLLLWVLLLWVPGSTGDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPCREEMTKNQVSLWCLVKGFYPSDIAVEWESNGQPENNYKTTPPVLDS
DGSFFLYSKLTVDKSRWQQGNVFSCSVMHEALHNHYTQKSLSLSPGKGGG
GSGGGGSGGGGSGGGGSRTPSDKPVAHVVANPQAEGQLQWLNRRANALLA
NGVELRDNQLVVPSEGLYLIYSQVLFKGQGCPSTHVLLTHTISRIAVSYQ
TKVNLLSAIKSPCQRETPEGAEAKPWYEPIYLGGVFQLEKGDRLSAEINR
PDYLDFAESGQVYFGIIALGGSGGSDKPVAHVVANPQAEGQLQWLNRRAN
ALLANGVELRDNQLVVPSEGLYLIYSQVLFKGQGCPSTHVLLTHTISRIA
VSYQTKVNLLSAIKSPCQRETPEGAEAKPWYEPIYLGGVFQLEKGDRLSA
EINRPDYLDFAESGQVYFGIIAL

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 27 (with or without the signal peptide METDTLLLWVLLLWVPGSTG (SEQ ID NO: 74)).

In embodiments, the other heavy chain sequence from the N-terminus to the C-terminus is comprised of an Ig kappa signal peptide (optional), an immunoglobulin constant heavy domain containing the T366S, L368A and Y408V mutations; a 4×4GS linker (SEQ ID NO: 190) and a TNFα monomer molecule:

(SEQ ID NO: 28)
METDTLLLWVLLLWVPGSTGDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVCT
LPPSREEMTKNQVSLSCAVKGFYPSDIAVEWESNGQPENNYKTTPPVLDS
DGSFFLVSKLTVDKSRWQQGNVFSCSVMHEALHNHYTQKSLSLSPGKGGG
GSGGGGSGGGGSGGGGSRTPSDKPVAHVVANPQAEGQLQWLNRRANALLA
NGVELRDNQLVVPSEGLYLIYSQVLFKGQGCPSTHVLLTHTISRIAVSYQ
TKVNLLSAIKSPCQRETPEGAEAKPWYEPIYLGGVFQLEKGDRLSAEINR
PDYLDFAESGQVYFGIIAL

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 28 (with or without the signal peptide METDTLLLWVLLLWVPGSTG (SEQ ID NO: 74)).

In another embodiment, the multifunctional molecule is a TNFSF fusion molecule that is comprised a heterodimeric KiH immunoglobulin IgG1 with a single TNFα in the Fab position of the molecule wherein the protein sequence for one heavy chain is comprised of an Ig kappa signal peptide (optional) and an immunoglobulin constant heavy domain containing the T366W mutation (SEQ ID NO: 20) (with or without the signal peptide METDTLLLWVLLLWVPGSTG (SEQ ID NO: 74)), while the other heavy chain is comprised of an Ig kappa signal peptide (optional), a TNFα monomer molecule, a GGSGG linker (SEQ ID NO: 81), a second TNFα monomer molecule, a 3×4GS linker (SEQ ID NO: 84) and a complete IgG1 heavy constant region composed of CH1, CH2 and CH3 with the T366S, L368A and Y408V mutations:

(SEQ ID NO: 29)
METDTLLLWVLLLWVPGSTGRTPSDKPVAHVVANPQAEGQLQWLNRRANA
LLANGVELRDNQLVVPSEGLYLIYSQVLFKGQGCPSTHVLLTHTISRIAV
SYQTKVNLLSAIKSPCQRETPEGAEAKPWYEPIYLGGVFQLEKGDRLSAE
INRPDYLDFAESGQVYFGIIALGGSGGSDKPVAHVVANPQAEGQLQWLNR
RANALLANGVELRDNQLVVPSEGLYLIYSQVLFKGQGCPSTHVLLTHTIS
RIAVSYQTKVNLLSAIKSPCQRETPEGAEAKPWYEPIYLGGVFQLEKGDR
LSAEINRPDYLDFAESGQVYFGIIALGGGGSGGGGSGGGGSASTKGPSVF
PLAPSSKSTSGGTAALGCLVKDYFPEPVTVSWNSGALTSGVHTFPAVLQS
SGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNK
ALPAPIEKTISKAKGQPREPQVCTLPPSREEMTKNQVSLSCAVKGFYPSD
IAVEWESNGQPENNYKTTPPVLDSDGSFFLVSKLTVDKSRWQQGNVFSCS
VMHEALHNHYTQKSLSLSPGK

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 29 (with or without the signal peptide METDTLLLWVLLLWVPGSTG (SEQ ID NO: 74)).

In embodiments, the light chain that pairs with SEQ ID NO: 29 (with or without the signal peptide METDTLLLWVLLLWVPGSTG (SEQ ID NO: 74)) is comprised of an Ig kappa signal peptide (optional), a TNFα monomer molecule, a 3×4GS linker (SEQ ID NO: 84) and an Ig kappa light chain constant domain:

(SEQ ID NO: 30)
METDTLLLWVLLLWVPGSTGRTPSDKPVAHVVANPQAEGQLQWLNRRANA
LLANGVELRDNQLVVPSEGLYLIYSQVLFKGQGCPSTHVLLTHTISRIAV
SYQTKVNLLSAIKSPCQRETPEGAEAKPWYEPIYLGGVFQLEKGDRLSAE
INRPDYLDFAESGQVYFGIIALGGGGSGGGGSGGGGSRTVAAPSVFIFPP
SDEQLKSGTASVVCLLNNFYPREAKVQWKVDNALQSGNSQESVTEQDSKD
STYSLSSTLTLSKADYEKHKVYACEVTHQGLSSPVTKSFNRGEC

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 30 (with or without the signal peptide METDTLLLWVLLLWVPGSTG (SEQ ID NO: 74)).

In another embodiment, the multifunctional molecule is a TNFSF fusion molecule that is comprised of a heterodimeric, KiH human immunoglobulin IgG1 Fc domain with a C-terminal FASL wherein the protein sequence from the N-terminus to the C-terminus of one of the heavy chains is comprised of an Ig kappa signal peptide (optional), an immunoglobulin constant heavy domain containing the T366W mutation, a 4×4GS linker (SEQ ID NO: 190), a FASL monomer molecule, a GGSGG linker (SEQ ID NO: 81) and a second FASL monomer molecule:

(SEQ ID NO: 31)
METDTLLLWVLLLWVPGSTGDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPCREEMTKNQVSLWCLVKGFYPSDIAVEWESNGQPENNYKTTPPVLDS
DGSFFLYSKLTVDKSRWQQGNVFSCSVMHEALHNHYTQKSLSLSPGKGGG
GSGGGGSGGGGSGGGGSLRKVAHLTGKSNSRSMPLEWEDTYGIVLLSGVK
YKKGGLVINETGLYFVYSKVYFRGQSCNNLPLSHKVYMRNSKYPQDLVMM
EGKMMSYCTTGQMWARSSYLGAVFNLTSADHLYVNVSELSLVNFEESQTF
FGLYKLGGSGGKVAHLTGKSNSRSMPLEWEDTYGIVLLSGVKYKKGGLVI
NETGLYFVYSKVYFRGQSCNNLPLSHKVYMRNSKYPQDLVMMEGKMMSYC
TTGQMWARSSYLGAVFNLTSADHLYVNVSELSLVNFEESQTFFGLYKL

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 31 (with or without the signal peptide METDTLLLWVLLLWVPGSTG (SEQ ID NO: 74)).

In embodiments, the other heavy chain sequence from the N-terminus to the C-terminus is comprised of an Ig kappa signal peptide (optional), an immunoglobulin constant heavy domain containing the T366S, L368A and Y408V mutations; a 4×4GS linker (SEQ ID NO: 190) and a FASL monomer molecule:

(SEQ ID NO: 32)
METDTLLLWVLLLWVPGSTGDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVCT
LPPSREEMTKNQVSLSCAVKGFYPSDIAVEWESNGQPENNYKTTPPVLDS
DGSFFLVSKLTVDKSRWQQGNVFSCSVMHEALHNHYTQKSLSLSPGKGGG
GSGGGGSGGGGSGGGGSLRKVAHLTGKSNSRSMPLEWEDTYGIVLLSGVK
YKKGGLVINETGLYFVYSKVYFRGQSCNNLPLSHKVYMRNSKYPQDLVMM
EGKMMSYCTTGQMWARSSYLGAVFNLTSADHLYVNVSELSLVNFEESQTF
FGLYKL

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 32 (with or without the signal peptide METDTLLLWVLLLWVPGSTG (SEQ ID NO: 74)).

In another embodiment, the TNFSF fusion molecule is comprised a heterodimeric KiH immunoglobulin IgG1 with a single FASL in the Fab position of the molecule wherein the protein sequence for one heavy chain is comprised of an Ig kappa signal peptide (optional) and an immunoglobulin constant heavy domain containing the T366W mutation (SEQ ID NO: 20) (with or without the signal peptide METDTLLLWVLLLWVPGSTG (SEQ ID NO: 74)), while the other heavy chain is comprised of an Ig kappa signal peptide (optional), a FASL monomer molecule, a GGSGG linker (SEQ ID NO: 81), a second FASL monomer molecule, a 3×4GS linker (SEQ ID NO: 84) and a complete IgG1 heavy constant region composed of CH1, CH2 and CH3 with the T366S, L368A and Y408V mutations:

(SEQ ID NO: 33)
METDTLLLWVLLLWVPGSTGLRKVAHLTGKSNSRSMPLEWEDTYGIVLLS
GVKYKKGGLVINETGLYFVYSKVYFRGQSCNNLPLSHKVYMRNSKYPQDL
VMMEGKMMSYCTTGQMWARSSYLGAVFNLTSADHLYVNVSELSLVNFEES
QTFFGLYKLGGSGGKVAHLTGKSNSRSMPLEWEDTYGIVLLSGVKYKKGG
LVINETGLYFVYSKVYFRGQSCNNLPLSHKVYMRNSKYPQDLVMMEGKMM
SYCTTGQMWARSSYLGAVFNLTSADHLYVNVSELSLVNFEESQTFFGLYK
LGGGGSGGGGSGGGGSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVCTL
PPSREEMTKNQVSLSCAVKGFYPSDIAVEWESNGQPENNYKTTPPVLDSD
GSFFLVSKLTVDKSRWQQGNVFSCSVMHEALHNHYTQKSLSLSPGK

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 33 (with or without the signal peptide METDTLLLWVLLLWVPGSTG (SEQ ID NO: 74)).

The light chain that pairs with SEQ ID NO: 33 (with or without the signal peptide METDTLLLWVLLLWVPGSTG (SEQ ID NO: 74)) is comprised of an Ig kappa signal peptide (optional), a FASL monomer molecule, a 3×4GS linker (SEQ ID NO: 84) and an Ig kappa light chain constant domain:

(SEQ ID NO: 34)
METDTLLLWVLLLWVPGSTGLRKVAHLTGKSNSRSMPLEWEDTYGIVLLS
GVKYKKGGLVINETGLYFVYSKVYFRGQSCNNLPLSHKVYMRNSKYPQDL
VMMEGKMMSYCTTGQMWARSSYLGAVFNLTSADHLYVNVSELSLVNFEES
QTFFGLYKLGGGGSGGGGSGGGGSRTVAAPSVFIFPPSDEQLKSGTASVV
CLLNNFYPREAKVQWKVDNALQSGNSQESVTEQDSKDSTYSLSSTLTLSK
ADYEKHKVYACEVTHQGLSSPVTKSFNRGEC

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 34 (with or without the signal peptide METDTLLLWVLLLWVPGSTG (SEQ ID NO: 74)).

In another embodiment, the TNFSF fusion molecule is comprised of a heterodimeric, KiH human immunoglobulin IgG1 Fc domain with a C-terminal LIGHT wherein the protein sequence from the N-terminus to the C-terminus of one of the heavy chains is comprised of an Ig kappa signal peptide (optional), an immunoglobulin constant heavy domain containing the T366W mutation, a 4×4GS linker (SEQ ID NO: 190), a LIGHT monomer molecule, a GGSGG linker (SEQ ID NO: 81) and a second LIGHT monomer molecule:

(SEQ ID NO: 35)
METDTLLLWVLLLWVPGSTGDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPCREEMTKNQVSLWCLVKGFYPSDIAVEWESNGQPENNYKTTPPVLDS
DGSFFLYSKLTVDKSRWQQGNVFSCSVMHEALHNHYTQKSLSLSPGKGGG
GSGGGGSGGGGSGGGGSNPAAHLTGANSSLTGSGGPLLWETQLGLAFLRG
LSYHDGALVVTKAGYYYIYSKVQLGGVGCPLGLASTITHGLYKRTPRYPE
ELELLVSQQSPCGRATSSSRVWWDSSFLGGVVHLEAGEKVVVRVLDERLV
RLRDGTRSYFGAFMVGGSGGNPAAHLTGANSSLTGSGGPLLWETQLGLAF
LRGLSYHDGALVVTKAGYYYIYSKVQLGGVGCPLGLASTITHGLYKRTPR
YPEELELLVSQQSPCGRATSSSRVWWDSSFLGGVVHLEAGEKVVVRVLDE
RLVRLRDGTRSYFGAFMV

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 35 (with or without the signal peptide METDTLLLWVLLLWVPGSTG (SEQ ID NO: 74)).

In embodiments, the other heavy chain sequence from the N-terminus to the C-terminus is comprised of an Ig kappa signal peptide (optional), an immunoglobulin constant heavy domain containing the T366S, L368A and Y408V mutations; a 4×4GS linker (SEQ ID NO: 190) and a LIGHT monomer molecule:

(SEQ ID NO: 36)
METDTLLLWVLLLWVPGSTGDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVCT
LPPSREEMTKNQVSLSCAVKGFYPSDIAVEWESNGQPENNYKTTPPVLDS
DGSFFLVSKLTVDKSRWQQGNVFSCSVMHEALHNHYTQKSLSLSPGKGGG
GSGGGGSGGGGSGGGGSNPAAHLTGANSSLTGSGGPLLWETQLGLAFLRG
LSYHDGALVVTKAGYYYIYSKVQLGGVGCPLGLASTITHGLYKRTPRYPE
ELELLVSQQSPCGRATSSSRVWWDSSFLGGVVHLEAGEKVVVRVLDERLV
RLRDGTRSYFGAFMV

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 36 (with or without the signal peptide METDTLLLWVLLLWVPGSTG (SEQ ID NO: 74)).

In another embodiment, the TNFSF fusion molecule is comprised a heterodimeric KiH immunoglobulin IgG1 with a single LIGHT in the Fab position of the molecule wherein the protein sequence for one heavy chain is comprised of an Ig kappa signal peptide (optional) and an immunoglobulin constant heavy domain containing the T366W mutation (SEQ ID NO: 20) (with or without the signal peptide METDTLLLWVLLLWVPGSTG (SEQ ID NO: 74)), while the other heavy chain is comprised of an Ig kappa signal peptide (optional), a LIGHT monomer molecule, a GGSGG linker (SEQ ID NO: 81), a second LIGHT monomer molecule, a 3×4GS linker (SEQ ID NO: 84) and a complete IgG1 heavy constant region composed of CH1, CH2 and CH3 with the T366S, L368A and Y408V mutations:

(SEQ ID NO: 37)
METDTLLLWVLLLWVPGSTGNPAAHLTGANSSLTGSGGPLLWETQLGLAF
LRGLSYHDGALVVTKAGYYYIYSKVQLGGVGCPLGLASTITHGLYKRTPR
YPEELELLVSQQSPCGRATSSSRVWWDSSFLGGVVHLEAGEKVVVRVLDE
RLVRLRDGTRSYFGAFMVGGSGGNPAAHLTGANSSLTGSGGPLLWETQLG
LAFLRGLSYHDGALVVTKAGYYYIYSKVQLGGVGCPLGLASTITHGLYKR
TPRYPEELELLVSQQSPCGRATSSSRVWWDSSFLGGVVHLEAGEKVVVRV
LDERLVRLRDGTRSYFGAFMVGGGGSGGGGSGGGGSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPA
PELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVCTLPPSREEMTKNQVSLSCAVKGFYPSDIAVEW
ESNGQPENNYKTTPPVLDSDGSFFLVSKLTVDKSRWQQGNVFSCSVMHEA
LHNHYTQKSLSLSPGK

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 37 (with or without the signal peptide METDTLLLWVLLLWVPGSTG (SEQ ID NO: 74)).

The light chain that pairs with SEQ ID NO: 37 (with or without the signal peptide METDTLLLWVLLLWVPGSTG (SEQ ID NO: 74)) is comprised of an Ig kappa signal peptide (optional), a LIGHT monomer molecule, a 3×4GS linker (SEQ ID NO: 84) and an Ig kappa light chain constant domain:

(SEQ ID NO: 38)
METDTLLLWVLLLWVPGSTGNPAAHLTGANSSLTGSGGPLLWETQLGLAF
LRGLSYHDGALVVTKAGYYYIYSKVQLGGVGCPLGLASTITHGLYKRTPR
YPEELELLVSQQSPCGRATSSSRVWWDSSFLGGVVHLEAGEKVVVRVLDE
RLVRLRDGTRSYFGAFMVGGGGSGGGGSGGGGSRTVAAPSVFIFPPSDEQ
LKSGTASVVCLLNNFYPREAKVQWKVDNALQSGNSQESVTEQDSKDSTYS
LSSTLTLSKADYEKHKVYACEVTHQGLSSPVTKSFNRGEC

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 38 (with or without the signal peptide METDTLLLWVLLLWVPGSTG (SEQ ID NO: 74)).

In one embodiment, the multifunctional molecule is a TNFSF fusion molecule that is comprised of a heterodimeric, knob-in-a-hole (KiH) human immunoglobulin IgG1 Fc domain with the hinge disulfides mutated from cysteine to serine with a C-terminal CD40L wherein the protein sequence from the N-terminus to the C-terminus of one of the heavy chains is comprised of an Ig kappa signal peptide (optional), an immunoglobulin constant heavy domain containing the T366W mutation, a 3×4GS linker (SEQ ID NO: 84), a CD40L monomer molecule containing a C194A mutation, a 4GS linker (SEQ ID NO: 80) and a second CD40L containing a C194A mutation. In some embodiments, the multifunctional molecule comprising the amino acid sequence of:

(SEQ ID NO: 122)
METDTLLLWVLLLWVPGSTGDKTHTSPPSPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSREEMTKNQVSLWCLVKGFYPSDIAVEWESNGQPENNYKTTPPVLDS
DGSFFLYSKLTVDKSRWQQGNVFSCSVMHEALHNHYTQKSLSLSPGKGGG
GSGGGGSGGGGSGDQNPQIAAHVISEASSKTTSVLQWAEKGYYTMSNNLV
TLENGKQLTVKRQGLYYIYAQVTFCSNREASSQAPFIASLALKSPGRFER
ILLRAANTHSSAKPCGQQSIHLGGVFELQPGASVFVNVTDPSQVSHGTGF
TSFGLLKLGGGGSGDQNPQIAAHVISEASSKTTSVLQWAEKGYYTMSNNL
VTLENGKQLTVKRQGLYYIYAQVTFCSNREASSQAPFIASLALKSPGRFE
RILLRAANTHSSAKPCGQQSIHLGGVFELQPGASVFVNVTDPSQVSHGTG
FTSFGLLKL

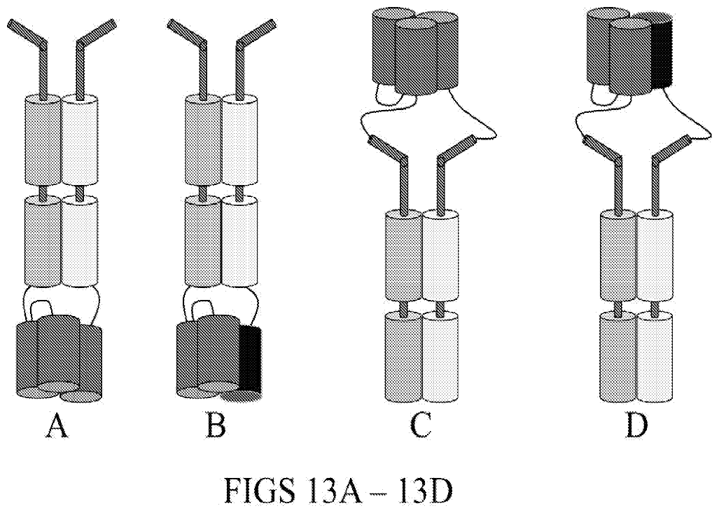

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 122 (with or without the signal peptide METDTLLLWVLLLWVPGSTG (SEQ ID NO: 74)). Underlined in SEQ ID NO: 122 above are positions where a cysteine residue is mutated to a serine residue to remove a disulfide bond. For a representative configuration, see, for example, FIG. 13A.

In embodiments, the other heavy chain sequence from the N-terminus to the C-terminus is comprised of an Ig kappa signal peptide (optional), an immunoglobulin constant heavy domain containing the T366S, L368A and Y408V mutations; a 3×4GS linker (SEQ ID NO: 84) and a CD40L monomer molecule containing a C194A mutation:

(SEQ ID NO: 123)
METDTLLLWVLLLWVPGSTGDKTHTSPPSPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSREEMTKNQVSLSCAVKGFYPSDIAVEWESNGQPENNYKTTPPVLDS
DGSFFLVSKLTVDKSRWQQGNVFSCSVMHEALHNHYTQKSLSLSPGKGGG
GSGGGGSGGGGSGDQNPQIAAHVISEASSKTTSVLQWAEKGYYTMSNNLV
TLENGKQLTVKRQGLYYIYAQVTFCSNREASSQAPFIASLALKSPGRFER
ILLRAANTHSSAKPCGQQSIHLGGVFELQPGASVFVNVTDPSQVSHGTGF
TSFGLLKL

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 123 (with or without the signal peptide METDTLLLWVLLLWVPGSTG (SEQ ID NO: 74)). Underlined in SEQ ID NO: 123 above are positions where a cysteine residue is mutated to a serine residue or a tyrosine residue to remove a disulfide bond.

In embodiments, the multifunctional molecule comprises a heavy chain comprising an Ig kappa signal peptide (optional), a GITRL monomer molecule, a glycine residue, a second GITRL monomer molecule, a 3×4GS linker (SEQ ID NO: 84) and a complete IgG1 heavy constant region composed of CH1 with the C-terminal cysteine used to make the interchain disulfide with the constant light domain removed, CH2 and CH3 with the T366S, L368A and Y408V mutations:

(SEQ ID NO: 124)
METDTLLLWVLLLWVPGSTGQLETAKEPCMAKFGPLPSKWQMASSEPPCV
NKVSDWKLEILQNGLYLIYGQVAPNANYNDVAPFEVRLYKNKDMIQTLTN
KSKIQNVGGTYELHVGDTIDLIFNSEHQVLKNNTYWGIILLANGQLETAK
EPCMAKFGPLPSKWQMASSEPPCVNKVSDWKLEILQNGLYLIYGQVAPNA
NYNDVAPFEVRLYKNKDMIQTLTNKSKIQNVGGTYELHVGDTIDLIFNSE
HQVLKNNTYWGIILLANPQFISGGGGSGGGGSGGGGSASTKGPSVFPLAP
SSKSTSGGTAALGCLVKDYFPEPVTVSWNSGALTSGVHTFPAVLQSSGLY
SLSSVVTVPSSSLGTQTYICNVNHKPSNTKVDKRVEPKSDKTHTCPPCPA
PELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVCTLPPSREEMTKNQVSLSCAVKGFYPSDIAVEW
ESNGQPENNYKTTPPVLDSDGSFFLVSKLTVDKSRWQQGNVFSCSVMHEA
LHNHYTQKSLSLSPGK

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 124 (with or without the signal peptide METDTLLLWVLLLWVPGSTG (SEQ ID NO: 74)). In SEQ ID NO: 124, a cysteine residue in the hinge domain has been deleted. SEQ ID NO: 124 comprises a hinge region of the amino acid sequence of EPKSDKTHTCP (SEQ ID NO: 181) whereas a corresponding wild type IgG1 comprises a hinge region of the amino acid sequence of EPKSCDKTHTCP (SEQ ID NO: 182.)

In other embodiments, the light chain that pairs with SEQ ID NO: 124 (with or without the signal peptide METDTLLLWVLLLWVPGSTG (SEQ ID NO: 74)) is comprised of an Ig kappa signal peptide (optional), a GITRL monomer molecule, a 3×4GS linker (SEQ ID NO: 84) and an Ig kappa light chain constant domain with the C-terminal cysteine used to make the interchain disulfide with CH1 removed:

(SEQ ID NO: 125)
METDTLLLWVLLLWVPGSTGQLETAKEPCMAKFGPLPSKWQMASSEPPCV
NKVSDWKLEILQNGLYLIYGQVAPNANYNDVAPFEVRLYKNKDMIQTLTN
KSKIQNVGGTYELHVGDTIDLIFNSEHQVLKNNTYWGIILLANPQFISGG
GGSGGGGSGGGGSRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGE

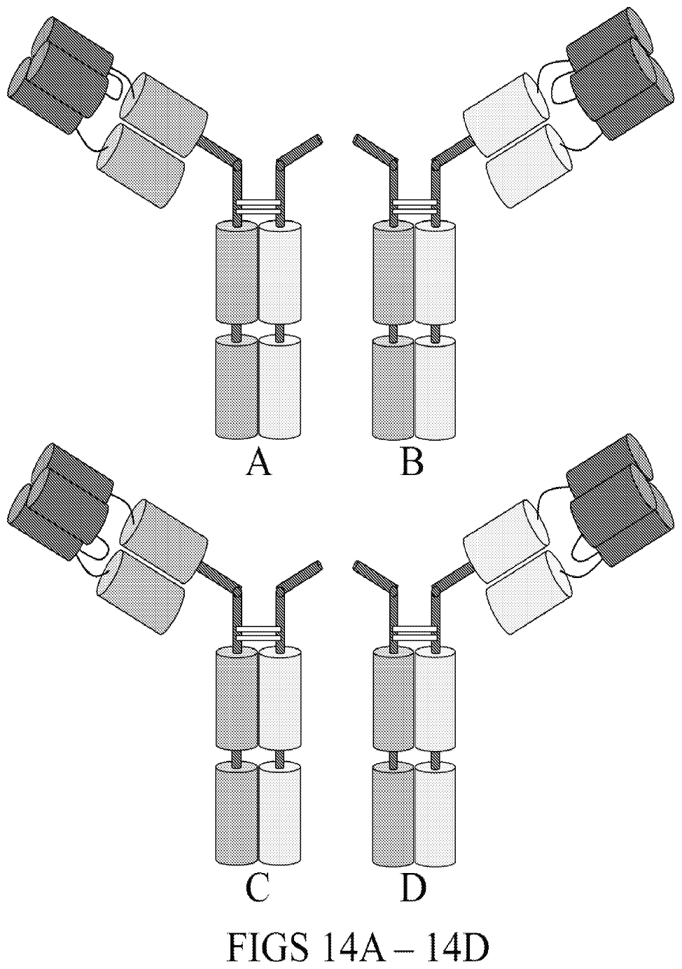

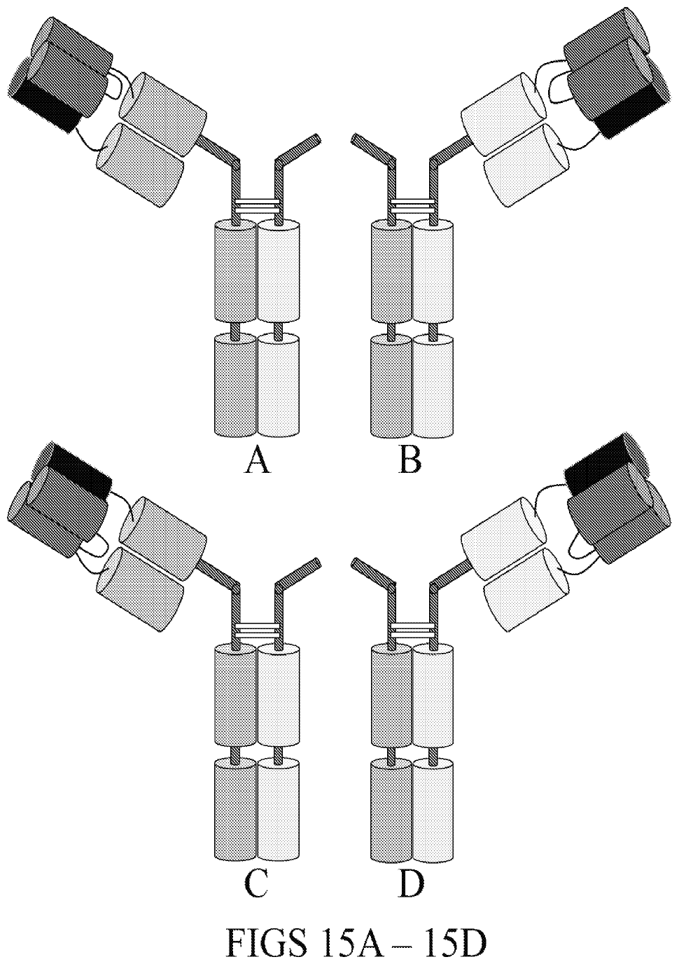

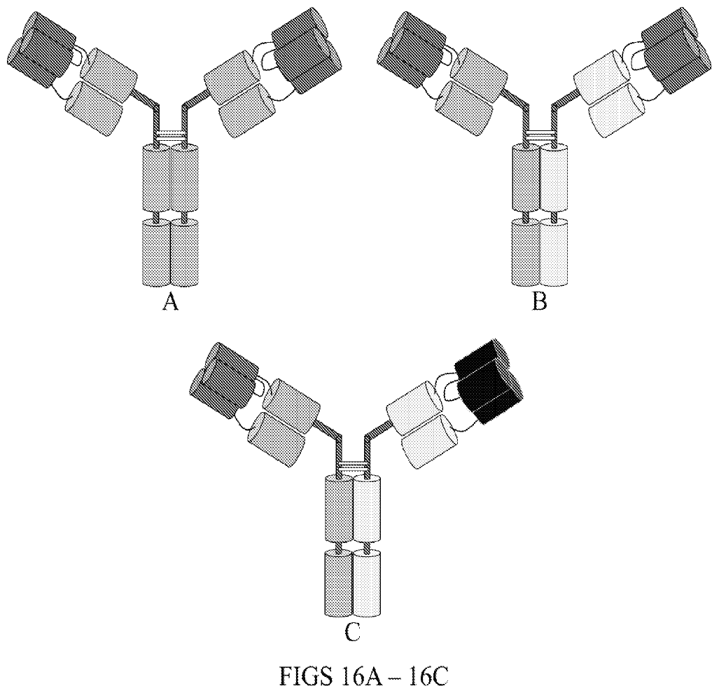

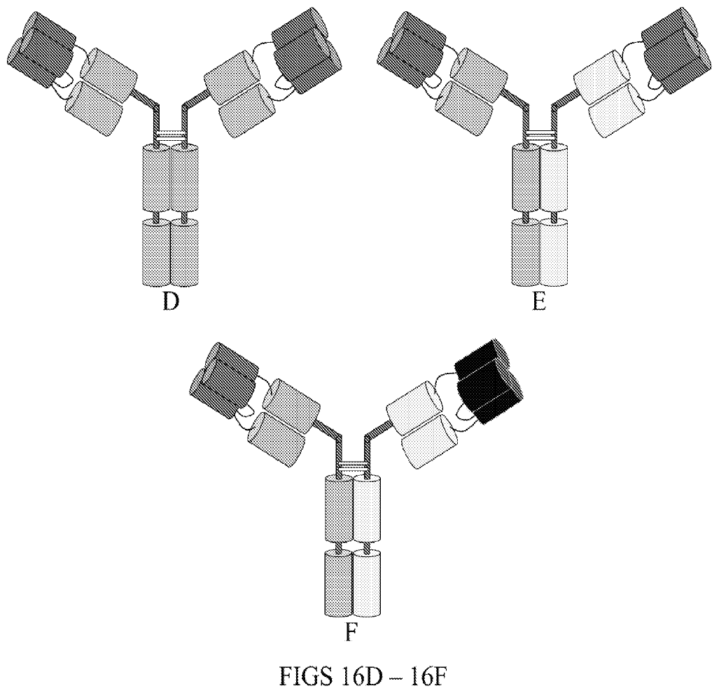

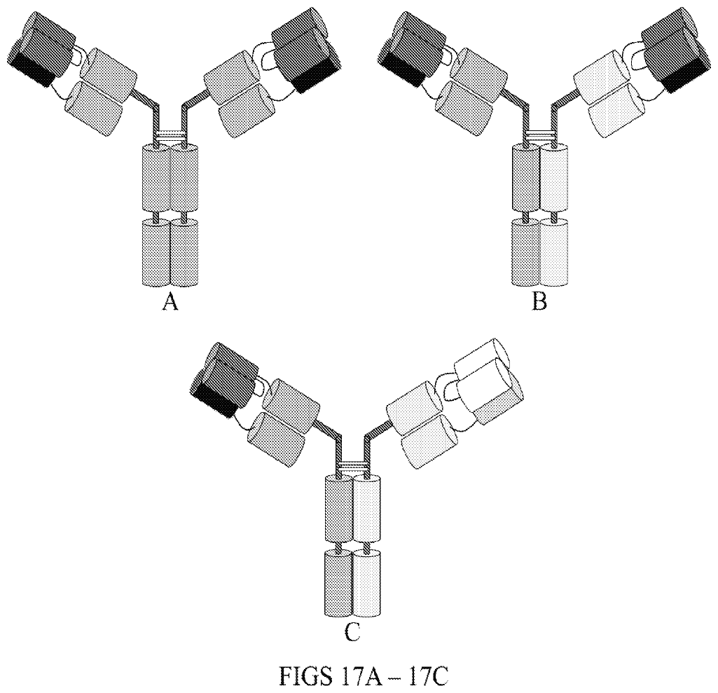

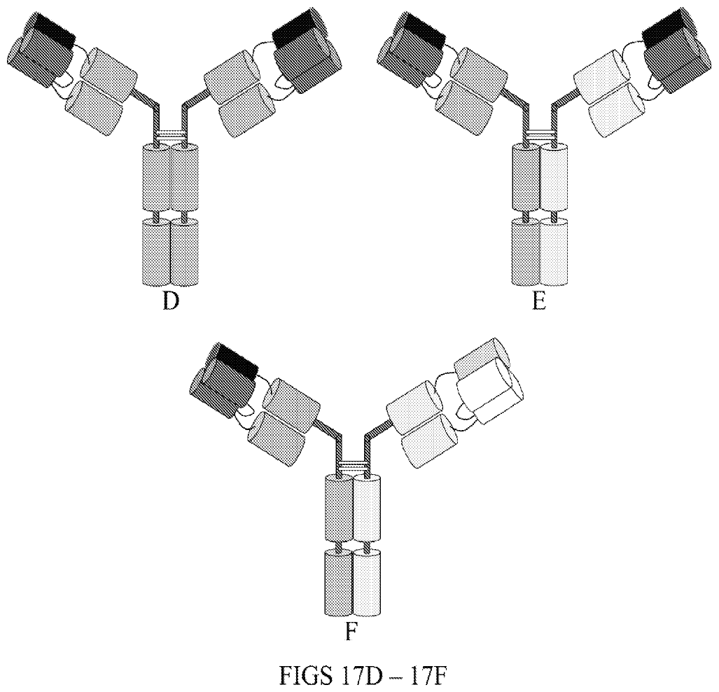

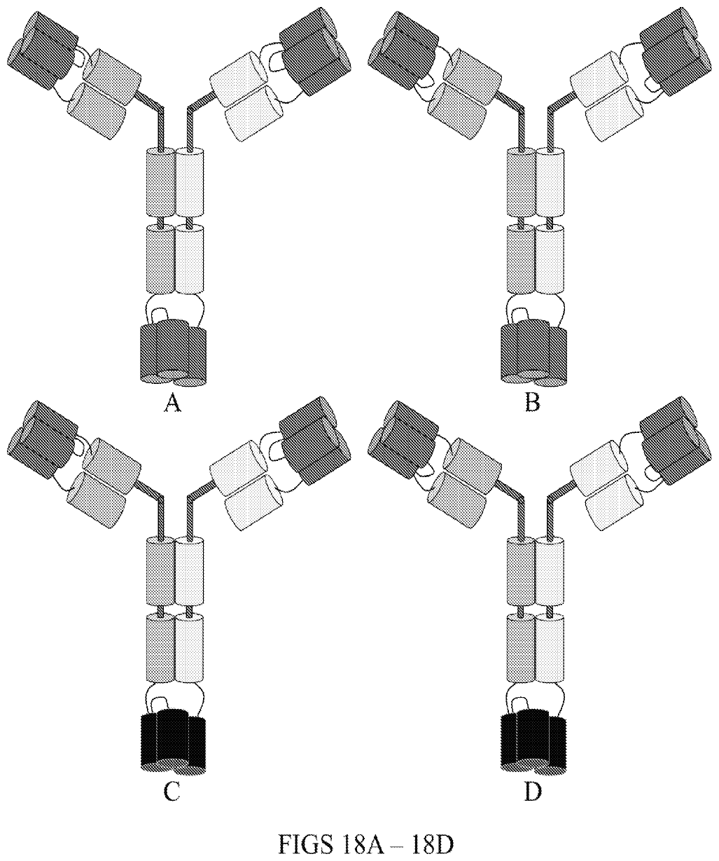

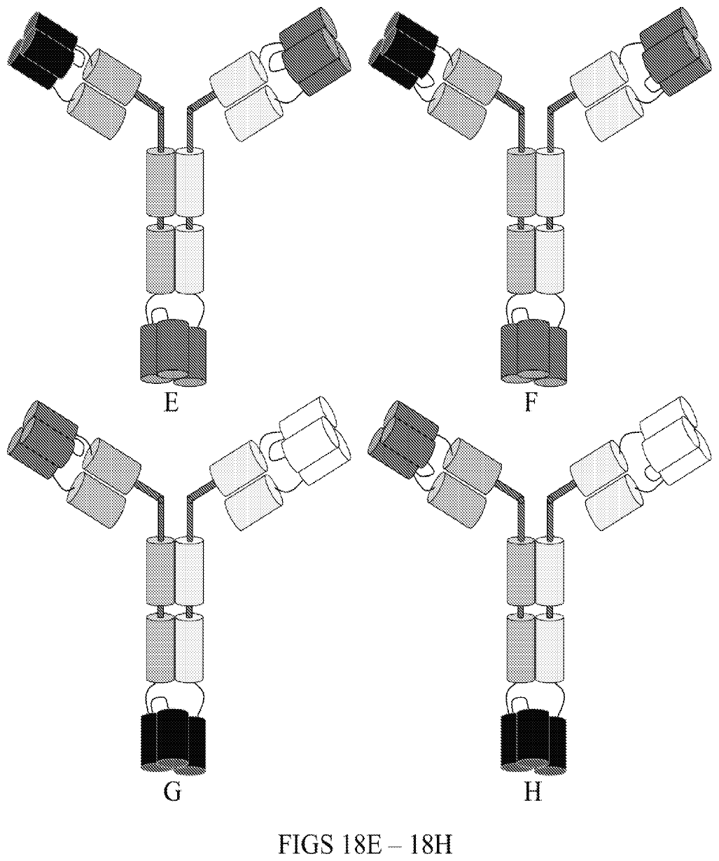

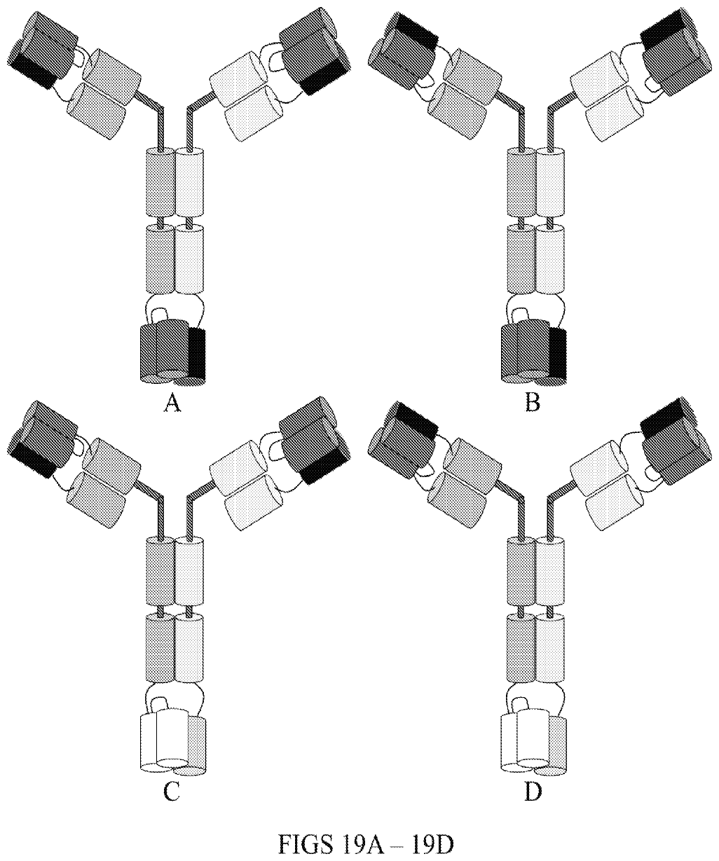

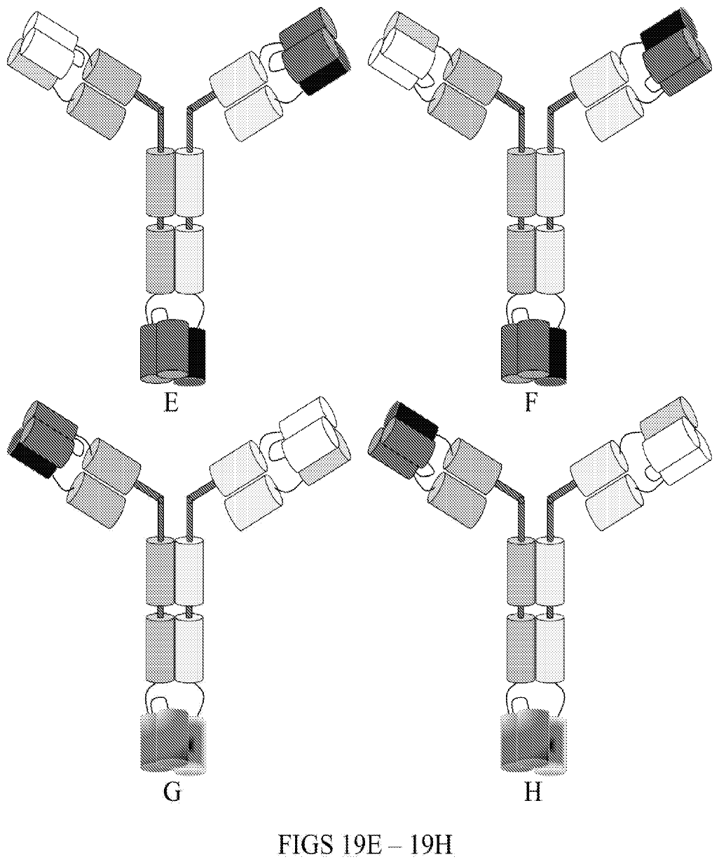

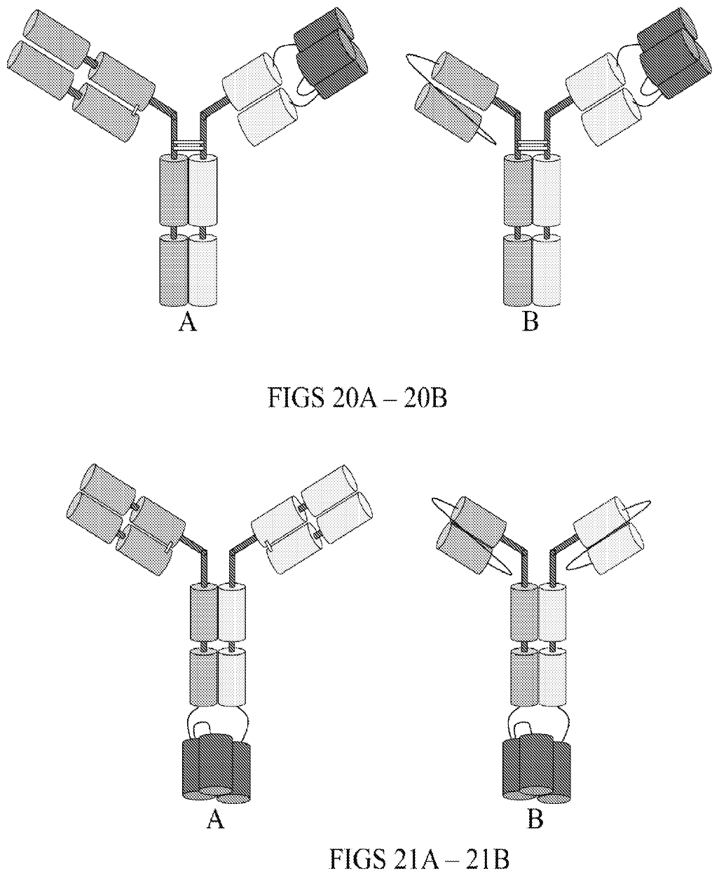

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 125 (with or without the signal peptide METDTLLLWVLLLWVPGSTG (SEQ ID NO: 74)). For representative configurations, see for example, FIGS. 14A-14B and 16A-16B.

In another embodiment, the GITRL homotrimer formed by SEQ ID NOs: 124 (with or without the signal peptide METDTLLLWVLLLWVPGSTG (SEQ ID NO: 74)) and 125 (with or without the signal peptide METDTLLLWVLLLWVPGSTG (SEQ ID NO: 74)) is combined with a complete Fab moiety fused to the N-terminus of a Fc heterodimer region.

(SEQ ID NO: 126)
METDTLLLWVLLLWVPGSTGEVQLLESGGGLVQPGGSLRLSCAASGFTFS
SYIMMWVRQAPGKGLEWVSSIYPSGGITFYADTVKGRFTISRDNSKNTLY
LQMNSLRAEDTAVYYCARIKLGTVTTVDYWGQGTLVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPCREEMTKNQVSLWCLVKGFYPSDI
AVEWESNGQPENNYKTTPPVLDSDGSFFLYSKLTVDKSRWQQGNVFSCSV
MHEALHNHYTQKSLSLSPGK

(with or without the signal peptide METDTLLLWVLLLWVPGSTG (SEQ ID NO: 74)) and

(SEQ ID NO: 127)
METDTLLLWVLLLWVPGSTGQSALTQPASVSGSPGQSITISCTGTSSDVG
GYNYVSWYQQHPGKAPKLMIYDVSNRPSGVSNRFSGSKSGNTASLTISGL
QAEDEADYYCSSYTSSSTRVFGTGTKVTVLGQPKANPTVTLFPPSSEELQ
ANKATLVCLISDFYPGAVTVAWKADGSPVKAGVETTKPSKQSNNKYAASS
YLSLTPEQWKSHRSYSCQVTHEGSTVEKTVAPTECS

(with or without the signal peptide METDTLLLWVLLLWVPGSTG (SEQ ID NO: 74)).

In another embodiment, the GITRL homotrimer formed by SEQ ID NOs: 124 (with or without the signal peptide METDTLLLWVLLLWVPGSTG (SEQ ID NO: 74)) and 125 (with or without the signal peptide METDTLLLWVLLLWVPGSTG (SEQ ID NO: 74)) is combined with a complete PDL1 binding scFv moiety fused to the N-terminus of an Fc heterodimer region.

(SEQ ID NO: 128)
METDTLLLWVLLLWVPGSTGEVQLLESGGGLVQPGGSLRLSCAASGFTFS
SYIMMWVRQAPGKGLEWVSSIYPSGGITFYADTVKGRFTISRDNSKNTLY
LQMNSLRAEDTAVYYCARIKLGTVTTVDYWGQGTLVTVSSGGGGSGGGGS
GGGGSGGGGSQSALTQPASVSGSPGQSITISCTGTSSDVGGYNYVSWYQQ
HPGKAPKLMIYDVSNRPSGVSNRFSGSKSGNTASLTISGLQAEDEADYYC
SSYTSSSTRVFGTGTKVTVLGGGGSDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPCREEMTKNQVSLWCLVKGFYPSDIAVEWESNGQPENNYKTTP
PVLDSDGSFFLYSKLTVDKSRWQQGNVFSCSVMHEALHNHYTQKSLSLSP
GK

(with or without the signal peptide METDTLLLWVLLLWVPGSTG (SEQ ID NO: 74)).

In one embodiment, the multifunctional molecule is a TNFSF fusion molecule that is comprised of a heterodimeric, knob-in-a-hole (KiH) human immunoglobulin IgG1 Fc domain with the hinge disulfides mutated from cysteine to serine with a C-terminal GITRL wherein the protein sequence from the N-terminus to the C-terminus of one of the heavy chains is comprised of an Ig kappa signal peptide (optional), a PDL1 binding VH region, an immunoglobulin constant heavy domain containing the T366W mutation, a 3×4GS linker (SEQ ID NO: 84), a GITRL monomer molecule a 4GS linker (SEQ ID NO: 80) and a second GITRL. In some embodiments, the multifunctional molecule comprising the amino acid sequence of:

(SEQ ID NO: 129)
METDTLLLWVLLLWVPGSTGEVQLLESGGGLVQPGGSLRLSCAASGFTFS
SYIMMWVRQAPGKGLEWVSSIYPSGGITFYADTVKGRFTISRDNSKNTLY
LQMNSLRAEDTAVYYCARIKLGTVTTVDYWGQGTLVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTSP
PSPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSREEMTKNQVSLWCLVKGFYPSDI
AVEWESNGQPENNYKTTPPVLDSDGSFFLYSKLTVDKSRWQQGNVFSCSV
MHEALHNHYTQKSLSLSPGKGGGGSGGGGSGGGGSKEPCMAKFGPLPSKW
QMASSEPPCVNKVSDWKLEILQNGLYLIYGQVAPNANYNDVAPFEVRLYK
NKDMIQTLTNKSKIQNVGGTYELHVGDTIDLIFNSEHQVLKNNTYWGIIL
LANPQFISGGSGGKEPCMAKFGPLPSKWQMASSEPPCVNKVSDWKLEILQ
NGLYLIYGQVAPNANYNDVAPFEVRLYKNKDMIQTLTNKSKIQNVGGTYE
LHVGDTIDLIFNSEHQVLKNNTYWGIILLANPQFIS

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 129 (with or without the signal peptide METDTLLLWVLLLWVPGSTG (SEQ ID NO: 74)). The light chain to complete the Fab is SEQ ID NO: 127 (with or without the signal peptide METDTLLLWVLLLWVPGSTG (SEQ ID NO: 74)). See for example, FIG. 21A.

In embodiments, the other heavy chain sequence from the N-terminus to the C-terminus is comprised of an Ig kappa signal peptide (optional), a PDL1 binding VH region, an immunoglobulin constant heavy domain containing the T366S, L368A and Y408V mutations; a 3×4GS linker (SEQ ID NO: 84) and a GITRL monomer molecule:

(SEQ ID NO: 130)
METDTLLLWVLLLWVPGSTGEVQLLESGGGLVQPGGSLRLSCAASGFTFS
SYIMMWVRQAPGKGLEWVSSIYPSGGITFYADTVKGRFTISRDNSKNTLY
LQMNSLRAEDTAVYYCARIKLGTVTTVDYWGQGTLVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTSP
PSPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSREEMTKNQVSLSCAVKGFYPSDI
AVEWESNGQPENNYKTTPPVLDSDGSFFLVSKLTVDKSRWQQGNVFSCSV
MHEALHNHYTQKSLSLSPGKGGGGSGGGGSGGGGSKEPCMAKFGPLPSKW
QMASSEPPCVNKVSDWKLEILQNGLYLIYGQVAPNANYNDVAPFEVRLYK
NKDMIQTLTNKSKIQNVGGTYELHVGDTIDLIFNSEHQVLKNNTYWGIIL
LANPQFIS

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 130 (with or without the signal peptide METDTLLLWVLLLWVPGSTG (SEQ ID NO: 74)). The light chain to complete the Fab is SEQ ID NO: 127 (with or without the signal peptide METDTLLLWVLLLWVPGSTG (SEQ ID NO: 74)).

In one embodiment, the multifunctional molecule is a TNFSF fusion molecule that is comprised of a heterodimeric, knob-in-a-hole (KiH) human immunoglobulin IgG1 Fc domain with the hinge disulfides mutated from cysteine to serine with a C-terminal GITRL wherein the protein sequence from the N-terminus to the C-terminus of one of the heavy chains is comprised of an Ig kappa signal peptide (optional), a PDL1 binding scFv domain, a 4GS linker (SEQ ID NO: 80), an immunoglobulin constant heavy domain containing the T366W mutation, a 3×4GS linker (SEQ ID NO: 84), a GITRL monomer molecule, a linker and a second GITRL monomer molecule. In some embodiments, the multifunctional molecule comprising the amino acid sequence of:

(SEQ ID NO: 131)
METDTLLLWVLLLWVPGSTGEVQLLESGGGLVQPGGSLRLSCAASGFTFS
SYIMMWVRQAPGKGLEWVSSIYPSGGITFYADTVKGRFTISRDNSKNTLY
LQMNSLRAEDTAVYYCARIKLGTVTTVDYWGQGTLVTVSSGGGGSGGGGS
GGGGSGGGGSQSALTQPASVSGSPGQSITISCTGTSSDVGGYNYVSWYQQ
HPGKAPKLMIYDVSNRPSGVSNRFSGSKSGNTASLTISGLQAEDEADYYC
SSYTSSSTRVFGTGTKVTVLGGGGSDKTHTSPPSPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLWCLVKGFYPSDIAVEWESNGQPENNYKTTP
PVLDSDGSFFLYSKLTVDKSRWQQGNVFSCSVMHEALHNHYTQKSLSLSP
GKGGGGSGGGGSGGGGSKEPCMAKFGPLPSKWQMASSEPPCVNKVSDWKL
EILQNGLYLIYGQVAPNANYNDVAPFEVRLYKNKDMIQTLTNKSKIQNVG
GTYELHVGDTIDLIFNSEHQVLKNNTYWGIILLANPQFISGGSGGKEPCM
AKFGPLPSKWQMASSEPPCVNKVSDWKLEILQNGLYLIYGQVAPNANYND
VAPFEVRLYKNKDMIQTLTNKSKIQNVGGTYELHVGDTIDLIFNSEHQVL
KNNTYWGIILLANPQFIS

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 131 (with or without the signal peptide METDTLLLWVLLLWVPGSTG (SEQ ID NO: 74)). See for example, FIG. 21B.

In embodiments, the other heavy chain sequence from the N-terminus to the C-terminus is comprised of an 1 g kappa signal peptide (optional), a PDL1 binding scFv region, a 4GS linker (SEQ ID NO: 80), an immunoglobulin constant heavy domain containing the T366S, L368A and Y408V mutations; a 3×4GS linker (SEQ ID NO: 84) and a GITRL monomer molecule:

(SEQ ID NO: 132)
METDTLLLWVLLLWVPGSTGEVQLLESGGGLVQPGGSLRLSCAASGFTFS
SYIMMWVRQAPGKGLEWVSSIYPSGGITFYADTVKGRFTISRDNSKNTLY
LQMNSLRAEDTAVYYCARIKLGTVTTVDYWGQGTLVTVSSGGGGSGGGGS
GGGGSGGGGSQSALTQPASVSGSPGQSITISCTGTSSDVGGYNYVSWYQQ
HPGKAPKLMIYDVSNRPSGVSNRFSGSKSGNTASLTISGLQAEDEADYYC
SSYTSSSTRVFGTGTKVTVLGGGGSDKTHTSPPSPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLSCAVKGFYPSDIAVEWESNGQPENNYKTTP
PVLDSDGSFFLVSKLTVDKSRWQQGNVFSCSVMHEALHNHYTQKSLSLSP
GKGGGGSGGGGSGGGGSKEPCMAKFGPLPSKWOMASSEPPCVNKVSDWKL
EILQNGLYLIYGQVAPNANYNDVAPFEVRLYKNKDMIQTLTNKSKIQNVG
GTYELHVGDTIDLIFNSEHQVLKNNTYWGIILLANPQFIS

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 132 (with or without the signal peptide METDTLLLWVLLLWVPGSTG (SEQ ID NO: 74)).

In one embodiment, the multifunctional molecule is a TNFSF fusion molecule that is comprised of a human IgG1 CH2 domain with the hinge disulfides mutated from cysteine to serine fused to a human T-Cell receptor alpha constant domain with a C-terminal GITRL wherein the protein sequence from the N-terminus to the C-terminus of one of the heavy chains is comprised of an Ig kappa signal peptide (optional), an immunoglobulin constant CH2 domain, a TCRα domain, a 3×4GS linker (SEQ ID NO: 84), a GITRL, a linker and a second GITRL monomer. In some embodiments, the multifunctional molecule comprising the amino acid sequence of:

(SEQ ID NO: 133)
METDTLLLWVLLLWVPGSTGDKTHTSPPSPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKPDIQNPDPAV
YQLRDSKSSDKSVCLFTDFDSQTNVSQSKDSDVYITDKTVLDMRSMDFKS
NSAVAWSNKSDFACANAFNNSIIPEDTFFPSPEGGGGSGGGGSGGGGSKE
PCMAKFGPLPSKWQMASSEPPCVNKVSDWKLEILQNGLYLIYGQVAPNAN
YNDVAPFEVRLYKNKDMIQTLTNKSKiuNVGUTYELHVGDTIDLIFNSEH
QVLKNNTYWGIILLANPQFISGGSGGKEPCMAKFGPLPSKWQMASSEPPC
VNKVSDWKLEILQNGLYLIYGQVAPNANYNDVAPFEVRLYKNKDMIQTLT
NKSKIQNVGGTYELHVGDTIDLIFNSEHQVLKNNTYWGIILLANPQFIS

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 133 (with or without the signal peptide METDTLLLWVLLLWVPGSTG (SEQ ID NO: 74)). See for example, FIG. 23A.

In one embodiment, the multifunctional molecule is a TNFSF fusion molecule that is comprised of a human IgG1 CH2 domain with the hinge disulfides mutated from cysteine to serine fused to a human T-Cell receptor beta constant domain with a C-terminal GITRL wherein the protein sequence from the N-terminus to the C-terminus of one of the heavy chains is comprised of an Ig kappa signal peptide (optional), an immunoglobulin constant CH2 domain, a TCRβ domain, a 3×4GS linker (SEQ ID NO: 84), a GITRL monomer, a linker, and a second GITRL monomer. In some embodiments, the multifunctional molecule comprises the amino acid sequence of:

(SEQ ID NO: 134)
METDTLLLWVLLLWVPGSTGDKTHTSPPSPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKEDLNKVFPPE
VALFEPSEAEISHTQKATLVCLATGFYPDHVELSWWVNGKEVHSGVSTDP
QPLKEQPALNDSRYALSSRLRVSATFWQDPRNHFRCQVQFYGLSEADEWT
QARAKPVTQIVSAEAWGRADGGGGSGGGGSGGGGSKEPCMAKFGPLPSKW
QMASSEPPCVNKVSDWKLEILQNGLYLIYGQVAPNANYNDVAPFEVRLYK
NKDMIQTLTNKSKIQNVGGTYELHVGDTIDLIFNSEHQVLKNNTYWGIIL
LANPQFIS

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 134 (with or without the signal peptide METDTLLLWVLLLWVPGSTG (SEQ ID NO: 74)).

In one embodiment, the multifunctional molecule is a TNFSF fusion molecule that is comprised of a human IgG1 CH2 domain with the hinge disulfides mutated from cysteine to serine fused to a human T-Cell receptor alpha constant domain with a C-terminal GITRL wherein the protein sequence from the N-terminus to the C-terminus of one of the heavy chains is comprised of an Ig kappa signal peptide (optional), a PDL1 binding VH region, an immunoglobulin constant heavy CH2 domain with no hinge disulfides, a TCRα constant domain, a 3×4GS linker (SEQ ID NO: 84), a GITRL monomer molecule a linker and a second GITRL monomer molecule. In some embodiments, the multifunctional molecule comprising the amino acid sequence of:

(SEQ ID NO: 135)
METDTLLLWVLLLWVPGSTGEVQLLESGGGLVQPGGSLRLSCAASGFTFS
SYIMMWVRQAPGKGLEWVSSIYPSGGITFYADTVKGRFTISRDNSKNTLY
LQMNSLRAEDTAVYYCARIKLGTVTTVDYWGQGTLVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTSP
PSPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKPDIQNPDPAVYQLRDSKSSDKSVCLFTDFDSQTNVSQ
SKDSDVYITDKTVLDMRSMDFKSNSAVAWSNKSDFACANAFNNSIIPEDT
FFPSPEGGGGSGGGGSGGGGSKEPCMAKFGPLPSKWQMASSEPPCVNKVS
DWKLEILQNGLYLIYGQVAPNANYNDVAPFEVRLYKNKDMIQTLTNKSKI
QNVGGTYELHVGDTIDLIFNSEHQVLKNNTYWGIILLANPQFISGGSGGK
EPCMAKFGPLPSKWQMASSEPPCVNKVSDWKLEILQNGLYLIYGQVAPNA
NYNDVAPFEVRLYKNKDMIQTLTNKSKIQNVGGTYELHVGDTIDLIFNSE
HQVLKNNTYWGIILLANPQFIS

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 135 (with or without the signal peptide METDTLLLWVLLLWVPGSTG (SEQ ID NO: 74)). The light chain to complete the Fab is SEQ ID NO: 127 (with or without the signal peptide METDTLLLWVLLLWVPGSTG (SEQ ID NO: 74)). See for example, FIG. 23B.

In embodiments, the other heavy chain sequence from the N-terminus to the C-terminus is comprised of an Ig kappa signal peptide (optional), a PDL1 binding VH region, an immunoglobulin constant heavy CH2 domain with no hinge disulfides, a TCR constant domain, a 3×4GS linker (SEQ ID NO: 84) and a GITRL monomer molecule:

(SEQ ID NO: 136)
METDTLLLWVLLLWVPGSTGEVQLLESGGGLVQPGGSLRLSCAASGFTFS
SYIMMWVRQAPGKGLEWVSSIYPSGGITFYADTVKGRFTISRDNSKNTLY
LQMNSLRAEDTAVYYCARIKLGTVTTVDYWGQGTLVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTSP
PSPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKEDLNKVFPPEVALFEPSEAEISHTQKATLVCLATGFY
PDHVELSWWVNGKEVHSGVSTDPQPLKEQPALNDSRYALSSRLRVSATFW
QDPRNHFRCQVQFYGLSEADEWTQARAKPVTQIVSAEAWGRADGGGGSGG
GGSGGGGSKEPCMAKFGPLPSKWQMASSEPPCVNKVSDWKLEILQNGLYL
IYGQVAPNANYNDVAPFEVRLYKNKDMIQTLTNKSKIQNVGGTYELHVGD
TIDLIFNSEHQVLKNNTYWGIILLANPQFIS

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 136 (with or without the signal peptide METDTLLLWVLLLWVPGSTG (SEQ ID NO: 74)). The light chain to complete the Fab is SEQ ID NO: 127 (with or without the signal peptide METDTLLLWVLLLWVPGSTG (SEQ ID NO: 74)).

In one embodiment, the multifunctional molecule is a TNFSF fusion molecule that is comprised of a human IgG1 CH2 domain with the hinge disulfides mutated from cysteine to serine fused to a human T-Cell receptor alpha constant domain with a C-terminal GITRL wherein the protein sequence from the N-terminus to the C-terminus of one of the heavy chains is comprised of an Ig kappa signal peptide (optional), a PDL1 binding scFv region, a 4GS linker (SEQ ID NO: 80), an immunoglobulin constant heavy CH2 domain with no hinge disulfides, a TCRα constant domain, a 3×4GS linker (SEQ ID NO: 84), a GITRL monomer molecule a linker and a second GITRL monomer molecule. In some embodiments, the multifunctional molecule comprising the amino acid sequence of:

(SEQ ID NO: 137)
METDTLLLWVLLLWVPGSTGEVQLLESGGGLVQPGGSLRLSCAASGFTFS
SYIMMWVRQAPGKGLEWVSSIYPSGGITFYADTVKGRFTISRDNSKNTLY
LQMNSLRAEDTAVYYCARIKLGTVTTVDYWGQGTLVTVSSGGGGSGGGGS
GGGGSGGGGSQSALTQPASVSGSPGQSITISCTGTSSDVGGYNYVSWYQQ
HPGKAPKLMIYDVSNRPSGVSNRFSGSKSGNTASLTISGLQAEDEADYYC
SSYTSSSTRVFGTGTKVTVLGGGGSDKTHTSPPSPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKPDIQN
PDPAVYQLRDSKSSDKSVCLFTDFDSQTNVSQSKDSDVYITDKTVLDMRS
MDFKSNSAVAWSNKSDFACANAFNNSIIPEDTFFPSPEGGGGSGGGGSGG
GGSKEPCMAKFGPLPSKWQMASSEPPCVNKVSDWKLEILQNGLYLIYGQV
APNANYNDVAPFEVRLYKNKDMIQTLTNKSKIQNVGGTYELHVGDTIDLI
FNSEHQVLKNNTYWGIILLANPQFISGGSGGKEPCMAKFGPLPSKWQMAS
SEPPCVNKVSDWKLEILQNGLYLIYGQVAPNANYNDVAPFEVRLYKNKDM
IQTLTNKSKIQNVGGTYELHVGDTIDLIFNSEHQVLKNNTYWGIILLANP
QFIS

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 137 (with or without the signal peptide METDTLLLWVLLLWVPGSTG (SEQ ID NO: 74)). See for example, FIG. 23C.

In embodiments, the other heavy chain sequence from the N-terminus to the C-terminus is comprised of an Ig kappa signal peptide (optional), a PDL1 binding scFv region, a 4GS linker (SEQ ID NO: 80), an immunoglobulin constant heavy CH2 domain with no hinge disulfides, a TCRβ constant domain, a 3×4GS linker (SEQ ID NO: 84) and a GITRL monomer molecule:

(SEQ ID NO: 138)
METDTLLLWVLLLWVPGSTGEVQLLESGGGLVQPGGSLRLSCAASGFTFS
SYIMMWVRQAPGKGLEWVSSIYPSGGITFYADTVKGRFTISRDNSKNTLY
LQMNSLRAEDTAVYYCARIKLGTVTTVDYWGQGTLVTVSSGGGGSGGGGS
GGGGSGGGGSQSALTQPASVSGSPGQSITISCTGTSSDVGGYNYVSWYQQ
HPGKAPKLMIYDVSNRPSGVSNRFSGSKSGNTASLTISGLQAEDEADYYC
SSYTSSSTRVFGTGTKVTVLGGGGSDKTHTSPPSPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKEDLNK
VFPPEVALFEPSEAEISHTQKATLVCLATGFYPDHVELSWWVNGKEVHSG
VSTDPQPLKEQPALNDSRYALSSRLRVSATFWQDPRNHFRCQVQFYGLSE
ADEWTQARAKPVTQIVSAEAWGRADGGGGSGGGGSGGGGSKEPCMAKFGP
LPSKWQMASSEPPCVNKVSDWKLEILQNGLYLIYGQVAPNANYNDVAPFE
VRLYKNKDMIQTLTNKSKIQNVGGTYELHVGDTIDLIFNSEHQVLKNNTY
WGIILLANPQFIS

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 138 (with or without the signal peptide METDTLLLWVLLLWVPGSTG (SEQ ID NO: 74)).

In embodiments, the multifunctional molecule comprises a heavy chain comprising an Ig kappa signal peptide (optional), a GITRL monomer molecule, a linker, a second GITRL monomer molecule, a 3×4GS linker (SEQ ID NO: 84) and a IgG1 heavy constant region composed of a CH1 domain with the C-terminal cysteine used to make the interchain disulfide with the constant light domain removed and a CH2 domain, and a T-cell receptor beta constant domain.

(SEQ ID NO: 139)
METDTLLLWVLLLWVPGSTGKEPCMAKFGPLPSKWQMASSEPPCVNKVSD
WKLEILQNGLYLIYGQVAPNANYNDVAPFEVRLYKNKDMIQTLTNKSKIQ
NVGGTYELHVGDTIDLIFNSEHQVLKNNTYWGIILLANPQFISGGSGGKE
PCMAKFGPLPSKWQMASSEPPCVNKVSDWKLEILQNGLYLIYGQVAPNAN
YNDVAPFEVRLYKNKDMIQTLTNKSKIQNVGGTYELHVGDTIDLIFNSEH
QVLKNNTYWGIILLANPQFISGGGGSGGGGSGGGGSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKRVEPKSDKTHTCPPCPAP
ELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKEDLNKVFPPEVALFEPSEAEISHTQKATLVCLATGFYPDHVE
LSWWVNGKEVHSGVSTDPQPLKEQPALNDSRYALSSRLRVSATFWQDPRN
HFRCQVQFYGLSEADEWTQARAKPVTQIVSAEAWGRAD

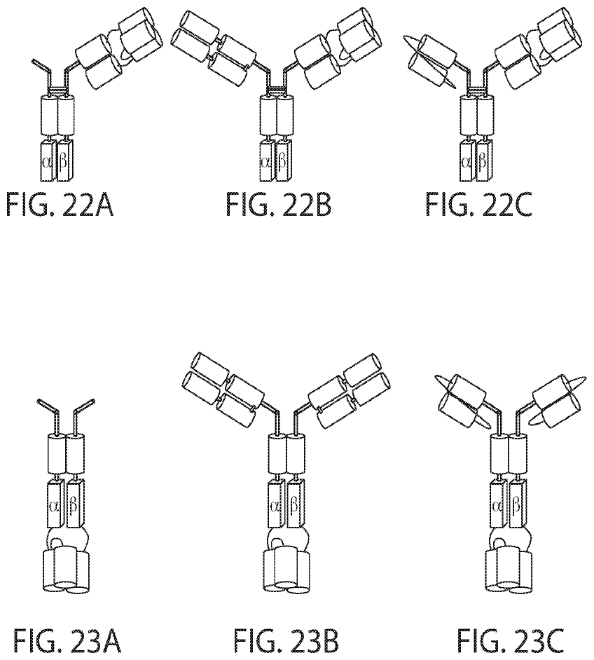

(with or without the signal peptide METDTLLLWVLLLWVPGSTG (SEQ ID NO: 74)), or an amino acid sequence substantially identical thereto, e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 139 (with or without the signal peptide METDTLLLWVLLLWVPGSTG (SEQ ID NO: 74)). See for example, FIGS. 22A, 22B, and 22C.

In other embodiments, the light chain that pairs with SEQ ID NO: 139 (with or without the signal peptide METDTLLLWVLLLWVPGSTG (SEQ ID NO: 74)) is SEQ ID NO: 125 (with or without the signal peptide METDTLLLWVLLLWVPGSTG (SEQ ID NO: 74)).

In another embodiment, the GITRL homotrimer formed by SEQ ID NOS: 139 (with or without the signal peptide METDTLLLWVLLLWVPGSTG (SEQ ID NO: 74)) and 125 (with or without the signal peptide METDTLLLWVLLLWVPGSTG (SEQ ID NO: 74)) is combined with a complete Fab moiety fused to the N-terminus of an IgG1 CH2 domain fused to the T-cell receptor alpha constant domain.

(SEQ ID NO: 140)
METDTLLLWVLLLWVPGSTGEVQLLESGGGLVQPGGSLRLSCAASGFTFS
SYIMMWVRQAPGKGLEWVSSIYPSGGITFYADTVKGRFTISRDNSKNTLY
LQMNSLRAEDTAVYYCARIKLGTVTTVDYWGQGTLVTVSSASTKGPSVFP
LAPSSKSTSGGTAALGCLVKDYFPEPVTVSWNSGALTSGVHTFPAVLQSS
GLYSLSSVVIVPSSSLGTQTYICNVNHKPSNTKVDKRVEPKSCDKTHTCP
PCPAPELLGGPSVFLFPPKPICDTLMISRTPEVTCVVVDVSHEDPEVICF
NWYVDGVEVHNAKTKPREEQYNSTYRVVSVLTVLHQDWLNGKEYKCKVSN
KALPAPIEKTISKAKPDIQNPDPAVYQLRDSKSSDKSVCLFTDFDSQTNV
SQSKDSDVYITDKTVLDMRSMDFKSNSAVAWSNKSDFACANAFNNSIIPE
DTFFPSPE

(with or without the signal peptide METDTLLLWVLLLWVPGSTG (SEQ ID NO: 74)) and SEQ ID NO: 127 (with or without the signal peptide METDTLLLWVLLLWVPGSTG (SEQ ID NO: 74)). See for example, FIG. 22B.

In another embodiment, the GITRL homotrimer formed by SEQ ID NOs: 139 (with or without the signal peptide METDTLLLWVLLLWVPGSTG (SEQ ID NO: 74)) and 125 (with or without the signal peptide METDTLLLWVLLLWVPGSTG (SEQ ID NO: 74)) is combined with a complete PDL1 binding scFv moiety fused to the N-terminus of an IgG1 CH2 domain fused to the T-cell receptor alpha constant domain.

(SEQ ID NO: 141)
METDTLLLWVLLLWVPGSTGEVQLLESGGGLVQPGGSLRLSCAASGFTFS
SYIMMWVRQAPGKGLEWVSSIYPSGGITFYADTVKGRFTISRDNSKNTLY
LQMNSLRAEDTAVYYCARIKLGTVTTVDYWGQGTLVTVSSGGGGSGGGGS
GGGGSGGGGSQSALTQPASVSGSPGQSITISCTGTSSDVGGYNYVSWYQQ
HPGKAPKLMIYDVSNRPSGVSNRFSGSKSGNTASLTISGLQAEDEADYYC
SSYTSSSTRVFGTGTKVTVLGGGGSDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKPDIQN
PDPAVYQLRDSKSSDKSVCLFTDFDSQTNVSQSKDSDVYITDKTVLDMRS
MDFKSNSAVAWSNKSDFACANAFNNSIIPEDTFFPSPE

(with or without the signal peptide METDTLLLWVLLLWVPGSTG (SEQ ID NO: 74)). See for example, FIG. 22C.

### Other Functionalities or Binding Specificities

The multifunctional molecules disclosed herein can further comprise one or more other binding specificities or functionalities. In embodiments, the other binding specificity or functionality is chosen from one, two or more of: a targeting moiety, e.g., a tumor targeting moiety; an immune cell engager (e.g., chosen from one, two, three, or all of an NK cell engager, a B cell engager, a dendritic cell engager, or a macrophage cell engager); and/or a cytokine molecule, e.g., as described herein.

### Targeting Moieties

In certain embodiments, the multifunctional molecules disclosed herein include a tumor-targeting moiety. The terms “tumor” and “cancer” are used interchangeably herein and include all malignant and pre-malignant cancerous conditions. The tumor targeting moiety can be chosen from an antibody molecule (e.g., an antigen binding domain as described herein), a receptor or a receptor fragment, or a ligand or a ligand fragment, or a combination thereof. In some embodiments, the tumor targeting moiety associates with, e.g., binds to, a tumor cell (e.g., a molecule, e.g., antigen, present on the surface of the tumor cell). In certain embodiments, the tumor targeting moiety targets, e.g., directs the multifunctional molecules disclosed herein to a cancer (e.g., a cancer or tumor cells). In some embodiments, the cancer is chosen from a hematological cancer, a solid cancer, a metastatic cancer, or a combination thereof.

In some embodiments, the multifunctional molecule, e.g., the tumor-targeting moiety, binds to a solid tumor antigen or a stromal antigen. The solid tumor antigen or stromal antigen can be present on a solid tumor, or a metastatic lesion thereof. In some embodiments, the solid tumor is chosen from one or more of pancreatic (e.g., pancreatic adenocarcinoma), breast, colorectal, lung (e.g., small or non-small cell lung cancer), skin, ovarian, or liver cancer. In one embodiment, the solid tumor is a fibrotic or desmoplastic solid tumor. For example, the solid tumor antigen or stromal antigen can be present on a tumor, e.g., a tumor of a class typified by having one or more of: limited tumor perfusion, compressed blood vessels, or fibrotic tumor interstitium.

In certain embodiments, the solid tumor antigen is chosen from one or more of: mesothelin, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pme117, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, α actinin-4, TRP1/gp75, TRP2, gp100, Melan-A/MART1, gangliosides, WT1, EphA3, Epidermal growth factor receptor (EGFR), CD20, MART-2, MART-1, MUC1, MUC2, MUM1, MUM2, MUM3, NA88-1, NPM, OA1, OGT, RCC, RUI1, RUI2, SAGE, TRG, TRP1, or TSTA. In some embodiments, the solid tumor antigen is chosen from: Mesothelin, GD2, PMSA, PSCA, CEA, Ron Kinase, or c-Met.

In one embodiment, the tumor-targeting moiety includes an antibody molecule (e.g., Fab or scFv) that binds to PD-L1.

In another embodiment, the tumor-targeting moiety includes an antibody molecule (e.g., Fab or scFv) that binds to mesothelin. In some embodiments, the antibody molecule to mesothelin comprises one, two, three CDRs from the heavy chain variable domain sequence of:

(SEQ ID NO: 248)
QVQLQQSGPELEKPGASVKISCKASGYSFTGYTMNWVKQSHGKSLEWIGL
ITPYNGASSYNQKFRGKATLTVDKSSSTAYMDLLSLTSEDSAVYFCARGG
YDGRGFDYWGQGTTVTVSS,

or a closely related CDR, e.g., CDRs which have at least one amino acid alteration, but not more than two, three or four alterations (e.g., substitutions, deletions, or insertions, e.g., conservative substitutions) from the CDR sequence of SEQ ID NO: 248.

In some embodiments, the antibody molecule to mesothelin comprises one, two, three CDRs selected from GYSFTGYTMN (SEQ ID NO: 249); LITPYNGASSYNQKFRG (SEQ ID NO: 250); and GGYDGRGFDY (SEQ ID NO: 251), or a closely related CDR, e.g., CDRs which have at least one amino acid alteration, but not more than two, three or four alterations (e.g., substitutions, deletions, or insertions, e.g., conservative substitutions).

In some embodiments, the antibody molecule to mesothelin consists of three CDRs, wherein CDR1 comprises GYSFTGYTMN (SEQ ID NO: 249); CDR2 comprises:

LITPYNGASSYNQKFRG (SEQ ID NO: 250); and CDR3 comprises GGYDGRGFDY (SEQ ID NO: 251), or a closely related CDR, e.g., CDRs which have at least one amino acid alteration, but not more than two, three or four alterations (e.g., substitutions, deletions, or insertions, e.g., conservative substitutions).

In some embodiments, the antibody molecule to mesothelin consists of three CDRs, wherein CDR1 consists of GYSFTGYTMN (SEQ ID NO: 249); CDR2 consists of LITPYNGASSYNQKFRG (SEQ ID NO: 250); and CDR3 consists of GGYDGRGFDY (SEQ ID NO: 251), or a closely related CDR, e.g., CDRs which have at least one amino acid alteration, but not more than two, three or four alterations (e.g., substitutions, deletions, or insertions, e.g., conservative substitutions).

In embodiments, the antibody molecule to mesothelin includes the heavy chain variable domain sequence of: SEQ ID NO: 248, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 248. In embodiments, the antibody molecule to mesothelin is a Fab and further comprises a heavy chain constant region (CH1) having the amino acid sequence:

(SEQ ID NO: 252)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHT,

or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 252. In some embodiments, the antibody molecule further comprises a signal peptide, e.g., a signal peptide comprising the amino acid sequence: MEFGLSWVFLVALFRGVQC (SEQ ID NO: 253).

Alternatively, or in combination with the heavy chain to mesothelin disclosed herein, the antibody molecule to mesothelin comprises one, two, three CDRs from the light chain variable domain sequence of:

(SEQ ID NO: 254)
DIELTQSPAIMSASPGEKVTMTCSASSSVSYNITIWYQQKSGTSPKRWIY
DTSKLASGVPGRFSGSGSGNSYSLTISSVEAEDDATYYCQQWSGYPLTFG
AGTKLEIK,

or a closely related CDR, e.g., CDRs which have at least one amino acid alteration, but not more than two, three or four alterations (e.g., substitutions, deletions, or insertions, e.g., conservative substitutions) from the CDR sequence of SEQ ID NO: 254.

In some embodiments, the antibody molecule to mesothelin comprises one, two, three CDRs from SASSSVSYMH (SEQ ID NO: 255); DTSKLAS (SEQ ID NO: 256); and QQWSGYPLT (SEQ ID NO: 257), or a closely related CDR, e.g., CDRs which have at least one amino acid alteration, but not more than two, three or four alterations (e.g., substitutions, deletions, or insertions, e.g., conservative substitutions).

In some embodiments, the antibody molecule to mesothelin consists of three CDRs, wherein CDR1 comprises SASSSVSYMH (SEQ ID NO: 255); CDR2 comprises: DTSKLAS (SEQ ID NO: 256); and CDR3 comprises QQWSGYPLT (SEQ ID NO: 257), or a closely related CDR, e.g., CDRs which have at least one amino acid alteration, but not more than two, three or four alterations (e.g., substitutions, deletions, or insertions, e.g., conservative substitutions).

In some embodiments, the antibody molecule to mesothelin consists of three CDRs, wherein CDR1 consists of SASSSVSYMH (SEQ ID NO: 255); CDR2 consists of DTSKLAS (SEQ ID NO: 256); and CDR3 consists of QQWSGYPLT (SEQ ID NO: 257), or a closely related CDR, e.g., CDRs which have at least one amino acid alteration, but not more than two, three or four alterations (e.g., substitutions, deletions, or insertions, e.g., conservative substitutions).

In some embodiments, the antibody molecule to mesothelin comprises the light chain variable domain sequence of:

(SEQ ID NO: 258)
DIELTQSPAIMSASPGEKVTMTCSASSSVSYMHWYQQKSGTSPKRWIYDT
SKLASGVPGRFSGSGSGNSYSLTISSVEAEDDATYYCQQWSGYPLTFGAG
TKLEIK,

or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 258.

In some embodiments, the antibody molecule to mesothelin is a Fab and further comprises a light chain constant region (CL1) having the amino acid sequence:

(SEQ ID NO: 259)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC,

or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 259 In embodiments, the antibody molecule further comprises a signal peptide, e.g., a signal peptide comprising the amino acid sequence: MKYLLPTAAAGLLLLAAQPAMA (SEQ ID NO: 260).

In other embodiments, the multispecific molecule, e.g., the tumor-targeting moiety, binds to a stromal antigen. In embodiments, the stromal antigen is chosen from one or more of: fibroblast activating protease (FAP), TGF-beta, hyaluronic acid, collagen, e.g., collagen IV, tenascin C, or tenascin W.

In one embodiment, the tumor-targeting moiety includes an antibody molecule (e.g., Fab or scFv) that binds to FAP, e.g., human FAP. In some embodiments, the antibody molecule to FAP comprises one, two, three CDRs from the heavy chain variable domain sequence of:

(SEQ ID NO: 261)
QVQLVQSGAEVKKPGASVKVSCKTSRYTFTEYTIHWVRQAPGQRLEWIGG
INPNNGIPNYNQKFKGRVTITVDTSASTAYMELSSLRSEDTAVYYCARRR
IAYGYDEGHAMDYWGQGTLVTVSS,

or a closely related CDR, e.g., CDRs which have at least one amino acid alteration, but not more than two, three or four alterations (e.g., substitutions, deletions, or insertions, e.g., conservative substitutions) from the CDR sequence of SEQ ID NO: 261. In some embodiments, the antibody molecule to FAP includes the heavy chain variable domain sequence of SEQ ID NO: 261, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 261.

In embodiments, the antibody molecule to FAP is a Fab and further comprises a heavy chain constant region (CH1) having the amino acid sequence:

(SEQ ID NO: 262)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSC,

or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 262. In embodiments, the antibody molecule further comprises a signal peptide, e.g., a signal peptide comprising the amino acid sequence:

(SEQ ID NO: 263)
MEFGLSWVFLVALFRGVQCEV.

Alternatively, or in combination with the heavy chain to FAP disclosed herein, the antibody molecule to FAP comprises one, two, three CDRs from the light chain variable domain sequence of:

(SEQ ID NO: 264)
DIVMTQSPDSLAVSLGERATINCKSSQSLLYSRNQKNYLAWYQQKPGQPP
KLLIFWASTRESGVPDRFSGSGFGTDFTLTISSLQAEDVAVYYCQQYFSY
PLTFGQGTKVEIK,

or a closely related CDR, e.g., CDRs which have at least one amino acid alteration, but not more than two, three or four alterations (e.g., substitutions, deletions, or insertions, e.g., conservative substitutions) from the CDR sequence of SEQ ID NO: 264. In some embodiments, the antibody molecule to FAP includes the light chain variable domain sequence of SEQ ID NO: 264, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 264.

In embodiments, the antibody molecule to FAP is a Fab and further comprises a light chain constant region (CL1) having the amino acid sequence:

(SEQ ID NO: 265)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC,

or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 265. In some embodiments, the antibody molecule further comprises a signal peptide, e.g., a signal peptide comprising the amino acid sequence: MKYLLPTAAAGLLLLAAQPAMA (SEQ ID NO: 266).

### Cytokine Molecules

In certain embodiments, the multifunctional antibody molecules disclosed herein can further include a cytokine molecule other than a TNFSF or TNF-like family member.

Cytokines are proteinaceous signaling compounds that are mediators of the immune response. They control many different cellular functions including proliferation, differentiation and cell survival/apoptosis; cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNgamma, IL-1, IL-6 and TNF-alpha, are predominantly derived from the innate immune cells and Th1 cells. Anti-inflammatory cytokines, including IL-10, IL-4, IL-13 and IL-5, are synthesized from Th2 immune cells.

The present disclosure provides, inter alia, multi-specific (e.g., bi-, tri-, quad-specific) proteins, that include, e.g., are engineered to contain, one or more cytokine molecules, e.g., immunomodulatory (e.g., proinflammatory) cytokines and variants, e.g., functional variants, thereof. Accordingly, in some embodiments, the cytokine molecule is an interleukin or a variant, e.g., a functional variant thereof. In some embodiments the interleukin is a proinflammatory interleukin. In some embodiments the interleukin is chosen from interleukin-2 (IL-2), interleukin-12 (IL-12), interleukin-15 (IL-15), interleukin-18 (IL-18), interleukin-21 (IL-21), or interferon gamma. In some embodiments, the cytokine molecule is a proinflammatory cytokine.

In some embodiments, the multifunctional molecules disclosed herein include a cytokine molecule. In embodiments, the cytokine molecule includes a full length, a fragment or a variant of a cytokine; a cytokine receptor domain, e.g., a cytokine receptor dimerizing domain; or an agonist of a cytokine receptor, e.g., an antibody molecule (e.g., an agonistic antibody) to a cytokine receptor.

In some embodiments the cytokine molecule is chosen from IL-2, IL-12, IL-15, IL-18, IL-21, or interferon gamma, or a fragment or variant thereof, or a combination of any of the aforesaid cytokines. The cytokine molecule can be a monomer molecule or a dimer. In embodiments, the cytokine molecule can further include a cytokine receptor dimerizing domain.

In other embodiments, the cytokine molecule is an agonist of a cytokine receptor, e.g., an antibody molecule (e.g., an agonistic antibody) to a cytokine receptor chosen from an IL-15Ra or IL-21R.

In one embodiment, the cytokine molecule is IL-15, e.g., human IL-15 (e.g., comprising the amino acid sequence:

(SEQ ID NO: 267)
NWVNVISDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVQMFINTS,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 267.

In some embodiments, the cytokine molecule comprises a receptor dimerizing domain, e.g., an IL15Ralpha dimerizing domain. In one embodiment, the IL15Ralpha dimerizing domain comprises the amino acid sequence:

(SEQ ID NO: 268)
MAPRRARGCRTLGLPALLLLLLLRPPATRGITCPPPMSVEHADIWVKSYS
LYSRERYICNSGFKRKAGTSSLTECVL,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 268. In some embodiments, the cytokine molecule (e.g., IL-15) and the receptor dimerizing domain (e.g., an IL15Ralpha dimerizing domain) of the multispecific molecule are covalently linked, e.g., via a linker (e.g., a Gly-Ser linker, e.g., a linker comprising the amino acid sequence SGGSGGGGSGGGSGGGGSLQ (SEQ ID NO: 269). In other embodiments, the cytokine molecule (e.g., IL-15) and the receptor dimerizing domain (e.g., an IL15Ralpha dimerizing domain) of the multispecific molecule are not covalently linked, e.g., are non-covalently associated.

In other embodiments, the cytokine molecule is IL-2, e.g., human IL-2 (e.g., comprising the amino acid sequence:

(SEQ ID NO: 270)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 270).

In other embodiments, the cytokine molecule is IL-18, e.g., human IL-18 (e.g., comprising the amino acid sequence:

(SEQ ID NO: 271)
YFGKLESKLSVIRNLNDQVLFIDQGNRPLFEDMTDSDCRDNAPRTIFIIS
MYKDSQPRGMAVTISVKCEKISTLSCENKIISFKEMNPPDNIKDTKSDII
FFQRSVPGRDNKMQFESSSYEGYFLACEKERDLFKLILKKEDELGDRSIM
FTVQNED,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 271).

In other embodiments, the cytokine molecule is IL-21, e.g., human IL-21 (e.g., comprising the amino acid sequence:

(SEQ ID NO: 272)
QGQDRHMIRMRQLIDIVDQLKNYVNDLVPEFLPAPEDVETNCEWSAFSCF
QKAQLKSANTGNNERIINVSIKKLKRKPPSTNAGRRQKHRLTCPSCDSYE
KKPPKEFLERFKSLLQKMIHQHLSSRTHGSEDS,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 272).

In yet other embodiments, the cytokine molecule is interferon gamma, e.g., human interferon gamma (e.g., comprising the amino acid sequence:

(SEQ ID NO: 273)
QDPYVKEAENLKKYFNAGHSDVADNGTLFLGILKNWKEESDRKIMQSQIV
SFYFKLFKNFKDDQSIQKSVETIKEDMNVKFFNSNKKKRDDFEKLTNYSV
TDLNVQRKAIHELIQVMAELSPAAKTGKRKRSQMLFRG,

or a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 273).

### Immune Cell Engagers

In certain embodiments, the multifunctional antibody molecules disclosed herein include an immune cell engager.

The immune cell engagers of the multifunctional molecules disclosed herein can mediate binding to, and/or activation of, an immune cell, e.g., an immune effector cell. In some embodiments, the immune cell is chosen from an NK cell, a B cell, a dendritic cell, or a macrophage cell engager, or a combination thereof. In some embodiments, the immune cell engager is chosen from one, two, three, or all of an NK cell engager, a B cell engager, a dendritic cell engager, or a macrophage cell engager, or a combination thereof. The immune cell engager can be an agonist of the immune system. In some embodiments, the immune cell engager can be an antibody molecule, a ligand molecule (e.g., a ligand that further comprises an immunoglobulin constant region, e.g., an Fc region), a small molecule, a nucleotide molecule.

### Natural Killer Cell Engagers

Natural Killer (NK) cells recognize and destroy tumors and virus-infected cells in an antibody-independent manner. The regulation of NK cells is mediated by activating and inhibiting receptors on the NK cell surface. One family of activating receptors is the natural cytotoxicity receptors (NCRs) which include NKp30, NKp44 and NKp46. The NCRs initiate tumor targeting by recognition of heparan sulfate on cancer cells. NKG2D is a receptor that provides both stimulatory and costimulatory innate immune responses on activated killer (NK) cells, leading to cytotoxic activity. DNAM1 is a receptor involved in intercellular adhesion, lymphocyte signaling, cytotoxicity and lymphokine secretion mediated by cytotoxic T-lymphocyte (CTL) and NK cell. DAP10 (also known as HCST) is a transmembrane adapter protein which associates with KLRK1 to form an activation receptor KLRK1-HCST in lymphoid and myeloid cells; this receptor plays a major role in triggering cytotoxicity against target cells expressing cell surface ligands such as MHC class I chain-related MICA and MICB, and U(optionally L1)6-binding proteins (ULBPs); it KLRK1-HCST receptor plays a role in immune surveillance against tumors and is required for cytolysis of tumors cells; indeed, melanoma cells that do not express KLRK1 ligands escape from immune surveillance mediated by NK cells. CD16 is a receptor for the Fc region of IgG, which binds complexed or aggregated IgG and also monomer moleculeic IgG and thereby mediates antibody-dependent cellular cytotoxicity (ADCC) and other antibody-dependent responses, such as phagocytosis.

In some embodiments, the NK cell engager is a viral hemagglutinin (HA), HA is a glycoprotein found on the surface of influenza viruses. It is responsible for binding the virus to cells with sialic acid on the membranes, such as cells in the upper respiratory tract or erythrocytes. HA has at least 18 different antigens. These subtypes are named H1 through H18. NCRs can recognize viral proteins. NKp46 has been shown to be able to interact with the HA of influenza and the HA-NA of Paramyxovirus, including Sendai virus and Newcastle disease virus. Besides NKp46, NKp44 can also functionally interact with HA of different influenza subtypes.

The present disclosure provides, inter alia, multi-specific (e.g., bi-, tri-, quad-specific) proteins, that are engineered to contain one or more NK cell engager that mediate binding to and/or activation of an NK cell. Accordingly, in some embodiments, the NK cell engager is selected from an antigen binding domain or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80 or CD244 (also known as SLAMF4 or 2B4).

In other embodiments, the NK cell engager is a ligand of NKp44 or NKp46, which is a viral HA. Viral hemagglutinins (HA) are glyco proteins which are on the surface of viruses. HA proteins allow viruses to bind to the membrane of cells via sialic acid sugar moieties which contributes to the fusion of viral membranes with the cell membranes (see e.g., Eur J Immunol. 2001 September; 31(9):2680-9 “Recognition of viral hemagglutinins by NKp44 but not by NKp30”; and Nature. 2001 Feb. 22; 409(6823):1055-60 “Recognition of haemagglutinins on virus-infected cells by NKp46 activates lysis by human NK cells” the contents of each of which are incorporated by reference herein).

In other embodiments, the NK cell engager is a ligand of NKG2D chosen from MICA, MICB, or ULBP1.

In other embodiments, the NK cell engager is a ligand of DNAM1 chosen from NECTIN2 or NECL5.

In yet other embodiments, the NK cell engager is a ligand of DAP10, which is an adapter for NKG2D (see e.g., Proc Natl Acad Sci USA. 2005 May 24; 102(21): 7641-7646; and Blood, 15 Sep. 2011 Volume 118, Number 11, the full contents of each of which is incorporated by reference herein).

In other embodiments, the NK cell engager is a ligand of CD16, which is a CD16a/b ligand, e.g., a CD16a/b ligand further comprising an antibody Fc region (see e.g., Front Immunol. 2013; 4: 76 discusses how antibodies use the Fc to trigger NK cells through CD16, the full contents of which are incorporated herein).

### B Cell, Macrophage & Dendritic Cell Engagers

Broadly, B cells, also known as B lymphocytes, are a type of white blood cell of the lymphocyte subtype. They function in the humoral immunity component of the adaptive immune system by secreting antibodies. Additionally, B cells present antigen (they are also classified as professional antigen-presenting cells (APCs)) and secrete cytokines. Macrophages are a type of white blood cell that engulfs and digests cellular debris, foreign substances, microbes, cancer cells via phagocytosis. Besides phagocytosis, they play important roles in nonspecific defense (innate immunity) and also help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines. Dendritic cells (DCs) are antigen-presenting cells that function in processing antigen material and present it on the cell surface to the T cells of the immune system.

The present disclosure provides, inter alia, multi-specific (e.g., bi-, tri-, quad-specific) proteins, that include, e.g., are engineered to contain, one or more B cell, macrophage, and/or dendritic cell engager that mediate binding to and/or activation of a B cell, macrophage, and/or dendritic cell.

Accordingly, in some embodiments, the immune cell engager comprises a B cell, macrophage, and/or dendritic cell engager chosen from one or more of CD40 ligand (CD40L) or a CD70 ligand; an antibody molecule that binds to CD40 or CD70; an antibody molecule to OX40; an OX40 ligand (OX40L); an agonist of a Toll-like receptor (e.g., as described herein, e.g., a TLR4, e.g., a constitutively active TLR4 (caTLR4), or a TLR9 agonists); a 41BB; a CD2; a CD47; or a STING agonist, or a combination thereof.

In some embodiments, the B cell engager is a CD40L, an OX40L, or a CD70 ligand, or an antibody molecule that binds to OX40, CD40 or CD70, e.g., also described herein as TNFSF family members.

In some embodiments, the macrophage engager is a CD2 agonist. In some embodiments, the macrophage engager is an antigen binding domain that binds to: CD40L or antigen binding domain or ligand that binds CD40, a Toll like receptor (TLR) agonist (e.g., as described herein), e.g., a TLR9 or TLR4 (e.g., caTLR4 (constitutively active TLR4), CD47, or a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In some embodiments, the dendritic cell engager is a CD2 agonist. In some embodiments, the dendritic cell engager is a ligand, a receptor agonist, or an antibody molecule that binds to one or more of: OX40L, 41BB, a TLR agonist (e.g., as described herein) (e.g., TLR9 agonist, TLR4 (e.g., caTLR4 (constitutively active TLR4)), CD47, or and a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In other embodiments, the immune cell engager mediates binding to, or activation of, one or more of a B cell, a macrophage, and/or a dendritic cell. Exemplary B cell, macrophage, and/or dendritic cell engagers can be chosen from one or more of CD40 ligand (CD40L) or a CD70 ligand; an antibody molecule that binds to CD40 or CD70; an antibody molecule to OX40; an OX40 ligand (OX40L); a Toll-like receptor agonist (e.g., a TLR4, e.g., a constitutively active TLR4 (caTLR4) or a TLR9 agonist); a 41BB agonist; a CD2; a CD47; or a STING agonist, or a combination thereof.

In some embodiments, the B cell engager is chosen from one or more of a CD40L, an OX40L, or a CD70 ligand, or an antibody molecule that binds to OX40, CD40 or CD70.

In other embodiments, the macrophage cell engager is chosen from one or more of a CD2 agonist; a CD40L; an OX40L; an antibody molecule that binds to OX40, CD40 or CD70; a Toll-like receptor agonist or a fragment thereof (e.g., a TLR4, e.g., a constitutively active TLR4 (caTLR4)); a CD47 agonist; or a STING agonist.

In other embodiments, the dendritic cell engager is chosen from one or more of a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist.

In yet other embodiments, the STING agonist comprises a cyclic dinucleotide, e.g., a cyclic di-GMP (cdGMP), a cyclic di-AMP (cdAMP), or a combination thereof, optionally with 2′,5′ or 3′,5′ phosphate linkages.

### Toll-Like Receptors

Toll-Like Receptors (TLRs) are evolutionarily conserved receptors are homologues of the Drosophila Toll protein, and recognize highly conserved structural motifs known as pathogen-associated microbial patterns (PAMPs), which are exclusively expressed by microbial pathogens, or danger-associated molecular patterns (DAMPs) that are endogenous molecules released from necrotic or dying cells. PAMPs include various bacterial cell wall components such as lipopolysaccharide (LPS), peptidoglycan (PGN) and lipopeptides, as well as flagellin, bacterial DNA and viral double-stranded RNA. DAMPs include intracellular proteins such as heat shock proteins as well as protein fragments from the extracellular matrix. Stimulation of TLRs by the corresponding PAMPs or DAMPs initiates signaling cascades leading to the activation of transcription factors, such as AP-1, NF-xB and interferon regulatory factors (IRFs). Signaling by TLRs results in a variety of cellular responses, including the production of interferons (IFNs), pro-inflammatory cytokines and effector cytokines that direct the adaptive immune response. TLRs are implicated in a number of inflammatory and immune disorders and play a role in cancer (Rakoff-Nahoum S. & Medzhitov R., 2009. Toll-like receptors and cancer. Nat Revs Cancer 9:57-63.)

TLRs are type I transmembrane proteins characterized by an extracellular domain containing leucine-rich repeats (LRRs) and a cytoplasmic tail that contains a conserved region called the Toll/IL-1 receptor (TIR) domain. Ten human and twelve murine TLRs have been characterized, TLR1 to TLR10 in humans, and TLR1 to TLR9, TLR11, TLR12 and TLR13 in mice, the homolog of TLR10 being a pseudogene. TLR2 is essential for the recognition of a variety of PAMPs from Gram-positive bacteria, including bacterial lipoproteins, lipomannans and lipoteichoic acids. TLR3 is implicated in virus-derived double-stranded RNA. TLR4 is predominantly activated by lipopolysaccharide. TLR5 detects bacterial flagellin and TLR9 is required for response to unmethylated CpG DNA. Finally, TLR7 and TLR8 recognize small synthetic antiviral molecules, and single-stranded RNA was reported to be their natural ligand. TLR11 has been reported to recognize uropathogenic E. coli and a profilin-like protein from Toxoplasma gondii. The repertoire of specificities of the TLRs is apparently extended by the ability of TLRs to heterodimerize with one another. For example, dimers of TLR2 and TLR6 are required for responses to diacylated lipoproteins while TLR2 and TLR1 interact to recognize triacylated lipoproteins. Specificities of the TLRs are also influenced by various adapter and accessory molecules, such as MD-2 and CD14 that form a complex with TLR4 in response to LPS.

TLR signaling consists of at least two distinct pathways: a MyD88-dependent pathway that leads to the production of inflammatory cytokines, and a MyD88-independent pathway associated with the stimulation of IFN-β and the maturation of dendritic cells. The MyD88-dependent pathway is common to all TLRs, except TLR3 (Adachi O. et al., 1998. Targeted disruption of the MyD88 gene results in loss of IL-1- and IL-18-mediated function. Immunity. 9(1):143-50). Upon activation by PAMPs or DAMPS, TLRs hetero- or homodimerize inducing the recruitment of adaptor proteins via the cytoplasmic TIR domain. Individual TLRs induce different signaling responses by usage of the different adaptor molecules. TLR4 and TLR2 signaling requires the adaptor TIRAP/Mal, which is involved in the MyD88-dependent pathway. TLR3 triggers the production of IFN-β in response to double-stranded RNA, in a MyD88-independent manner, through the adaptor TRIF/TICAM-1. TRAM/TICAM-2 is another adaptor molecule involved in the MyD88-independent pathway which function is restricted to the TLR4 pathway.

TLR3, TLR7, TLR8 and TLR9 recognize viral nucleic acids and induce type I IFNs. The signaling mechanisms leading to the induction of type I IFNs differ depending on the TLR activated. They involve the interferon regulatory factors, IRFs, a family of transcription factors known to play a critical role in antiviral defense, cell growth and immune regulation. Three IRFs (IRF3, IRF5 and IRF7) function as direct transducers of virus-mediated TLR signaling. TLR3 and TLR4 activate IRF3 and IRF7, while TLR7 and TLR8 activate IRF5 and IRF7 (Doyle S. et al., 2002. IRF3 mediates a TLR3/TLR4-specific antiviral gene program. Immunity. 17(3):251-63). Furthermore, type I IFN production stimulated by TLR9 ligand CpG-A has been shown to be mediated by PI(3)K and mTOR (Costa-Mattioli M. & Sonenberg N. 2008. RAPping production of type I interferon in pDCs through mTOR. Nature Immunol. 9: 1097-1099).

### TLR-9

TLR9 recognizes unmethylated CpG sequences in DNA molecules. CpG sites are relatively rare (˜1%) on vertebrate genomes in comparison to bacterial genomes or viral DNA. TLR9 is expressed by numerous cells of the immune system such as B lymphocytes, monocytes, natural killer (NK) cells, and plasmacytoid dendritic cells. TLR9 is expressed intracellularly, within the endosomal compartments and functions to alert the immune system of viral and bacterial infections by binding to DNA rich in CpG motifs. TLR9 signals leads to activation of the cells initiating pro-inflammatory reactions that result in the production of cytokines such as type-I interferon and IL-12.

### TLR Agonists

A TLR agonist can agonize one or more TLR, e.g., one or more of human TLR-1, 2, 3, 4, 5, 6, 7, 8, 9, or 10. In some embodiments, an adjunctive agent described herein is a TLR agonist. In some embodiments, the TLR agonist specifically agonizes human TLR-9. In some embodiments, the TLR-9 agonist is a CpG moiety. As used herein, a CpG moiety, is a linear dinucleotide having the sequence: 5′-C-phosphate-G-3′, that is, cytosine and guanine separated by only one phosphate.

In some embodiments, the CpG moiety comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more CpG dinucleotides. In some embodiments, the CpG moiety consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 CpG dinucleotides. In some embodiments, the CpG moiety has 1-5, 1-10, 1-20, 1-30, 1-40, 1-50, 5-10, 5-20, 5-30, 10-20, 10-30, 10-40, or 10-50 CpG dinucleotides.

In some embodiments, the TLR-9 agonist is a synthetic ODN (oligodeoxynucleotides). CpG ODNs are short synthetic single-stranded DNA molecules containing unmethylated CpG dinucleotides in particular sequence contexts (CpG motifs). CpG ODNs possess a partially or completely phosphorothioated (PS) backbone, as opposed to the natural phosphodiester (PO) backbone found in genomic bacterial DNA. There are three major classes of CpG ODNs: classes A, B and C, which differ in their immunostimulatory activities. CpG-A ODNs are characterized by a PO central CpG-containing palindromic motif and a PS-modified 3′ poly-G string. They induce high IFN-α production from pDCs but are weak stimulators of TLR9-dependent NF-xB signaling and pro-inflammatory cytokine (e.g. IL-6) production. CpG-B ODNs contain a full PS backbone with one or more CpG dinucleotides. They strongly activate B cells and TLR9-dependent NF-κB signaling but weakly stimulate IFN-α secretion. CpG-C ODNs combine features of both classes A and B. They contain a complete PS backbone and a CpG-containing palindromic motif. C-Class CpG ODNs induce strong IFN-α production from pDC as well as B cell stimulation.

Exemplary amino acid sequences for immune cell engagers:

In one embodiment, the NK cell engager is a ligand of NKp30 is a B7-6, e.g., comprises the amino acid sequence of:

(SEQ ID NO: 274)
DLKVEMMAGGTQITPLNDNVTIFCNIFYSQPLNITSMGITWFWKSLTFDK
EVKVFEFFGDHQEAFRPGAIVSPWRLKSGDASLRLPGIQLEEAGEYRCEV
VVTPLKAQGTVQLEVVASPASRLLLDQVGMKENEDKYMCESSGFYPEAIN
ITWEKQTQKFPHPIEISEDVITGPTIKNMDGTFNVTSCLKLNSSQEDPGT
VYQCVVRHASLHTPLRSNFTLTAARHSLSETEKTDNFS,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 274.

In other embodiments, the NK cell engager is a ligand of NKp44 or NKp46, which is a viral HA. Viral hemagglutinins (HA) are glyco proteins which are on the surface of viruses. HA proteins allow viruses to bind to the membrane of cells via sialic acid sugar moieties which contributes to the fusion of viral membranes with the cell membranes (see e.g., Eur J Immunol. 2001 September; 31(9):2680-9 “Recognition of viral hemagglutinins by NKp44 but not by NKp30”; and Nature. 2001 Feb. 22; 409(6823):1055-60 “Recognition of haemagglutinins on virus-infected cells by NKp46 activates lysis by human NK cells” the contents of each of which are incorporated by reference herein).

In other embodiments, the NK cell engager is a ligand of NKG2D chosen from MICA, MICB, or ULBP1, e.g., wherein:

(i) MICA comprises the amino acid sequence:

(SEQ ID NO: 275)
EPHSLRYNLTVLSWDGSVQSGFLTEVHLDGQPFLRCDRQKCRAKPQGQWA
EDVLGNKTWDRETRDLTGNGKDLRMTLAHIKDQKEGLHSLQEIRVCEIHE
DNSTRSSQHFYYDGELFLSQNLETKEWTMPQSSRAQTLAMNVRNFLKEDA
MKTKTHYHAMHADCLQELRRYLKSGVVLRRTVPPMVNVTRSEASEGNITV
TCRASGFYPWNITLSWRQDGVSLSHDTQQWGDVLPDGNGTYQTWVATRIC
QGEEQRFTCYMEHSGNHSTHPVPSGKVLVLQSHW,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 275;

(ii) MICB comprises the amino acid sequence:

(SEQ ID NO: 276)
AEPHSLRYNLMVLSQDESVQSGFLAEGHLDGQPFLRYDRQKRRAKPQGQW
AEDVLGAKTWDTETEDLTENGQDLRRTLTHIKDQKGGLHSLQEIRVCEIH
EDSSTRGSRHFYYDGELFLSQNLETQESTVPQSSRAQTLAMNVTNFWKED
AMKTKTHYRAMQADCLQKLQRYLKSGVAIRRTVPPMVNVTCSEVSEGNIT
VTCRASSFYPRNITLTWRQDGVSLSHNTQQWGDVLPDGNGTYQTWVATRI
RQGEEQRFTCYMEHSGNHGTHPVPSGKVLVLQSQRTD,

a fragment thereof, or an amino acid sequence substantially identical thereto (e.g., at least 80%, 85%, 90%, 95%, 99% or more identical identical thereto, or having at least one amino acid alteration, but not more than five, ten or fifteen alterations (e.g., substitutions, deletions, or insertions, e.g., conservative substitutions) to the amino acid sequence of SEQ ID NO: 276; or

(iii) ULBP1 comprises the amino acid sequence:

(SEQ ID NO: 277)
GWVDTHCLCYDFIITPKSRPEPQWCEVQGLVDERPFLHYDCVNHKAKAF
ASLGKKVNVTKTWEEQTETLRDVVDFLKGQLLDIQVENLIPIEPLTLQA
RMSCEHEAHGHGRGSWQFLFNGQKFLLFDSNNRKWTALHPGAKKMTEKW
EKNRDVTMFFQKISLGDCKMWLEEFLMYWEQMLDPTKPPSLAPG,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 277.

In other embodiments, the NK cell engager is a ligand of DNAM1 chosen from NECTIN2 or NECL5, e.g., wherein:

(i) NECTIN2 comprises the amino acid sequence:

(SEQ ID NO: 278)
QDVRVQVLPEVRGQLGGTVELPCHLLPPVPGLYISLVTWQRPDAPANHQ
NVAAFHPKMGPSFPSPKPGSERLSFVSAKQSTGQDTEAELQDATLALHG
LTVEDEGNYTCEFATFPKGSVRGMTWLRVIAKPKNQAEAQKVTFSQDPT
TVALCISKEGRPPARISWLSSLDWEAKETQVSGTLAGTVTVTSRFTLVP
SGRADGVTVTCKVEHESFEEPALIPVTLSVRYPPEVSISGYDDNWYLGR
TDATLSCDVRSNPEPTGYDWSTTSGTFPTSAVAQGSQLVIHAVDSLFNT
TFVCTVTNAVGMGRAEQVIFVRETPNTAGAGATGG,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 278; or

(ii) NECL5 comprises the amino acid sequence:

(SEQ ID NO: 279)
WPPPGTGDVVVQAPTQVPGFLGDSVTLPCYLQVPNMEVTHVSQLTWARH
GESGSMAVFHQTQGPSYSESKRLEFVAARLGAELRNASLRMFGLRVEDE
GNYTCLFVTFPQGSRSVDIWLRVLAKPQNTAEVQKVQLTGEPVPMARCV
STGGRPPAQITWHSDLGGMPNTSQVPGFLSGTVTVTSLWILVPSSQVDG
KNVTCKVEHESFEKPQLLTVNLTVYYPPEVSISGYDNNWYLGQNEATLT
CDARSNPEPTGYNWSTTMGPLPPFAVAQGAQLLIRPVDKPINTTLICNV
TNALGARQAELTVQVKEGPPSEHSGISRN,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 279.

In yet other embodiments, the NK cell engager is a ligand of DAP10, which is an adapter for NKG2D (see e.g., Proc Natl Acad Sci USA. 2005 May 24; 102(21): 7641-7646; and Blood, 15 Sep. 2011 Volume 118, Number 11, the full contents of each of which is incorporated by reference herein).

In other embodiments, the NK cell engager is a ligand of CD16, which is a CD16a/b ligand, e.g., a CD16a/b ligand further comprising an antibody Fc region (see e.g., Front Immunol. 2013; 4: 76 discusses how antibodies use the Fc to trigger NK cells through CD16, the full contents of which are incorporated herein).

In other embodiments, the NK cell engager is a ligand of CRTAM, which is NECL2, e.g., wherein NECL2 comprises the amino acid sequence:

(SEQ ID NO: 280)
QNLFTKDVTVIEGEVATISCQVNKSDDSVIQLLNPNRQTIYFRDFRPLK
DSRFQLLNFSSSELKVSLTNVSISDEGRYFCQLYTDPPQESYTTITVLV
PPRNLMIDIQKDTAVEGEEIEVNCTAMASKPATTIRWFKGNTELKGKSE
VEEWSDMYTVTSQLMLKVHKEDDGVPVICQVEHPAVTGNLQTQRYLEVQ
YKPQVHIQMTYPLQGLTREGDALELTCEAIGKPQPVMVTWVRVDDEMPQ
HAVLSGPNLFINNLNKTDNGTYRCEASNIVGKAHSDYMLYVYDPPTTIP
PPTTTTTTTTTTTTTILTIITDSRAGEEGSIRAVDH,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 280.

In other embodiments, the NK cell engager is a ligand of CD27, which is CD70, e.g., wherein CD70 comprises the amino acid sequence:

(SEQ ID NO: 281)
QRFAQAQQQLPLESLGWDVAELQLNHTGPQQDPRLYWQGGPALGRSFLH
GPELDKGQLRIHRDGIYMVHIQVTLAICSSTTASRHHPTTLAVGICSPA
SRSISLLRLSFHQGCTIASQRLTPLARGDTLCTNLTGTLLPSRNTDETF
FGVQWVRP,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 281.

In other embodiments, the NK cell engager is a ligand of PSGL1, which is L-selectin (CD62L), e.g., wherein L-selectin comprises the amino acid sequence:

(SEQ ID NO: 282)
WTYHYSEKPMNWQRARRFCRDNYTDLVAIQNKAEIEYLEKTLPFSRSYY
WIGIRKIGGIWTWVGTNKSLTEEAENWGDGEPNNKKNKEDCVEIYIKRN
KDAGKWNDDACHKLKAALCYTASCQPWSCSGHGECVEIINNYTCNCDVG
YYGPQCQFVIQCEPLEAPELGTMDCTHPLGNFSFSSQCAFSCSEGTNLT
GIEETTCGPFGNWSSPEPTCQVIQCEPLSAPDLGIMNCSHPLASFSFTS
ACTFICSEGTELIGKKKTICESSGIWSNPSPICQKLDKSFSMIKEGDY
N,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 282.

In other embodiments, the NK cell engager is a ligand of CD96, which is NECL5, e.g., wherein NECL5 comprises the amino acid sequence:

(SEQ ID NO: 283)
WPPPGTGDVVVQAPTQVPGFLGDSVTLPCYLQVPNMEVTHVSQLTWARH
GESGSMAVFHQTQGPSYSESKRLEFVAARLGAELRNASLRMFGLRVEDE
GNYTCLFVTFPQGSRSVDIWLRVLAKPQNTAEVQKVQLTGEPVPMARCV
STGGRPPAQITWHSDLGGMPNTSQVPGFLSGTVTVTSLWILVPSSQVDG
KNVTCKVEHESFEKPQLLTVNLTVYYPPEVSISGYDNNWYLGQNEATLT
CDARSNPEPTGYNWSTTMGPLPPFAVAQGAQLLIRPVDKPINTTLICNV
TNALGARQAELTVQVKEGPPSEHSGISRN,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 283.

In other embodiments, the NK cell engager is a ligand of CD100 (SEMA4D), which is CD72, e.g., wherein CD72 comprises the amino acid sequence:

(SEQ ID NO: 284)
RYLQVSQQLQQTNRVLEVTNSSLRQQLRLKITQLGQSAEDLQGSRRELA
QSQEALQVEQRAHQAAEGQLQACQADRQKTKETLQSEEQQRRALEQKLS
NMENRLKPFFTCGSADTCCPSGWIMHQKSCFYISLTSKNWQESQKQCET
LSSKLATFSEIYPQSHSYYFLNSLLPNGGSGNSYWTGLSSNKDWKLTDD
TQRTRTYAQSSKCNKVHKTWSWWTLESESCRSSLPYICEMTAFRFPD,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 284.

In other embodiments, the NK cell engager is a ligand of NKp80, which is CLEC2B (AICL), e.g., wherein CLEC2B (AICL) comprises the amino acid sequence:

(SEQ ID NO: 285)
KLTRDSQSLCPYDWIGFQNKCYYFSKEEGDWNSSKYNCSTQHADLTIID
NIEEMNFLRRYKCSSDHWIGLKMAKNRTGQWVDGATFTKSFGMRGSEGC
AYLSDDGAATARCYTERKWICRKRIH,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 285.

In other embodiments, the NK cell engager is a ligand of CD244, which is CD48, e.g., wherein CD48 comprises the amino acid sequence:

(SEQ ID NO: 286)
QGHLVHMTVVSGSNVTLNISESLPENYKQLTWFYTFDQKIVEWDSRKSK
YFESKFKGRVRLDPQSGALYISKVQKEDNSTYIMRVLKKTGNEQEWKIK
LQVLDPVPKPVIKIEKIEDMDDNCYLKLSCVIPGESVNYTWYGDKRPFP
KELQNSVLETTLMPHNYSRCYTCQVSNSVSSKNGTVCLSPPCTLARS,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 286.

In other embodiments, the immune cell engager mediates binding to, or activation of, one or more of a B cell, a macrophage, and/or a dendritic cell. Exemplary B cell, macrophage, and/or dendritic cell engagers can be chosen from one or more of CD40 ligand (CD40L) or a CD70 ligand; an antibody molecule that binds to CD40 or CD70; an antibody molecule to OX40; an OX40 ligand (OX40L); a Toll-like receptor agonist (e.g., a TLR4, e.g., a constitutively active TLR4 (caTLR4) or a TLR9 agonist); a 41BB agonist; a CD2; a CD47; or a STING agonist, or a combination thereof.

In some embodiments, the B cell engager is chosen from one or more of a CD40L, an OX40L, or a CD70 ligand, or an antibody molecule that binds to OX40, CD40 or CD70.

In other embodiments, the macrophage cell engager is chosen from one or more of a CD2 agonist; a CD40L; an OX40L; an antibody molecule that binds to OX40, CD40 or CD70; a Toll-like receptor agonist or a fragment thereof (e.g., a TLR4, e.g., a constitutively active TLR4 (caTLR4)); a CD47 agonist; or a STING agonist.

In other embodiments, the dendritic cell engager is chosen from one or more of a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist.

In one embodiment, the OX40L comprises the amino acid sequence:

(SEQ ID NO: 287)
QVSHRYPRIQSIKVQFTEYKKEKGFILTSQKEDEIMKVQNNSVIINCDG
FYLISLKGYFSQEVNISLHYQKDEEPLFQLKKVRSVNSLMVASLTYKDK
VYLNVTTDNTSLDDFHVNGGELILIHQNPGEFCVL,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 287.

In another embodiment, the CD40L comprises the amino acid sequence:

(SEQ ID NO: 288)
MQKGDQNPQIAAHVISEASSKTTSVLQWAEKGYYTMSNNLVTLENGKQL
TVKRQGLYYIYAQVTFCSNREASSQAPFIASLCLKSPGRFERILLRAAN
THSSAKPCGQQSIHLGGVFELQPGASVFVNVTDPSQVSHGTGFTSFGLL
KL,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 288.

In yet other embodiments, the STING agonist comprises a cyclic dinucleotide, e.g., a cyclic di-GMP (cdGMP), a cyclic di-AMP (cdAMP), or a combination thereof, optionally with 2′,5′ or 3′,5′ phosphate linkages.

In one embodiment, the immune cell engager includes 41BB ligand, e.g., comprising the amino acid sequence:

(SEQ ID NO: 289)
ACPWAVSGARASPGSAASPRLREGPELSPDDPAGLLDLRQGMFAQLVAQ
NVLLIDGPLSWYSDPGLAGVSLTGGLSYKEDTKELVVAKAGVYYVFFQL
ELRRVVAGEGSGSVSLALHLQPLRSAAGAAALALTVDLPPASSEARNSA
FGFQGRLLHLSAGQRLGVHLHTEARARHAWQLTQGATVLGLFRVTPEIP
AGLPSPRSE,

a fragment thereof, or an amino acid sequence substantially identical thereto (e.g., at least 80%, 85%, 90%, 95%, 99% or more identical thereto, or having at least one amino acid alteration, but not more than five, ten or fifteen alterations (e.g., substitutions, deletions, or insertions, e.g., conservative substitutions) to the amino acid sequence of SEQ ID NO: 289.

### Linkers

The multifunctional molecule disclosed herein can further include a linker, e.g., a linker between one or more of: the trimer ligand, the targeting moiety, the cytokine molecule, the immune cell engager, and the dimerization module, e.g., the immunoglobulin chain constant region (e.g., the Fc region). In embodiments, the linker chosen from: a cleavable linker, a non-cleavable linker, a peptide linker, a flexible linker, a rigid linker, a helical linker, or a non-helical linker, or a combination thereof.

In one embodiment, the multispecific molecule can include one, two, three or four linkers, e.g., a peptide linker. In one embodiment, the peptide linker includes Gly and Ser. Exemplary peptide linkers are depicted in the figures disclosed herein, e.g., a peptide linker chosen from:

(SEQ ID NO: 290)
GGGGS;
(SEQ ID NO: 291)
GGGGSGGGGS;
(SEQ ID NO: 292)
GGGGSGGGGSGGGGS;
or
(SEQ ID NO: 293)
DVPSGPGGGGGSGGGGS.

### Nucleic Acids

The invention also features nucleic acids comprising nucleotide sequences that encode the multifunctional molecules described herein. For example, the invention features a first and second nucleic acid encoding heavy and light chain variable regions, respectively, of an antibody molecule chosen from one or more of the antibody molecules disclosed herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In another aspect, the application features host cells and vectors containing the nucleic acids described herein. The nucleic acids may be present in a single vector or separate vectors present in the same host cell or separate host cell, as described in more detail hereinbelow.

### Vectors

Further provided herein are vectors comprising the nucleotide sequences encoding an antibody molecule described herein. In one embodiment, the vectors comprise nucleotides encoding an antibody molecule described herein. In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

In another aspect, the application features host cells and vectors containing the nucleic acids described herein. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

The invention also provides host cells comprising a nucleic acid encoding an antibody molecule as described herein.

In one embodiment, the host cells are genetically engineered to comprise nucleic acids encoding the antibody molecule.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The invention also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### Methods of Making the Multifunctional Molecules

The multifunctional antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). In one embodiment, the host cell is a mammalian cell, a stable mammalian cell, e.g., a CHO cell. Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification, e.g., histidine-containing tag, myc tag, or streptavidin tag.

In some embodiments, a method for generating bispecific antibodies disclosed herein comprises: generating a human antibody with a light chain of a lambda subtype; generating a human antibody with a light chain of kappa subtype; transfecting cells with DNA of both antibody arms; purifying the antibody with Protein A resin; confirming the presence of both lambda and kappa light chains with KappaSelect and LambdaFabSelect resin; analyzing the correct lambda and kappa heavy and light chain pairing by cleaving Fab arms with papain and running mass spectrometry.

### Methods of Making Dimerized Molecules

### Heavy Chain Pairing

Various methods of producing multispecific antibodies have been disclosed to address the problem of incorrect heavy chain pairing. Exemplary methods are described below. Exemplary multispecific antibody formats and methods of making said multispecific antibodies are also disclosed in e.g., Speiss et al. Molecular Immunology 67 (2015) 95-106; and Klein et al mAbs 4:6, 653-663; November/December 2012; the entire contents of each of which are incorporated by reference herein.

### Heterodimerized Antibody Molecules

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

### Knob-In-Hole

Knob-in-Hole as described in U.S. Pat. Nos. 5,731,116, 7,476,724 and Ridgway, J. et al. (1996) Prot. Engineering 9(7): 617-621, broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, L368A and/or Y407V).

For bispecific antibodies including an Fc domain, introduction of specific mutations into the constant region of the heavy chains to promote the correct heterodimerization of the Fc portion can be utilized. Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228).

Exemplary KiH mutations include S354C, T366W in the “knob” heavy chain and Y349C, T366S, L368A, Y407V in the “hole” heavy chain. Other exemplary KiH mutations are provided in Table 1, with additional optional stabilizing Fc cysteine mutations.

TABLE 8
Exemplary Fc KiH mutations and
optional Cysteine mutations

| Position | Knob Mutation | Hole Mutation |
|---|---|---|
| T366 | T366W | T366S |
| L368 | — | L368A |
| Y407 | — | Y407V |
| Additional Cysteine Mutations to form a | | |
| stabilizing disulfide bridge | | |
| Position | Knob CH3 | Hole CH3 |
| S354 | S354C | — |
| Y349 | — | Y349C |

Other Fc mutations are provided by Igawa and Tsunoda who identified 3 negatively charged residues in the CH3 domain of one chain that pair with three positively charged residues in the CH3 domain of the other chain. These specific charged residue pairs are: E356-K439, E357-K370, D399-K409 and vice versa. By introducing at least two of the following three mutations in chain A: E356K, E357K and D399K, as well as K370E, K409D, K439E in chain B, alone or in combination with newly identified disulfide bridges, they were able to favor very efficient heterodimerization while suppressing homodimerization at the same time (Martens T et al. A novel one-armed antic-Met antibody inhibits glioblastoma growth in vivo. Clin Cancer Res 2006; 12:6144-52; PMID:17062691). Xencor defined 41 variant pairs based on combining structural calculations and sequence information that were subsequently screened for maximal heterodimerization, defining the combination of S364H, F405A (HA) on chain A and Y349T, T394F on chain B (TF) (Moore G L et al. A novel bispecific antibody format enables simultaneous bivalent and monovalent co-engagement of distinct target antigens. MAbs 2011; 3:546-57; PMID: 22123055).

Other exemplary Fc mutations to promote heterodimerization of multispecific antibodies include those described in the following references, the contents of each of which is incorporated by reference herein, WO2016071377A1, US20140079689A1, US20160194389A1, US20160257763, WO2016071376A2, WO2015107026A1, WO2015107025A1, WO2015107015A1, US20150353636A1, US20140199294A1, U.S. Pat. No. 7,750,128B2, US20160229915A1, US20150344570A1, U.S. Pat. No. 8,003,774A1, US20150337049A1, US20150175707A1, US20140242075A1, US20130195849A1, US20120149876A1, US20140200331A1, U.S. Pat. No. 9,309,311B2, U.S. Pat. No. 8,586,713, US20140037621A1, US20130178605A1, US20140363426A1, US20140051835A1 and US20110054151A1.

Stabilizing cysteine mutations have also been used in combination with KiH and other Fc heterodimerization promoting variants, see e.g., U.S. Pat. No. 7,183,076. Other exemplary cysteine modifications include, e.g., those disclosed in US20140348839A1, U.S. Pat. No. 7,855,275B2, and U.S. Pat. No. 9,000,130B2.

### Strand Exchange Engineered Domains (SEED)

Heterodimeric Fc platform that support the design of bispecific and asymmetric fusion proteins by devising strand-exchange engineered domain (SEED) C(H)3 heterodimers are known. These derivatives of human IgG and IgA C(H)3 domains create complementary human SEED C(H)3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgG1 hinge]-C(H)2-[SEED C(H)3], that may be genetically linked to one or more fusion partners (see e.g., Davis J H et al. SEEDbodies: fusion proteins based on strand exchange engineered domain (SEED) CH3 heterodimers in an Fc analogue platform for asymmetric binders or immunofusions and bispecific antibodies. Protein Eng Des Sel 2010; 23:195-202; PMID:20299542 and U.S. Pat. No. 8,871,912. The contents of each of which are incorporated by reference herein).

### Duobody

“Duobody” technology to produce bispecific antibodies with correct heavy chain pairing are known. The DuoBody technology involves three basic steps to generate stable bispecific human IgG1 antibodies in a post-production exchange reaction. In a first step, two IgG1s, each containing single matched mutations in the third constant (CH3) domain, are produced separately using standard mammalian recombinant cell lines. Subsequently, these IgG1 antibodies are purified according to standard processes for recovery and purification. After production and purification (post-production), the two antibodies are recombined under tailored laboratory conditions resulting in a bispecific antibody product with a very high yield (typically >95%) (see e.g., Labrijn et al, PNAS 2013; 110(13):5145-5150 and Labrijn et al. Nature Protocols 2014; 9(10):2450-63, the contents of each of which are incorporated by reference herein).

### Electrostatic Interactions

Methods of making multispecific antibodies using CH3 amino acid changes with charged amino acids such that homodimer formation is electrostatically unfavorable are disclosed. EP1870459 and WO 2009089004 describe other strategies for favoring heterodimer formation upon co-expression of different antibody domains in a host cell. In these methods, one or more residues that make up the heavy chain constant domain 3 (CH3), CH3-CH3 interfaces in both CH3 domains are replaced with a charged amino acid such that homodimer formation is electrostatically unfavorable and heterodimerization is electrostatically favorable. Additional methods of making multispecific molecules using electrostatic interactions are described in the following references, the contents of each of which is incorporated by reference herein, include US20100015133, U.S. Pat. No. 8,592,562B2, U.S. Pat. No. 9,200,060B2, US20140154254A1, and U.S. Pat. No. 9,358,286A1.

### Light Chain Pairing

### Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., U.S. Pat. No. 7,183,076B2, US20110177073A1, EP2847231A1, WO2016079081A1, and EP3055329A1, the contents of each of which is incorporated by reference herein.

### CrossMab

Another option to reduce light chain mispairing is the CrossMab technology which avoids non-specific L chain mispairing by exchanging CH1 and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain—heavy chain pairs, a two-step modification process is applied. First, a dimerization interface is engineered into the C-terminus of each heavy chain using a heterodimerization approach, e.g., Knob-into-hole (KiH) technology, to ensure that only a heterodimer of two distinct heavy chains from one antibody (e.g., Antibody A) and a second antibody (e.g., Antibody B) is efficiently formed. Next, the constant heavy 1 (CH1) and constant light (CL) domains of one antibody are exchanged (Antibody A), keeping the variable heavy (VH) and variable light (VL) domains consistent. The exchange of the CH1 and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain, C. SciBX 4(28); doi:10.1038/scibx.2011.783, the contents of which are incorporated by reference herein).

### Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A1, the contents of each of which is incorporated by reference herein.

### Amino Acid Modifications

Alternative compositions and methods of producing multispecific antibodies with correct light chain pairing include various amino acid modifications. For example, Zymeworks describes heterodimers with one or more amino acid modifications in the CH1 and/or CL domains, one or more amino acid modifications in the VH and/or VL domains, or a combination thereof, which are part of the interface between the light chain and heavy chain and create preferential pairing between each heavy chain and a desired light chain such that when the two heavy chains and two light chains of the heterodimer pair are co-expressed in a cell, the heavy chain of the first heterodimer preferentially pairs with one of the light chains rather than the other (see e.g., WO2015181805). Other exemplary methods are described in WO2016026943 (Argen-X), US20150211001, US20140072581A1, US20160039947A1, and US20150368352.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

Other exemplary multispecific antibody formats include, e.g., those described in the following US20160114057A1, US20130243775A1, US20140051833, US20130022601, US20150017187A1, US20120201746A1, US20150133638A1, US20130266568A1, US20160145340A1, WO2015127158A1, US20150203591A1, US20140322221A1, US20130303396A1, US20110293613, US20130017200A1, US20160102135A1, WO2015197598A2, WO2015197582A1, U.S. Pat. No. 9,359,437, US20150018529, WO2016115274A1, WO2016087416A1, US20080069820A1, U.S. Pat. Nos. 9,145,588B, 7,919,257, and US20150232560A1. Exemplary multispecific molecules utilizing a full antibody-Fab/scFab format include those described in the following, U.S. Pat. No. 9,382,323B2, US20140072581A1, US20140308285A1, US20130165638A1, US20130267686A1, US20140377269A1, U.S. Pat. No. 7,741,446B2, and WO1995009917A1. Exemplary multispecific molecules utilizing a domain exchange format include those described in the following, US20150315296A1, WO2016087650A1, US20160075785A1, WO2016016299A1, US20160130347A1, US20150166670, U.S. Pat. No. 8,703,132B2, US20100316645, U.S. Pat. No. 8,227,577B2, US20130078249.

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable domains (without peptide linker) fused to the C-terminus of CH3 domains of IgGs.

### Fc-Containing Multipecific Molecules

In some embodiments, the multispecific molecules disclosed herein includes an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1, IgG2, IgG3, or IgG4.

In some embodiments, the immunoglobulin chain constant region (e.g., the Fc region) is altered, e.g., mutated, to increase or decrease one or more of: Fc receptor binding, antibody glycosylation, the number of cysteine residues, effector cell function, or complement function.

In other embodiments, an interface of a first and second immunoglobulin chain constant regions (e.g., a first and a second Fc region) is altered, e.g., mutated, to increase or decrease dimerization, e.g., relative to a non-engineered interface, e.g., a naturally-occurring interface. For example, dimerization of the immunoglobulin chain constant region (e.g., the Fc region) can be enhanced by providing an Fc interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

In some embodiments, the multispecific molecules include a paired amino acid substitution at a position chosen from one or more of 347, 349, 350, 351, 366, 368, 370, 392, 394, 395, 397, 398, 399, 405, 407, or 409, e.g., of the Fc region of human IgG1 For example, the immunoglobulin chain constant region (e.g., Fc region) can include a paired amino acid substitution chosen from: T366S, L368A, or Y407V (e.g., corresponding to a cavity or hole), and T366W (e.g., corresponding to a protuberance or knob).

In other embodiments, the multifunctional molecule includes a half-life extender, e.g., a human serum albumin or an antibody molecule to human serum albumin.

### Lambda/Kappa Formats

Multispecific molecules (e.g., multispecific antibody molecules) that include the lambda light chain polypeptide and a kappa light chain polypeptides, can be used to allow for heterodimerization. Methods for generating bispecific antibody molecules comprising the lambda light chain polypeptide and a kappa light chain polypeptides are disclosed in U.S. Ser. No. 62/399,319 filed on Sep. 23, 2016, incorporated herein by reference in its entirety.

In embodiments, the multispecific molecules includes a multispecific antibody molecule, e.g., an antibody molecule comprising two binding specificities, e.g., a bispecific antibody molecule. The multispecific antibody molecule includes:

a lambda light chain polypeptide 1 (LLCP1) specific for a first epitope;

a heavy chain polypeptide 1 (HCP1) specific for the first epitope;

a kappa light chain polypeptide 2 (KLCP2) specific for a second epitope; and

a heavy chain polypeptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In an embodiment it comprises all or a fragment of a CH1 region. In an embodiment, an LLCP1 comprises LC-CDR1, LC-CDR2, LC-CDR3, FR1, FR2, FR3, FR4, and CH1, or sufficient sequence therefrom to mediate specific binding of its epitope and complex with an HCP1. LLCP1, together with its HCP1, provide specificity for a first epitope (while KLCP2, together with its HCP2, provide specificity for a second epitope). As described elsewhere herein, LLCP1 has a higher affinity for HCP1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In an embodiments it comprises all or a fragment of a CH1 region. In an embodiment, a KLCP2 comprises LC-CDR1, LC-CDR2, LC-CDR3, FR1, FR2, FR3, FR4, and CH1, or sufficient sequence therefrom to mediate specific binding of its epitope and complex with an HCP2. KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiments it comprises all or a fragment of a CH1 region. In an embodiment, it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1. HCP1, together with its LLCP1, provide specificity for a first epitope (while KLCP2, together with its HCP2, provide specificity for a second epitope).

“Heavy chain polypeptide 2 (HCP2)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiments it comprises all or a fragment of a CH1 region. In an embodiments it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an KLCP2, (ii) to complex preferentially, as described herein to KLCP2 as opposed to LLCP1; and (iii) to complex preferentially, as described herein, to an HCP1, as opposed to another molecule of HCP2. HCP2, together with its KLCP2, provide specificity for a second epitope (while LLCP1, together with its HCP1, provide specificity for a first epitope).

In some embodiments of the multispecific antibody molecule disclosed herein:

LLCP1 has a higher affinity for HCP1 than for HCP2; and/or

KLCP2 has a higher affinity for HCP2 than for HCP1.

In embodiments, the affinity of LLCP1 for HCP1 is sufficiently greater than its affinity for HCP2, such that under preselected conditions, e.g., in aqueous buffer, e.g., at pH 7, in saline, e.g., at pH 7, or under physiological conditions, at least 75%, 80, 90, 95, 98, 99, 99.5, or 99.9% of the multispecific antibody molecule molecules have a LLCP1 complexed, or interfaced with, a HCP1.

In some embodiments of the multispecific antibody molecule disclosed herein:

the HCP1 has a greater affinity for HCP2, than for a second molecule of HCP1; and/or

the HCP2 has a greater affinity for HCP1, than for a second molecule of HCP2.

In embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99 99.5 or 99.9% of the multispecific antibody molecule molecules have a HCP1 complexed, or interfaced with, a HCP2.

In another aspect, disclosed herein is a method for making, or producing, a multispecific antibody molecule. The method includes:

(i) providing a first heavy chain polypeptide (e.g., a heavy chain polypeptide comprising one, two, three or all of a first heavy chain variable region (first VH), a first CH1, a first heavy chain constant region (e.g., a first CH2, a first CH3, or both));

(ii) providing a second heavy chain polypeptide (e.g., a heavy chain polypeptide comprising one, two, three or all of a second heavy chain variable region (second VH), a second CH1, a second heavy chain constant region (e.g., a second CH2, a second CH3, or both));

(iii) providing a lambda chain polypeptide (e.g., a lambda light variable region (VLλ), a lambda light constant chain (VLλ), or both) that preferentially associates with the first heavy chain polypeptide (e.g., the first VH); and

(iv) providing a kappa chain polypeptide (e.g., a lambda light variable region (VLλ), a lambda light constant chain (VLκ), or both) that preferentially associates with the second heavy chain polypeptide (e.g., the second VH),

under conditions where (i)-(iv) associate.

In embodiments, the first and second heavy chain polypeptides form an Fc interface that enhances heterodimerization.

In embodiments, (i)-(iv) (e.g., nucleic acid encoding (i)-(iv)) are introduced in a single cell, e.g., a single mammalian cell, e.g., a CHO cell. In embodiments, (i)-(iv) are expressed in the cell.

In embodiments, (i)-(iv) (e.g., nucleic acid encoding (i)-(iv)) are introduced in different cells, e.g., different mammalian cells, e.g., two or more CHO cell. In embodiments, (i)-(iv) are expressed in the cells.

In one embodiments, the method further comprises purifying a cell-expressed antibody molecule, e.g., using a lambda- and/or- kappa-specific purification, e.g., affinity chromatography.

In embodiments, the method further comprises evaluating the cell-expressed multispecific antibody molecule. For example, the purified cell-expressed multispecific antibody molecule can be analyzed by techniques known in the art, include mass spectrometry. In one embodiment, the purified cell-expressed antibody molecule is cleaved, e.g., digested with papain to yield the Fab moieties and evaluated using mass spectrometry.

In embodiments, the method produces correctly paired kappa/lambda multispecific, e.g., bispecific, antibody molecules in a high yield, e.g., at least 75%, 80, 90, 95, 98, 99 99.5 or 99.9%.

In other embodiments, the multispecific, e.g., a bispecific, antibody molecule that includes:

(i) a first heavy chain polypeptide (HCP1) (e.g., a heavy chain polypeptide comprising one, two, three or all of a first heavy chain variable region (first VH), a first CH1, a first heavy chain constant region (e.g., a first CH2, a first CH3, or both)), e.g., wherein the HCP1 binds to a first epitope;

(ii) a second heavy chain polypeptide (HCP2) (e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope;

(iii) a lambda light chain polypeptide (LLCP1) (e.g., a lambda light variable region (VL1), a lambda light constant chain (VL1), or both) that preferentially associates with the first heavy chain polypeptide (e.g., the first VH), e.g., wherein the LLCP1 binds to a first epitope; and

(iv) a kappa light chain polypeptide (KLCP2) (e.g., a lambda light variable region (VLk), a lambda light constant chain (VLk), or both) that preferentially associates with the second heavy chain polypeptide (e.g., the second VH), e.g., wherein the KLCP2 binds to a second epitope.

In embodiments, the first and second heavy chain polypeptides form an Fc interface that enhances heterodimerization. In embodiments, the multispecific antibody molecule has a first binding specificity that includes a hybrid VL1-CL1 heterodimerized to a first heavy chain variable region connected to the Fc constant, CH2-CH3 domain (having a knob modification) and a second binding specificity that includes a hybrid VLk-CLk heterodimerized to a second heavy chain variable region connected to the Fc constant, CH2-CH3 domain (having a hole modification).

Experimental conditions for making and testing the multifunctional molecules are provided in the Examples below.

### Uses and Combination Therapies

Methods described herein include treating a cancer in a subject by using a multifunctional molecule described herein, e.g., using a pharmaceutical composition described herein. Also provided are methods for reducing or ameliorating a symptom of a cancer in a subject, as well as methods for inhibiting the growth of a cancer and/or killing one or more cancer cells. In embodiments, the methods described herein decrease the size of a tumor and/or decrease the number of cancer cells in a subject administered with a described herein or a pharmaceutical composition described herein.

In embodiments, the cancer is a hematological cancer. In embodiments, the hematological cancer is a leukemia or a lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

In embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In embodiments, the multifunctional molecules (or pharmaceutical composition) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or multifunctional molecules) to be administered can be determined by a physician with consideration of individual differences in tumor size, extent of infection or metastasis, age, weight, and condition of the subject. In embodiments, the pharmaceutical composition described herein can be administered at a dosage of 10⁴ to 10⁹ cells/kg body weight, e.g., 10⁵ to 10⁶ cells/kg body weight, including all integer values within those ranges. In embodiments, the pharmaceutical composition described herein can be administered multiple times at these dosages. In embodiments, the pharmaceutical composition described herein can be administered using infusion techniques described in immunotherapy (see, e.g., Rosenberg et al., New Eng. J. of Med. 319:1676, 1988).

In embodiments, the multifunctional molecules or pharmaceutical composition is administered to the subject parenterally. In embodiments, the cells are administered to the subject intravenously, subcutaneously, intratumorally, intranodally, intramuscularly, intradermally, or intraperitoneally. In embodiments, the cells are administered, e.g., injected, directly into a tumor or lymph node. In embodiments, the cells are administered as an infusion (e.g., as described in Rosenberg et al., New Eng. J. of Med. 319:1676, 1988) or an intravenous push. In embodiments, the cells are administered as an injectable depot formulation. In embodiments, the subject is a mammal. In embodiments, the subject is a human, monkey, pig, dog, cat, cow, sheep, goat, rabbit, rat, or mouse. In embodimnets, the subject is a human. In embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Combination Therapies

The multifunctional molecules disclosed herein can be used in combination with a second therapeutic agent or procedure.

In embodiments, the multifunctional molecule and the second therapeutic agent or procedure are administered/performed after a subject has been diagnosed with a cancer, e.g., before the cancer has been eliminated from the subject. In embodiments, the multifunctional molecule and the second therapeutic agent or procedure are administered/performed simultaneously or concurrently. For example, the delivery of one treatment is still occurring when the delivery of the second commences, e.g., there is an overlap in administration of the treatments. In other embodiments, the multifunctional molecule and the second therapeutic agent or procedure are administered/performed sequentially. For example, the delivery of one treatment ceases before the delivery of the other treatment begins.

In embodiments, combination therapy can lead to more effective treatment than monotherapy with either agent alone. In embodiments, the combination of the first and second treatment is more effective (e.g., leads to a greater reduction in symptoms and/or cancer cells) than the first or second treatment alone. In embodiments, the combination therapy permits use of a lower dose of the first or the second treatment compared to the dose of the first or second treatment normally required to achieve similar effects when administered as a monotherapy. In embodiments, the combination therapy has a partially additive effect, wholly additive effect, or greater than additive effect.

In one embodiment, the multifunctional molecule is administered in combination with a therapy, e.g., a cancer therapy (e.g., one or more of anti-cancer agents, immunotherapy, photodynamic therapy (PDT), surgery and/or radiation). The terms “chemotherapeutic,” “chemotherapeutic agent,” and “anti-cancer agent” are used interchangeably herein. The administration of the multifunctional molecule and the therapy, e.g., the cancer therapy, can be sequential (with or without overlap) or simultaneous. Administration of the multifunctional molecule can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P. et al. (2011) CA Cancer J. Clin. 61:250-281).

### Anti-Cancer Therapies

In other embodiments, the multifunctional molecule is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTA®), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In another embodiment, the multifunctional molecule is administered in conjunction with a biologic. Biologics useful in the treatment of cancers are known in the art and a binding molecule of the invention may be administered, for example, in conjunction with such known biologics. For example, the FDA has approved the following biologics for the treatment of breast cancer: HERCEPTIN® (trastuzumab, Genentech Inc., South San Francisco, Calif.; a humanized monoclonal antibody that has anti-tumor activity in HER2-positive breast cancer); FASLODEX® (fulvestrant, AstraZeneca Pharmaceuticals, LP, Wilmington, Del.; an estrogen-receptor antagonist used to treat breast cancer); ARIMIDEX® (anastrozole, AstraZeneca Pharmaceuticals, LP; a nonsteroidal aromatase inhibitor which blocks aromatase, an enzyme needed to make estrogen); Aromasin® (exemestane, Pfizer Inc., New York, N.Y.; an irreversible, steroidal aromatase inactivator used in the treatment of breast cancer); FEMARA® (letrozole, Novartis Pharmaceuticals, East Hanover, N.J.; a nonsteroidal aromatase inhibitor approved by the FDA to treat breast cancer); and NOLVADEX® (tamoxifen, AstraZeneca Pharmaceuticals, LP; a nonsteroidal antiestrogen approved by the FDA to treat breast cancer), Other biologics with which the binding molecules of the invention may be combined include: AVASTIN® (bevacizumab, Genentech Inc.; the first FDA-approved therapy designed to inhibit angiogenesis); and ZEVALIN® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: AVASTIN®; ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC® (imatinib mesylate; a protein kinase inhibitor); and ERGAMISOL® (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include VELCADE® Velcade (bortezomib, Millennium Pharmaceuticals, Cambridge Mass.; a proteasome inhibitor). Additional biologics include THALIDOMID® (thalidomide, Clegene Corporation, Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDER)), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

In other embodiments, the multifunctional molecule is administered in combination with a viral cancer therapeutic agent. Exemplary viral cancer therapeutic agents include, but not limited to, vaccinia virus (vvDD-CDSR), carcinoembryonic antigen-expressing measles virus, recombinant vaccinia virus (TK-deletion plus GM-CSF), Seneca Valley virus-001, Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/AS04 vaccine, MVA-EBNA1/LMP2 Inj. vaccine, quadrivalent HPV vaccine, quadrivalent human papillomavirus (types 6, 11, 16, 18) recombinant vaccine (GARDASIL®), recombinant fowlpox-CEA(6D)/TRICOM vaccine; recombinant vaccinia-CEA(6D)-TRICOM vaccine, recombinant modified vaccinia Ankara-5T4 vaccine, recombinant fowlpox-TRICOM vaccine, oncolytic herpes virus NV1020, HPV L1 VLP vaccine V504, human papillomavirus bivalent (types 16 and 18) vaccine (CERVARIX®), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type I (HSV-1) vector expressing human Preproenkephalin (NP2), wild-type reovirus, reovirus type 3 Dearing (REOLYSIN®), oncolytic virus HSV 1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD, HPV vaccine 580299, JX-594 (thymidine kinase-deleted vaccinia virus plus GM-CSF), HPV-16/18 L1/AS04, fowlpox virus vaccine vector, vaccinia-tyrosinase vaccine, MEDI-517 HPV-16/18 VLP AS04 vaccine, adenoviral vector containing the thymidine kinase of herpes simplex virus TK99UN, HspE7, FP253/Fludarabine, ALVAC(2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-1NFg, Ad-ISF35, and coxsackievirus A21 (CVA21, CAVATAK®).

In other embodiments, the multifunctional molecule is administered in combination with a nanopharmaceutical. Exemplary cancer nanopharmaceuticals include, but not limited to, ABRAXANE® (paclitaxel bound albumin nanoparticles), CRLX101 (CPT conjugated to a linear cyclodextrin-based polymer), CRLX288 (conjugating docetaxel to the biodegradable polymer poly (lactic-co-glycolic acid)), cytarabine liposomal (liposomal Ara-C, DEPOCYT™), daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN®).

In some embodiments, the multifunctional molecule is administered in combination with paclitaxel or a paclitaxel formulation, e.g., TAXOL®, protein-bound paclitaxel (e.g., ABRAXANE®). Exemplary paclitaxel formulations include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

Exemplary RNAi and antisense RNA agents for treating cancer include, but not limited to, CALAA-01, siG12D LODER (Local Drug EluteR), and ALN-VSP02.

Other cancer therapeutic agents include, but not limited to, cytokines (e.g., aldesleukin (IL-2, Interleukin-2, PROLEUKIN®), alpha Interferon (IFN-alpha, Interferon alfa, INTRON® A (Interferon alfa-2b), ROFERON-A® (Interferon alfa-2a)), Epoetin alfa (PROCRIT®), filgrastim (G-CSF, Granulocyte—Colony Stimulating Factor, NEUPOGEN®), GM-CSF (Granulocyte Macrophage Colony Stimulating Factor, sargramostim, LEUKINE™), IL-11 (Interleukin-11, oprelvekin, NEUMEGA®), Interferon alfa-2b (PEG conjugate) (PEG interferon, PEG-INTRON™), and pegfilgrastim (NEULASTA™)), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®), goserelin (ZOLADEX®), letrozole (FEMARA®), leuprolide (ELIGARD™, LUPRON®, LUPRON DEPOT®, VIADUR™), megestrol (megestrol acetate, MEGACE®), nilutamide (ANANDRON®, NILANDRON®), octreotide (octreotide acetate, SANDOSTATIN®, SANDOSTATIN LAR®), raloxifene (EVISTA®), romiplostim (NPLATE®), tamoxifen (NOVALDEX®), and toremifene (FARESTON®)), phospholipase A2 inhibitors (e.g., anagrelide (AGRYLIN®)), biologic response modifiers (e.g., BCG (THERACYS®, TICE®), and Darbepoetin alfa (ARANESP®)), target therapy agents (e.g., bortezomib (VELCADE®), dasatinib (SPRYCEL™), denileukin diftitox (ONTAK®), erlotinib (TARCEVA®), everolimus (AFINITOR®), gefitinib (IRESSA®), imatinib mesylate (STI-571, GLEEVEC™), lapatinib (TYKERB®), sorafenib (NEXAVAR®), and SU11248 (sunitinib, SUTENT®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PRELONE®), and prednisone (DELTASONE®, LIQUID PRED®, METICORTEN®, ORASONE®)), and bisphosphonates (e.g., pamidronate (AREDIA®), and zoledronic acid (ZOMETA®))

In some embodiments, the multifunctional molecule is used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-B inhibitor)), a RAF-1 inhibitor, a KIT inhibitor and a RET inhibitor. In some embodiments, the anti-cancer agent used in combination with the AHCM agent is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951(tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (ABI010), MGCD-265, motesanib diphosphate (AMG-706), MP-470, OSI-930, Pazopanib Hydrochloride, PD173074, nSorafenib Tosylate(Bay 43-9006), SU 5402, TSU-68(SU6668), vatalanib, XL880 (GSK1363089, EXEL-2880). Selected tyrosine kinase inhibitors are chosen from sunitinib, erlotinib, gefitinib, or sorafenib. In one embodiment, the tyrosine kinase inhibitor is sunitinib.

In one embodiment, the multifunctional molecule is administered in combination with one of more of: an anti-angiogenic agent, or a vascular targeting agent or a vascular disrupting agent. Exemplary anti-angiogenic agents include, but are not limited to, VEGF inhibitors (e.g., anti-VEGF antibodies (e.g., bevacizumab); VEGF receptor inhibitors (e.g., itraconazole); inhibitors of cell proliferatin and/or migration of endothelial cells (e.g., carboxyamidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe, P. E. (2004) Clin. Cancer Res. Vol. 10:415-427). VTAs can be small-molecule. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

### Immune Checkpoint Inhibitors

In other embodiments, methods described herein comprise use of an immune checkpoint inhibitor in combination with the multifunctional molecule. The methods can be used in a therapeutic protocol in vivo.

In embodiments, an immune checkpoint inhibitor inhibits a checkpoint molecule. Exemplary checkpoint molecules include but are not limited to CTLA4, PD1, PD-L1, PD-L2, TIM3, LAG3, CD160, 2B4, CD80, CD86, B7-H3 (CD276), B7-H4 (VTCN1), HVEM (TNFRSF14 or CD270), BTLA, KIR, MHC class I, MHC class II, GAL9, VISTA, BTLA, TIGIT, LAIR1, and A2aR. See, e.g., Pardoll. Nat. Rev. Cancer 12.4(2012):252-64, incorporated herein by reference.

In embodiments, the immune checkpoint inhibitor is a PD-1 inhibitor, e.g., an anti-PD-1 antibody such as Nivolumab, Pembrolizumab or Pidilizumab. Nivolumab (also called MDX-1106, MDX-1106-04, ONO-4538, or BMS-936558) is a fully human IgG4 monoclonal antibody that specifically inhibits PD1. See, e.g., U.S. Pat. No. 8,008,449 and WO2006/121168. Pembrolizumab (also called Lambrolizumab, MK-3475, MK03475, SCH-900475 or KEYTRUDA®; Merck) is a humanized IgG4 monoclonal antibody that binds to PD-1. See, e.g., Hamid, O. et al. (2013) New England Journal of Medicine 369 (2): 134-44, U.S. Pat. No. 8,354,509 and WO2009/114335. Pidilizumab (also called CT-011 or Cure Tech) is a humanized IgG1k monoclonal antibody that binds to PD1. See, e.g., WO2009/101611. In one embodiment, the inhibitor of PD-1 is an antibody molecule having a sequence substantially identical or similar thereto, e.g., a sequence at least 85%, 90%, 95% identical or higher to the sequence of Nivolumab, Pembrolizumab or Pidilizumab. Additional anti-PD1 antibodies, e.g., AMP 514 (Amplimmune), are described, e.g., in U.S. Pat. No. 8,609,089, US 2010028330, and/or US 20120114649.

In some embodiments, the PD-1 inhibitor is an immunoadhesin, e.g., an immunoadhesin comprising an extracellular/PD-1 binding portion of a PD-1 ligand (e.g., PD-L1 or PD-L2) that is fused to a constant region (e.g., an Fc region of an immunoglobulin). In embodiments, the PD-1 inhibitor is AMP-224 (B7-DCIg, e.g., described in WO2011/066342 and WO2010/027827), a PD-L2 Fc fusion soluble receptor that blocks the interaction between B7-H1 and PD-1.

In embodiments, the immune checkpoint inhibitor is a PD-L1 inhibitor, e.g., an antibody molecule. In some embodiments, the PD-L1 inhibitor is YW243.55.570, MPDL3280A, MEDI-4736, MSB-0010718C, or MDX-1105. In some embodiments, the anti-PD-L1 antibody is MSB0010718C (also called A09-246-2; Merck Serono), which is a monoclonal antibody that binds to PD-L1. Exemplary humanized anti-PD-L1 antibodies are described, e.g., in WO2013/079174. In one embodiment, the PD-L1 inhibitor is an anti-PD-L1 antibody, e.g., YW243.55.S70. The YW243.55.570 antibody is described, e.g., in WO 2010/077634. In one embodiment, the PD-L1 inhibitor is MDX-1105 (also called BMS-936559), which is described, e.g., in WO2007/005874. In one embodiment, the PD-L1 inhibitor is MDPL3280A (Genentech/Roche), which is a human Fc-optimized IgG1 monoclonal antibody against PD-L1. See, e.g., U.S. Pat. No. 7,943,743 and U.S. Publication No.: 20120039906. In one embodiment, the inhibitor of PD-L1 is an antibody molecule having a sequence substantially identical or similar thereto, e.g., a sequence at least 85%, 90%, 95% identical or higher to the sequence of YW243.55.570, MPDL3280A, MEDI-4736, MSB-0010718C, or MDX-1105.

In embodiments, the immune checkpoint inhibitor is a PD-L2 inhibitor, e.g., AMP-224 (which is a PD-L2 Fc fusion soluble receptor that blocks the interaction between PD1 and B7-H1. See, e.g., WO2010/027827 and WO2011/066342.

In one embodiment, the immune checkpoint inhibitor is a LAG-3 inhibitor, e.g., an anti LAG-3 antibody molecule. In embodiments, the anti-LAG-3 antibody is BMS-986016 (also called BMS986016; Bristol-Myers Squibb). BMS-986016 and other humanized anti-LAG-3 antibodies are described, e.g., in US 2011/0150892, WO2010/019570, and WO2014/008218.

In embodiments, the immune checkpoint inhibitor is a TIM-3 inhibitor, e.g., anti-TIM3 antibody molecule, e.g., described in U.S. Pat. No. 8,552,156, WO 2011/155607, EP 2581113 and U.S. Publication No.: 2014/044728.

In embodiments, the immune checkpoint inhibitor is a CTLA-4 inhibitor, e.g., anti-CTLA-4 antibody molecule. Exemplary anti-CTLA4 antibodies include Tremelimumab (IgG2 monoclonal antibody from Pfizer, formerly known as ticilimumab, CP-675,206); and Ipilimumab (also called MDX-010, CAS No. 477202-00-9). Other exemplary anti-CTLA-4 antibodies are described, e.g., in U.S. Pat. No. 5,811,097.

### EXAMPLES

### Example 1: Construction of a Plasmid Encoding the Gene for SEQ ID NO: 18

The DNA encoding the protein sequences was optimized for expression in Cricetulus griseus, synthesized, and cloned into the pcDNA3.4-TOPO (Life Technologies A14697) using Gateway cloning. All constructs contained an Ig Kappa leader sequence. The nucleic acid sequences for the individual components are shown in Table 3 and the complete ORF comprised of the components is shown in Table 4.

TABLE 3
Nucleic acid sequences for components

| SEQ ID NO | Nucleic Acid Sequence |
|---|---|
| SEQ ID NO: 39 | ATGGAAACCGATACACTGCTGCTGTGGGTGCTGCTCCTCTGGGTGCCA |
| Ig Kappa Signal | GGCAGCACCGGC |
| Peptide | |
| SEQ ID NO: 40 | AGAACAGTGGCTGCCCCTTCCGTGTTCATCTTCCCACCTTCCGACGAG |
| CLIg_vk | CAGCTGAAGTCTGGCACAGCCTCTGTCGTGTGTCTGCTGAACAACTTC |
| | TACCCTCGGGAAGCCAAGGTGCAGTGGAAGGTGGACAATGCCCTGCAG |
| | TCCGGCAACTCCCAAGAGTCTGTGACCGAGCAGGACTCCAAGGACAGC |
| | ACCTACAGCCTGTCCTCCACACTGACCCTGTCCAAGGCCGACTACGAG |
| | AAGCACAAGGTGTACGCCTGCGAAGTGACCCATCAGGGACTGTCTAGC |
| | CCCGTGACCAAGTCTTTCAACCGGGGCGAGTGC |
| SEQ ID NO: 41 | GCTTCTACCAAGGGACCTAGCGTGTTCCCTCTGGCTCCTTCCAGCAAG |
| CH1 | TCTACCTCTGGTGGCACAGCTGCTCTGGGCTGCCTGGTCAAGGATTAC |
| Human IgG1 A118- | TTCCCTGAGCCTGTGACCGTGTCCTGGAATTCTGGCGCTCTGACATCC |
| C220 | GGCGTGCACACCTTTCCAGCTGTCCTGCAATCCTCCGGCCTGTACAGC |
| | CTGTCCTCTGTCGTGACCGTGCCTTCTAGCTCTCTGGGCACCCAGACC |
| | TACATCTGCAATGTGAACCACAAGCCTTCCAACACCAAGGTGGACAAG |
| | AGAGTGGAACCCAAGTCCTGC |
| SEQ ID NO: 42 | GATAAGACCCACACCTGTCCTCCATGTCCTGCCCCTGAGCTGCTGGGC |
| Fc_Knob | GGACCTAGCGTGTTCCTGTTCCCTCCAAAGCCCAAGGACACCCTGATG |
| Human IgG1 (D221- | ATCAGCCGGACCCCTGAAGTGACCTGCGTGGTGGTGGATGTGTCCCAC |
| K447) S354C, T366W | GAGGATCCCGAAGTGAAGTTCAATTGGTACGTGGACGGCGTGGAAGTG |
| | CACAACGCCAAGACCAAGCCCAGAGAGGAACAGTACAACAGCACCTAC |
| | CGGGTGGTGTCCGTGCTGACCGTGCTGCACCAGGACTGGCTGAACGGC |
| | AAAGAGTACAAGTGCAAGGTGTCCAACAAGGCCCTGCCTGCCCCTATC |
| | GAGAAAACCATCAGCAAGGCCAAGGGCCAGCCCCGCGAACCTCAGGTG |
| | TACACACTGCCTCCCTGCCGGGAAGAGATGACCAAGAACCAGGTGTCC |
| | CTGTGGTGCCTGGTCAAGGGCTTCTACCCCTCCGATATCGCCGTGGAA |
| | TGGGAGAGCAACGGCCAGCCCGAGAACAACTACAAGACCACCCCTCCC |
| | GTGCTGGACAGCGACGGCAGCTTCTTCCTGTACTCCAAACTGACCGTG |
| | GACAAGAGCCGGTGGCAGCAGGGCAATGTGTTCAGCTGTAGCGTGATG |
| | CACGAGGCCCTGCACAACCACTACACCCAGAAGTCTCTGAGCCTGAGC |
| | CCCGGCAAG |
| SEQ ID NO: 43 | GATAAGACCCACACCTGTCCTCCATGTCCTGCCCCTGAGCTGCTGGGC |
| Fc_Hole | GGACCTAGCGTGTTCCTGTTCCCTCCAAAGCCCAAGGACACCCTGATG |
| Human IgG1 (D221- | ATCAGCCGGACCCCTGAAGTGACCTGCGTGGTGGTGGATGTGTCCCAC |
| K447) Y349C, T366S, | GAGGATCCCGAAGTGAAGTTCAATTGGTACGTGGACGGCGTGGAAGTG |
| L368A, Y407V | CACAACGCCAAGACCAAGCCCAGAGAGGAACAGTACAACAGCACCTAC |
| | CGGGTGGTGTCCGTGCTGACCGTGCTGCACCAGGACTGGCTGAACGGC |
| | AAAGAGTACAAGTGCAAGGTGTCCAACAAGGCCCTGCCTGCCCCTATC |
| | GAGAAAACCATCAGCAAGGCCAAGGGCCAGCCTAGAGAGCCTCAGGTC |
| | TGCACCCTGCCTCCCAGCCGGGAAGAGATGACCAAGAACCAGGTGTCC |
| | CTGTCCTGCGCCGTGAAGGGCTTCTACCCCTCCGATATCGCCGTGGAA |
| | TGGGAGAGCAACGGCCAGCCCGAGAACAACTACAAGACCACCCCTCCC |
| | GTGCTGGACAGCGACGGCAGCTTCTTCCTGGTGTCCAAACTGACCGTG |
| | GACAAGAGCCGGTGGCAGCAGGGCAATGTGTTCAGCTGTAGCGTGATG |
| | CACGAGGCCCTGCACAACCACTACACCCAGAAGTCTCTGAGCCTGAGC |
| | CCCGGCAAG |
| SEQ ID NO: 44 | GGC |
| G Linker | |
| SEQ ID NO: 45 | GGAGGCGGTGGCTCT |
| 4GS Linker (SEQ ID | |
| NO: 80) | |
| SEQ ID NO: 46 | GGAGGCTCCGGCGGA |
| GGSGG Linker (SEQ | |
| ID NO: 81) | |
| SEQ ID NO: 47 | GGTGGCTCCGGCGGCTCT |
| GGSGGS Linker (SEQ | |
| ID NO: 82) | |
| SEQ ID NO: 48 | GGTGGTGGTAGTGGCGGAGGC |
| GGGSGGG Linker (SEQ | |
| ID NO: 83) | |
| SEQ ID NO: 49 | GGCGGCGGAGGATCTGGCGGAGGCGGTTCTGGTGGTGGCGGATCC |
| 3×4GS Linker (SEQ | |
| ID NO: 84) | |
| SEQ ID NO: 50 | GGCGATCAGAACCCTCAGATCGCCGCCCACGTGATCTCCGAGGCCAGC |
| hCD40L | TCTAAGACCACCAGCGTGCTGCAGTGGGCCGAGAAGGGCTACTATACC |
| TNFSF5, C142A | ATGAGCAACAACCTCGTGACCCTGGAAAACGGCAAGCAGCTGACTGTG |
| | AAGAGACAGGGCCTGTACTACATCTACGCCCAAGTGACCTTCTGCTCC |
| | AACAGAGAGGCCTCCTCCCAGGCCCCCTTTATCGCCTCTCTGGCCCTG |
| | AAGTCCCCCGGCAGATTCGAGCGGATCCTGCTGAGAGCCGCCAACACC |
| | CACTCCTCCGCTAAGCCTTGTGGCCAGCAGTCCATCCATCTGGGCGGC |
| | GTGTTCGAACTGCAGCCTGGCGCCTCTGTGTTCGTGAACGTGACCGAC |
| | CCCTCTCAGGTGTCACACGGCACCGGCTTCACCAGCTTCGGCCTGCTG |
| | AAGCTG |
| SEQ ID NO: 51 | AGAATCCAGTCCATCAAGGTGCAGTTCACCGAGTACAAGAAAGAGAAG |
| hOX40L | GGCTTCATCCTGACCAGCCAGAAAGAGGACGAGATCATGAAGGTGCAG |
| TNFSF4 | AACAACTCCGTGATCATCAACTGCGACGGCTTTTACCTGATCTCCCTG |
| | AAGGGCTACTTCTCCCAAGAAGTGAACATCTCCCTGCACTACCAGAAG |
| | GACGAGGAACCCCTGTTCCAGCTGAAGAAAGTGCGGAGCGTGAACTCC |
| | CTGATGGTGGCCTCTCTGACCTACAAGGACAAGGTGTACCTGAACGTG |
| | ACCACCGACAACACCAGCCTGGACGACTTCCATGTGAATGGCGGCGAG |
| | CTGATCCTGATCCATCAGAACCCTGGCGAGTTCTGCGTTCTC |
| SEQ ID NO: 52 | AGAACACCTTCTGACAAGCCTGTGGCTCACGTGGTGGCCAATCCTCAG |
| hTNFα | GCTGAAGGACAGCTGCAGTGGCTGAACAGAAGGGCCAACGCTCTGCTG |
| TNFSF2 | GCCAATGGCGTGGAACTGAGAGACAATCAGCTGGTGGTGCCTTCCGAG |
| | GGCCTGTACCTGATCTATAGCCAGGTGCTGTTCAAAGGCCAGGGCTGC |
| | CCTTCTACTCACGTGCTGCTGACCCACACCATCAGCAGAATCGCCGTG |
| | TCCTACCAGACCAAAGTGAACCTGCTGTCCGCCATCAAGAGCCCCTGC |
| | CAGAGAGAAACACCTGAGGGCGCTGAAGCCAAGCCTTGGTACGAGCCT |
| | ATCTATCTCGGCGGCGTGTTCCAGCTCGAGAAGGGCGATAGACTGAGC |
| | GCCGAGATCAACCGGCCTGACTACCTGGATTTTGCCGAGTCCGGCCAG |
| | GTGTACTTCGGCATTATTGCCCTT |
| SEQ ID NO: 53 | GATAAGCCAGTGGCACATGTCGTGGCTAACCCACAGGCAGAGGGTCAA |
| hTNFα | CTCCAGTGGCTCAATCGGAGAGCTAATGCACTGCTGGCTAACGGCGTT |
| TNFSF2 | GAGCTGCGGGATAACCAGCTCGTGGTCCCAAGCGAGGGACTCTATCTC |
| | ATCTACAGTCAGGTCTTGTTTAAAGGACAGGGGTGTCCCAGCACACAT |
| | GTTCTGCTGACACATACAATCTCCCGGATCGCCGTCAGCTATCAGACA |
| | AAAGTCAATCTGCTGAGCGCTATCAAGTCTCCCTGCCAGCGCGAAACT |
| | CCAGAAGGCGCAGAGGCTAAACCTTGGTATGAACCCATCTACCTTGGC |
| | GGAGTGTTTCAGTTGGAAAAAGGCGACCGGCTGTCCGCTGAGATCAAC |
| | AGACCCGATTATCTGGATTTCGCTGAGAGCGGACAGGTCTACTTTGGG |
| | ATCATTGCTCTG |
| SEQ ID NO: 54 | CAACTGGAAACCGCCAAAGAACCCTGCATGGCCAAGTTCGGCCCTCTG |
| hGITRL | CCTTCTAAGTGGCAGATGGCCTCTTCCGAGCCTCCTTGCGTGAACAAA |
| TNFSF18 | GTGTCCGACTGGAAGCTGGAAATCCTGCAGAACGGCCTGTACCTGATC |
| | TACGGACAGGTGGCCCCTAACGCCAACTACAACGATGTGGCCCCTTTC |
| | GAAGTGCGGCTGTACAAGAACAAGGACATGATCCAGACACTGACCAAC |
| | AAGTCCAAGATCCAGAACGTCGGCGGCACCTACGAGCTGCATGTGGGC |
| | GATACCATCGACCTGATCTTCAACTCCGAGCACCAGGTGCTGAAGAAC |
| | AACACCTACTGGGGCATCATCCTGCTGGCTAAC |
| SEQ ID NO: 55 | CAGCTGGAAACAGCTAAAGAACCTTGTATGGCTAAATTTGGGCCCCTG |
| hGITRL | CCTAGCAAATGGCAAATGGCTAGCAGCGAGCCACCATGTGTGAACAAG |
| TNFSF18 | GTTTCAGATTGGAAACTCGAGATCCTCCAGAATGGGCTCTATCTGATC |
| | TATGGCCAGGTCGCACCCAATGCTAACTACAATGACGTCGCACCATTT |
| | GAAGTCCGCCTCTATAAGAACAAAGATATGATTCAAACCCTCACAAAC |
| | AAGAGCAAGATTCAGAATGTTGGCGGGACATATGAACTGCACGTGGGA |
| | GACACAATCGATCTCATCTTCAATAGCGAGCATCAGGTCCTCAAAAAC |
| | AATACTTATTGGGGAATCATTCTGCTCGCCAATCCTCAGTTCATCTCC |
| SEQ ID NO: 56 | AATCCTGCAGCTCATCTGACCGGCGCCAACTCCTCTCTGACTGGTTCT |
| hLIGHT | GGTGGACCCCTGCTCTGGGAAACTCAGCTGGGACTCGCTTTCCTGCGG |
| TNFSF14 | GGCCTGTCTTATCATGATGGCGCTCTGGTGGTCACCAAGGCCGGCTAC |
| | TACTACATCTACTCCAAGGTGCAGCTCGGCGGCGTGGGATGTCCTCTT |
| | GGACTGGCTTCTACAATCACCCACGGCCTGTACAAGCGGACCCCTAGA |
| | TACCCCGAGGAACTGGAACTGCTGGTGTCCCAGCAGTCTCCTTGTGGC |
| | AGAGCCACCTCCTCCAGCAGAGTTTGGTGGGACTCTTCTTTCCTTGGT |
| | GGCGTGGTGCACCTGGAAGCTGGCGAGAAAGTGGTCGTCAGAGTGCTG |
| | GACGAAAGACTCGTGCGGCTGAGAGATGGCACCCGGTCTTACTTCGGC |
| | GCCTTTATGGTT |
| SEQ ID NO: 57 | TTGAGAAAAGTGGCTCACCTGACCGGCAAGTCCAACAGCAGATCCATG |
| hFASL | CCTCTCGAGTGGGAAGATACCTACGGCATCGTGCTGCTGTCCGGCGTG |
| TNFSF6 | AAGTACAAGAAAGGCGGCCTGGTCATCAACGAGACTGGCCTGTACTTC |
| | GTGTACAGCAAGGTGTACTTCCGGGGCCAGTCCTGCAACAACCTGCCT |
| | CTGAGCCATAAGGTGTACATGCGGAACAGCAAGTACCCTCAGGACCTG |
| | GTCATGATGGAAGGCAAGATGATGTCCTACTGCACCACCGGCCAGATG |
| | TGGGCCAGATCTTCTTATCTGGGCGCCGTGTTCAACCTGACCAGCGCC |
| | GATCACCTGTACGTGAACGTGTCCGAGCTGTCCCTGGTCAACTTCGAG |
| | GAATCCCAGACCTTCTTCGGCCTGTACAAGCTT |
| SEQ ID NO: 58 | AAGGTGGCCCATCTGACTGGAAAGTCTAACTCCCGGTCTATGCCCCTT |
| hFASL | GAATGGGAAGATACTTATGGGATTGTCCTGCTGAGCGGGGTCAAGTAT |
| TNFSF6 | AAGAAAGGTGGACTCGTGATTAACGAAACCGGGCTCTACTTCGTCTAC |
| | TCTAAGGTCTACTTCAGAGGGCAGAGCTGTAACAATCTGCCCCTGTCT |
| | CACAAGGTCTACATGAGAAACTCCAAGTATCCCCAGGATCTCGTGATG |
| | ATGGAAGGGAAAATGATGAGCTACTGTACAACAGGGCAAATGTGGGCT |
| | AGAAGCTCCTACCTGGGAGCTGTGTTTAATCTGACCTCTGCTGACCAC |
| | CTCTATGTGAATGTGTCTGAACTGAGCCTCGTGAACTTCGAAGAGAGC |
| | CAGACCTTTTTTGGGCTCTATAAGCTG |

TABLE 4
DNA sequences encoding protein constructs

| | Corresponding | |
|---|---|---|
| | Protein | |
| SEQ ID NO | SEQ ID NO | Sequence |
| SEQ ID NO: 59 | SEQ ID NO: 94 | ATGGAAACCGACACCCTGCTGCTGTGGGTGCTGCTGCTGTGGG |
| hFc_Knob- | | TGCCAGGCTCTACCGGCGACAAGACCCACACCTGTCCTCCCTG |
| 2X_CD40L | | TCCTGCCCCTGAACTGCTGGGCGGACCTTCCGTGTTCCTGTTC |
| | | CCCCCAAAGCCCAAGGACACCCTGATGATCTCCCGGACCCCCG |
| | | AAGTGACCTGCGTGGTGGTGGATGTGTCCCACGAGGACCCTGA |
| | | AGTGAAGTTCAATTGGTACGTGGACGGCGTGGAAGTGCACAAC |
| | | GCCAAGACCAAGCCCAGAGAGGAACAGTACAACTCCACCTACC |
| | | GGGTGGTGTCCGTGCTGACCGTGCTGCACCAGGATTGGCTGAA |
| | | CGGCAAAGAGTACAAGTGCAAGGTGTCCAACAAGGCCCTGCCA |
| | | GCCCCCATCGAAAAGACCATCTCCAAGGCCAAGGGCCAGCCCC |
| | | GGGAACCCCAGGTGTACACACTGCCCCCTTGCCGGGAAGAGAT |
| | | GACCAAGAACCAGGTGTCCCTGTGGTGCCTCGTGAAGGGCTTC |
| | | TACCCCTCCGATATCGCCGTGGAATGGGAGTCCAACGGCCAGC |
| | | CTGAGAACAACTACAAGACCACCCCCCCTGTGCTGGACTCCGA |
| | | CGGCTCATTCTTCCTGTACTCCAAGCTGACAGTGGACAAGTCC |
| | | CGGTGGCAGCAGGGCAACGTGTTCTCCTGCTCCGTGATGCACG |
| | | AGGCCCTGCACAACCACTACACCCAGAAGTCCCTGTCCCTGAG |
| | | CCCTGGAAAAGGCGGCGGAGGATCTGGCGGAGGCGGTTCTGGT |
| | | GGTGGCGGATCCGGCGATCAGAACCCTCAGATCGCCGCCCACG |
| | | TGATCTCCGAGGCCAGCTCTAAGACCACCAGCGTGCTGCAGTG |
| | | GGCCGAGAAGGGCTACTACACCATGAGCAACAACCTCGTGACA |
| | | CTGGAAAACGGCAAGCAGCTGACTGTGAAGAGACAGGGCCTGT |
| | | ACTACATCTACGCCCAAGTGACCTTCTGCTCCAACAGAGAGGC |
| | | CTCCTCCCAGGCCCCCTTTATCGCCTCTCTGGCCCTGAAGTCC |
| | | CCCGGCAGATTCGAGCGGATCCTGCTGAGAGCCGCCAACACCC |
| | | ACTCCTCCGCTAAGCCTTGTGGCCAGCAGTCCATCCATCTGGG |
| | | CGGCGTGTTCGAACTGCAGCCTGGCGCCTCTGTGTTCGTGAAC |
| | | GTGACCGACCCCTCTCAGGTGTCACACGGCACCGGCTTCACCA |
| | | GCTTCGGCCTGCTGAAACTGGGAGGCGGTGGCTCTGGCGACCA |
| | | GAATCCACAGATTGCTGCTCATGTGATCAGCGAGGCCTCCAGC |
| | | AAGACCACTTCTGTGCTGCAGTGGGCTGAAAAAGGGTATTATA |
| | | CCATGTCTAACAATCTCGTGACCCTGGAAAATGGGAAACAGCT |
| | | GACCGTGAAACGGCAGGGGCTGTATTATATCTATGCTCAAGTG |
| | | ACATTTTGTTCTAACCGCGAGGCTAGCAGCCAGGCCCCTTTCA |
| | | TTGCTAGCCTGGCTCTGAAAAGCCCTGGCCGCTTCGAGAGAAT |
| | | TCTGCTGCGGGCTGCCAATACCCACAGCTCTGCCAAGCCATGC |
| | | GGACAGCAGAGCATCCACCTGGGGGGAGTGTTTGAGCTGCAGC |
| | | CAGGTGCCAGTGTGTTTGTGAATGTGACAGATCCCAGCCAGGT |
| | | GTCCCATGGCACAGGCTTTACATCCTTTGGGCTGCTGAAGCTG |
| SEQ ID NO: 60 | SEQ ID NO: 95 | ATGGAAACCGACACCCTGCTGCTGTGGGTGCTGCTGCTGTGGG |
| hFc_Hole- | | TGCCAGGCTCTACCGGCGACAAGACCCACACCTGTCCTCCCTG |
| 1X_CD40L | | TCCTGCCCCTGAACTGCTGGGCGGACCTTCCGTGTTCCTGTTC |
| | | CCCCCAAAGCCCAAGGACACCCTGATGATCTCCCGGACCCCCG |
| | | AAGTGACCTGCGTGGTGGTGGATGTGTCCCACGAGGACCCTGA |
| | | AGTGAAGTTCAATTGGTACGTGGACGGCGTGGAAGTGCACAAC |
| | | GCCAAGACCAAGCCCAGAGAGGAACAGTACAACTCCACCTACC |
| | | GGGTGGTGTCCGTGCTGACCGTGCTGCACCAGGATTGGCTGAA |
| | | CGGCAAAGAGTACAAGTGCAAGGTGTCCAACAAGGCCCTGCCA |
| | | GCCCCCATCGAAAAGACCATCTCCAAGGCCAAGGGCCAGCCCC |
| | | GGGAACCTCAAGTGTGCACCCTGCCCCCTAGCCGGGAAGAGAT |
| | | GACCAAGAACCAGGTGTCCCTGAGCTGCGCCGTGAAGGGCTTC |
| | | TACCCCTCCGATATCGCCGTGGAATGGGAGTCCAACGGCCAGC |
| | | CTGAGAACAACTACAAGACCACCCCCCCTGTGCTGGACTCCGA |
| | | CGGCTCATTCTTCCTGGTGTCCAAGCTGACAGTGGACAAGTCC |
| | | CGGTGGCAGCAGGGCAACGTGTTCTCCTGCTCCGTGATGCACG |
| | | AGGCCCTGCACAACCACTACACCCAGAAGTCCCTGTCCCTGTC |
| | | TCCCGGAAAAGGCGGCGGAGGATCTGGCGGAGGCGGTTCTGGT |
| | | GGTGGCGGATCCGGCGATCAGAACCCTCAGATCGCCGCCCACG |
| | | TGATCTCCGAGGCCAGCTCTAAGACCACCAGCGTGCTGCAGTG |
| | | GGCCGAGAAGGGCTACTATACCATGAGCAACAACCTCGTGACC |
| | | CTGGAAAACGGCAAGCAGCTGACTGTGAAGAGACAGGGCCTGT |
| | | ACTACATCTACGCCCAAGTGACCTTCTGCTCCAACAGAGAGGC |
| | | CTCCTCCCAGGCCCCCTTTATCGCCTCTCTGGCCCTGAAGTCC |
| | | CCCGGCAGATTCGAGCGGATCCTGCTGAGAGCCGCCAACACCC |
| | | ACTCCTCCGCTAAGCCTTGTGGCCAGCAGTCCATCCATCTGGG |
| | | CGGCGTGTTCGAACTGCAGCCTGGCGCCTCTGTGTTCGTGAAC |
| | | GTGACCGACCCCTCTCAGGTGTCACACGGCACCGGCTTCACCA |
| | | GCTTCGGCCTGCTGAAGCTG |
| SEQ ID NO: 61 | SEQ ID NO: 96 | ATGGAAACCGATACACTGCTGCTGTGGGTGCTGCTCCTCTGGG |
| hFc_Knob | | TGCCAGGCAGCACCGGCGATAAGACCCACACCTGTCCTCCATG |
| | | TCCTGCCCCTGAGCTGCTGGGCGGACCTAGCGTGTTCCTGTTC |
| | | CCTCCAAAGCCCAAGGACACCCTGATGATCAGCCGGACCCCTG |
| | | AAGTGACCTGCGTGGTGGTGGATGTGTCCCACGAGGATCCCGA |
| | | AGTGAAGTTCAATTGGTACGTGGACGGCGTGGAAGTGCACAAC |
| | | GCCAAGACCAAGCCCAGAGAGGAACAGTACAACAGCACCTACC |
| | | GGGTGGTGTCCGTGCTGACCGTGCTGCACCAGGACTGGCTGAA |
| | | CGGCAAAGAGTACAAGTGCAAGGTGTCCAACAAGGCCCTGCCT |
| | | GCCCCTATCGAGAAAACCATCAGCAAGGCCAAGGGCCAGCCCC |
| | | GCGAACCTCAGGTGTACACACTGCCTCCCTGCCGGGAAGAGAT |
| | | GACCAAGAACCAGGTGTCCCTGTGGTGCCTGGTCAAGGGCTTC |
| | | TACCCCTCCGATATCGCCGTGGAATGGGAGAGCAACGGCCAGC |
| | | CCGAGAACAACTACAAGACCACCCCTCCCGTGCTGGACAGCGA |
| | | CGGCAGCTTCTTCCTGTACTCCAAACTGACCGTGGACAAGAGC |
| | | CGGTGGCAGCAGGGCAATGTGTTCAGCTGTAGCGTGATGCACG |
| | | AGGCCCTGCACAACCACTACACCCAGAAGTCTCTGAGCCTGAG |
| | | CCCCGGCAAG |
| SEQ ID NO: 62 | SEQ ID NO: 97 | ATGGAAACCGACACACTGCTGCTGTGGGTGCTGCTCTTGTGGG |
| 2X_CD40L- | | TGCCAGGATCTACAGGCGGCGATCAGAACCCTCAGATCGCCGC |
| hCHIg_Hole | | TCATGTGATCTCCGAGGCCTCTTCCAAGACCACCTCTGTGCTG |
| | | CAGTGGGCCGAGAAGGGCTACTACACCATGTCCAACAACCTGG |
| | | TCACCCTGGAAAACGGCAAGCAGCTGACCGTGAAGAGACAGGG |
| | | CCTGTACTACATCTACGCCCAAGTGACCTTCTGCTCCAACAGA |
| | | GAGGCCTCCTCTCAGGCCCCTTTTATCGCCTCTCTGGCCCTGA |
| | | AGTCCCCTGGCAGATTCGAGAGAATCCTGCTGAGAGCCGCTAA |
| | | CACCCACTCCTCTGCTAAGCCTTGTGGCCAGCAGTCTATCCAT |
| | | CTCGGCGGAGTGTTTGAGCTGCAGCCTGGCGCTTCTGTGTTCG |
| | | TGAACGTGACCGATCCTAGCCAGGTGTCCCACGGCACCGGCTT |
| | | CACATCTTTTGGCCTGCTGAAACTCGGAGGCGGCGGAAGCGGA |
| | | GATCAGAATCCACAAATTGCTGCCCACGTGATCAGCGAGGCCA |
| | | GCTCTAAGACAACCAGCGTCCTCCAGTGGGCTGAAAAGGGGTA |
| | | TTATACAATGAGCAACAATCTCGTGACCCTCGAGAATGGGAAA |
| | | CAGCTGACAGTCAAGCGGCAGGGGCTCTATTACATCTATGCTC |
| | | AAGTCACATTTTGCTCTAACCGCGAGGCTTCCAGCCAGGCTCC |
| | | TTTCATTGCTTCCCTGGCTCTGAAAAGCCCCGGCAGATTTGAG |
| | | CGGATTCTGCTGCGGGCTGCCAACACACACTCTAGCGCCAAAC |
| | | CATGCGGCCAGCAGAGCATTCATCTTGGCGGCGTTTTCGAATT |
| | | GCAGCCAGGCGCAAGCGTGTTCGTCAATGTGACAGACCCCTCT |
| | | CAGGTTTCCCATGGCACAGGGTTTACCAGCTTCGGACTGCTCA |
| | | AACTTGGCGGTGGTGGATCTGGTGGCGGAGGATCTGGCGGTGG |
| | | CGGTTCTGCTTCTACCAAGGGACCTAGCGTGTTCCCTCTGGCT |
| | | CCTTCCAGCAAGTCTACCTCTGGTGGCACAGCTGCTCTGGGCT |
| | | GCCTGGTCAAGGATTACTTCCCTGAGCCTGTGACCGTGTCCTG |
| | | GAATTCTGGCGCTCTGACATCCGGCGTGCACACCTTTCCAGCT |
| | | GTCCTGCAATCCTCCGGCCTGTACAGCCTGTCCTCTGTCGTGA |
| | | CCGTGCCTTCTAGCTCTCTGGGCACCCAGACCTACATCTGCAA |
| | | TGTGAACCACAAGCCTTCCAACACCAAGGTGGACAAGAGAGTG |
| | | GAACCCAAGTCCTGCGACAAGACCCACACCTGTCCTCCATGTC |
| | | CTGCTCCAGAACTGCTCGGCGGACCTTCCGTGTTCCTGTTTCC |
| | | TCCAAAGCCTAAGGACACCCTGATGATCTCTCGGACCCCTGAA |
| | | GTGACCTGCGTGGTGGTCGATGTGTCTCACGAGGATCCCGAAG |
| | | TGAAGTTCAATTGGTACGTGGACGGCGTGGAAGTGCACAACGC |
| | | CAAGACCAAGCCTAGAGAGGAACAGTACAACTCCACCTACAGA |
| | | GTGGTGTCCGTGCTGACAGTGCTGCACCAGGATTGGCTGAACG |
| | | GCAAAGAGTACAAGTGCAAGGTTTCCAACAAGGCCCTGCCTGC |
| | | TCCTATCGAAAAGACCATCTCCAAGGCCAAGGGCCAGCCTCGG |
| | | GAACCTCAAGTCTGTACCCTGCCTCCTAGCCGGGAAGAGATGA |
| | | CCAAGAATCAAGTGTCCCTGTCCTGCGCCGTGAAGGGCTTTTA |
| | | CCCTTCCGATATCGCCGTGGAATGGGAGAGCAATGGCCAGCCT |
| | | GAGAACAACTACAAGACAACCCCTCCTGTGCTGGACTCCGACG |
| | | GCTCATTCTTCCTGGTGTCCAAGCTGACTGTGGACAAGTCCAG |
| | | ATGGCAGCAGGGCAATGTGTTCTCCTGCTCCGTGATGCACGAG |
| | | GCCCTGCACAATCACTACACCCAGAAGTCTCTGTCTCTGAGCC |
| | | CTGGCAAG |
| SEQ ID NO: 63 | SEQ ID NO: 98 | ATGGAAACCGACACACTGCTGCTGTGGGTGCTGCTCTTGTGGG |
| 1X_CD40L- | | TGCCAGGATCTACAGGCGGCGATCAGAACCCTCAGATCGCCGC |
| hCLIg_vk | | TCATGTGATCTCCGAGGCCTCTTCCAAGACCACCTCTGTGCTG |
| | | CAGTGGGCCGAGAAGGGCTACTACACCATGTCCAACAACCTGG |
| | | TCACCCTGGAAAACGGCAAGCAGCTGACCGTGAAGAGACAGGG |
| | | CCTGTACTACATCTACGCCCAAGTGACCTTCTGCTCCAACAGA |
| | | GAGGCCTCCTCTCAGGCCCCTTTTATCGCCTCTCTGGCCCTGA |
| | | AGTCCCCTGGCAGATTCGAGAGAATCCTGCTGAGAGCCGCTAA |
| | | CACCCACTCCTCTGCTAAGCCTTGTGGCCAGCAGTCTATCCAT |
| | | CTCGGCGGAGTGTTTGAGCTGCAGCCTGGCGCTTCTGTGTTCG |
| | | TGAACGTGACCGATCCTAGCCAGGTGTCCCACGGCACCGGCTT |
| | | CACATCTTTTGGCCTGCTGAAACTCGGAGGCGGCGGATCTGGT |
| | | GGCGGAGGTTCTGGCGGAGGCGGATCTAGAACAGTGGCTGCCC |
| | | CTTCCGTGTTCATCTTCCCACCTTCCGACGAGCAGCTGAAGTC |
| | | TGGCACAGCCTCTGTCGTGTGTCTGCTGAACAACTTCTACCCT |
| | | CGGGAAGCCAAGGTGCAGTGGAAGGTGGACAATGCCCTGCAGT |
| | | CCGGCAACTCCCAAGAGTCTGTGACCGAGCAGGACTCCAAGGA |
| | | CAGCACCTACAGCCTGTCCTCCACACTGACCCTGTCCAAGGCC |
| | | GACTACGAGAAGCACAAGGTGTACGCCTGCGAAGTGACCCATC |
| | | AGGGACTGTCTAGCCCCGTGACCAAGTCTTTCAACCGGGGCGA |
| | | GTGC |
| SEQ ID NO: 64 | SEQ ID NO: 99 | ATGGAAACCGACACACTGCTGCTGTGGGTGCTGCTCTTGTGGG |
| hFc_Knob- | | TGCCAGGATCTACCGGCGACAAGACCCATACCTGTCCTCCATG |
| 2X_OX40L | | TCCTGCTCCAGAGCTGCTCGGAGGCCCTTCCGTGTTTCTGTTC |
| | | CCTCCAAAGCCTAAGGACACCCTGATGATCTCTCGGACCCCTG |
| | | AAGTGACCTGCGTGGTGGTGGATGTGTCTCACGAGGATCCCGA |
| | | AGTGAAGTTCAATTGGTACGTGGACGGCGTGGAAGTGCACAAC |
| | | GCCAAGACCAAGCCTAGAGAGGAACAGTACAACTCCACCTACA |
| | | GAGTGGTGTCCGTGCTGACCGTGCTGCACCAGGATTGGCTGAA |
| | | CGGCAAAGAGTACAAGTGCAAGGTGTCCAACAAGGCCCTGCCT |
| | | GCTCCTATCGAAAAGACCATCTCCAAGGCCAAGGGCCAGCCTA |
| | | GGGAACCCCAGGTTTACACCCTGCCTCCATGCCGGGAAGAGAT |
| | | GACCAAGAACCAGGTGTCCCTGTGGTGCCTGGTCAAGGGCTTC |
| | | TACCCTTCCGATATCGCCGTGGAATGGGAGAGCAATGGCCAGC |
| | | CTGAGAACAACTACAAGACCACACCTCCTGTGCTGGACTCCGA |
| | | CGGCTCATTCTTCCTGTACTCCAAGCTGACAGTGGACAAGTCC |
| | | AGATGGCAGCAGGGCAACGTGTTCTCCTGCTCCGTGATGCACG |
| | | AGGCCCTGCACAATCACTACACCCAGAAGTCCCTGTCTCTGTC |
| | | CCCTGGAAAAGGCGGCGGAGGATCTGGCGGAGGTGGAAGCGGA |
| | | GGCGGAGGAAGCGGTGGCGGCGGATCTAGAATCCAGTCCATCA |
| | | AGGTGCAGTTCACCGAGTACAAGAAAGAGAAGGGCTTCATCCT |
| | | GACCAGCCAGAAAGAGGACGAGATCATGAAGGTGCAGAACAAC |
| | | TCCGTGATCATCAACTGCGACGGCTTTTACCTGATCTCCCTGA |
| | | AGGGCTACTTCTCCCAAGAAGTGAACATCTCCCTGCACTACCA |
| | | GAAGGACGAGGAACCCCTGTTCCAGCTGAAGAAAGTGCGGAGC |
| | | GTGAACTCCCTGATGGTGGCCTCTCTGACCTACAAGGACAAGG |
| | | TGTACCTGAACGTGACCACCGACAACACCAGCCTGGACGACTT |
| | | CCATGTGAATGGCGGCGAGCTGATCCTGATCCATCAGAACCCT |
| | | GGCGAGTTCTGCGTTCTCGGTGGTGGTAGTGGCGGAGGCAGAA |
| | | TCCAGAGTATTAAGGTCCAGTTTACAGAGTATAAGAAAGAAAA |
| | | AGGTTTTATCCTCACCTCTCAGAAAGAAGATGAGATTATGAAG |
| | | GTCCAAAACAATAGCGTCATCATTAACTGTGATGGGTTCTACC |
| | | TCATCAGCCTCAAGGGCTATTTCAGTCAAGAAGTCAATATCAG |
| | | CCTCCATTATCAGAAGGATGAAGAACCACTCTTTCAACTCAAA |
| | | AAAGTTCGCTCCGTGAACAGCCTCATGGTGGCTAGCCTGACTT |
| | | ATAAGGACAAAGTCTATCTCAATGTCACCACAGATAACACATC |
| | | CCTCGACGATTTCCACGTCAACGGCGGAGAACTGATTCTCATC |
| | | CACCAAAATCCAGGCGAGTTTTGTGTGCTG |
| SEQ ID NO: 65 | SEQ ID NO: | ATGGAAACCGACACACTGCTGCTGTGGGTGCTGCTCTTGTGGG |
| hFc_Hole- | 100 | TGCCAGGATCTACCGGCGACAAGACCCATACCTGTCCTCCATG |
| 1X_OX40L | | TCCTGCTCCAGAGCTGCTCGGAGGCCCTTCCGTGTTTCTGTTC |
| | | CCTCCAAAGCCTAAGGACACCCTGATGATCTCTCGGACCCCTG |
| | | AAGTGACCTGCGTGGTGGTGGATGTGTCTCACGAGGATCCCGA |
| | | AGTGAAGTTCAATTGGTACGTGGACGGCGTGGAAGTGCACAAC |
| | | GCCAAGACCAAGCCTAGAGAGGAACAGTACAACTCCACCTACA |
| | | GAGTGGTGTCCGTGCTGACCGTGCTGCACCAGGATTGGCTGAA |
| | | CGGCAAAGAGTACAAGTGCAAGGTGTCCAACAAGGCCCTGCCT |
| | | GCTCCTATCGAAAAGACCATCTCCAAGGCCAAGGGCCAGCCTC |
| | | GGGAACCTCAAGTCTGTACCCTGCCTCCTAGCCGGGAAGAGAT |
| | | GACCAAGAACCAGGTGTCCCTGTCCTGTGCCGTGAAGGGCTTC |
| | | TACCCTTCCGATATCGCCGTGGAATGGGAGAGCAATGGCCAGC |
| | | CTGAGAACAACTACAAGACCACACCTCCTGTGCTGGACTCCGA |
| | | CGGCTCATTCTTCCTGGTGTCCAAGCTGACAGTGGACAAGTCC |
| | | AGATGGCAGCAGGGCAACGTGTTCTCCTGCTCCGTGATGCACG |
| | | AGGCCCTGCACAATCACTACACCCAGAAGTCCCTGAGCCTGTC |
| | | TCCTGGAAAAGGCGGCGGAGGATCTGGCGGAGGTGGAAGCGGA |
| | | GGCGGAGGAAGCGGTGGCGGCGGATCTAGAATCCAGTCCATCA |
| | | AGGTGCAGTTCACCGAGTACAAGAAAGAGAAGGGCTTTATCCT |
| | | GACCAGCCAGAAAGAGGACGAGATCATGAAGGTGCAGAACAAC |
| | | TCCGTGATCATCAACTGCGACGGCTTTTACCTGATCTCCCTGA |
| | | AGGGCTACTTCTCCCAAGAAGTGAACATCTCCCTGCACTACCA |
| | | GAAGGACGAGGAACCCCTGTTCCAGCTGAAGAAAGTGCGGAGC |
| | | GTGAACTCCCTGATGGTGGCCTCTCTGACCTACAAGGACAAGG |
| | | TGTACCTGAACGTGACCACCGACAACACCAGCCTGGACGACTT |
| | | CCATGTGAATGGCGGCGAGCTGATCCTGATCCATCAGAACCCT |
| | | GGCGAGTTCTGCGTGCTG |
| SEQ ID NO: 66 | SEQ ID NO: | ATGGAAACCGACACACTGCTGCTGTGGGTGCTGCTCTTGTGGG |
| hFc_Knob- | 101 | TGCCAGGATCTACCGGCGACAAGACCCATACCTGTCCTCCATG |
| 2X_TNFα | | TCCTGCTCCAGAGCTGCTCGGAGGCCCTTCCGTGTTTCTGTTC |
| | | CCTCCAAAGCCTAAGGACACCCTGATGATCTCTCGGACCCCTG |
| | | AAGTGACCTGCGTGGTGGTGGATGTGTCTCACGAGGATCCCGA |
| | | AGTGAAGTTCAATTGGTACGTGGACGGCGTGGAAGTGCACAAC |
| | | GCCAAGACCAAGCCTAGAGAGGAACAGTACAACTCCACCTACA |
| | | GAGTGGTGTCCGTGCTGACCGTGCTGCACCAGGATTGGCTGAA |
| | | CGGCAAAGAGTACAAGTGCAAGGTGTCCAACAAGGCCCTGCCT |
| | | GCTCCTATCGAAAAGACCATCTCCAAGGCCAAGGGCCAGCCTA |
| | | GGGAACCCCAGGTTTACACCCTGCCTCCATGCCGGGAAGAGAT |
| | | GACCAAGAACCAGGTGTCCCTGTGGTGCCTGGTCAAGGGCTTC |
| | | TACCCTTCCGATATCGCCGTGGAATGGGAGAGCAATGGCCAGC |
| | | CTGAGAACAACTACAAGACCACACCTCCTGTGCTGGACTCCGA |
| | | CGGCTCATTCTTCCTGTACTCCAAGCTGACAGTGGACAAGTCC |
| | | AGATGGCAGCAGGGCAACGTGTTCTCCTGCTCCGTGATGCACG |
| | | AGGCCCTGCACAATCACTACACCCAGAAGTCCCTGTCTCTGTC |
| | | CCCTGGAAAAGGCGGCGGAGGATCTGGCGGAGGTGGAAGCGGA |
| | | GGCGGAGGAAGCGGTGGCGGCGGATCTAGAACACCTTCTGACA |
| | | AGCCTGTGGCTCACGTGGTGGCCAATCCTCAGGCTGAAGGACA |
| | | GCTGCAGTGGCTGAACAGAAGGGCCAACGCTCTGCTGGCCAAT |
| | | GGCGTGGAACTGAGAGACAATCAGCTGGTGGTGCCTTCCGAGG |
| | | GCCTGTACCTGATCTATAGCCAGGTGCTGTTCAAAGGCCAGGG |
| | | CTGCCCTTCTACTCACGTGCTGCTGACCCACACCATCAGCAGA |
| | | ATCGCCGTGTCCTACCAGACCAAAGTGAACCTGCTGTCCGCCA |
| | | TCAAGAGCCCCTGCCAGAGAGAAACACCTGAGGGCGCTGAAGC |
| | | CAAGCCTTGGTACGAGCCTATCTATCTCGGCGGCGTGTTCCAG |
| | | CTCGAGAAGGGCGATAGACTGAGCGCCGAGATCAACCGGCCTG |
| | | ACTACCTGGATTTTGCCGAGTCCGGCCAGGTGTACTTCGGCAT |
| | | TATTGCCCTTGGTGGCTCCGGCGGCTCTGATAAGCCAGTGGCA |
| | | CATGTCGTGGCTAACCCACAGGCAGAGGGTCAACTCCAGTGGC |
| | | TCAATCGGAGAGCTAATGCACTGCTGGCTAACGGCGTTGAGCT |
| | | GCGGGATAACCAGCTCGTGGTCCCAAGCGAGGGACTCTATCTC |
| | | ATCTACAGTCAGGTCTTGTTTAAAGGACAGGGGTGTCCCAGCA |
| | | CACATGTTCTGCTGACACATACAATCTCCCGGATCGCCGTCAG |
| | | CTATCAGACAAAAGTCAATCTGCTGAGCGCTATCAAGTCTCCC |
| | | TGCCAGCGCGAAACTCCAGAAGGCGCAGAGGCTAAACCTTGGT |
| | | ATGAACCCATCTACCTTGGCGGAGTGTTTCAGTTGGAAAAAGG |
| | | CGACCGGCTGTCCGCTGAGATCAACAGACCCGATTATCTGGAT |
| | | TTCGCTGAGAGCGGACAGGTCTACTTTGGGATCATTGCTCTG |
| SEQ ID NO: 67 | SEQ ID NO: | ATGGAAACCGACACACTGCTGCTGTGGGTGCTGCTCTTGTGGG |
| hFc_Hole- | 102 | TGCCAGGATCTACCGGCGACAAGACCCATACCTGTCCTCCATG |
| 1X_TNFα | | TCCTGCTCCAGAGCTGCTCGGAGGCCCTTCCGTGTTTCTGTTC |
| | | CCTCCAAAGCCTAAGGACACCCTGATGATCTCTCGGACCCCTG |
| | | AAGTGACCTGCGTGGTGGTGGATGTGTCTCACGAGGATCCCGA |
| | | AGTGAAGTTCAATTGGTACGTGGACGGCGTGGAAGTGCACAAC |
| | | GCCAAGACCAAGCCTAGAGAGGAACAGTACAACTCCACCTACA |
| | | GAGTGGTGTCCGTGCTGACCGTGCTGCACCAGGATTGGCTGAA |
| | | CGGCAAAGAGTACAAGTGCAAGGTGTCCAACAAGGCCCTGCCT |
| | | GCTCCTATCGAAAAGACCATCTCCAAGGCCAAGGGCCAGCCTC |
| | | GGGAACCTCAAGTCTGTACCCTGCCTCCTAGCCGGGAAGAGAT |
| | | GACCAAGAACCAGGTGTCCCTGTCCTGTGCCGTGAAGGGCTTC |
| | | TACCCTTCCGATATCGCCGTGGAATGGGAGAGCAATGGCCAGC |
| | | CTGAGAACAACTACAAGACCACACCTCCTGTGCTGGACTCCGA |
| | | CGGCTCATTCTTCCTGGTGTCCAAGCTGACAGTGGACAAGTCC |
| | | AGATGGCAGCAGGGCAACGTGTTCTCCTGCTCCGTGATGCACG |
| | | AGGCCCTGCACAATCACTACACCCAGAAGTCCCTGAGCCTGTC |
| | | TCCTGGAAAAGGCGGCGGAGGATCTGGCGGAGGTGGAAGCGGA |
| | | GGCGGAGGAAGCGGTGGCGGCGGATCTAGAACACCTTCTGACA |
| | | AGCCTGTGGCTCACGTGGTGGCCAATCCTCAGGCTGAAGGACA |
| | | GCTGCAGTGGCTGAACAGAAGGGCCAACGCTCTGCTGGCCAAT |
| | | GGCGTGGAACTGAGAGACAATCAGCTGGTGGTGCCTTCCGAGG |
| | | GCCTGTACCTGATCTATAGCCAGGTGCTGTTCAAAGGCCAGGG |
| | | CTGCCCTTCTACTCACGTGCTGCTGACCCACACCATCAGCAGA |
| | | ATCGCCGTGTCCTACCAGACCAAAGTGAACCTGCTGTCCGCCA |
| | | TCAAGAGCCCCTGCCAGAGAGAAACACCTGAGGGCGCTGAAGC |
| | | CAAGCCTTGGTACGAGCCTATCTATCTCGGCGGCGTGTTCCAG |
| | | CTCGAGAAGGGCGATAGACTGAGCGCCGAGATCAACCGGCCTG |
| | | ACTACCTGGATTTTGCCGAGTCCGGCCAGGTGTACTTCGGCAT |
| | | CATTGCCCTG |
| SEQ ID NO: 68 | SEQ ID NO: | ATGGAAACCGACACACTGCTGCTGTGGGTGCTGCTCTTGTGGG |
| hFc_Knob- | 103 | TGCCAGGATCTACCGGCGACAAGACCCATACCTGTCCTCCATG |
| 2X_FASL | | TCCTGCTCCAGAGCTGCTCGGAGGCCCTTCCGTGTTTCTGTTC |
| | | CCTCCAAAGCCTAAGGACACCCTGATGATCTCTCGGACCCCTG |
| | | AAGTGACCTGCGTGGTGGTGGATGTGTCTCACGAGGATCCCGA |
| | | AGTGAAGTTCAATTGGTACGTGGACGGCGTGGAAGTGCACAAC |
| | | GCCAAGACCAAGCCTAGAGAGGAACAGTACAACTCCACCTACA |
| | | GAGTGGTGTCCGTGCTGACCGTGCTGCACCAGGATTGGCTGAA |
| | | CGGCAAAGAGTACAAGTGCAAGGTGTCCAACAAGGCCCTGCCT |
| | | GCTCCTATCGAAAAGACCATCTCCAAGGCCAAGGGCCAGCCTA |
| | | GGGAACCCCAGGTTTACACCCTGCCTCCATGCCGGGAAGAGAT |
| | | GACCAAGAACCAGGTGTCCCTGTGGTGCCTGGTCAAGGGCTTC |
| | | TACCCTTCCGATATCGCCGTGGAATGGGAGAGCAATGGCCAGC |
| | | CTGAGAACAACTACAAGACCACACCTCCTGTGCTGGACTCCGA |
| | | CGGCTCATTCTTCCTGTACTCCAAGCTGACAGTGGACAAGTCC |
| | | AGATGGCAGCAGGGCAACGTGTTCTCCTGCTCCGTGATGCACG |
| | | AGGCCCTGCACAATCACTACACCCAGAAGTCCCTGTCTCTGTC |
| | | CCCTGGAAAAGGCGGCGGAGGATCTGGCGGAGGTGGAAGCGGA |
| | | GGCGGAGGAAGCGGTGGCGGCGGATCTTTGAGAAAAGTGGCTC |
| | | ACCTGACCGGCAAGTCCAACAGCAGATCCATGCCTCTCGAGTG |
| | | GGAAGATACCTACGGCATCGTGCTGCTGTCCGGCGTGAAGTAC |
| | | AAGAAAGGCGGCCTGGTCATCAACGAGACTGGCCTGTACTTCG |
| | | TGTACAGCAAGGTGTACTTCCGGGGCCAGTCCTGCAACAACCT |
| | | GCCTCTGAGCCATAAGGTGTACATGCGGAACAGCAAGTACCCT |
| | | CAGGACCTGGTCATGATGGAAGGCAAGATGATGTCCTACTGCA |
| | | CCACCGGCCAGATGTGGGCCAGATCTTCTTATCTGGGCGCCGT |
| | | GTTCAACCTGACCAGCGCCGATCACCTGTACGTGAACGTGTCC |
| | | GAGCTGTCCCTGGTCAACTTCGAGGAATCCCAGACCTTCTTCG |
| | | GCCTGTACAAGCTTGGTGGCTCTGGCGGAAAGGTGGCCCATCT |
| | | GACTGGAAAGTCTAACTCCCGGTCTATGCCCCTTGAATGGGAA |
| | | GATACTTATGGGATTGTCCTGCTGAGCGGGGTCAAGTATAAGA |
| | | AAGGTGGACTCGTGATTAACGAAACCGGGCTCTACTTCGTCTA |
| | | CTCTAAGGTCTACTTCAGAGGGCAGAGCTGTAACAATCTGCCC |
| | | CTGTCTCACAAGGTCTACATGAGAAACTCCAAGTATCCCCAGG |
| | | ATCTCGTGATGATGGAAGGGAAAATGATGAGCTACTGTACAAC |
| | | AGGGCAAATGTGGGCTAGAAGCTCCTACCTGGGAGCTGTGTTT |
| | | AATCTGACCTCTGCTGACCACCTCTATGTGAATGTGTCTGAAC |
| | | TGAGCCTCGTGAACTTCGAAGAGAGCCAGACCTTTTTTGGGCT |
| | | CTATAAGCTG |
| SEQ ID NO: 69 | SEQ ID NO: | ATGGAAACCGACACACTGCTGCTGTGGGTGCTGCTCTTGTGGG |
| hFc_Hole- | 104 | TGCCAGGATCTACCGGCGACAAGACCCATACCTGTCCTCCATG |
| 1X_FASL | | TCCTGCTCCAGAGCTGCTCGGAGGCCCTTCCGTGTTTCTGTTC |
| | | CCTCCAAAGCCTAAGGACACCCTGATGATCTCTCGGACCCCTG |
| | | AAGTGACCTGCGTGGTGGTGGATGTGTCTCACGAGGATCCCGA |
| | | AGTGAAGTTCAATTGGTACGTGGACGGCGTGGAAGTGCACAAC |
| | | GCCAAGACCAAGCCTAGAGAGGAACAGTACAACTCCACCTACA |
| | | GAGTGGTGTCCGTGCTGACCGTGCTGCACCAGGATTGGCTGAA |
| | | CGGCAAAGAGTACAAGTGCAAGGTGTCCAACAAGGCCCTGCCT |
| | | GCTCCTATCGAAAAGACCATCTCCAAGGCCAAGGGCCAGCCTC |
| | | GGGAACCTCAAGTCTGTACCCTGCCTCCTAGCCGGGAAGAGAT |
| | | GACCAAGAACCAGGTGTCCCTGTCCTGTGCCGTGAAGGGCTTC |
| | | TACCCTTCCGATATCGCCGTGGAATGGGAGAGCAATGGCCAGC |
| | | CTGAGAACAACTACAAGACCACACCTCCTGTGCTGGACTCCGA |
| | | CGGCTCATTCTTCCTGGTGTCCAAGCTGACAGTGGACAAGTCC |
| | | AGATGGCAGCAGGGCAACGTGTTCTCCTGCTCCGTGATGCACG |
| | | AGGCCCTGCACAATCACTACACCCAGAAGTCCCTGAGCCTGTC |
| | | TCCTGGAAAAGGCGGCGGAGGATCTGGCGGAGGTGGAAGCGGA |
| | | GGCGGAGGAAGCGGTGGCGGCGGATCTTTGAGAAAAGTGGCTC |
| | | ACCTGACCGGCAAGTCCAACAGCAGATCCATGCCTCTCGAGTG |
| | | GGAAGATACCTACGGCATCGTGCTGCTGTCCGGCGTGAAGTAC |
| | | AAGAAAGGCGGCCTGGTCATCAACGAGACTGGCCTGTACTTCG |
| | | TGTACTCCAAGGTGTACTTCCGGGGCCAGTCCTGCAACAACCT |
| | | GCCTCTGAGCCATAAGGTGTACATGCGGAACAGCAAGTACCCT |
| | | CAGGACCTGGTCATGATGGAAGGCAAGATGATGTCCTACTGCA |
| | | CCACCGGCCAGATGTGGGCCAGATCTTCTTATCTGGGCGCCGT |
| | | GTTCAACCTGACCAGCGCCGATCACCTGTACGTGAACGTGTCC |
| | | GAGCTGTCCCTGGTCAACTTCGAGGAATCCCAGACCTTCTTCG |
| | | GCCTGTACAAGCTG |
| SEQ ID NO: 70 | SEQ ID NO: | ATGGAAACCGACACACTGCTGCTGTGGGTGCTGCTCTTGTGGG |
| hFc_Knob- | 105 | TGCCAGGATCTACCGGCGACAAGACCCATACCTGTCCTCCATG |
| 2X_LIGHT | | TCCTGCTCCAGAGCTGCTCGGAGGCCCTTCCGTGTTTCTGTTC |
| | | CCTCCAAAGCCTAAGGACACCCTGATGATCTCTCGGACCCCTG |
| | | AAGTGACCTGCGTGGTGGTGGATGTGTCTCACGAGGATCCCGA |
| | | AGTGAAGTTCAATTGGTACGTGGACGGCGTGGAAGTGCACAAC |
| | | GCCAAGACCAAGCCTAGAGAGGAACAGTACAACTCCACCTACA |
| | | GAGTGGTGTCCGTGCTGACCGTGCTGCACCAGGATTGGCTGAA |
| | | CGGCAAAGAGTACAAGTGCAAGGTGTCCAACAAGGCCCTGCCT |
| | | GCTCCTATCGAAAAGACCATCTCCAAGGCCAAGGGCCAGCCTA |
| | | GGGAACCCCAGGTTTACACCCTGCCTCCATGCCGGGAAGAGAT |
| | | GACCAAGAACCAGGTGTCCCTGTGGTGCCTGGTCAAGGGCTTC |
| | | TACCCTTCCGATATCGCCGTGGAATGGGAGAGCAATGGCCAGC |
| | | CTGAGAACAACTACAAGACCACACCTCCTGTGCTGGACTCCGA |
| | | CGGCTCATTCTTCCTGTACTCCAAGCTGACAGTGGACAAGTCC |
| | | AGATGGCAGCAGGGCAACGTGTTCTCCTGCTCCGTGATGCACG |
| | | AGGCCCTGCACAATCACTACACCCAGAAGTCCCTGTCTCTGTC |
| | | CCCTGGAAAAGGCGGCGGAGGATCTGGCGGAGGTGGAAGCGGA |
| | | GGCGGAGGAAGCGGTGGCGGCGGATCTAATCCTGCAGCTCATC |
| | | TGACCGGCGCCAACTCCTCTCTGACTGGTTCTGGTGGACCCCT |
| | | GCTCTGGGAAACTCAGCTGGGACTCGCTTTCCTGCGGGGCCTG |
| | | TCTTATCATGATGGCGCTCTGGTGGTCACCAAGGCCGGCTACT |
| | | ACTACATCTACTCCAAGGTGCAGCTCGGCGGCGTGGGATGTCC |
| | | TCTTGGACTGGCTTCTACAATCACCCACGGCCTGTACAAGCGG |
| | | ACCCCTAGATACCCCGAGGAACTGGAACTGCTGGTGTCCCAGC |
| | | AGTCTCCTTGTGGCAGAGCCACCTCCTCCAGCAGAGTTTGGTG |
| | | GGACTCTTCTTTCCTTGGTGGCGTGGTGCACCTGGAAGCTGGC |
| | | GAGAAAGTGGTCGTCAGAGTGCTGGACGAAAGACTCGTGCGGC |
| | | TGAGAGATGGCACCCGGTCTTACTTCGGCGCCTTTATGGTTGG |
| | | AGGCTCCGGCGGAAATCCAGCCGCTCATTTGACAGGGGCCAAT |
| | | TCCAGCTTGACCGGCTCTGGCGGACCTCTCCTGTGGGAGACTC |
| | | AACTCGGACTGGCCTTTCTGAGAGGCCTGTCCTACCACGACGG |
| | | TGCTCTGGTCGTTACAAAGGCTGGGTACTATTATATCTACAGC |
| | | AAAGTCCAACTCGGCGGAGTCGGCTGTCCACTCGGACTCGCTA |
| | | GCACCATCACACATGGCCTGTATAAGAGAACCCCACGCTATCC |
| | | TGAGGAACTCGAGCTTCTCGTCAGCCAGCAGAGCCCTTGTGGA |
| | | CGGGCTACCTCCAGCTCTCGAGTGTGGTGGGATAGCTCTTTTC |
| | | TTGGCGGGGTCGTGCATTTGGAGGCCGGCGAAAAGGTCGTCGT |
| | | TCGCGTGTTGGATGAGAGGCTCGTCAGACTCAGAGATGGAACC |
| | | AGAAGCTACTTTGGCGCTTTCATGGTC |
| SEQ ID NO: 71 | SEQ ID NO: | ATGGAAACCGACACACTGCTGCTGTGGGTGCTGCTCTTGTGGG |
| hFc_Hole- | 106 | TGCCAGGATCTACCGGCGACAAGACCCATACCTGTCCTCCATG |
| 1X_LIGHT | | TCCTGCTCCAGAGCTGCTCGGAGGCCCTTCCGTGTTTCTGTTC |
| | | CCTCCAAAGCCTAAGGACACCCTGATGATCTCTCGGACCCCTG |
| | | AAGTGACCTGCGTGGTGGTGGATGTGTCTCACGAGGATCCCGA |
| | | AGTGAAGTTCAATTGGTACGTGGACGGCGTGGAAGTGCACAAC |
| | | GCCAAGACCAAGCCTAGAGAGGAACAGTACAACTCCACCTACA |
| | | GAGTGGTGTCCGTGCTGACCGTGCTGCACCAGGATTGGCTGAA |
| | | CGGCAAAGAGTACAAGTGCAAGGTGTCCAACAAGGCCCTGCCT |
| | | GCTCCTATCGAAAAGACCATCTCCAAGGCCAAGGGCCAGCCTC |
| | | GGGAACCTCAAGTCTGTACCCTGCCTCCTAGCCGGGAAGAGAT |
| | | GACCAAGAACCAGGTGTCCCTGTCCTGTGCCGTGAAGGGCTTC |
| | | TACCCTTCCGATATCGCCGTGGAATGGGAGAGCAATGGCCAGC |
| | | CTGAGAACAACTACAAGACCACACCTCCTGTGCTGGACTCCGA |
| | | CGGCTCATTCTTCCTGGTGTCCAAGCTGACAGTGGACAAGTCC |
| | | AGATGGCAGCAGGGCAACGTGTTCTCCTGCTCCGTGATGCACG |
| | | AGGCCCTGCACAATCACTACACCCAGAAGTCCCTGAGCCTGTC |
| | | TCCTGGAAAAGGCGGCGGAGGATCTGGCGGAGGTGGAAGCGGA |
| | | GGCGGAGGAAGCGGTGGCGGCGGATCTAATCCTGCAGCTCATC |
| | | TGACCGGCGCCAACTCCTCTCTGACTGGTTCTGGTGGACCCCT |
| | | GCTCTGGGAAACTCAGCTGGGACTCGCTTTCCTGCGGGGCCTG |
| | | TCTTATCATGATGGCGCTCTGGTGGTCACCAAGGCCGGCTACT |
| | | ACTACATCTACTCCAAGGTGCAGCTCGGCGGCGTGGGATGTCC |
| | | TCTTGGACTGGCTTCTACAATCACCCACGGCCTGTACAAGCGG |
| | | ACCCCTAGATACCCCGAGGAACTGGAACTGCTGGTGTCCCAGC |
| | | AGTCTCCTTGTGGCAGAGCCACCTCCTCCAGCAGAGTTTGGTG |
| | | GGACTCTTCTTTCCTTGGTGGCGTGGTGCACCTGGAAGCTGGC |
| | | GAGAAAGTGGTCGTCAGAGTGCTGGACGAAAGACTCGTGCGGC |
| | | TGAGAGATGGCACCCGGTCTTACTTCGGCGCCTTCATGGTG |
| SEQ ID NO: 72 | SEQ ID NO: | ATGGAAACCGACACACTGCTGCTGTGGGTGCTGCTCTTGTGGG |
| hFc_Knob- | 107 | TGCCAGGATCTACCGGCGACAAGACCCATACCTGTCCTCCATG |
| 2X_GITRL | | TCCTGCTCCAGAGCTGCTCGGAGGCCCTTCCGTGTTTCTGTTC |
| | | CCTCCAAAGCCTAAGGACACCCTGATGATCTCTCGGACCCCTG |
| | | AAGTGACCTGCGTGGTGGTGGATGTGTCTCACGAGGATCCCGA |
| | | AGTGAAGTTCAATTGGTACGTGGACGGCGTGGAAGTGCACAAC |
| | | GCCAAGACCAAGCCTAGAGAGGAACAGTACAACTCCACCTACA |
| | | GAGTGGTGTCCGTGCTGACCGTGCTGCACCAGGATTGGCTGAA |
| | | CGGCAAAGAGTACAAGTGCAAGGTGTCCAACAAGGCCCTGCCT |
| | | GCTCCTATCGAAAAGACCATCTCCAAGGCCAAGGGCCAGCCTA |
| | | GGGAACCCCAGGTTTACACCCTGCCTCCATGCCGGGAAGAGAT |
| | | GACCAAGAACCAGGTGTCCCTGTGGTGCCTGGTCAAGGGCTTC |
| | | TACCCTTCCGATATCGCCGTGGAATGGGAGAGCAATGGCCAGC |
| | | CTGAGAACAACTACAAGACCACACCTCCTGTGCTGGACTCCGA |
| | | CGGCTCATTCTTCCTGTACTCCAAGCTGACAGTGGACAAGTCC |
| | | AGATGGCAGCAGGGCAACGTGTTCTCCTGCTCCGTGATGCACG |
| | | AGGCCCTGCACAATCACTACACCCAGAAGTCCCTGTCTCTGTC |
| | | CCCTGGAAAAGGCGGCGGAGGATCTGGCGGAGGTGGAAGCGGA |
| | | GGCGGAGGAAGCGGTGGCGGCGGATCTCAACTGGAAACCGCCA |
| | | AAGAACCCTGCATGGCCAAGTTCGGCCCTCTGCCTTCTAAGTG |
| | | GCAGATGGCCTCTTCCGAGCCTCCTTGCGTGAACAAAGTGTCC |
| | | GACTGGAAGCTGGAAATCCTGCAGAACGGCCTGTACCTGATCT |
| | | ACGGACAGGTGGCCCCTAACGCCAACTACAACGATGTGGCCCC |
| | | TTTCGAAGTGCGGCTGTACAAGAACAAGGACATGATCCAGACA |
| | | CTGACCAACAAGTCCAAGATCCAGAACGTCGGCGGCACCTACG |
| | | AGCTGCATGTGGGCGATACCATCGACCTGATCTTCAACTCCGA |
| | | GCACCAGGTGCTGAAGAACAACACCTACTGGGGCATCATCCTG |
| | | CTGGCTAACGGCCAGCTGGAAACAGCTAAAGAACCTTGTATGG |
| | | CTAAATTTGGGCCCCTGCCTAGCAAATGGCAAATGGCTAGCAG |
| | | CGAGCCACCATGTGTGAACAAGGTTTCAGATTGGAAACTCGAG |
| | | ATCCTCCAGAATGGGCTCTATCTGATCTATGGCCAGGTCGCAC |
| | | CCAATGCTAACTACAATGACGTCGCACCATTTGAAGTCCGCCT |
| | | CTATAAGAACAAAGATATGATTCAAACCCTCACAAACAAGAGC |
| | | AAGATTCAGAATGTTGGCGGGACATATGAACTGCACGTGGGAG |
| | | ACACAATCGATCTCATCTTCAATAGCGAGCATCAGGTCCTCAA |
| | | AAACAATACTTATTGGGGAATCATTCTGCTCGCCAATCCTCAG |
| | | TTCATCTCC |
| SEQ ID NO: 73 | SEQ ID NO: | ATGGAAACCGACACACTGCTGCTGTGGGTGCTGCTCTTGTGGG |
| hFc_Hole- | 108 | TGCCAGGATCTACCGGCGACAAGACCCATACCTGTCCTCCATG |
| 1X_GITRL | | TCCTGCTCCAGAGCTGCTCGGAGGCCCTTCCGTGTTTCTGTTC |
| | | CCTCCAAAGCCTAAGGACACCCTGATGATCTCTCGGACCCCTG |
| | | AAGTGACCTGCGTGGTGGTGGATGTGTCTCACGAGGATCCCGA |
| | | AGTGAAGTTCAATTGGTACGTGGACGGCGTGGAAGTGCACAAC |
| | | GCCAAGACCAAGCCTAGAGAGGAACAGTACAACTCCACCTACA |
| | | GAGTGGTGTCCGTGCTGACCGTGCTGCACCAGGATTGGCTGAA |
| | | CGGCAAAGAGTACAAGTGCAAGGTGTCCAACAAGGCCCTGCCT |
| | | GCTCCTATCGAAAAGACCATCTCCAAGGCCAAGGGCCAGCCTC |
| | | GGGAACCTCAAGTCTGTACCCTGCCTCCTAGCCGGGAAGAGAT |
| | | GACCAAGAACCAGGTGTCCCTGTCCTGTGCCGTGAAGGGCTTC |
| | | TACCCTTCCGATATCGCCGTGGAATGGGAGAGCAATGGCCAGC |
| | | CTGAGAACAACTACAAGACCACACCTCCTGTGCTGGACTCCGA |
| | | CGGCTCATTCTTCCTGGTGTCCAAGCTGACAGTGGACAAGTCC |
| | | AGATGGCAGCAGGGCAACGTGTTCTCCTGCTCCGTGATGCACG |
| | | AGGCCCTGCACAATCACTACACCCAGAAGTCCCTGAGCCTGTC |
| | | TCCTGGAAAAGGCGGCGGAGGATCTGGCGGAGGTGGAAGCGGA |
| | | GGCGGAGGAAGCGGTGGCGGCGGATCTCAACTGGAAACCGCCA |
| | | AAGAACCCTGCATGGCCAAGTTCGGCCCTCTGCCTTCTAAGTG |
| | | GCAGATGGCCTCTTCCGAGCCTCCTTGCGTGAACAAAGTGTCC |
| | | GACTGGAAGCTGGAAATCCTGCAGAACGGCCTGTACCTGATCT |
| | | ACGGACAGGTGGCCCCTAACGCCAACTACAACGATGTGGCCCC |
| | | TTTCGAAGTGCGGCTGTACAAGAACAAGGACATGATCCAGACA |
| | | CTGACCAACAAGTCCAAGATCCAGAACGTCGGCGGCACCTACG |
| | | AGCTGCATGTGGGCGATACCATCGACCTGATCTTCAACTCCGA |
| | | GCACCAGGTGCTGAAGAACAACACCTACTGGGGCATCATCCTG |
| | | CTGGCCAATCCTCAGTTCATCTCC |

### Example 2: Protein Sequences

The protein sequences encoded from the DNA listed in Tables 3 and 4 are in shown in Tables 5 and 6 respectively. Table 7 gives the Fc_Knob and Fc_Hole pairs which make complete constructs.

TABLE 5
Component Protein Sequences

| | Corresponding | |
|---|---|---|
| | DNA SEQ | |
| SEQ ID NO | ID | Sequence |
| SEQ ID NO: 74 | SEQ ID | METDTLLLWVLLLWVPGSTG |
| Ig Kappa Signal | NO: 39 | |
| Peptide | | |
| SEQ ID NO: 75 | SEQ ID | RTVAAPSVFIFPPSDEQLKSGTASVVCLLNNFYPREAKVQWKVD |
| CLIg_vk | NO: 40 | NALQSGNSQESVTEQDSKDSTYSLSSTLTLSKADYEKHKVYACE |
| | | VTHQGLSSPVTKSFNRGEC |
| SEQ ID NO: 183 | | RTVAAPSVFIFPPSDEQLKSGTASVVCLLNNFYPREAKVQWKVD |
| CLIg_vk without the | | NALQSGNSQESVTEQDSKDSTYSLSSTLTLSKADYEKHKVYACE |
| C-terminal cysteine | | VTHQGLSSPVTKSFNRGE |
| SEQ ID NO: 191 | | RTVAAPSVFIFPPSDEQLKSGTASVVCLLNNFYPREAKVQWKVD |
| CLIg_vk C107S | | NALQSGNSQESVTEQDSKDSTYSLSSTLTLSKADYEKHKVYACE |
| | | VTHQGLSSPVTKSFNRGES |
| SEQ ID NO: 76 | SEQ ID | ASTKGPSVFPLAPSSKSTSGGTAALGCLVKDYFPEPVTVSWNSG |
| CH1 | NO: 41 | ALTSGVHTFPAVLQSSGLYSLSSVVTVPSSSLGTQTYICNVNHK |
| Human IgG1 A118- | | PSNTKVDKRVEPKSC |
| C220 | | |
| SEQ ID NO: 186 | | ASTKGPSVFPLAPSSKSTSGGTAALGCLVKDYFPEPVTVSWNSG |
| CH1 | | ALTSGVHTFPAVLQSSGLYSLSSVVTVPSSSLGTQTYICNVNHK |
| Human IgG1 A118- | | PSNTKVDKRVEPKS |
| S219 | | |
| SEQ ID NO: 192 | | ASTKGPSVFPLAPSSKSTSGGTAALGCLVKDYFPEPVTVSWNSG |
| CH1 Human IgG1 | | ALTSGVHTFPAVLQSSGLYSLSSVVTVPSSSLGTQTYICNVNHK |
| A118-C220, C220S | | PSNTKVDKRVEPKSS |
| SEQ ID NO: 182 | | EPKSCDKTHTCP |
| Wild type IgG1 hinge | | |
| domain | | |
| SEQ ID NO: 181 | | EPKSDKTHTCP |
| IgG1 hinge domain | | |
| with C220 removed | | |
| SEQ ID NO: 197 | | EPKSSDKTHTCP |
| IgG1 hinge domain | | |
| C220S | | |
| SEQ ID NO: 187 | | ASTKGPSVFPLAPSSKSTSGGTAALGCLVKDYFPEPVTVSWNSG |
| Human IgG1 Y349C, | | ALTSGVHTFPAVLQSSGLYSLSSVVTVPSSSLGTQTYICNVNHK |
| T366S, L368A, | | PSNTKVDKRVEPKSDKTHTCPPCPAPELLGGPSVFLFPPKPKDT |
| Y407V with C220 | | LMISRTPEVTCVVVDVSHEDPEVKFNWYVDGVEVHNAKTKPREE |
| removed | | QYNSTYRVVSVLTVLHQDWLNGKEYKCKVSNKALPAPIEKTISK |
| | | AKGQPREPQVCTLPPSREEMTKNQVSLSCAVKGFYPSDIAVEWE |
| | | SNGQPENNYKTTPPVLDSDGSFFLVSKLTVDKSRWQQGNVFSCS |
| | | VMHEALHNHYTQKSLSLSPGK |
| SEQ ID NO: 188 | | ASTKGPSVFPLAPSSKSTSGGTAALGCLVKDYFPEPVTVSWNSG |
| Human IgG1 S354S, | | ALTSGVHTFPAVLQSSGLYSLSSVVTVPSSSLGTQTYICNVNHK |
| T366W with C220 | | PSNTKVDKRVEPKSDKTHTCPPCPAPELLGGPSVFLFPPKPKDT |
| removed | | LMISRTPEVTCVVVDVSHEDPEVKFNWYVDGVEVHNAKTKPREE |
| | | QYNSTYRVVSVLTVLHQDWLNGKEYKCKVSNKALPAPIEKTISK |
| | | AKGQPREPQVYTLPPCREEMTKNQVSLWCLVKGFYPSDIAVEWE |
| | | SNGQPENNYKTTPPVLDSDGSFFLYSKLTVDKSRWQQGNVFSCS |
| | | VMHEALHNHYTQKSLSLSPGK |
| SEQ ID NO: 193 | | ASTKGPSVFPLAPSSKSTSGGTAALGCLVKDYFPEPVTVSWNSG |
| Human IgG1 C220S, | | ALTSGVHTFPAVLQSSGLYSLSSVVTVPSSSLGTQTYICNVNHK |
| Y349C, T366S, | | PSNTKVDKRVEPKSSDKTHTCPPCPAPELLGGPSVFLFPPKPKD |
| L368A, Y407V | | TLMISRTPEVTCVVVDVSHEDPEVKFNWYVDGVEVHNAKTKPRE |
| | | EQYNSTYRVVSVLTVLHQDWLNGKEYKCKVSNKALPAPIEKTIS |
| | | KAKGQPREPQVCTLPPSREEMTKNQVSLSCAVKGFYPSDIAVEW |
| | | ESNGQPENNYKTTPPVLDSDGSFFLVSKLTVDKSRWQQGNVFSC |
| | | SVMHEALHNHYTQKSLSLSPGK |
| SEQ ID NO: 194 | | ASTKGPSVFPLAPSSKSTSGGTAALGCLVKDYFPEPVTVSWNSG |
| Human IgG1 C220S, | | ALTSGVHTFPAVLQSSGLYSLSSVVTVPSSSLGTQTYICNVNHK |
| S354C, T366W | | PSNTKVDKRVEPKSSDKTHTCPPCPAPELLGGPSVFLFPPKPKD |
| | | TLMISRTPEVTCVVVDVSHEDPEVKFNWYVDGVEVHNAKTKPRE |
| | | EQYNSTYRVVSVLTVLHQDWLNGKEYKCKVSNKALPAPIEKTIS |
| | | KAKGQPREPQVYTLPPCREEMTKNQVSLWCLVKGFYPSDIAVEW |
| | | ESNGQPENNYKTTPPVLDSDGSFFLYSKLTVDKSRWQQGNVFSC |
| | | SVMHEALHNHYTQKSLSLSPGK |
| SEQ ID NO: 77 | SEQ ID | DKTHTCPPCPAPELLGGPSVFLFPPKPKDTLMISRTPEVTCVVV |
| Fc_Knob | NO: 42 | DVSHEDPEVKFNWYVDGVEVHNAKTKPREEQYNSTYRVVSVLTV |
| Human IgG1 (D221- | | LHQDWLNGKEYKCKVSNKALPAPIEKTISKAKGQPREPQVYTLP |
| K447) S354C, T366W | | PCREEMTKNQVSLWCLVKGFYPSDIAVEWESNGQPENNYKTTPP |
| | | VLDSDGSFFLYSKLTVDKSRWQQGNVFSCSVMHEALHNHYTQKS |
| | | LSLSPGK |
| SEQ ID NO: 184 | | DKTHTSPPSPAPELLGGPSVFLFPPKPKDTLMISRTPEVTCVVV |
| Fc_Knob Human IgG1 | | DVSHEDPEVKFNWYVDGVEVHNAKTKPREEQYNSTYRVVSVLTV |
| (D221-K447) C226S, | | LHQDWLNGKEYKCKVSNKALPAPIEKTISKAKGQPREPQVYTLP |
| C229S, T366W | | PSREEMTKNQVSLWCLVKGFYPSDIAVEWESNGQPENNYKTTPP |
| | | VLDSDGSFFLYSKLTVDKSRWQQGNVFSCSVMHEALHNHYTQKS |
| | | LSLSPGK |
| SEQ ID NO: 78 | SEQ ID | DKTHTCPPCPAPELLGGPSVFLFPPKPKDTLMISRTPEVTCVVV |
| Fc_Hole | NO: 43 | DVSHEDPEVKFNWYVDGVEVHNAKTKPREEQYNSTYRVVSVLTV |
| Human IgG1 (D221- | | LHQDWLNGKEYKCKVSNKALPAPIEKTISKAKGQPREPQVCTLP |
| K447) Y349C, T366S, | | PSREEMTKNQVSLSCAVKGFYPSDIAVEWESNGQPENNYKTTPP |
| L368A, Y407V. | | VLDSDGSFFLVSKLTVDKSRWQQGNVFSCSVMHEALHNHYTQKS |
| | | LSLSPGK |
| SEQ ID NO: 185 | | DKTHTSPPSPAPELLGGPSVFLFPPKPKDTLMISRTPEVTCVVV |
| Fc_Hole | | DVSHEDPEVKFNWYVDGVEVHNAKTKPREEQYNSTYRVVSVLTV |
| Human IgG1 (D221- | | LHQDWLNGKEYKCKVSNKALPAPIEKTISKAKGQPREPQVYTLP |
| K447) C226S, C229S, | | PSREEMTKNQVSLSCAVKGFYPSDIAVEWESNGQPENNYKTTPP |
| T366S, L368A, | | VLDSDGSFFLVSKLTVDKSRWQQGNVFSCSVMHEALHNHYTQKS |
| Y407V | | LSLSPGK |
| SEQ ID NO: 79 | SEQ ID | G |
| G Linker | NO: 44 | |
| SEQ ID NO: 80 | SEQ ID | GGGGS |
| 4GSLinker | NO: 45 | |
| SEQ ID NO: 81 | SEQ ID | GGSGG |
| GGSGG Linker | NO: 46 | |
| SEQ ID NO: 82 | SEQ ID | GGSGGS |
| GGSGGSLinker | NO: 47 | |
| SEQ ID NO: 83 | SEQ ID | GGGSGGG |
| GGGSGGG Linker | NO: 48 | |
| SEQ ID NO: 189 | | GGGGSGGGG |
| GGGGSGGGG Linker | | |
| SEQ ID NO: 84 | SEQ ID | GGGGSGGGGSGGGGS |
| 3 × 4GSLinker | NO: 49 | |
| SEQ ID NO: 190 | | GGGGSGGGGSGGGGSGGGGS |
| 4 × 4GSLinker | | |
| SEQ ID NO: 85 | SEQ ID | GDQNPQIAAHVISEASSKTTSVLQWAEKGYYTMSNNLVTLENGK |
| hCD40L | NO: 50 | QLTVKRQGLYYIYAQVTFCSNREASSQAPFIASLALKSPGRFER |
| TNFSF5, C142A | | ILLRAANTHSSAKPCGQQSIHLGGVFELQPGASVFVNVTDPSQV |
| | | SHGTGFTSFGLLKL |
| SEQ ID NO: 86 | SEQ ID | RIQSIKVQFTEYKKEKGFILTSQKEDEIMKVQNNSVIINCDGFY |
| hOX40L | NO: 51 | LISLKGYFSQEVNISLHYQKDEEPLFQLKKVRSVNSLMVASLTY |
| TNFSF4 | | KDKVYLNVTTDNTSLDDFHVNGGELILIHQNPGEFCVL |
| SEQ ID NO: 87 | SEQ ID | RTPSDKPVAHVVANPQAEGQLQWLNRRANALLANGVELRDNQLV |
| hTNFα | NO: 52 | VPSEGLYLIYSQVLFKGQGCPSTHVLLTHTISRIAVSYQTKVNL |
| TNFSF2 | | LSAIKSPCQRETPEGAEAKPWYEPIYLGGVFQLEKGDRLSAEIN |
| | | RPDYLDFAESGQVYFGIIAL |
| SEQ ID NO: 88 | SEQ ID | DKPVAHVVANPQAEGQLQWLNRRANALLANGVELRDNQLVVPSE |
| hTNFα | NO: 53 | GLYLIYSQVLFKGQGCPSTHVLLTHTISRIAVSYQTKVNLLSAI |
| TNFSF2 | | KSPCQRETPEGAEAKPWYEPIYLGGVFQLEKGDRLSAEINRPDY |
| | | LDFAESGQVYFGIIAL |
| SEQ ID NO: 89 | SEQ ID | QLETAKEPCMAKFGPLPSKWQMASSEPPCVNKVSDWKLEILQNG |
| hGITRL | NO: 54 | LYLIYGQVAPNANYNDVAPFEVRLYKNKDMIQTLTNKSKIQNVG |
| TNFSF18 | | GTYELHVGDTIDLIFNSEHQVLKNNTYWGIILLAN |
| SEQ ID NO: 90 | SEQ ID | QLETAKEPCMAKFGPLPSKWQMASSEPPCVNKVSDWKLEILQNG |
| hGITRL | NO: 55 | LYLIYGQVAPNANYNDVAPFEVRLYKNKDMIQTLTNKSKIQNVG |
| TNFSF18 | | GTYELHVGDTIDLIFNSEHQVLKNNTYWGIILLANPQFIS |
| SEQ ID NO: 91 | SEQ ID | NPAAHLTGANSSLTGSGGPLLWETQLGLAFLRGLSYHDGALVVT |
| hLIGHT | NO: 56 | KAGYYYIYSKVQLGGVGCPLGLASTITHGLYKRTPRYPEELELL |
| TNFSF14 | | VSQQSPCGRATSSSRVWWDSSFLGGVVHLEAGEKVVVRVLDERL |
| | | VRLRDGTRSYFGAFMV |
| SEQ ID NO: 92 | SEQ ID | LRKVAHLTGKSNSRSMPLEWEDTYGIVLLSGVKYKKGGLVINET |
| hFASL | NO: 57 | GLYFVYSKVYFRGQSCNNLPLSHKVYMRNSKYPQDLVMMEGKMM |
| TNFSF6 | | SYCTTGQMWARSSYLGAVFNLTSADHLYVNVSELSLVNFEESQT |
| | | FFGLYKL |
| SEQ ID NO: 93 | SEQ ID | KVAHLTGKSNSRSMPLEWEDTYGIVLLSGVKYKKGGLVINETGL |
| hFASL | NO: 58 | YFVYSKVYFRGQSCNNLPLSHKVYMRNSKYPQDLVMMEGKMMSY |
| TNFSF6 | | CTTGQMWARSSYLGAVFNLTSADHLYVNVSELSLVNFEESQTFF |
| | | GLYKL |
| SEQ ID NO: 195 | | REGPELSPDDPAGLLDLRQGMFAQLVAQNVLLIDGPLSWYSDPG |
| 41BBL | | LAGVSLTGGLSYKEDTKELVVAKAGVYYVFFQLELRRVVAGEGS |
| TNFSF9 | | GSVSLALHLQPLRSAAGAAALALTVDLPPASSEARNSAFGFQGR |
| | | LLHLSAGQRLGVHLHTEARARHAWQLTQGATVLGLFRVTPEIPA |
| | | GLPSPRSE |
| SEQ ID NO: 196 | | REGPELSPDDPAGLLDLRQGMFAQLVAQNVLLIDGPLSWYSDPG |
| 41BBL | | LAGVSLTGGLSYKEDTKELVVAKAGVYYVFFQLELRRVVAGEGS |
| TNFSF9 | | GSVSLALHLQPLRSAAGAAALALTVDLPPASSEARNSAFGFQGR |
| | | LLHLSAGQRLGVHLHTEARARHAWQLTQGATVLGLFRVTPEIPA |
| | | GLPSPRS |

TABLE 6
Individual protein chains (the signal peptide is underlined)

| | Corresponding | |
|---|---|---|
| SEQ ID NO | DNA SEQ ID | Sequence |
| SEQ ID NO: 94 | SEQ ID NO: 59 | METDTLLLWVLLLWVPGSTGDKTHTCPPCPAPELLGGPSVFLFP |
| hFc_Knob- | | PKPKDTLMISRTPEVTCVVVDVSHEDPEVKFNWYVDGVEVHNAK |
| 2X_CD40L | | TKPREEQYNSTYRVVSVLTVLHQDWLNGKEYKCKVSNKALPAPI |
| | | EKTISKAKGQPREPQVYTLPPCREEMTKNQVSLWCLVKGFYPSD |
| | | IAVEWESNGQPENNYKTTPPVLDSDGSFFLYSKLTVDKSRWQQG |
| | | NVFSCSVMHEALHNHYTQKSLSLSPGKGGGGSGGGGSGGGGSGD |
| | | QNPQIAAHVISEASSKTTSVLQWAEKGYYTMSNNLVTLENGKQL |
| | | TVKRQGLYYIYAQVTFCSNREASSQAPFIASLALKSPGRFERIL |
| | | LRAANTHSSAKPCGQQSIHLGGVFELQPGASVFVNVTDPSQVSH |
| | | GTGFTSFGLLKLGGGGSGDQNPQIAAHVISEASSKTTSVLQWAE |
| | | KGYYTMSNNLVTLENGKQLTVKRQGLYYIYAQVTFCSNREASSQ |
| | | APFIASLALKSPGRFERILLRAANTHSSAKPCGQQSIHLGGVFE |
| | | LQPGASVFVNVTDPSQVSHGTGFTSFGLLKL |
| SEQ ID NO: 95 | SEQ ID NO: 60 | METDTLLLWVLLLWVPGSTGDKTHTCPPCPAPELLGGPSVFLFP |
| hFc_Hole- | | PKPKDTLMISRTPEVTCVVVDVSHEDPEVKFNWYVDGVEVHNAK |
| 1X_CD40L | | TKPREEQYNSTYRVVSVLTVLHQDWLNGKEYKCKVSNKALPAPI |
| | | EKTISKAKGQPREPQVCTLPPSREEMTKNQVSLSCAVKGFYPSD |
| | | IAVEWESNGQPENNYKTTPPVLDSDGSFFLVSKLTVDKSRWQQG |
| | | NVFSCSVMHEALHNHYTQKSLSLSPGKGGGGSGGGGSGGGGSGD |
| | | QNPQIAAHVISEASSKTTSVLQWAEKGYYTMSNNLVTLENGKQL |
| | | TVKRQGLYYIYAQVTFCSNREASSQAPFIASLALKSPGRFERIL |
| | | LRAANTHSSAKPCGQQSIHLGGVFELQPGASVFVNVTDPSQVSH |
| | | GTGFTSFGLLKL |
| SEQ ID NO: 96 | SEQ ID NO: 61 | METDTLLLWVLLLWVPGSTGDKTHTCPPCPAPELLGGPSVFLFP |
| hFc_Knob | | PKPKDTLMISRTPEVTCVVVDVSHEDPEVKFNWYVDGVEVHNAK |
| | | TKPREEQYNSTYRVVSVLTVLHQDWLNGKEYKCKVSNKALPAPI |
| | | EKTISKAKGQPREPQVYTLPPCREEMTKNQVSLWCLVKGFYPSD |
| | | IAVEWESNGQPENNYKTTPPVLDSDGSFFLYSKLTVDKSRWQQG |
| | | NVFSCSVMHEALHNHYTQKSLSLSPGK |
| SEQ ID NO: 97 | SEQ ID NO: 62 | METDTLLLWVLLLWVPGSTGGDQNPQIAAHVISEASSKTTSVLQ |
| 2X_CD40L- | | WAEKGYYTMSNNLVTLENGKQLTVKRQGLYYIYAQVTFCSNREA |
| hCHIg_Hole | | SSQAPFIASLALKSPGRFERILLRAANTHSSAKPCGQQSIHLGG |
| | | VFELQPGASVFVNVTDPSQVSHGTGFTSFGLLKLGGGGSGDQNP |
| | | QIAAHVISEASSKTTSVLQWAEKGYYTMSNNLVTLENGKQLTVK |
| | | RQGLYYIYAQVTFCSNREASSQAPFIASLALKSPGRFERILLRA |
| | | ANTHSSAKPCGQQSIHLGGVFELQPGASVFVNVTDPSQVSHGTG |
| | | FTSFGLLKLGGGGSGGGGSGGGGSASTKGPSVFPLAPSSKSTSG |
| | | GTAALGCLVKDYFPEPVTVSWNSGALTSGVHTFPAVLQSSGLYS |
| | | LSSVVTVPSSSLGTQTYICNVNHKPSNTKVDKRVEPKSCDKTHT |
| | | CPPCPAPELLGGPSVFLFPPKPKDTLMISRTPEVTCVVVDVSHE |
| | | DPEVKFNWYVDGVEVHNAKTKPREEQYNSTYRVVSVLTVLHQDW |
| | | LNGKEYKCKVSNKALPAPIEKTISKAKGQPREPQVCTLPPSREE |
| | | MTKNQVSLSCAVKGFYPSDIAVEWESNGQPENNYKTTPPVLDSD |
| | | GSFFLVSKLTVDKSRWQQGNVFSCSVMHEALHNHYTQKSLSLSP |
| | | GK |
| SEQ ID NO: 98 | SEQ ID NO: 63 | METDTLLLWVLLLWVPGSTGGDQNPQIAAHVISEASSKTTSVLQ |
| 1X_CD40L- | | WAEKGYYTMSNNLVTLENGKQLTVKRQGLYYIYAQVTFCSNREA |
| hCLIg_vk | | SSQAPFIASLALKSPGRFERILLRAANTHSSAKPCGQQSIHLGG |
| | | VFELQPGASVFVNVTDPSQVSHGTGFTSFGLLKLGGGGSGGGGS |
| | | GGGGSRTVAAPSVFIFPPSDEQLKSGTASVVCLLNNFYPREAKV |
| | | QWKVDNALQSGNSQESVTEQDSKDSTYSLSSTLTLSKADYEKHK |
| | | VYACEVTHQGLSSPVTKSFNRGEC |
| SEQ ID NO: 99 | SEQ ID NO: 64 | METDTLLLWVLLLWVPGSTGDKTHTCPPCPAPELLGGPSVFLFP |
| hFc_Knob- | | PKPKDTLMISRTPEVTCVVVDVSHEDPEVKFNWYVDGVEVHNAK |
| 2X_OX40L | | TKPREEQYNSTYRVVSVLTVLHQDWLNGKEYKCKVSNKALPAPI |
| | | EKTISKAKGQPREPQVYTLPPCREEMTKNQVSLWCLVKGFYPSD |
| | | IAVEWESNGQPENNYKTTPPVLDSDGSFFLYSKLTVDKSRWQQG |
| | | NVFSCSVMHEALHNHYTQKSLSLSPGKGGGGSGGGGSGGGGSGG |
| | | GGSRIQSIKVQFTEYKKEKGFILTSQKEDEIMKVQNNSVIINCD |
| | | GFYLISLKGYFSQEVNISLHYQKDEEPLFQLKKVRSVNSLMVAS |
| | | LTYKDKVYLNVTTDNTSLDDFHVNGGELILIHQNPGEFCVLGGG |
| | | SGGGRIQSIKVQFTEYKKEKGFILTSQKEDEIMKVQNNSVIINC |
| | | DGFYLISLKGYFSQEVNISLHYQKDEEPLFQLKKVRSVNSLMVA |
| | | SLTYKDKVYLNVTTDNTSLDDFHVNGGELILIHQNPGEFCVL |
| SEQ ID NO: 100 | SEQ ID NO: 65 | METDTLLLWVLLLWVPGSTGDKTHTCPPCPAPELLGGPSVFLFP |
| hFc_Hole- | | PKPKDTLMISRTPEVTCVVVDVSHEDPEVKFNWYVDGVEVHNAK |
| 1X_OX40L | | TKPREEQYNSTYRVVSVLTVLHQDWLNGKEYKCKVSNKALPAPI |
| | | EKTISKAKGQPREPQVCTLPPSREEMTKNQVSLSCAVKGFYPSD |
| | | IAVEWESNGQPENNYKTTPPVLDSDGSFFLVSKLTVDKSRWQQG |
| | | NVFSCSVMHEALHNHYTQKSLSLSPGKGGGGSGGGGSGGGGSGG |
| | | GGSRIQSIKVQFTEYKKEKGFILTSQKEDEIMKVQNNSVIINCD |
| | | GFYLISLKGYFSQEVNISLHYQKDEEPLFQLKKVRSVNSLMVAS |
| | | LTYKDKVYLNVTTDNTSLDDFHVNGGELILIHQNPGEFCVL |
| SEQ ID NO: 101 | SEQ ID NO: 66 | METDTLLLWVLLLWVPGSTGDKTHTCPPCPAPELLGGPSVFLFP |
| hFc_Knob- | | PKPKDTLMISRTPEVTCVVVDVSHEDPEVKFNWYVDGVEVHNAK |
| 2X_TNFα | | TKPREEQYNSTYRVVSVLTVLHQDWLNGKEYKCKVSNKALPAPI |
| | | EKTISKAKGQPREPQVYTLPPCREEMTKNQVSLWCLVKGFYPSD |
| | | IAVEWESNGQPENNYKTTPPVLDSDGSFFLYSKLTVDKSRWQQG |
| | | NVFSCSVMHEALHNHYTQKSLSLSPGKGGGGSGGGGSGGGGSGG |
| | | GGSRTPSDKPVAHVVANPQAEGQLQWLNRRANALLANGVELRDN |
| | | QLVVPSEGLYLIYSQVLFKGQGCPSTHVLLTHTISRIAVSYQTK |
| | | VNLLSAIKSPCQRETPEGAEAKPWYEPIYLGGVFQLEKGDRLSA |
| | | EINRPDYLDFAESGQVYFGIIALGGSGGSDKPVAHVVANPQAEG |
| | | QLQWLNRRANALLANGVELRDNQLVVPSEGLYLIYSQVLFKGQG |
| | | CPSTHVLLTHTISRIAVSYQTKVNLLSAIKSPCQRETPEGAEAK |
| | | PWYEPIYLGGVFQLEKGDRLSAEINRPDYLDFAESGQVYFGIIA |
| | | L |
| SEQ ID NO: 102 | SEQ ID NO: 67 | METDTLLLWVLLLWVPGSTGDKTHTCPPCPAPELLGGPSVFLFP |
| hFc_Hole- | | PKPKDTLMISRTPEVTCVVVDVSHEDPEVKFNWYVDGVEVHNAK |
| 1X_TNFα | | TKPREEQYNSTYRVVSVLTVLHQDWLNGKEYKCKVSNKALPAPI |
| | | EKTISKAKGQPREPQVCTLPPSREEMTKNQVSLSCAVKGFYPSD |
| | | IAVEWESNGQPENNYKTTPPVLDSDGSFFLVSKLTVDKSRWQQG |
| | | NVFSCSVMHEALHNHYTQKSLSLSPGKGGGGSGGGGSGGGGSGG |
| | | GGSRTPSDKPVAHVVANPQAEGQLQWLNRRANALLANGVELRDN |
| | | QLVVPSEGLYLIYSQVLFKGQGCPSTHVLLTHTISRIAVSYQTK |
| | | VNLLSAIKSPCQRETPEGAEAKPWYEPIYLGGVFQLEKGDRLSA |
| | | EINRPDYLDFAESGQVYFGIIAL |
| SEQ ID NO: 103 | SEQ ID NO: 68 | METDTLLLWVLLLWVPGSTGDKTHTCPPCPAPELLGGPSVFLFP |
| hFc_Knob- | | PKPKDTLMISRTPEVTCVVVDVSHEDPEVKFNWYVDGVEVHNAK |
| 2X_FASL | | TKPREEQYNSTYRVVSVLTVLHQDWLNGKEYKCKVSNKALPAPI |
| | | EKTISKAKGQPREPQVYTLPPCREEMTKNQVSLWCLVKGFYPSD |
| | | IAVEWESNGQPENNYKTTPPVLDSDGSFFLYSKLTVDKSRWQQG |
| | | NVFSCSVMHEALHNHYTQKSLSLSPGKGGGGSGGGGSGGGGSGG |
| | | GGSLRKVAHLTGKSNSRSMPLEWEDTYGIVLLSGVKYKKGGLVI |
| | | NETGLYFVYSKVYFRGQSCNNLPLSHKVYMRNSKYPQDLVMMEG |
| | | KMMSYCTTGQMWARSSYLGAVFNLTSADHLYVNVSELSLVNFEE |
| | | SQTFFGLYKLGGSGGKVAHLTGKSNSRSMPLEWEDTYGIVLLSG |
| | | VKYKKGGLVINETGLYFVYSKVYFRGQSCNNLPLSHKVYMRNSK |
| | | YPQDLVMMEGKMMSYCTTGQMWARSSYLGAVFNLTSADHLYVNV |
| | | SELSLVNFEESQTFFGLYKL |
| SEQ ID NO: 104 | SEQ ID NO: 69 | METDTLLLWVLLLWVPGSTGDKTHTCPPCPAPELLGGPSVFLFP |
| hFc_Hole- | | PKPKDTLMISRTPEVTCVVVDVSHEDPEVKFNWYVDGVEVHNAK |
| 1X_FASL | | TKPREEQYNSTYRVVSVLTVLHQDWLNGKEYKCKVSNKALPAPI |
| | | EKTISKAKGQPREPQVCTLPPSREEMTKNQVSLSCAVKGFYPSD |
| | | IAVEWESNGQPENNYKTTPPVLDSDGSFFLVSKLTVDKSRWQQG |
| | | NVFSCSVMHEALHNHYTQKSLSLSPGKGGGGSGGGGSGGGGSGG |
| | | GGSLRKVAHLTGKSNSRSMPLEWEDTYGIVLLSGVKYKKGGLVI |
| | | NETGLYFVYSKVYFRGQSCNNLPLSHKVYMRNSKYPQDLVMMEG |
| | | KMMSYCTTGQMWARSSYLGAVFNLTSADHLYVNVSELSLVNFEE |
| | | SQTFFGLYKL |
| SEQ ID NO: 105 | SEQ ID NO: 70 | METDTLLLWVLLLWVPGSTGDKTHTCPPCPAPELLGGPSVFLFP |
| hFc_Knob- | | PKPKDTLMISRTPEVTCVVVDVSHEDPEVKFNWYVDGVEVHNAK |
| 2X_LIGHT | | TKPREEQYNSTYRVVSVLTVLHQDWLNGKEYKCKVSNKALPAPI |
| | | EKTISKAKGQPREPQVYTLPPCREEMTKNQVSLWCLVKGFYPSD |
| | | IAVEWESNGQPENNYKTTPPVLDSDGSFFLYSKLTVDKSRWQQG |
| | | NVFSCSVMHEALHNHYTQKSLSLSPGKGGGGSGGGGSGGGGSGG |
| | | GGSNPAAHLTGANSSLTGSGGPLLWETQLGLAFLRGLSYHDGAL |
| | | VVTKAGYYYIYSKVQLGGVGCPLGLASTITHGLYKRTPRYPEEL |
| | | ELLVSQQSPCGRATSSSRVWWDSSFLGGVVHLEAGEKVVVRVLD |
| | | ERLVRLRDGTRSYFGAFMVGGSGGNPAAHLTGANSSLTGSGGPL |
| | | LWETQLGLAFLRGLSYHDGALVVTKAGYYYIYSKVQLGGVGCPL |
| | | GLASTITHGLYKRTPRYPEELELLVSQQSPCGRATSSSRVWWDS |
| | | SFLGGVVHLEAGEKVVVRVLDERLVRLRDGTRSYFGAFMV |
| SEQ ID NO: 106 | SEQ ID NO: 71 | METDTLLLWVLLLWVPGSTGDKTHTCPPCPAPELLGGPSVFLFP |
| hfc_Hole- | | PKPKDTLMISRTPEVTCVVVDVSHEDPEVKFNWYVDGVEVHNAK |
| 1X_LIGHT | | TKPREEQYNSTYRVVSVLTVLHQDWLNGKEYKCKVSNKALPAPI |
| | | EKTISKAKGQPREPQVCTLPPSREEMTKNQVSLSCAVKGFYPSD |
| | | IAVEWESNGQPENNYKTTPPVLDSDGSFFLVSKLTVDKSRWQQG |
| | | NVFSCSVMHEALHNHYTQKSLSLSPGKGGGGSGGGGSGGGGSGG |
| | | GGSNPAAHLTGANSSLTGSGGPLLWETQLGLAFLRGLSYHDGAL |
| | | VVTKAGYYYIYSKVQLGGVGCPLGLASTITHGLYKRTPRYPEEL |
| | | ELLVSQQSPCGRATSSSRVWWDSSFLGGVVHLEAGEKVVVRVLD |
| | | ERLVRLRDGTRSYFGAFMV |
| SEQ LD NO: 107 | SEQ ID NO: 72 | METDTLLLWVLLLWVPGSTGDKTHTCPPCPAPELLGGPSVFLFP |
| hFc_Knob- | | PKPKDTLMISRTPEVTCVVVDVSHEDPEVKFNWYVDGVEVHNAK |
| 2X_GITRL | | TKPREEQYNSTYRVVSVLTVLHQDWLNGKEYKCKVSNKALPAPI |
| | | ERTISKAKGQPREPQVYTLPPCREEMTKNQVSLWCLVKGFYPSD |
| | | IAVEWESNGQPENNYKTTPPVLDSDGSFFLYSKLTVDKSRWQQG |
| | | NVFSCSVMHEALHNHYTQKSLSLSPGKGGGGSGGGGSGGGGSGG |
| | | GGSQLETAKEPCMAKFGPLPSKWQMASSEPPCVNKVSDWKLEIL |
| | | QNGLYLIYGQVAPNANYNDVAPFEVRLYKNKDMIQTLTNKSKIQ |
| | | NVGGTYELHVGDTIDLIFNSEHQVLKNNTYWGIILLANGQLETA |
| | | KEPCMAKFGPLPSKWQMASSEPPCVNKVSDWKLEILQNGLYLIY |
| | | GQVAPNANYNDVAPFEVRLYKNKDMIQTLTNKSKIQNVGGTYEL |
| | | HVGDTIDLIFNSEHQVLKNNTYWGIILLANPQFIS |
| SEQ ID NO: 108 | SEQ ID NO: 73 | METDTLLLWVLLLWVPGSTGDKTHTCPPCPAPELLGGPSVFLFP |
| hFc_Hole- | | PKPKDTLMISRTPEVTCVVVDVSHEDPEVKFNWYVDGVEVHNAK |
| 1X_GITRL | | TKPREEQYNSTYRVVSVLTVLHQDWLNGKEYKCKVSNKALPAPI |
| | | ERTISKAKGQPREPQVCTLPPSREEMTKNQVSLSCAVKGFYPSD |
| | | IAVEWESNGQPENNYKTTPPVLDSDGSFFLVSKLTVDKSRWQQG |
| | | NVFSCSVMHEALHNHYTQKSLSLSPGKGGGGSGGGGSGGGGSGG |
| | | GGSQLETAKEPCMAKFGPLPSKWQMASSEPPCVNKVSDWKLEIL |
| | | QNGLYLIYGQVAPNANYNDVAPFEVRLYKNKDMIQTLTNKSKIQ |
| | | NVGGTYELHVGDTIDLIFNSEHQVLKNNTYWGIILLANPQFIS |
| SEQ ID NO: 158 | | DKTHTSPPSPAPELLGGPSVFLFPPKPKDTLMISRTPEVTCVVV |
| hFc_Knob(ΔCys)- | | DVSHEDPEVKFNWYVDGVEVHNAKTKPREEQYNSTYRVVSVLTV |
| 2X_GITRL | | LHQDWLNGKEYKCKVSNKALPAPIEKTISKAKGQPREPQVYTLP |
| | | PSREEMTKNQVSLWCLVKGFYPSDIAVEWESNGQPENNYKTTPP |
| | | VLDSDGSFFLYSKLTVDKSRWQQGNVFSCSVMHEALHNHYTQKS |
| | | LSLSPGKGGGGSGGGGSGGGGSGGGGSQLETAKEPCMAKFGPLP |
| | | SKWQMASSEPPCVNKVSDWKLEILQNGLYLIYGQVAPNANYNDV |
| | | APFEVRLYKNKDMIQTLTNKSKIQNVGGTYELHVGDTIDLIFNS |
| | | EHQVLKNNTYWGIILLANGGGGSGGGGQLETAKEPCMAKFGPLP |
| | | SKWQMASSEPPCVNKVSDWKLEILQNGLYLIYGQVAPNANYNDV |
| | | APFEVRLYKNKDMIQTLTNKSKIQNVGGTYELHVGDTIDLIFNS |
| | | EHQVLKNNTYWGIILLANPQFIS |
| SEQ ID NO: 159 | | DKTHTSPPSPAPELLGGPSVFLFPPKPKDTLMISRTPEVTCVVV |
| hFc_Hole(ΔCys)- | | DVSHEDPEVKFNWYVDGVEVHNAKTKPREEQYNSTYRVVSVLTV |
| 1X_-GITRL | | LHQDWLNGKEYKCKVSNKALPAPIEKTISKAKGQPREPQVYTLP |
| | | PSREEMTKNQVSLSCAVKGFYPSDIAVEWESNGQPENNYKTTPP |
| | | VLDSDGSFFLVSKLTVDKSRWQQGNVFSCSVMHEALHNHYTQKS |
| | | LSLSPGKGGGGSGGGGSGGGGSGGGGSQLETAKEPCMAKFGPLP |
| | | SKWQMASSEPPCVNKVSDWKLEILQNGLYLIYGQVAPNANYNDV |
| | | APFEVRLYKNKDMIQTLTNKSKIQNVGGTYELHVGDTIDLIFNS |
| | | EHQVLKNNTYWGIILLANPQFIS |
| SEQ ID NO: 160 | | DKTHTCPPCPAPELLGGPSVFLFPPKPKDTLMISRTPEVTCVVV |
| hFc_Knob_Cys- | | DVSHEDPEVKFNWYVDGVEVHNAKTKPREEQYNSTYRVVSVLTV |
| 2X_GITRL | | LHQDWLNGKEYKCKVSNKALPAPIEKTISKAKGQPREPQVYTLP |
| | | PCREEMTKNQVSLWCLVKGFYPSDIAVEWESNGQPENNYKTTPP |
| | | VLDSDGSFFLYSKLTVDKSRWQQGNVFSCSVMHEALHNHYTQKS |
| | | LSLSPGKGGGGSGGGGSGGGGSGGGGSQLETAKEPCMAKFGPLP |
| | | SKWQMASSEPPCVNKVSDWKLEILQNGLYLIYGQVAPNANYNDV |
| | | APFEVRLYKNKDMIQTLTNKSKIQNVGGTYELHVGDTIDLIFNS |
| | | EHQVLKNNTYWGIILLANGGGGSGGGGQLETAKEPCMAKFGPLP |
| | | SKWQMASSEPPCVNKVSDWKLEILQNGLYLIYGQVAPNANYNDV |
| | | APFEVRLYKNKDMIQTLTNKSKIQNVGGTYELHVGDTIDLIFNS |
| | | EHQVLKNNTYWGIILLANPQFIS |
| SEQ ID NO: 161 | | DKTHTCPPCPAPELLGGPSVFLFPPKPKDTLMISRTPEVTCVVV |
| hFc_Hole_Cys- | | DVSHEDPEVKFNWYVDGVEVHNAKTKPREEQYNSTYRVVSVLTV |
| 1X_GITRL | | LHQDWLNGKEYKCKVSNKALPAPIEKTISKAKGQPREPQVCTLP |
| | | PSREEMTKNQVSLSCAVKGFYPSDIAVEWESNGQPENNYKTTPP |
| | | VLDSDGSFFLVSKLTVDKSRWQQGNVFSCSVMHEALHNHYTQKS |
| | | LSLSPGKGGGGSGGGGSGGGGSGGGGSQLETAKEPCMAKFGPLP |
| | | SKWQMASSEPPCVNKVSDWKLEILQNGLYLIYGQVAPNANYNDV |
| | | APFEVRLYKNKDMIQTLTNKSKIQNVGGTYELHVGDTIDLIFNS |
| | | EHQVLKNNTYWGIILLANPQFIS |
| SEQ ID NO: 162 | | QLETAKEPCMAKFGPLPSKWQMASSEPPCVNKVSDWKLEILQNG |
| 2X_GITRL- | | LYLIYGQVAPNANYNDVAPFEVRLYKNKDMIQTLTNKSKIQNVG |
| hCH1(ΔCys)- | | GTYELHVGDTIDLIFNSEHQVLKNNTYWGIILLANGGGGSGGGG |
| Fc_Knob_Cys | | QLETAKEPCMAKFGPLPSKWQMASSEPPCVNKVSDWKLEILQNG |
| | | LYLIYGQVAPNANYNDVAPFEVRLYKNKDMIQTLTNKSKIQNVG |
| | | GTYELHVGDTIDLIFNSEHQVLKNNTYWGIILLANPQFISGGGG |
| | | SGGGGSGGGGSASTKGPSVFPLAPSSKSTSGGTAALGCLVKDYF |
| | | PEPVTVSWNSGALTSGVHTFPAVLQSSGLYSLSSVVTVPSSSLG |
| | | TQTYICNVNHKPSNTKVDKRVEPKSSDKTHTCPPCPAPELLGGP |
| | | SVFLFPPKPKDTLMISRTPEVTCVVVDVSHEDPEVKFNWYVDGV |
| | | EVHNAKTKPREEQYNSTYRVVSVLTVLHQDWLNGKEYKCKVSNK |
| | | ALPAPIEKTISKAKGQPREPQVYTLPPCREEMTKNQVSLWCLVK |
| | | GFYPSDIAVEWESNGQPENNYKTTPPVLDSDGSFFLYSKLTVDK |
| | | SRWQQGNVFSCSVMHEALHNHYTQKSLSLSPGK |
| SEQ ID NO: 163 | | QLETAKEPCMAKFGPLPSKWQMASSEPPCVNKVSDWKLEILQNG |
| 1X_GITRL- | | LYLIYGQVAPNANYNDVAPFEVRLYKNKDMIQTLTNKSKIQNVG |
| hCLIg_vk(ΔCys) | | GTYELHVGDTIDLIFNSEHQVLKNNTYWGIILLANPQFISGGGG |
| | | SGGGGSGGGGSRTVAAPSVFIFPPSDEQLKSGTASVVCLLNNFY |
| | | PREAKVQWKVDNALQSGNSQESVTEQDSKDSTYSLSSTLTLSKA |
| | | DYEKHKVYACEVTHQGLSSPVTKSFNRGES |
| SEQ ID NO: 164 | | DKTHTCPPCPAPELLGGPSVFLFPPKPKDTLMISRTPEVTCVVV |
| hFc_Hole_Cys | | DVSHEDPEVKFNWYVDGVEVHNAKTKPREEQYNSTYRVVSVLTV |
| | | LHQDWLNGKEYKCKVSNKALPAPIEKTISKAKGQPREPQVCTLP |
| | | PSREEMTKNQVSLSCAVKGFYPSDIAVEWESNGQPENNYKTTPP |
| | | VLDSDGSFFLVSKLTVDKSRWQQGNVFSCSVMHEALHNHYTQKS |
| | | LSLSPGK |
| SEQ ID NO: 165 | | QLETAKEPCMAKFGPLPSKWQMASSEPPCVNKVSDWKLEILQNG |
| 2X_GITRL- | | LYLIYGQVAPNANYNDVAPFEVRLYKNKDMIQTLTNKSKIQNVG |
| hCHIg_Knob_Cys | | GTYELHVGDTIDLIFNSEHQVLKNNTYWGIILLANGGGGSGGGG |
| | | QLETAKEPCMAKFGPLPSKWQMASSEPPCVNKVSDWKLEILQNG |
| | | LYLIYGQVAPNANYNDVAPFEVRLYKNKDMIQTLTNKSKIQNVG |
| | | GTYELHVGDTIDLIFNSEHQVLKNNTYWGIILLANPQFISGGGG |
| | | SGGGGSGGGGSASTKGPSVFPLAPSSKSTSGGTAALGCLVKDYF |
| | | PEPVTVSWNSGALTSGVHTFPAVLQSSGLYSLSSVVTVPSSSLG |
| | | TQTYICNVNHKPSNTKVDKRVEPKSCDKTHTCPPCPAPELLGGP |
| | | SVFLFPPKPKDTLMISRTPEVTCVVVDVSHEDPEVKFNWYVDGV |
| | | EVHNAKTKPREEQYNSTYRVVSVLTVLHQDWLNGKEYKCKVSNK |
| | | ALPAPIEKTISKAKGQPREPQVYTLPPCREEMTKNQVSLWCLVK |
| | | GFYPSDIAVEWESNGQPENNYKTTPPVLDSDGSFFLYSKLTVDK |
| | | SRWQQGNVFSCSVMHEALHNHYTQKSLSLSPGK |
| SEQ ID NO: 166 | | QLETAKEPCMAKFGPLPSKWQMASSEPPCVNKVSDWKLEILQNG |
| 1X_GITRL- | | LYLIYGQVAPNANYNDVAPFEVRLYKNKDMIQTLTNKSKIQNVG |
| hCLIg_vk | | GTYELHVGDTIDLIFNSEHQVLKNNTYWGIILLANPQFISGGGG |
| | | SGGGGSGGGGSRTVAAPSVFIFPPSDEQLKSGTASVVCLLNNFY |
| | | PREAKVQWKVDNALQSGNSQESVTEQDSKDSTYSLSSTLTLSKA |
| | | DYEKHKVYACEVTHQGLSSPVTKSFNRGEC |
| SEQ ID NO: 167 | | DKTHTCPPCPAPELLGGPSVFLFPPKPKDTLMISRTPEVTCVVV |
| hFc_Knob_Cys- | | DVSHEDPEVKFNWYVDGVEVHNAKTKPREEQYNSTYRVVSVLTV |
| 2X_FASL | | LHQDWLNGKEYKCKVSNKALPAPIEKTISKAKGQPREPQVYTLP |
| | | PCREEMTKNQVSLWCLVKGFYPSDIAVEWESNGQPENNYKTTPP |
| | | VLDSDGSFFLYSKLTVDKSRWQQGNVFSCSVMHEALHNHYTQKS |
| | | LSLSPGKGGGGSGGGGSGGGGSGGGGSLRKVAHLTGKSNSRSMP |
| | | LEWEDTYGIVLLSGVKYKKGGLVINETGLYFVYSKVYFRGQSCN |
| | | NLPLSHKVYMRNSKYPQDLVMMEGKMMSYCTTGQMWARSSYLGA |
| | | VFNLTSADHLYVNVSELSLVNFEESQTFFGLYKLGGSGGKVAHL |
| | | TGKSNSRSMPLEWEDTYGIVLLSGVKYKKGGLVINETGLYFVYS |
| | | KVYFRGQSCNNLPLSHKVYMRNSKYPQDLVMMEGKMMSYCTTGQ |
| | | MWARSSYLGAVFNLTSADHLYVNVSELSLVNFEESQTFFGLYKL |
| SEQ ID NO: 168 | | DKTHTCPPCPAPELLGGPSVFLFPPKPKDTLMISRTPEVTCVVV |
| hFc_Hole_Cys- | | DVSHEDPEVKFNWYVDGVEVHNAKTKPREEQYNSTYRVVSVLTV |
| 1X_FASL | | LHQDWLNGKEYKCKVSNKALPAPIEKTISKAKGQPREPQVCTLP |
| | | PSREEMTKNQVSLSCAVKGFYPSDIAVEWESNGQPENNYKTTPP |
| | | VLDSDGSFFLVSKLTVDKSRWQQGNVFSCSVMHEALHNHYTQKS |
| | | LSLSPGKGGGGSGGGGSGGGGSGGGGSLRKVAHLTGKSNSRSMP |
| | | LEWEDTYGIVLLSGVKYKKGGLVINETGLYFVYSKVYFRGQSCN |
| | | NLPLSHKVYMRNSKYPQDLVMMEGKMMSYCTTGQMWARSSYLGA |
| | | VFNLTSADHLYVNVSELSLVNFEESQTFFGLYKL |
| SEQ ID NO: 169 | | DKTHTSPPSPAPELLGGPSVFLFPPKPKDTLMISRTPEVTCVVV |
| hFc_Knob(ΔCys)- | | DVSHEDPEVKFNWYVDGVEVHNAKTKPREEQYNSTYRVVSVLTV |
| 2X_FASL | | LHQDWLNGKEYKCKVSNKALPAPIEKTISKAKGQPREPQVYTLP |
| | | PSREEMTKNQVSLWCLVKGFYPSDIAVEWESNGQPENNYKTTPP |
| | | VLDSDGSFFLYSKLTVDKSRWQQGNVFSCSVMHEALHNHYTQKS |
| | | LSLSPGKGGGGSGGGGSGGGGSGGGGSLRKVAHLTGKSNSRSMP |
| | | LEWEDTYGIVLLSGVKYKKGGLVINETGLYFVYSKVYFRGQSCN |
| | | NLPLSHKVYMRNSKYPQDLVMMEGKMMSYCTTGQMWARSSYLGA |
| | | VFNLTSADHLYVNVSELSLVNFEESQTFFGLYKLGGSGGKVAHL |
| | | TGKSNSRSMPLEWEDTYGIVLLSGVKYKKGGLVINETGLYFVYS |
| | | KVYFRGQSCNNLPLSHKVYMRNSKYPQDLVMMEGKMMSYCTTGQ |
| | | MWARSSYLGAVFNLTSADHLYVNVSELSLVNFEESQTFFGLYKL |
| SEQ ID NO: 170 | | DKTHTSPPSPAPELLGGPSVFLFPPKPKDTLMISRTPEVTCVVV |
| hFc_Hole(ΔCys)- | | DVSHEDPEVKFNWYVDGVEVHNAKTKPREEQYNSTYRVVSVLTV |
| 1X_FASL | | LHQDWLNGKEYKCKVSNKALPAPTEKTISKAKGQPREPQVYTLP |
| | | PSREEMTKNQVSLSCAVKGFYPSDIAVEWESNGQPENNYKTTPP |
| | | VLDSDGSFFLVSKLTVDKSRWQQGNVFSCSVMHEALHNHYTQKS |
| | | LSLSPGKGGGGSGGGGSGGGGSGGGGSLRKVAHLTGKSNSRSMP |
| | | LEWEDTYGIVLLSGVKYKKGGLVINETGLYFVYSKVYFRGQSCN |
| | | NLPLSHKVYMRNSKYPQDLVMMEGKMMSYCTTGQMWARSSYLGA |
| | | VFNLTSADHLYVNVSELSLVNFEESQTFFGLYKL |
| SEQ ID NO: 171 | | DKTHTSPPSPAPELLGGPSVFLFPPKPKDTLMISRTPEVTCVVV |
| hFc_Knob(ΔCys)- | | DVSHEDPEVKFNWYVDGVEVHNAKTKPREEQYNSTYRVVSVLTV |
| 2X_4-1BBL | | LHQDWLNGKEYKCKVSNKALPAPIEKTISKAKGQPREPQVYTLP |
| | | PSREEMTKNQVSLWCLVKGFYPSDIAVEWESNGQPENNYKTTPP |
| | | VLDSDGSFFLYSKLTVDKSRWQQGNVFSCSVMHEALHNHYTQKS |
| | | LSLSPGKGGGGSGGGGSGGGGSGGGGSREGPELSPDDPAGLLDL |
| | | RQGMFAQLVAQNVLLIDGPLSWYSDPGLAGVSLTGGLSYKEDTK |
| | | ELVVAKAGVYYVFFQLELRRVVAGEGSGSVSLALHLQPLRSAAG |
| | | AAALALTVDLPPASSEARNSAFGFQGRLLHLSAGQRLGVHLHTE |
| | | ARARHAWQLTQGATVLGLFRVTPEIPAGLPSPRSEGGGGSGGGG |
| | | REGPELSPDDPAGLLDLRQGMFAQLVAQNVLLIDGPLSWYSDPG |
| | | LAGVSLTGGLSYKEDTKELVVAKAGVYYVFFQLELRRVVAGEGS |
| | | GSVSLALHLQPLRSAAGAAALALTVDLPPASSEARNSAFGFQGR |
| | | LLHLSAGQRLGVHLHTEARARHAWQLTQGATVLGLFRVTPEIPA |
| | | GLPSPRS |
| SEQ ID NO: 172 | | DKTHTSPPSPAPELLGGPSVFLFPPKPKDTLMISRTPEVTCVVV |
| hFc_Hole(ΔCys)- | | DVSHEDPEVKFNWYVDGVEVHNAKTKPREEQYNSTYRVVSVLTV |
| 1X_4-1BBL | | LHQDWLNGKEYKCKVSNKALPAPIEKTISKAKGQPREPQVYTLP |
| | | PSREEMTKNQVSLSCAVKGFYPSDIAVEWESNGQPENNYKTTPP |
| | | VLDSDGSFFLVSKLTVDKSRWQQGNVFSCSVMHEALHNHYTQKS |
| | | LSLSPGKGGGGSGGGGSGGGGSGGGGSREGPELSPDDPAGLLDL |
| | | RQGMFAQLVAQNVLLIDGPLSWYSDPGLAGVSLTGGLSYKEDTK |
| | | ELVVAKAGVYYVFFQLELRRVVAGEGSGSVSLALHLQPLRSAAG |
| | | AAALALTVDLPPASSEARNSAFGFQGRLLHLSAGQRLGVHLHTE |
| | | ARARHAWQLTQGATVLGLFRVTPEIPAGLPSPRS |
| SEQ ID NO: 173 | | DKTHTCPPCPAPELLGGPSVFLFPPKPKDTLMISRTPEVTCVVV |
| hFc_Knob_Cys- | | DVSHEDPEVKFNWYVDGVEVHNAKTKPREEQYNSTYRVVSVLTV |
| 2X_4-1BBL | | LHQDWLNGKEYKCKVSNKALPAPIEKTISKAKGQPREPQVYTLP |
| | | PCREEMTKNQVSLWCLVKGFYPSDIAVEWESNGQPENNYKTTPP |
| | | VLDSDGSFFLYSKLTVDKSRWQQGNVFSCSVMHEALHNHYTQKS |
| | | LSLSPGKGGGGSGGGGSGGGGSGGGGSREGPELSPDDPAGLLDL |
| | | RQGMFAQLVAQNVLLIDGPLSWYSDPGLAGVSLTGGLSYKEDTK |
| | | ELVVAKAGVYYVFFQLELRRVVAGEGSGSVSLALHLQPLRSAAG |
| | | AAALALTVDLPPASSEARNSAFGFQGRLLHLSAGQRLGVHLHTE |
| | | ARARHAWQLTQGATVLGLFRVTPEIPAGLPSPRSEGGGGSGGGG |
| | | REGPELSPDDPAGLLDLRQGMFAQLVAQNVLLIDGPLSWYSDPG |
| | | LAGVSLTGGLSYKEDTKELVVAKAGVYYVFFQLELRRVVAGEGS |
| | | GSVSLALHLQPLRSAAGAAALALTVDLPPASSEARNSAFGFQGR |
| | | LLHLSAGQRLGVHLHTEARARHAWQLTQGATVLGLFRVTPETPA |
| | | GLPSPRS |
| SEQ ID NO: 174 | | DKTHTCPPCPAPELLGGPSVFLFPPKPKDTLMISRTPEVTCVVV |
| hFc_Hole_Cys- | | DVSHEDPEVKFNWYVDGVEVHNAKTKPREEQYNSTYRVVSVLTV |
| 1X_4-1BBL | | LHQDWLNGKEYKCKVSNKALPAPIEKTISKAKGQPREPQVCTLP |
| | | PSREEMTKNQVSLSCAVKGFYPSDIAVEWESNGQPENNYKTTPP |
| | | VLDSDGSFFLVSKLTVDKSRWQQGNVFSCSVMHEALHNHYTQKS |
| | | LSLSPGKGGGGSGGGGSGGGGSGGGGSREGPELSPDDPAGLLDL |
| | | RQGMFAQLVAQNVLLIDGPLSWYSDPGLAGVSLTGGLSYKEDTK |
| | | ELVVAKAGVYYVFFQLELRRVVAGEGSGSVSLALHLQPLRSAAG |
| | | AAALALTVDLPPASSEARNSAFGFQGRLLHLSAGQRLGVHLHTE |
| | | ARARHAWQLTQGATVLGLFRVTPEIPAGLPSPRS |
| SEQ ID NO: 175 | | DKTHTCPPCPAPELLGGPSVFLFPPKPKDTLMISRTPEVTCVVV |
| hFc_Knob_Cys- | | DVSHEDPEVKFNWYVDGVEVHNAKTKPREEQYNSTYRVVSVLTV |
| 2X_BAFF | | LHQDWLNGKEYKCKVSNKALPAPIEKTISKAKGQPREPQVYTLP |
| | | PCREEMTKNQVSLWCLVKGFYPSDIAVEWESNGQPENNYKTTPP |
| | | VLDSDGSFFLYSKLTVDKSRWQQGNVFSCSVMHEALHNHYTQKS |
| | | LSLSPGKGGGGSGGGGSGGGGSGGGGSETVTQDCLQLIADSETP |
| | | TIQKGSYTFVPWLLSFKRGSALEEKENKILVKETGYFFIYGQVL |
| | | YTDKTYAMGHLIQRKKVHVFGDELSLVTLFRCIQNMPETLPNNS |
| | | CYSAGIAKLEEGDELQLAIPRENAQISLDGDVTFFGALKLLGGG |
| | | GSETVTQDCLQLIADSETPTIQKGSYTFVPWLLSFKRGSALEEK |
| | | ENKILVKETGYFFIYGQVLYTDKTYAMGHLIQRKKVHVFGDELS |
| | | LVTLFRCIQNMPETLPNNSCYSAGIAKLEEGDELQLAIPRENAQ |
| | | ISLDGDVTFFGALKLL |
| SEQ ID NO: 176 | | DKTHTCPPCPAPELLGGPSVFLFPPKPKDTLMISRTPEVTCVVV |
| hFc_Hole_Cys- | | DVSHEDPEVKFNWYVDGVEVHNAKTKPREEQYNSTYRVVSVLTV |
| 1X_APRIL | | LHQDWLNGKEYKCKVSNKALPAPIEKTISKAKGQPREPQVCTLP |
| | | PSREEMTKNQVSLSCAVKGFYPSDIAVEWESNGQPENNYKTTPP |
| | | VLDSDGSFFLVSKLTVDKSRWQQGNVFSCSVMHEALHNHYTQKS |
| | | LSLSPGKGGGGSGGGGSGGGGSGGGGSHSVLHLVPINAASKDDS |
| | | DVTEVMWQPALRRGRGLQAQGYGVRIQDAGVYLLYSQVLFQDVT |
| | | FTMGQVVSREGQGRQETLFRCIRSMPSHPDRAYNSCYSAGVFHL |
| | | HQGDILSVIIPRARAKLNLSPHGTFLGFVKL |
| SEQ ID NO: 177 | | DKTHTSPPSPAPELLGGPSVFLFPPKPKDTLMISRTPEVTCVVV |
| hFc_Knob(ΔCys-) | | DVSHEDPEVKFNWYVDGVEVHNAKTKPREEQYNSTYRVVSVLTV |
| 2X_BAFF | | LHQDWLNGKEYKCKVSNKALPAPIEKTISKAKGQPREPQVYTLP |
| | | PSREEMTKNQVSLWCLVKGFYPSDIAVEWESNGQPENNYKTTPP |
| | | VLDSDGSFFLYSKLTVDKSRWQQGNVFSCSVMHEALHNHYTQKS |
| | | LSLSPGKGGGGSGGGGSGGGGSGGGGSETVTQDCLQLIADSETP |
| | | TIQKGSYTFVPWLLSFKRGSALEEKENKILVKETGYFFIYGQVL |
| | | YTDKTYAMGHLIQRKKVHVFGDELSLVTLFRCIQNMPETLPNNS |
| | | CYSAGIAKLEEGDELQLAIPRENAQISLDGDVTFFGALKLLGGG |
| | | GSETVTQDCLQLIADSETPTIQKGSYTFVPWLLSFKRGSALEEK |
| | | ENKILVKETGYFFIYGQVLYTDKTYAMGHLIQRKKVHVFGDELS |
| | | LVTLFRCIQNMPETLPNNSCYSAGIAKLEEGDELQLAIPRENAQ |
| | | ISLDGDVTFFGALKLL |
| SEQ ID NO: 178 | | DKTHTSPPSPAPELLGGPSVFLFPPKPKDTLMISRTPEVTCVVV |
| hFc_Hole(ΔCys)- | | DVSHEDPEVKFNWYVDGVEVHNAKTKPREEQYNSTYRVVSVLTV |
| 1X_APRIL | | LHQDWLNGKEYKCKVSNKALPAPIEKTISKAKGQPREPQVYTLP |
| | | PSREEMTKNQVSLSCAVKGFYPSDIAVEWESNGQPENNYKTTPP |
| | | VLDSDGSFFLVSKLTVDKSRWQQGNVFSCSVMHEALHNHYTQKS |
| | | LSLSPGKGGGGSGGGGSGGGGSGGGGSHSVLHLVPINAASKDDS |
| | | DVTEVMWQPALRRGRGLQAQGYGVRIQDAGVYLLYSQVLFQDVT |
| | | FTMGQVVSREGQGRQETLFRCIRSMPSHPDRAYNSCYSAGVFHL |
| | | HQGDILSVIIPRARAKLNLSPHGTFLGFVKL |

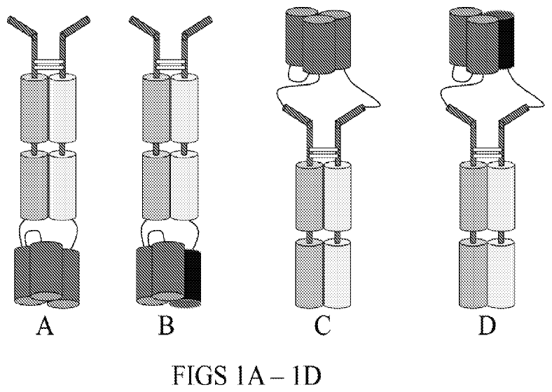

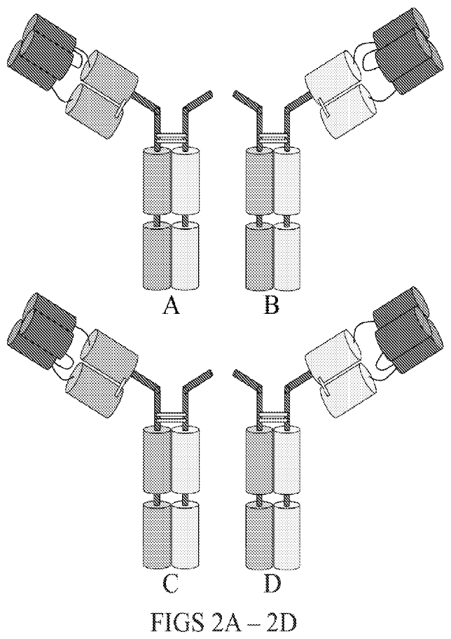

TABLE 7
Knob and hole pairs comprising complete constructs

| | Construct | | Exemplary |
|---|---|---|---|
| Molecule ID | Name | Comprising SEQ IDs | Configuration |
| Molecule ID: 109 | Fc-CD40L | SEQ ID NO: 94 | FIG. 1A |
| | | (without the signal peptide SEQ ID NO: 74), | |
| | | SEQ ID NO: 95 | |
| | | (without the signal peptide SEQ ID NO: 74) | |
| Molecule ID: 110 | CD40L-Fc | SEQ ID NO: 96 | FIG. 2A |
| | | (without the signal peptide SEQ ID NO: 74), | |
| | | SEQ ID NO: 97 | |
| | | (without the signal peptide SEQ ID NO: 74), | |
| | | SEQ ID NO: 98 | |
| | | (without the signal peptide SEQ ID NO: 74) | |
| Molecule ID: 111 | Fc-OX40L | SEQ ID NO: 99 | FIG. 1A |
| | | (without the signal peptide SEQ ID NO: 74), | |
| | | SEQ ID NO: 100 | |
| | | (without the signal peptide SEQ ID NO: 74) | |
| Molecule ID: 112 | Fc-TNFα | SEQ ID NO: 101 | FIG. 1A |
| | | (without the signal peptide SEQ ID NO: 74), | |
| | | SEQ ID NO: 102 | |
| | | (without the signal peptide SEQ ID NO: 74) | |
| Molecule ID: 113 | Fc-FASL | SEQ ID NO: 103 | FIG. 1A |
| | | (without the signal peptide SEQ ID NO: 74), | |
| | | SEQ ID NO: 104 | |
| | | (without the signal peptide SEQ ID NO: 74) | |
| Molecule ID: 114 | Fc-LIGHT | SEQ ID NO: 105 | FIG. 1A |
| | | (without the signal peptide SEQ ID NO: 74), | |
| | | SEQ ID NO: 106 | |
| | | (without the signal peptide SEQ ID NO: 74) | |
| Molecule ID: 115 | Fc-GITRL | SEQ ID NO: 107 | FIG. 1A |
| | | (without the signal peptide SEQ ID NO: 74), | |
| | | SEQ ID NO: 108 | |
| | | (without the signal peptide SEQ ID NO: 74) | |
| Molecule ID: 186 | Fc ΔCys- | SEQ ID NO: 158, SEQ ID NO: 159 | FIG. 13A |
| | GITRL | | |
| Molecule ID: 187 | Fc- GITRL | SEQ ID NO: 160, SEQ ID NO: 161 | FIG. 1A |
| Molecule ID: 188 | GITRL CH1 | SEQ ID NO: 162, SEQ ID NO: 163, | FIG. 14A |
| | ΔCys- Fc | SEQ ID NO: 164 | |
| Molecule ID: 189 | GITRL CH1- | SEQ ID NO: 165, SEQ ID NO: 166, | FIG. 2A |
| | Fc | SEQ ID NO: 164 | |
| Molecule ID: 190 | Fc- FASL | SEQ ID NO: 167, SEQ ID NO: 168 | FIG. 1A |
| Molecule ID: 191 | Fc ΔCys- | SEQ ID NO: 169, SEQ ID NO: 170 | FIG. 13A |
| | FASL | | |
| Molecule ID: 192 | Fc ΔCys- | SEQ ID NO: 171 SEQ ID NO: 172 | FIG. 13A |
| | 41BBL | | |
| Molecule ID: 193 | Fc - 41BBL | SEQ ID NO: 173, SEQ ID NO: 174 | FIG. 1A |
| Molecule ID: 194 | Fc- | SEQ ID NO: 175, SEQ ID NO: 176 | FIG. IB |
| | BAFF/ARPIL | | |
| Molecule ID: 195 | Fc ΔCys- | SEQ ID NO: 177, SEQ ID NO: 178 | FIG. 13B |
| | BAFF/ARPIL | | |

### Example 3: Expression and Purification of Fc-CD40L (Molecule ID: 109)

The plasmids encoding SEQ ID NO: 94 and SEQ ID NO: 95 were co-transfected into Expi293F cells (Life Technologies A14527) using linear 25,000 Da polyethylenimine (PEI, Polysciences Inc 23966). Per liter, 500 μg of each plasmid were combined in 50 mL of OptiMem (Life Technologies 31985088) medium and sterile filtered. 3 mg of PEI were added to another 50 mL of OptiMem and sterile filtered. The DNA and PEI were combined for 10 minutes and added to Expi293 cells with a cell density of 1.8-2.8×10⁶ cells/mL and a viability of at least 95%. The cells were grown in a humidified incubator at 37° C. with 8% CO₂ for 5-7 days after transfection. The cells were pelleted by centrifugation at 4500×g and the supernatant was filtered through a 0.2 μm membrane. Protein A resin (GE 17-1279-03) was added to the filtered supernatant and incubated for 1-3 hours at room temperature. The resin was packed into a column washed with 3×10 column volumes of Dulbecco's phosphate-buffered saline (DPBS, Life Technologies 14190-144). The bound protein was eluted from the column with 20 mM citrate, 100 mM NaCl, pH 2.9. The TNFSF fusion molecule, Fc-CD40L (represented by FIG. 1A), was polished by size exclusion on a Superdex 200 column with a running buffer of DPBS. Fractions containing monomer molecule Fc-CD40L were pooled.

### Example 4: Functional Assay of Fc-CD40L

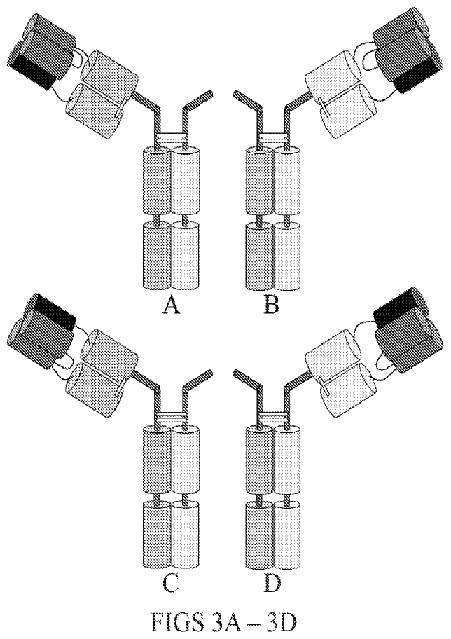

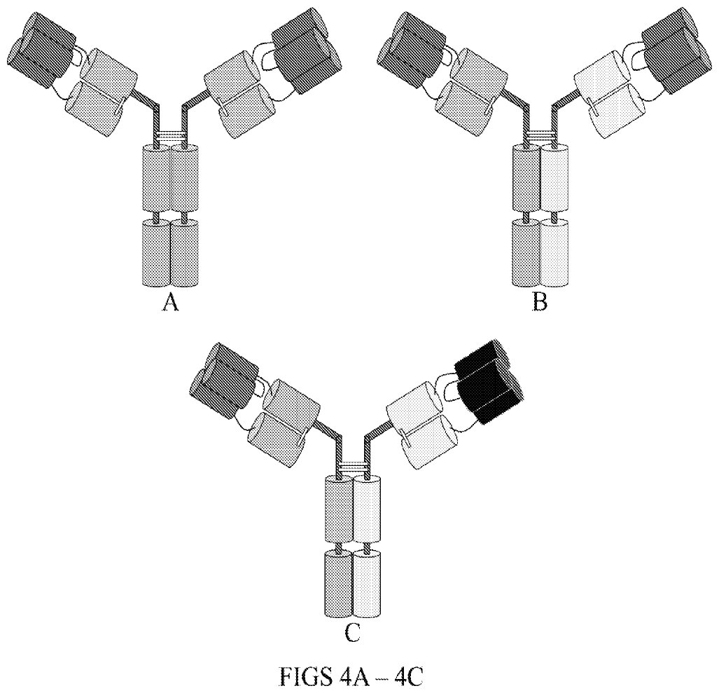

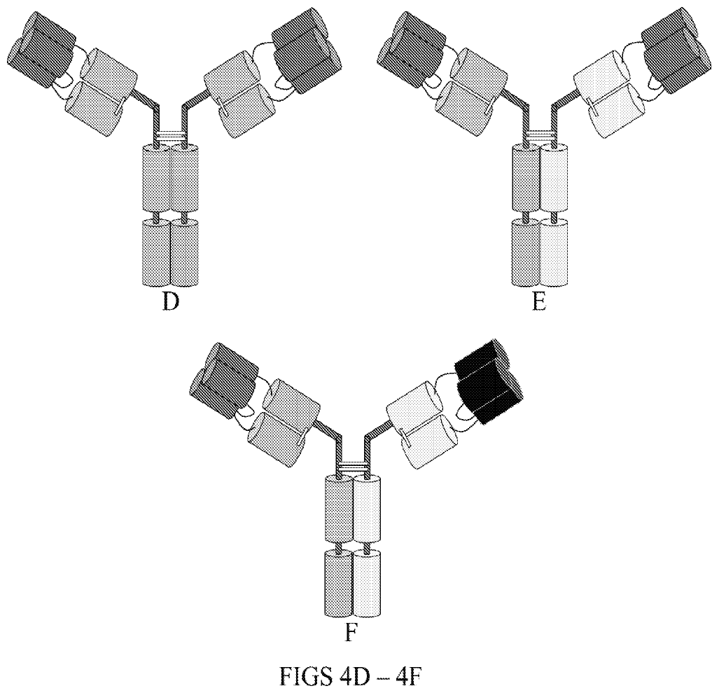

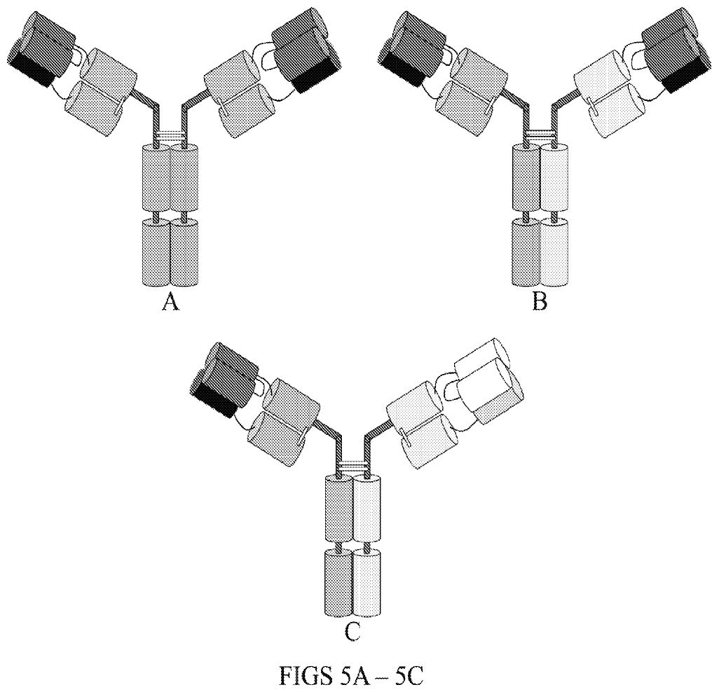

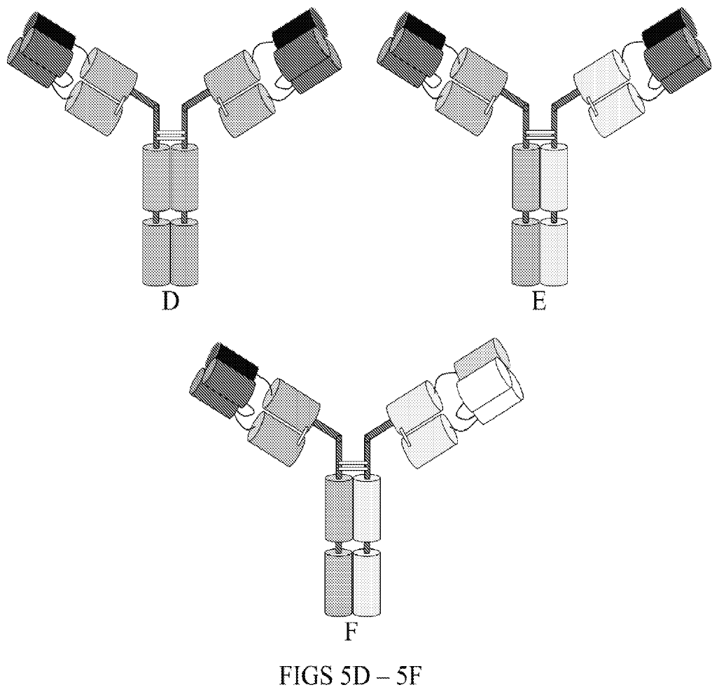

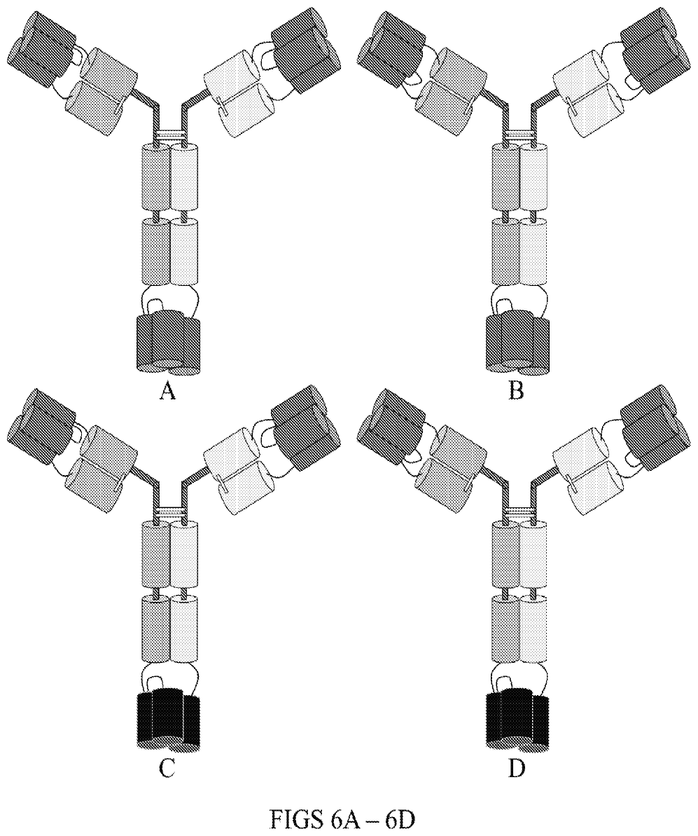

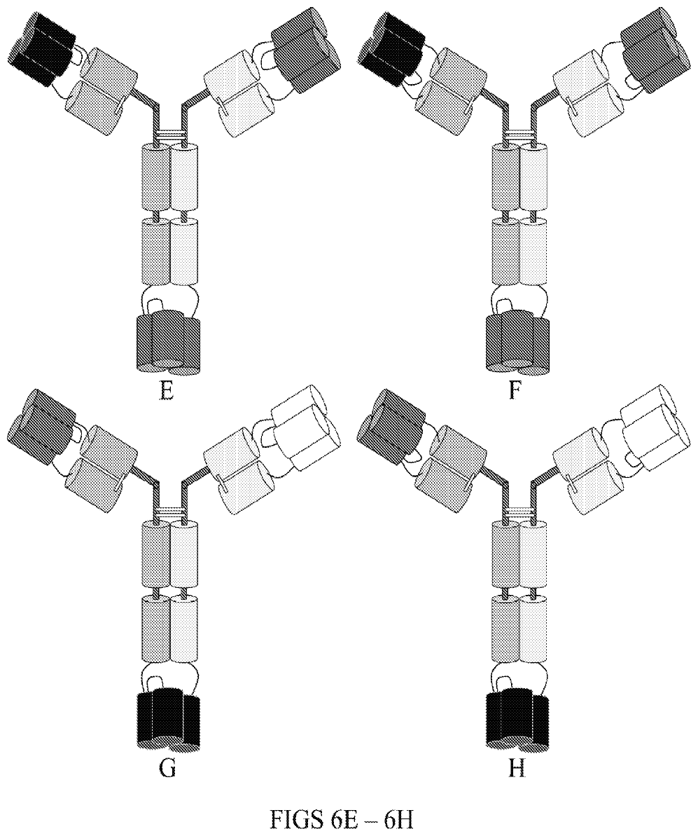

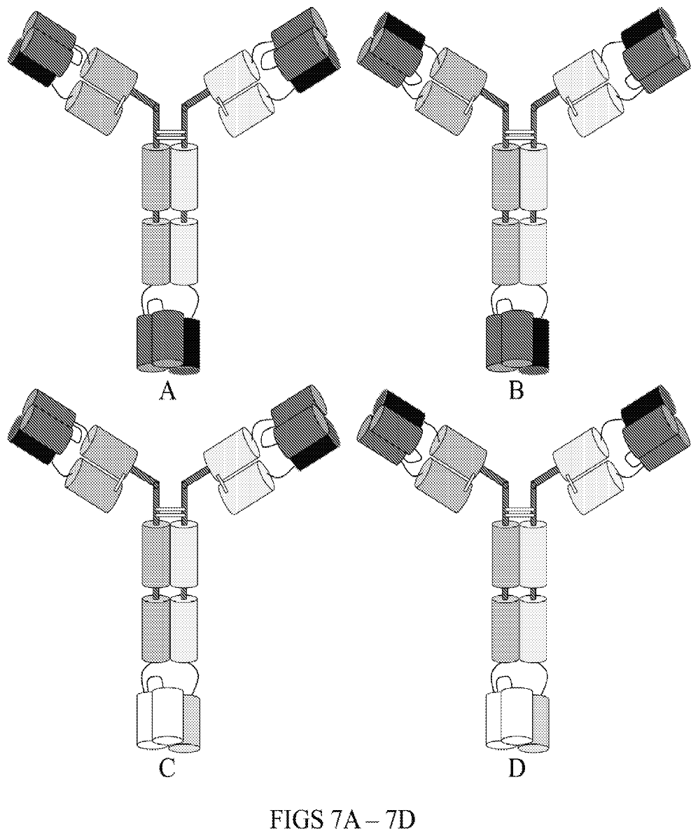

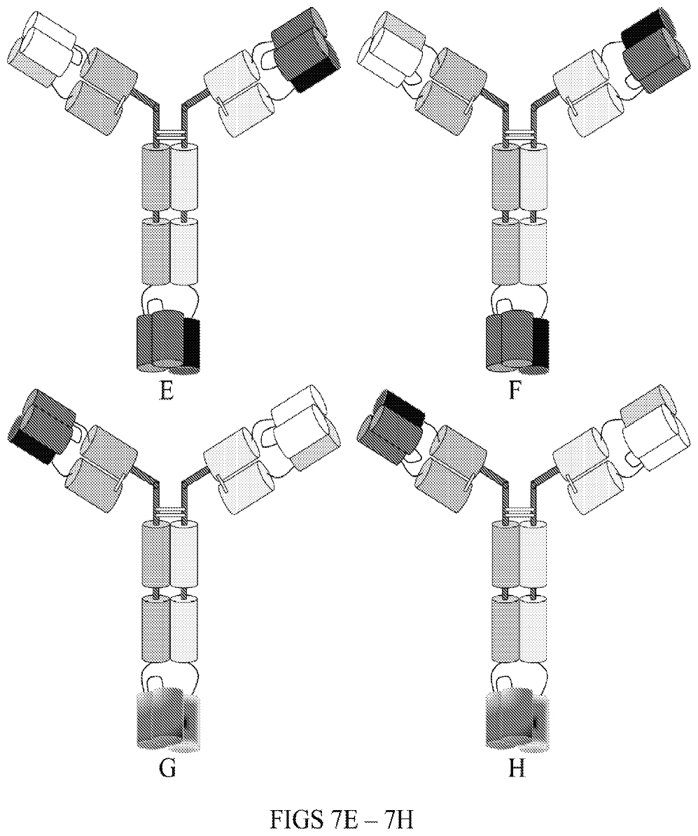

The Fc-CD40L was assayed for activity using HEK-Blue CD40L cells (Invivogen hkb-cd40) according to the manufacturer's instructions using the Chi Lob 7/4 anti-CD40 agonist monoclonal antibody (US 2009/0074711 A1 FIG. 3) as a comparator. The heavy chain for Chi Lob 7/4 is given in SEQ ID NO: 116 and the light chain is given in SEQ ID NO: 117.

SEQ ID NO: 116
EVQLQQSGPDLVKPGASVKISCKTSGYTFTEYIMHWVKQSHGKSLEWIGG
IIPNNGGTSYNQKFKDKATMTVDKSSSTGYMELRSLTSEDSAVYYCTRRE
VYGRNYYALDYWGQGTLVTVSS
SEQ ID NO: 117
DIQMTQTTSSLSASLGDRVTITCSASQGINNYLNWYQQKPDGTVKLLIYY
TSSLHSGVPSRFSGSGSGTDYSLTISNLEPEDIATYYCQQYSNLPYTFGG
GTKLEIK

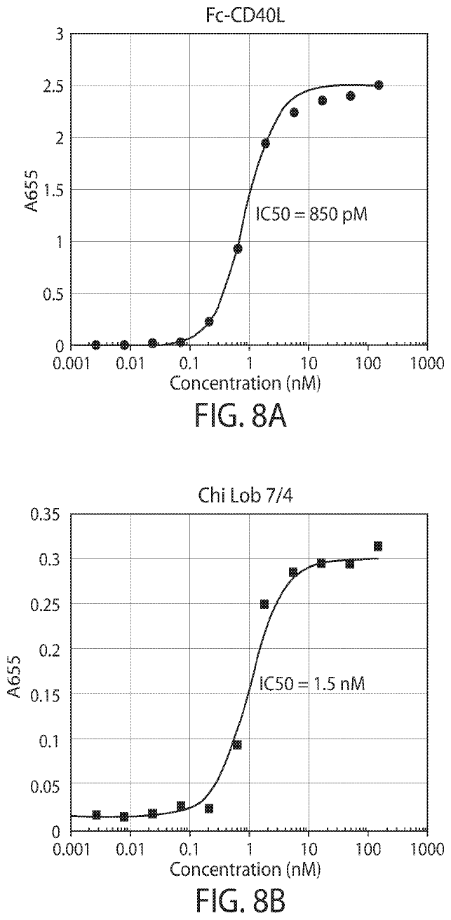

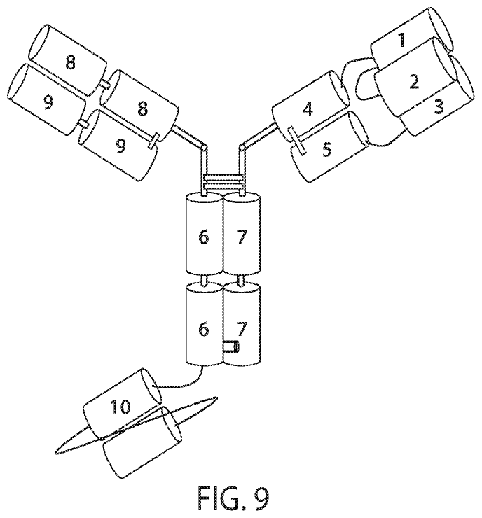

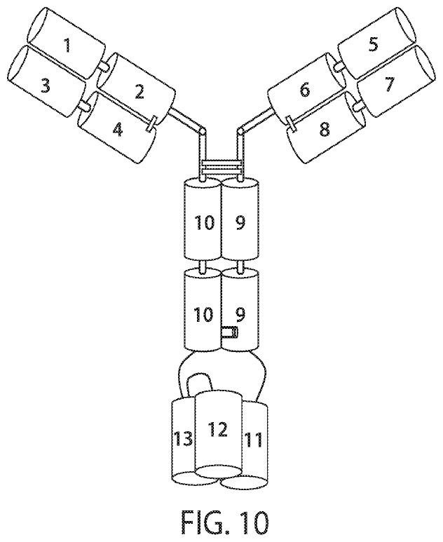

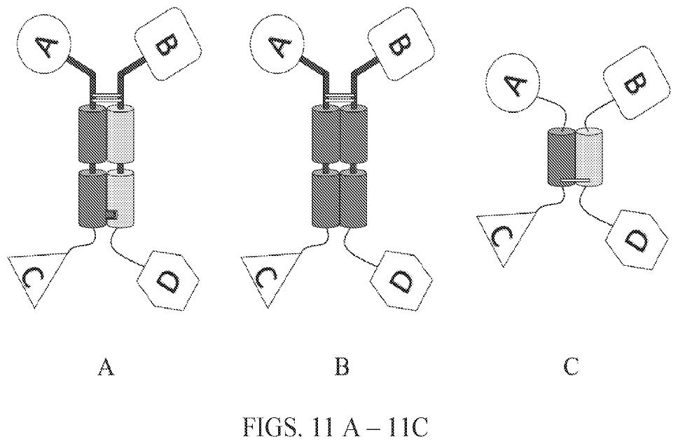

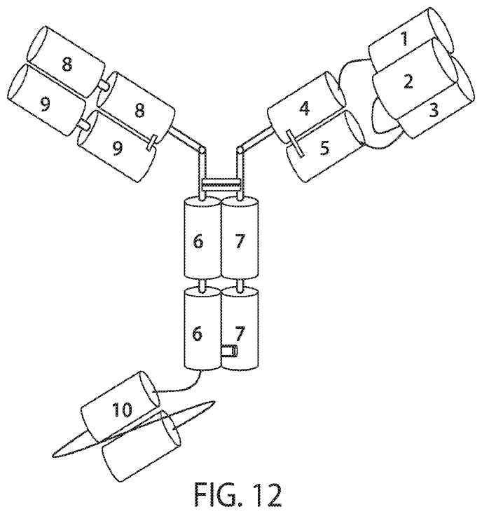

Each protein was incubated with 50,000 cells in a concentration range of 2.5 pM to 150 nM and the resulting signal was assessed at 655 nM with the results shown in FIGS. 8A and 8B. The IC50 for the Fc-CD40L and the Chi Lob 7/4 antibody were each about 1 nM.

### Example 5: Expression and Purification of Fc (ΔCys)-GITRL (Molecule ID: 186)

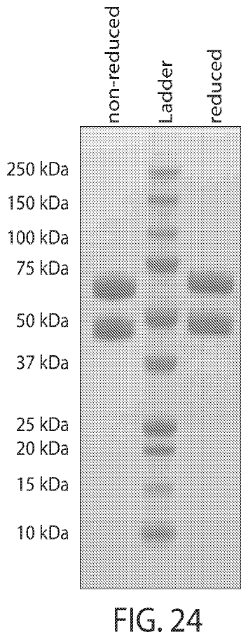

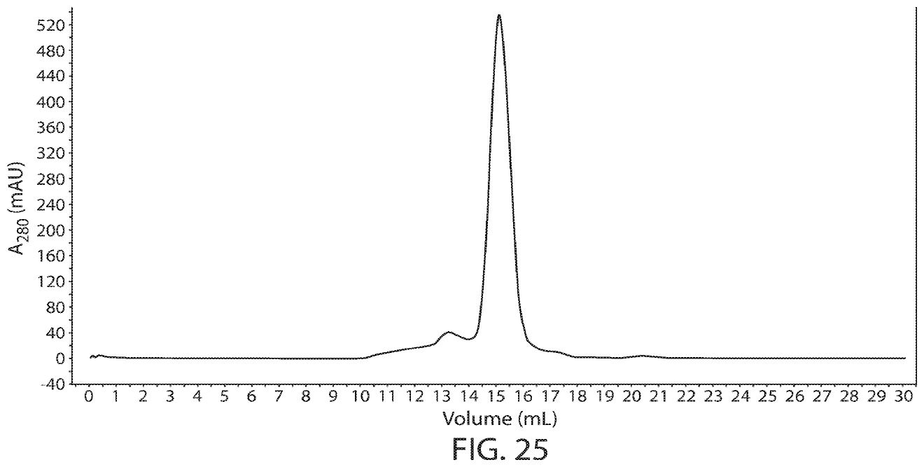

The plasmids encoding SEQ ID NO: 158 and SEQ ID NO: 159 were co-transfected into ExpiCHO cells according to manufacturer's instructions. The TNFSF fusion molecule, Fc (ΔCys)-GITRL (represented by FIG. 13A) was polished by size exclusion on a Superdex 200 column with a running buffer of 20 mM citrate, 100 mM sodium chloride, pH 5.5. Fractions containing monomer molecule Fc (ΔCys)-GITRL were pooled. The gel of Molecule ID: 186 is shown in FIG. 24. The non-reduced protein sample showed two distinct bands, equivalent to the bands in the reduced sample. In solution, Fc (ΔCys)-GITRL forms a stable heterodimer, as seen by the analytical size exclusion chromatogram shown in FIG. 25, without the inter-chain disulfide bonds.

### Example 6: Expression and Purification of Fc-GITRL (Molecule ID: 187)

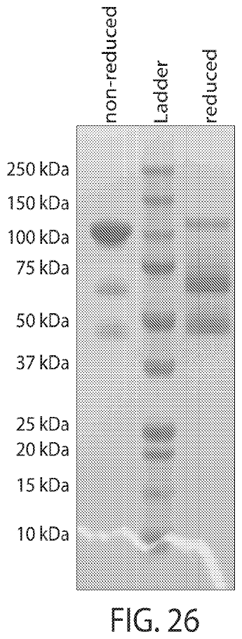

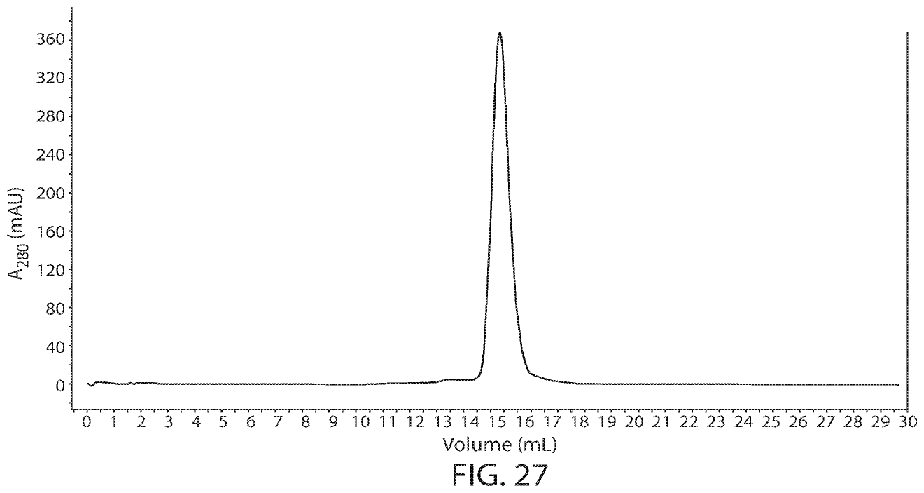

The plasmids encoding SEQ ID NO: 160 and SEQ ID NO: 161 were co-transfected into ExpiCHO cells according to manufacturer's instructions. The TNFSF fusion molecule, Fc-GITRL (represented by FIG. 1A) was polished by size exclusion on a Superdex 200 column with a running buffer of 20 mM citrate, 100 mM sodium chloride, pH 5.5. Fractions containing monomer molecule Fc-GITRL were pooled. The gel of Molecule ID: 187 is shown in FIG. 26. The gel showed the two bands that were seen for Fc (ΔCys)-GITRL in the reduced sample. The analytical size exclusion chromatogram is shown in FIG. 27, which has the same retention time as the Fc (ΔCys)-GITRL construct.

### Example 7: Expression and Purification of GITRL-Fc (ΔCys) (Molecle ID: 188)

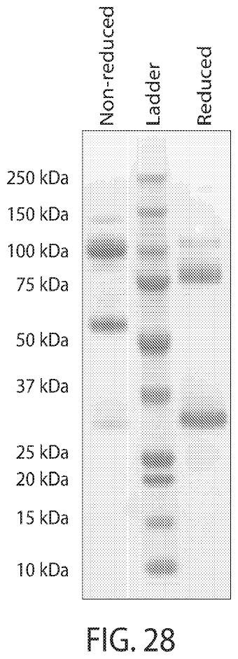

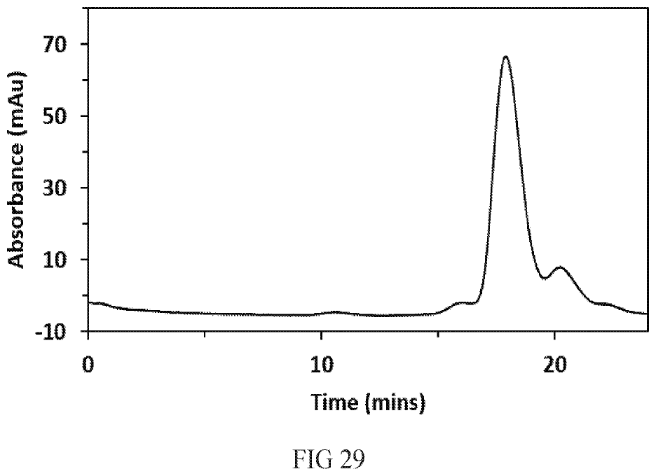

The plasmids encoding SEQ ID NO: 162, SEQ ID NO: 163, and SEQ ID NO: 164 were co-transfected into ExpiCHO cells according to manufacturer's instructions. The TNFSF fusion molecule, GITRL-Fc (ΔCys) (represented by FIG. 14A) was polished by size exclusion on a Superdex 200 column with a running buffer of 20 mM citrate, 100 mM sodium chloride, pH 5.5. Fractions containing monomer molecule GITRL-Fc (ΔCys) were pooled. The gel of Molecule ID: 188 is shown in FIG. 28. In solution, Fc (ΔCys)-GITRL forms a stable heterodimer, as seen by the analytical size exclusion chromatogram shown in FIG. 29.

### Example 8: Expression and Purification of GITRL-Fc (Molecule ID: 189)

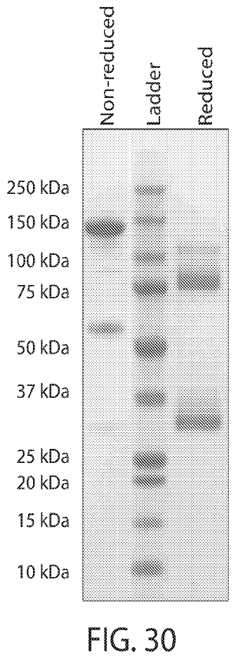

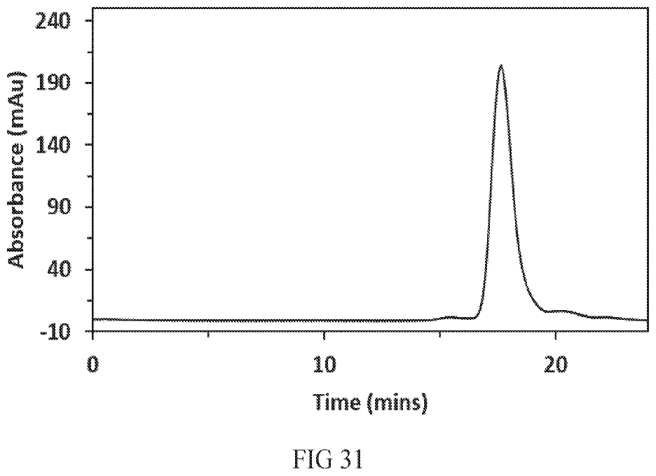

The plasmids encoding SEQ ID NO: 165, SEQ ID NO: 166, and SEQ ID NO: 164 were co-transfected into ExpiCHO cells according to manufacturer's instructions. The TNFSF fusion molecule, GITRL-Fc (represented by FIG. 2A) was polished by size exclusion on a Superdex 200 column with a running buffer of 20 mM citrate, 100 mM sodium chloride, pH 5.5. Fractions containing monomer molecule GITRL-Fc were pooled. The gel of Molecule ID: 189 is shown in FIG. 30. The analytical size exclusion chromatogram is shown in FIG. 31, which has the same retention time as the GITRL-Fc (ΔCys) construct.

### Example 9: Expression and Purification of Fc-FasL (Molecule ID: 190)

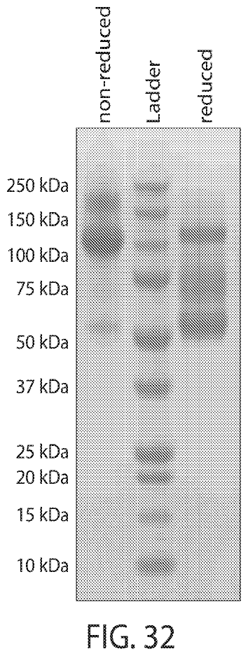

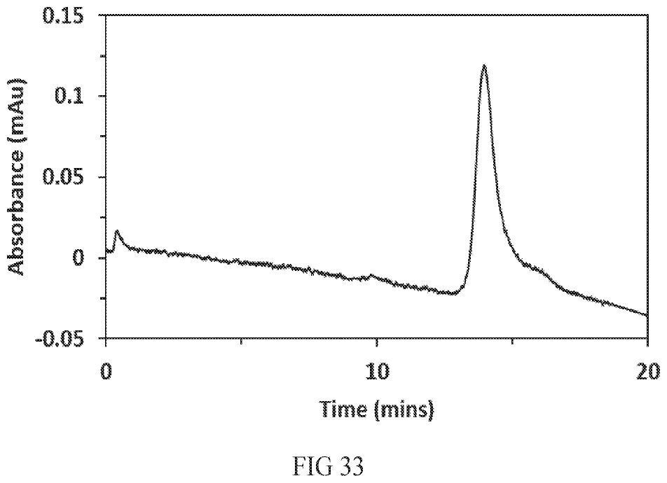

The plasmids encoding SEQ ID NO: 167 and SEQ ID NO: 168 were co-transfected into Expi293F cells using linear 25,000 Da polyethylenimine (PEI, Polysciences Inc 23966). After four days of transfection, the cells were at 41% viability and were harvested. The viability was unusually low as was the duration of the transfection, possibly due to the cytotoxic activity of the FASL molecule. The TNFSF fusion molecule, Fc-FasL (represented by FIG. 1A) was polished by size exclusion on a Superdex 200 column with a running buffer of 20 mM citrate, 100 mM sodium chloride, pH 5.5. Fractions containing monomer molecule Fc-FASL were pooled. The gel of Molecule ID: 190 is shown in FIG. 32. The analytical size exclusion chromatogram is shown in FIG. 33.

### Example 10: Expression and Purification of Fc (ΔCys)-FasL (Molecule ID: 191)

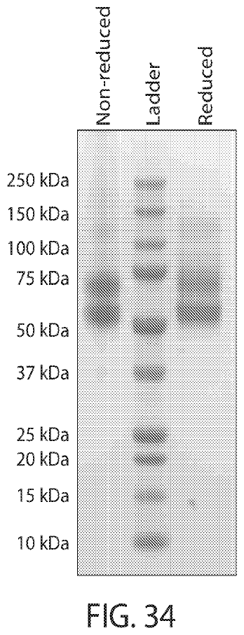

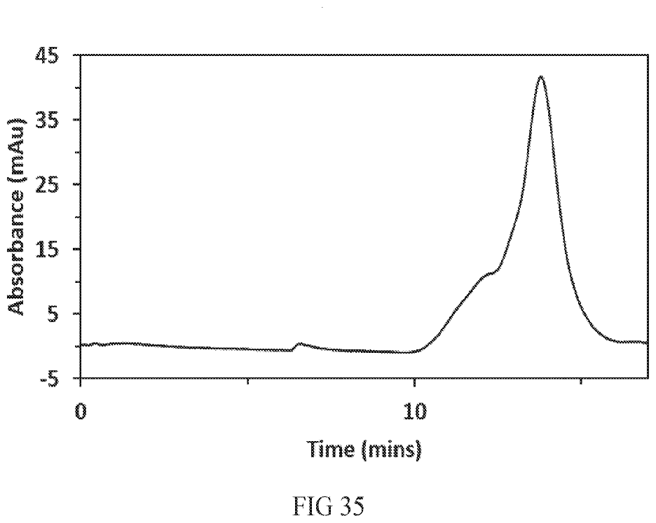

The plasmids encoding SEQ ID NO: 169 and SEQ ID NO: 170 were co-transfected into Expi293F cells using linear 25,000 Da polyethylenimine (PEI, Polysciences Inc 23966). After four days of transfection, the cells were at 26% viability and were harvested. The viability was unusually low as was the duration of the transfection, possibly due to the cytotoxic activity of the FASL molecule. The TNFSF fusion molecule, Fc (ΔCys)-FASL (represented by FIG. 13A) was polished by size exclusion on a Superdex 200 column with a running buffer of 20 mM citrate, 100 mM sodium chloride, pH 5.5. Fractions containing monomer molecule Fc (ΔCys)-FASL were pooled. The gel of Molecule ID: 191 is shown in FIG. 34. The non-reduced protein sample showed two distinct bands, equivalent to the bands in the reduced sample. In solution, Fc (ΔCys)-FASL forms a stable heterodimer, as seen by the analytical size exclusion chromatogram shown in FIG. 35. Fc (ΔCys)-FASL has the same retention in the size exclusion column as the construct containing the inter-chain disulfides (Fc-FASL).

### Example 11: Expression and Purification of Fc (ΔCys)-41BBL (Molecule ID: 192)

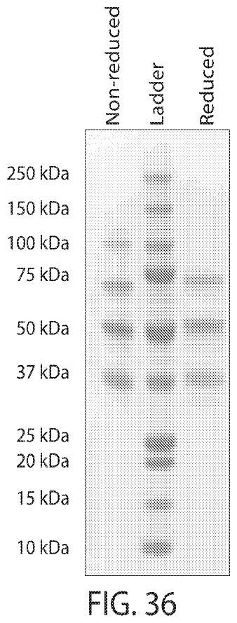

The plasmids encoding SEQ ID NO: 171 and SEQ ID NO: 172 were co-transfected into ExpiCHO cells according to manufacturer's instructions. The TNFSF fusion molecule, Fc (ΔCys)-41BBL (represented by FIG. 13A) was polished by size exclusion on a Superdex 200 column with a running buffer of 20 mM citrate, 100 mM sodium chloride, pH 5.5. Fractions containing monomer molecule Fc (ΔCys)-41BBL were pooled. The gel of Molecule ID: 192 is shown in FIG. 36. The non-reduced protein sample has the same bands as the reduced sample, because of the absence of the inter-chain disulfide bonds.

### Example 12: Expression and Purification of Fc-41BBL (Molecule ID: 193)

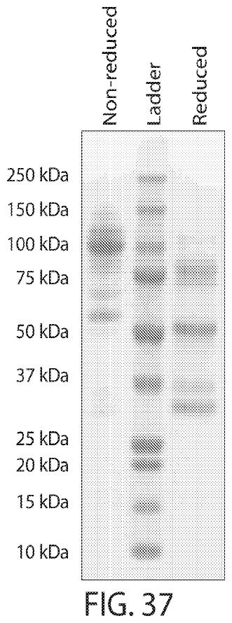

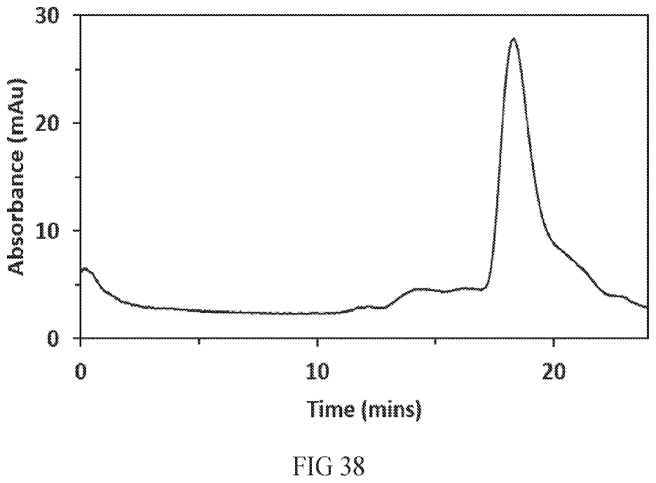

The plasmids encoding SEQ ID NO: 173 and SEQ ID NO: 174 were co-transfected into ExpiCHO cells according to manufacturer's instructions. The TNFSF fusion molecule, Fc-41BBL (represented by FIG. 1A) was polished by size exclusion on a Superdex 200 column with a running buffer of 20 mM citrate, 100 mM sodium chloride, pH 5.5. Fractions containing monomer molecule Fc-41BBL were pooled. The gel of Molecule ID: 193 is shown in FIG. 37. The gel showed the two bands that were seen for Fc (ΔCys)-41BBL in the reduced sample. The analytical size exclusion chromatogram is shown in FIG. 38.

### Example 13: Expression and Purification of Fc-BAFF/APRIL (Molecule ID: 194)

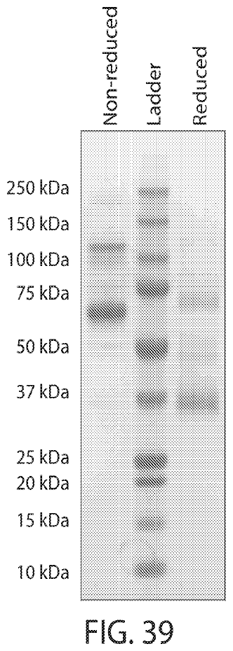

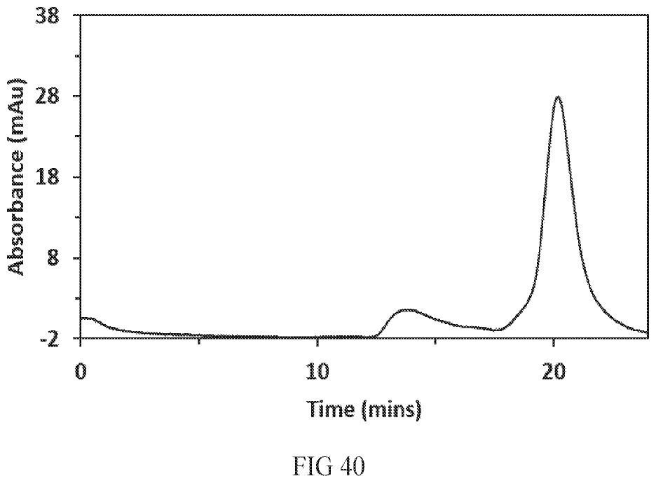

The plasmids encoding SEQ ID NO: 175 and SEQ ID NO: 176 were co-transfected into ExpiCHO cells according to manufacturer's instructions. The TNFSF fusion molecule, Fc-2×BAFF/1×APRIL (represented by FIG. 1B) was polished by size exclusion on a Superdex 200 column with a running buffer of 20 mM citrate, 100 mM sodium chloride, pH 5.5. Fractions containing monomer molecule Fc-2×BAFF/1×APRIL were pooled. The gel of Molecule ID: 194 is shown in FIG. 39. Two bands were seen in the reduced sample as expected. The analytical size exclusion chromatogram is shown in FIG. 40.

### Example 14: Expression and Purification of Fc (ΔCys)-BAFF/APRIL (Molecule ID: 195)

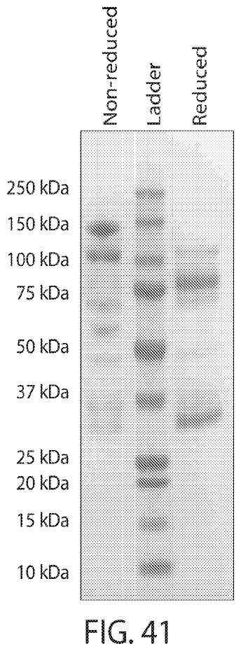

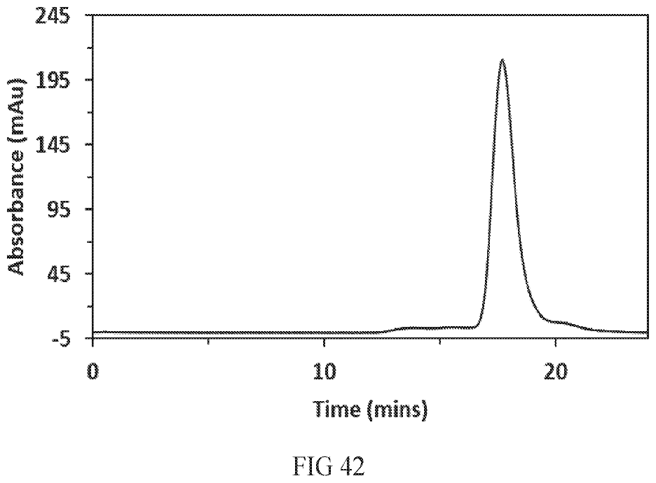

The plasmids encoding SEQ ID NO: 177 and SEQ ID NO: 178 were co-transfected into ExpiCHO cells according to manufacturer's instructions. The TNFSF fusion molecule, Fc (ΔCys)-2×BAFF/1×APRIL (represented by FIG. 13B) was polished by size exclusion on a Superdex 200 column with a running buffer of 20 mM citrate, 100 mM sodium chloride, pH 5.5. Fractions containing monomer molecule Fc (ΔCys)-2×BAFF/1×APRIL were pooled. The gel of Molecule ID: 195 is shown in FIG. 41. In solution, Fc (ΔCys)-GITRL forms a stable heterodimer, as seen by the analytical size exclusion chromatogram shown in FIG. 42, without the inter-chain disulfide bonds. This was shown by the similar retention time on the size exclusion column to that of Fc-BAFF/APRIL.

### INCORPORATION BY REFERENCE

All publications and patents mentioned herein are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A multifunctional molecule, comprising a trimeric ligand, wherein said trimeric ligand comprises a first monomer molecule, a second monomer molecule, and a third monomer molecule, wherein:
a) the first, second, or third monomer molecule is chosen from a tumor necrosis factor superfamily (TNFSF) member or TNSF-like member, or a combination thereof,
b) the first and second monomer molecules are covalently linked to one another, and the third monomer molecule is non-covalently associated to the first and second monomer molecules, and
c) the first and second monomer molecules are covalently linked to a first dimerization molecule, and the third monomer molecule is covalently linked to a second dimerization molecule, wherein the first dimerization molecule is non-covalently associated with the second dimerization molecule.

2. The multifunctional molecule of claim 1, wherein the first dimerization molecule is not linked to the second dimerization molecule by a disulfide bond.

3. The multifunctional molecule of claim 1, wherein:
a) the first dimerization molecule comprises a Fab heavy chain constant region, and the second dimerization molecule comprises a Fab light chain constant region; or
b) the first dimerization molecule comprises a Fab light chain constant region, and the second dimerization molecule comprises a Fab heavy chain constant region, wherein:
the first and second monomer molecules are covalently linked to the N-terminus of the first dimerization molecule, and the third monomer molecule is covalently linked to the N-terminus of the second dimerization molecule.

4. The multifunctional molecule of claim 3, wherein:
(i) the Fab heavy chain constant region is of IgG1 isotype and
a) lacks a cysteine residue at position 220 according to the EU-index numbering system,
b) comprises a deletion at position 220 according to the EU-index numbering system,
c) has a non-cysteine amino acid residue at position 220 according to the EU-index numbering system,
d) comprises a C220S substitution according to the EU-index numbering system, or
e) comprises the amino acid sequence of SEQ ID NO: 192;
(ii) the Fab light chain constant region is of kappa isotype and
a) lacks a cysteine residue at position 214 according to the Kabat numbering system,
b) comprises a deletion at position 214 according to the Kabat numbering system,
c) has a non-cysteine amino acid residue at position 214 according to the Kabat numbering system,
d) comprises a C214S substitution according to the Kabat numbering system, or
e) comprises the amino acid sequence of SEQ ID NO: 191; or
(iii) the Fab light chain constant region is of lambda isotype and
a) lacks a cysteine residue at position 214 according to the Kabat numbering system,
b) comprises a deletion at position 214 according to the Kabat numbering system,
c) has a non-cysteine amino acid residue at position 214 according to the Kabat numbering system, or
d) comprises a C214S substitution according to the Kabat numbering system.

5-6. (canceled)

7. The multifunctional molecule of claim 1, wherein:
a) the first dimerization molecule comprises an amino acid sequence chosen from SEQ ID NO: 181, 197, 186, 192, 187, 188, 193, or 194, and the second dimerization molecule comprises the amino acid sequence of SEQ ID NO: 183 or 191; or
b) the first dimerization molecule comprises the amino acid sequence of SEQ ID NO: 183 or 191, and the second dimerization molecule comprises an amino acid sequence chosen from SEQ ID NO: 181, 197, 186, 192, 187, 188, 193, or 194.

8. (canceled)

9. The multifunctional molecule of claim 1, wherein:
the first dimerization molecule comprises a first heavy chain constant region and the second dimerization molecule comprises a second heavy chain constant region, wherein:
the first and second monomer molecules are covalently linked to the C-terminus of the first dimerization molecule, and the third monomer molecule is covalently linked to the C-terminus of the second dimerization molecule.

10. The multifunctional molecule of claim 9, wherein:
(i) one or more cysteine residues in the first and/or second heavy chain constant region have been substituted with a non-cysteine amino acid residue;
(ii) one or more cysteine residues in the first and/or second heavy chain constant region have been substituted with a serine;
(iii) the first and second heavy chain constant regions are of IgG1 isotype, wherein the first and/or second heavy chain constant region has a non-cysteine amino acid residue at positions 226 and 229 according to the EU-index numbering system;
(iv) the first and second heavy chain constant regions are of IgG1 isotype, wherein the first heavy chain constant region comprises a C226S substitution and a C229S substitution, and/or the second heavy chain constant region comprises a C226S substitution and a C229S substitution;
(v) the first and second heavy chain constant regions comprise the amino acid sequences of SEQ ID NOs: 184 and 185, respectively;
(vi) the first and second heavy chain constant regions comprise the amino acid sequences of SEQ ID NOs: 185 and 184, respectively;
(vii) the first and second heavy chain constant regions comprise the amino acid sequences of SEQ ID NOs: 184 and 43, respectively;
(viii) the first and second heavy chain constant regions comprise the amino acid sequences of SEQ ID NOs: 43 and 184, respectively;
(ix) the first and second heavy chain constant regions comprise the amino acid sequences of SEQ ID NOs: 77 and 185, respectively; or
(x) the first and second heavy chain constant regions comprise the amino acid sequences of SEQ ID NOs: 185 and 77, respectively.

11. (canceled)

12. The multifunctional molecule of claim 1, wherein:
(i) a) the first dimerization molecule comprises a TCRα constant domain and the second dimerization molecule comprises a TCRβ constant domain, or
b) the first dimerization molecule comprises a TCRβ constant domain and the second dimerization molecule comprises a TCRα constant domain;
(ii) a) the first dimerization molecule comprises an immunoglobulin CH2 domain connected to a TCRα constant domain and the second dimerization molecule comprises an immunoglobulin CH2 domain connected to a TCRβ constant domain, or
b) the first dimerization molecule comprises an immunoglobulin CH2 domain connected to a TCRβ constant domain and the second dimerization molecule comprises an immunoglobulin CH2 domain connected to a TCRα constant domain.

13. (canceled)

14. The multifunctional molecule of claim 12, wherein:
(i) a) the TCRα constant domain comprises or consists of the amino acid sequence of SEQ ID NO: 118 (or a sequence having 1 or more (e.g., 1, 2, 3, 4, 5, 5, 6, 7, 8, 9, 10, or more) amino acid substitutions relative to the amino acid sequence of SEQ ID NO: 118, or a sequence having no more than 10 (e.g., 10, 9, 8, 7, 6, 5, 4, 3, 2, or 1) amino acid substitutions relative to the amino acid sequence of SEQ ID NO: 118) and/or
b) the TCRβ constant domain comprises or consists of the amino acid sequence of SEQ ID NO: 120 (or a sequence having 1 or more (e.g., 1, 2, 3, 4, 5, 5, 6, 7, 8, 9, 10, or more) amino acid substitutions relative to the amino acid sequence of SEQ ID NO: 120, or a sequence having no more than 10 (e.g., 10, 9, 8, 7, 6, 5, 4, 3, 2, or 1) amino acid substitutions relative to the amino acid sequence of SEQ ID NO: 120);
(ii) a) the TCRα constant domain comprises or consists of the amino acid sequence of SEQ ID NO: 119 (or a sequence having 1 or more (e.g., 1, 2, 3, 4, 5, 5, 6, 7, 8, 9, 10, or more) amino acid substitutions relative to the amino acid sequence of SEQ ID NO: 119, or a sequence having no more than 10 (e.g., 10, 9, 8, 7, 6, 5, 4, 3, 2, or 1) amino acid substitutions relative to the amino acid sequence of SEQ ID NO: 119) and/or
b) the TCRβ constant domain comprises or consists of the amino acid sequence of SEQ ID NO: 121 (or a sequence having 1 or more (e.g., 1, 2, 3, 4, 5, 5, 6, 7, 8, 9, 10, or more) amino acid substitutions relative to the amino acid sequence of SEQ ID NO: 121, or a sequence having no more than 10 (e.g., 10, 9, 8, 7, 6, 5, 4, 3, 2, or 1) amino acid substitutions relative to the amino acid sequence of SEQ ID NO: 121); or
(iii) a) the first dimerization molecule comprises a TCRα variable domain connected to a TCRα constant domain, and the second dimerization molecule comprises a TCRβ variable domain connected to a TCRβ constant domain, or
b) the first dimerization molecule comprises a TCRβ variable domain connected to a TCRβ constant domain, and the second dimerization molecule comprises a TCRα variable domain connected to a TCRα constant domain.

15-16. (canceled)

17. The multifunctional molecule of claim 1, wherein:
(i) a) neither the first nor the second dimerization molecule comprises an immunoglobulin CH3 domain; or
b) neither the first nor the second dimerization molecule comprises any portion of an immunoglobulin CH3 domain capable of stable self-association; or
(ii) a) neither the first nor the second dimerization molecule comprises more than 50, 25, 10, or 5 amino acids of an immunoglobulin CH2 domain and/or more than 50, 25, 10, or 5 amino acids of an immunoglobulin CH3 domain, or
b) neither the first nor the second dimerization molecule comprises an immunoglobulin CH2 and/or CH3 domain (e.g., any portion of a CH2 and/or CH3 domain).

18. (canceled)

19. The multifunctional molecule of claim 1, wherein the trimeric ligand is a heterotrimer.

20. The multifunctional molecule of claim 19, wherein:
(i) a) the first monomer molecule, the second monomer molecule, and the third monomer molecule are not identical,
b) the first and second monomer molecules are the same (or sharing no less than 70%, 75%, 80%, 85%, or 90% sequence identity), and the third monomer molecule is different from the first and second monomer molecules,
c) the first and third monomer molecules are the same (or sharing no less than 70%, 75%, 80%, 85%, or 90% sequence identity), and the second monomer molecule is different from the first and third monomer molecules, or
d) the second and third monomer molecules are the same (or sharing no less than 70%, 75%, 80%, 85%, or 90% sequence identity), and the first monomer molecule is different from the second and third monomer molecules;
(ii) a) the first, second, and third monomer molecules are independently chosen from BAFF or APRIL, wherein at least one of the three monomer molecules is BAFF and at least one of the three monomer molecules is APRIL,
b) the first, second, and third monomer molecules independently comprise the amino acid sequence of SEQ ID NO: 14 (or a sequence having at least 70%, 75%, 80%, 85%, 90%, or 95% sequence identity thereto) or the amino acid sequence of SEQ ID NO: 13 or 180 (or a sequence having at least 70%, 75%, 80%, 85%, 90%, or 95% sequence identity thereto),
c) the first monomer molecule is BAFF, the second monomer molecule is BAFF, and the third monomer molecule is APRIL, or
d) the first monomer molecule is APRIL, the second monomer molecule is APRIL, and the third monomer molecule is BAFF; or
(iii) a) the first, second, and third monomer molecules are independently chosen from lymphotoxin-alpha or lymphotoxin-beta, wherein at least one of the three monomer molecules is lymphotoxin-alpha, and at least one of the three monomer molecules is lymphotoxin-beta,
b) the first, second, and third monomer molecules independently comprise the amino acid sequence of SEQ ID NO: 1 (or a sequence having at least 70%, 75%, 80%, 85%, 90%, or 95% sequence identity thereto) or SEQ ID NO: 3 (or a sequence having at least 70%, 75%, 80%, 85%, 90%, or 95% sequence identity thereto),
c) the first monomer molecule is lymphotoxin-alpha (or a functional variant thereof), the second monomer molecule is lymphotoxin-alpha (or a functional variant thereof), and the third monomer molecule is lymphotoxin-beta (or a functional variant thereof), or
d) the first monomer molecule is lymphotoxin-beta (or a functional variant thereof), the second monomer molecule is lymphotoxin-beta (or a functional variant thereof), and the third monomer molecule is lymphotoxin-alpha (or a functional variant thereof).

21-22. (canceled)

23. A multifunctional molecule, comprising a trimeric ligand, wherein said trimeric ligand comprises a first monomer molecule, a second monomer molecule, and a third monomer molecule, wherein:
a) the first, second, or third monomer molecule is chosen from a tumor necrosis factor superfamily (TNFSF) member or TNSF-like member, or a combination thereof,
b) the first and second monomer molecules are covalently linked to one another, and the third monomer molecule is non-covalently associated to the first and second monomer molecules, and
c) the multifunctional molecule further comprises a TCR constant domain.

24-25. (canceled)

26. The multifunctional molecule of claim 23, wherein:
(i) a) the first and second monomer molecules are covalently linked to the C-terminus of a TCRα constant domain, and the third monomer molecule is covalently linked to the C-terminus of a TCRβ constant domain, or
b) the first and second monomer molecules are covalently linked to the C-terminus of a TCRβ constant domain, and the third monomer molecule is covalently linked to the C-terminus of a TCRα constant domain;
(ii) a) the first and second monomer molecules are covalently linked to the N-terminus of a CH1 domain, and the third monomer molecule is covalently linked to the N-terminus of a CL domain, or
b) the first and second monomer molecules are covalently linked to the N-terminus of a CL domain, and the third monomer molecule is covalently linked to the N-terminus of a CH1 domain,
wherein the CH1 domain is further covalently linked to a CH2 domain, which is further covalently linked to a TCRα constant domain or a TCRβ constant domain;
(iii) the multifunctional molecule comprises an immunoglobulin CH2 domain connected to a TCRα constant domain or a TCRβ constant domain;
(iv) the multifunctional molecule comprises:
a) a TCRα constant domain comprising or consisting of the amino acid sequence of SEQ ID NO: 118 (or a sequence having 1 or more (e.g., 1, 2, 3, 4, 5, 5, 6, 7, 8, 9, 10, or more) amino acid substitutions relative to the amino acid sequence of SEQ ID NO: 118, or a sequence having no more than 10 (e.g., 10, 9, 8, 7, 6, 5, 4, 3, 2, or 1) amino acid substitutions relative to the amino acid sequence of SEQ ID NO: 118) and/or
b) a TCRβ constant domain comprising or consisting of the amino acid sequence of SEQ ID NO: 120 (or a sequence having 1 or more (e.g., 1, 2, 3, 4, 5, 5, 6, 7, 8, 9, 10, or more) amino acid substitutions relative to the amino acid sequence of SEQ ID NO: 120, or a sequence having no more than 10 (e.g., 10, 9, 8, 7, 6, 5, 4, 3, 2, or 1) amino acid substitutions relative to the amino acid sequence of SEQ ID NO: 120);
(v) the multifunctional molecule comprises:
a) a TCRα constant domain comprising or consisting of the amino acid sequence of SEQ ID NO: 119 (or a sequence having 1 or more (e.g., 1, 2, 3, 4, 5, 5, 6, 7, 8, 9, 10, or more) amino acid substitutions relative to the amino acid sequence of SEQ ID NO: 119, or a sequence having no more than 10 (e.g., 10, 9, 8, 7, 6, 5, 4, 3, 2, or 1) amino acid substitutions relative to the amino acid sequence of SEQ ID NO: 119) and/or
b) a TCRβ constant domain comprising or consisting of the amino acid sequence of SEQ ID NO: 121 (or a sequence having 1 or more (e.g., 1, 2, 3, 4, 5, 5, 6, 7, 8, 9, 10, or more) amino acid substitutions relative to the amino acid sequence of SEQ ID NO: 121, or a sequence having no more than 10 (e.g., 10, 9, 8, 7, 6, 5, 4, 3, 2, or 1) amino acid substitutions relative to the amino acid sequence of SEQ ID NO: 121);
(vi) the multifunctional molecule comprises:
a) a first dimerization molecule comprising a TCRα variable domain connected to a TCRα constant domain, and a second dimerization molecule comprising a TCRβ variable domain connected to a TCRβ constant domain, or
b) a first dimerization molecule comprising a TCRβ variable domain connected to a TCRβ constant domain, and a second dimerization molecule comprising a TCRα variable domain connected to a TCRα constant domain;
(vii) the multifunctional molecule does not comprise an immunoglobulin CH3 domain or the multifunctional molecule does not comprise any portion of an immunoglobulin CH3 domain capable of stable self-association; or
(viii) a) the multifunctional molecule does not comprise more than 50, 25, 10, or 5 amino acids of an immunoglobulin CH2 domain and/or more than 50, 25, 10, or 5 amino acids of an immunoglobulin CH3 domain, or
b) the multifunctional molecule does not comprise an immunoglobulin CH2 and/or CH3 domain.

27-35. (canceled)

36. The multifunctional molecule of claim 23, wherein the trimeric ligand is a heterotrimer.

37. The multifunctional molecule of claim 36, wherein:
(i) a) the first monomer molecule, the second monomer molecule, and the third monomer molecule are not identical,
b) the first and second monomer molecules are the same (or sharing no less than 70%, 75%, 80%, 85%, or 90% sequence identity), and the third monomer molecule is different from the first and second monomer molecules,
c) the first and third monomer molecules are the same (or sharing no less than 70%, 75%, 80%, 85%, or 90% sequence identity), and the second monomer molecule is different from the first and third monomer molecules, or
d) the second and third monomer molecules are the same (or sharing no less than 70%, 75%, 80%, 85%, or 90% sequence identity), and the first monomer molecule is different from the second and third monomer molecules;
(ii) the first, second, and third monomer molecules are independently chosen from BAFF or APRIL, wherein at least one of the three monomer molecules is BAFF, and at least one of the three monomer molecules is APRIL;
(iii) the first, second, and third monomer molecules independently comprise the amino acid sequence of SEQ ID NO: 14 (or a sequence having at least 70%, 75%, 80%, 85%, 90%, or 95% sequence identity thereof) or the amino acid sequence of SEQ ID NO: 13 or 180 (or a sequence having at least 70%, 75%, 80%, 85%, 90%, or 95% sequence identity);
(iv) the first monomer molecule is BAFF, the second monomer molecule is BAFF, and the third monomer molecule is APRIL;
(v) the first monomer molecule is APRIL, the second monomer molecule is APRIL, and the third monomer molecule is BAFF;
(vi) the first, second, and third monomer molecules are independently chosen from lymphotoxin-alpha (or a functional variant thereof) or lymphotoxin-beta (or a functional variant thereof), wherein at least one of the three monomer molecules is lymphotoxin-alpha (or a functional variant thereof), and at least one of the three monomer molecules is lymphotoxin-beta (or a functional variant thereof);
(vii) the first, second, and third monomer molecules independently comprise the amino acid sequence of SEQ ID NO: 1 (or a sequence having at least 70%, 75%, 80%, 85%, 90%, or 95% sequence identity thereof) or SEQ ID NO: 3 (or a sequence having at least 70%, 75%, 80%, 85%, 90%, or 95% sequence identity);
(viii) the first monomer molecule is lymphotoxin-alpha (or a functional variant thereof), the second monomer molecule is lymphotoxin-alpha (or a functional variant thereof), and the third monomer molecule is lymphotoxin-beta (or a functional variant thereof); or
(ix) the first monomer molecule is lymphotoxin-beta (or a functional variant thereof), the second monomer molecule is lymphotoxin-beta (or a functional variant thereof), and the third monomer molecule is lymphotoxin-alpha (or a functional variant thereof).

38-39. (canceled)

40. A multifunctional molecule, comprising a trimeric ligand, wherein said trimeric ligand comprises a first monomer molecule, a second monomer molecule, and a third monomer molecule, wherein:
a) the first, second, or third monomer molecule is chosen from a tumor necrosis factor superfamily (TNFSF) member or TNSF-like member, or a combination thereof,
b) the first and second monomer molecules are covalently linked to one another, and the third monomer molecule is non-covalently associated to the first and second monomer molecules, and
c) the trimeric ligand is a heterotrimer comprising a combination of monomer molecules from two or three TNFSF or TNF-like family members.

41. The multifunctional molecule of claim 40, wherein:
(i) a) the first monomer molecule, the second monomer molecule, and the third monomer molecule are not identical,
b) the first and second monomer molecules are the same (or sharing no less than 70%, 75%, 80%, 85%, or 90% sequence identity), and the third monomer molecule is different from the first and second monomer molecules,
c) the first and third monomer molecules are the same (or sharing no less than 70%, 75%, 80%, 85%, or 90% sequence identity), and the second monomer molecule is different from the first and third monomer molecules, or
d) the second and third monomer molecules are the same (or sharing no less than 70%, 75%, 80%, 85%, or 90% sequence identity), and the first monomer molecule is different from the second and third monomer molecules;
(ii) the first, second, and third monomer molecules are independently chosen from BAFF or APRIL, wherein at least one of the three monomer molecules is BAFF, and at least one of the three monomer molecules is APRIL;
(iii) the first, second, and third monomer molecules independently comprise the amino acid sequence of SEQ ID NO: 14 (or a sequence having at least 70%, 75%, 80%, 85%, 90%, or 95% sequence identity thereof) or the amino acid sequence of SEQ ID NO: 13 or 180 (or a sequence having at least 70%, 75%, 80%, 85%, 90%, or 95% sequence identity);
(iv) the first monomer molecule is BAFF, the second monomer molecule is BAFF, and the third monomer molecule is APRIL;
(v) the first monomer molecule is APRIL, the second monomer molecule is APRIL, and the third monomer molecule is BAFF;
(vi) the first, second, and third monomer molecules are independently chosen from lymphotoxin-alpha (or a functional variant thereof) or lymphotoxin-beta (or a functional variant thereof), wherein at least one of the three monomer molecules is lymphotoxin-alpha (or a functional variant thereof), and at least one of the three monomer molecules is lymphotoxin-beta (or a functional variant thereof);
(vii) the first, second, and third monomer molecules independently comprise the amino acid sequence of SEQ ID NO: 1 (or a sequence having at least 70%, 75%, 80%, 85%, 90%, or 95% sequence identity thereof) or SEQ ID NO: 3 (or a sequence having at least 70%, 75%, 80%, 85%, 90%, or 95% sequence identity);
(viii) the first monomer molecule is lymphotoxin-alpha (or a functional variant thereof), the second monomer molecule is lymphotoxin-alpha (or a functional variant thereof), and the third monomer molecule is lymphotoxin-beta (or a functional variant thereof); or
(ix) the first monomer molecule is lymphotoxin-beta (or a functional variant thereof), the second monomer molecule is lymphotoxin-beta (or a functional variant thereof), and the third monomer molecule is lymphotoxin-alpha (or a functional variant thereof).

42-43. (canceled)

44. The multifunctional molecule of claim 1, wherein:
(i) the first, second, or third monomer molecule is chosen from a TNFSF family member or a TNF-like family member, or a combination thereof, described in Tables 1 and 2;
(ii) the first, second, or third monomer molecule is chosen from BAFF, APRIL, lymphotoxin-alpha, lymphotoxin-beta, CD40L, or GITRL;
(iii) the trimeric ligand is a homotrimer of the same TNFSF family member or the same TNF-like family member;
(iv) the trimeric ligand is a heterotrimer;
(v) the trimeric ligand is a heterotrimer comprising two or three TNFSF or TNF-like family members;
(vi) the trimeric ligand comprises the amino acid sequence of a monomer molecule chosen from a TNFSF family member or a TNF-like family member in Table 1 or 2, or an amino acid sequence at least 85%, 90%, 95%, 99% or more identical to an amino acid sequence in Table 1 or 2;
(vii) the trimeric ligand comprises any of the TNFSF amino acid sequences for TNSF1, TNSF2, TNSF3, TNSF4, TNSF5, TNSF6, TNSF7, TNSF8, TNSF9, TNSF10, TNSF1, TNSF11, TNSF12, TNSF13, TNSF13B, TNSF14, TNSF15, TNF18 or EDA;
(vii) the trimeric ligand comprises any of the TNF-like amino acid sequences for Complement C1Q, C1QL1, C1QL2, C1QL3, Caprin-2 C1q domain, cerebellin-1 C1q domain or adiponectin;
(viii) the trimeric ligand comprises a combination of TNFSF monomer molecules and TNF-like monomer molecules, wherein the trimer ligand is a heterotrimer comprising any combination of two or three monomer molecules comprising the amino acid sequence of any of SEQ ID NOs: 1-17, 218, and 239-247;
(ix) the trimeric ligand comprises a TNFSF family member chosen from CD40L, GITRL, FasL, 4-1BBL, BAFF, APRIL, OX40L, TNF alpha, LIGHT, lymphotoxin-alpha, or lymphotoxin-beta;
(x) the multifunctional molecule comprises one trimeric ligand, two trimeric ligands, or three trimeric ligands;
(xi) the multifunctional molecule further comprises one or more other binding specificities or functionalities chosen from one, two or more of: a targeting moiety, an NK cell engager, a B cell engager, a dendritic cell engager, a macrophage cell engager, and/or a cytokine molecule; or
(xii) the multifunctional molecule comprises:
a) the amino acid sequence of SEQ ID NO: 94 (without the signal peptide SEQ ID NO: 74) (or a sequence having at least 70%, 75%, 80%, 85%, 90%, or 95% sequence identity thereto), and the amino acid sequence of SEQ ID NO: 95 (without the signal peptide SEQ ID NO: 74) (or a sequence having at least 70%, 75%, 80%, 85%, 90%, or 95% sequence identity thereto);
b) the amino acid sequence of SEQ ID NO: 96 (without the signal peptide SEQ ID NO: 74) (or a sequence having at least 70%, 75%, 80%, 85%, 90%, or 95% sequence identity thereto), the amino acid sequence of SEQ ID NO: 97 (without the signal peptide SEQ ID NO: 74) (or a sequence having at least 70%, 75%, 80%, 85%, 90%, or 95% sequence identity thereto), and the amino acid sequence of SEQ ID NO: 98 (without the signal peptide SEQ ID NO: 74) (or a sequence having at least 70%, 75%, 80%, 85%, 90%, or 95% sequence identity thereto);
c) the amino acid sequence of SEQ ID NO: 99 (without the signal peptide SEQ ID NO: 74) (or a sequence having at least 70%, 75%, 80%, 85%, 90%, or 95% sequence identity thereto), and the amino acid sequence of SEQ ID NO: 100 (without the signal peptide SEQ ID NO: 74) (or a sequence having at least 70%, 75%, 80%, 85%, 90%, or 95% sequence identity thereto);
d) the amino acid sequence of SEQ ID NO: 101 (without the signal peptide SEQ ID NO: 74) (or a sequence having at least 70%, 75%, 80%, 85%, 90%, or 95% sequence identity thereto), and the amino acid sequence of SEQ ID NO: 102 (without the signal peptide SEQ ID NO: 74) (or a sequence having at least 70%, 75%, 80%, 85%, 90%, or 95% sequence identity thereto);
e) the amino acid sequence of SEQ ID NO: 103 (without the signal peptide SEQ ID NO: 74) (or a sequence having at least 70%, 75%, 80%, 85%, 90%, or 95% sequence identity thereto), and the amino acid sequence of SEQ ID NO: 104 (without the signal peptide SEQ ID NO: 74) (or a sequence having at least 70%, 75%, 80%, 85%, 90%, or 95% sequence identity thereto);
f) the amino acid sequence of SEQ ID NO: 105 (without the signal peptide SEQ ID NO: 74) (or a sequence having at least 70%, 75%, 80%, 85%, 90%, or 95% sequence identity thereto), and the amino acid sequence of SEQ ID NO: 106 (without the signal peptide SEQ ID NO: 74) (or a sequence having at least 70%, 75%, 80%, 85%, 90%, or 95% sequence identity thereto);
g) the amino acid sequence of SEQ ID NO: 107 (without the signal peptide SEQ ID NO: 74) (or a sequence having at least 70%, 75%, 80%, 85%, 90%, or 95% sequence identity thereto), and the amino acid sequence of SEQ ID NO: 108 (without the signal peptide SEQ ID NO: 74) (or a sequence having at least 70%, 75%, 80%, 85%, 90%, or 95% sequence identity thereto);
h) the amino acid sequence of SEQ ID NO: 158 (without the signal peptide SEQ ID NO: 74) (or a sequence having at least 70%, 75%, 80%, 85%, 90%, or 95% sequence identity thereto), and the amino acid sequence of SEQ ID NO: 159 (without the signal peptide SEQ ID NO: 74) (or a sequence having at least 70%, 75%, 80%, 85%, 90%, or 95% sequence identity thereto);
i) the amino acid sequence of SEQ ID NO: 160 (without the signal peptide SEQ ID NO: 74) (or a sequence having at least 70%, 75%, 80%, 85%, 90%, or 95% sequence identity thereto), and the amino acid sequence of SEQ ID NO: 161 (without the signal peptide SEQ ID NO: 74) (or a sequence having at least 70%, 75%, 80%, 85%, 90%, or 95% sequence identity thereto);
j) the amino acid sequence of SEQ ID NO: 162 (without the signal peptide SEQ ID NO: 74) (or a sequence having at least 70%, 75%, 80%, 85%, 90%, or 95% sequence identity thereto), the amino acid sequence of SEQ ID NO: 163 (without the signal peptide SEQ ID NO: 74) (or a sequence having at least 70%, 75%, 80%, 85%, 90%, or 95% sequence identity thereto), and the amino acid sequence of SEQ ID NO: 164 (without the signal peptide SEQ ID NO: 74) (or a sequence having at least 70%, 75%, 80%, 85%, 90%, or 95% sequence identity thereto);
k) the amino acid sequence of SEQ ID NO: 165 (without the signal peptide SEQ ID NO: 74) (or a sequence having at least 70%, 75%, 80%, 85%, 90%, or 95% sequence identity thereto), the amino acid sequence of SEQ ID NO: 166 (without the signal peptide SEQ ID NO: 74) (or a sequence having at least 70%, 75%, 80%, 85%, 90%, or 95% sequence identity thereto), and the amino acid sequence of SEQ ID NO: 164 (without the signal peptide SEQ ID NO: 74) (or a sequence having at least 70%, 75%, 80%, 85%, 90%, or 95% sequence identity thereto);
l) the amino acid sequence of SEQ ID NO: 167 (without the signal peptide SEQ ID NO: 74) (or a sequence having at least 70%, 75%, 80%, 85%, 90%, or 95% sequence identity thereto), and the amino acid sequence of SEQ ID NO: 168 (without the signal peptide SEQ ID NO: 74) (or a sequence having at least 70%, 75%, 80%, 85%, 90%, or 95% sequence identity thereto);
m) the amino acid sequence of SEQ ID NO: 169 (without the signal peptide SEQ ID NO: 74) (or a sequence having at least 70%, 75%, 80%, 85%, 90%, or 95% sequence identity thereto), and the amino acid sequence of SEQ ID NO: 170 (without the signal peptide SEQ ID NO: 74) (or a sequence having at least 70%, 75%, 80%, 85%, 90%, or 95% sequence identity thereto);
n) the amino acid sequence of SEQ ID NO: 171 (without the signal peptide SEQ ID NO: 74) (or a sequence having at least 70%, 75%, 80%, 85%, 90%, or 95% sequence identity thereto), and the amino acid sequence of SEQ ID NO: 172 (without the signal peptide SEQ ID NO: 74) (or a sequence having at least 70%, 75%, 80%, 85%, 90%, or 95% sequence identity thereto);
o) the amino acid sequence of SEQ ID NO: 173 (without the signal peptide SEQ ID NO: 74) (or a sequence having at least 70%, 75%, 80%, 85%, 90%, or 95% sequence identity thereto), and the amino acid sequence of SEQ ID NO: 174 (without the signal peptide SEQ ID NO: 74) (or a sequence having at least 70%, 75%, 80%, 85%, 90%, or 95% sequence identity thereto);
p) the amino acid sequence of SEQ ID NO: 175 (without the signal peptide SEQ ID NO: 74) (or a sequence having at least 70%, 75%, 80%, 85%, 90%, or 95% sequence identity thereto), and the amino acid sequence of SEQ ID NO: 176 (without the signal peptide SEQ ID NO: 74) (or a sequence having at least 70%, 75%, 80%, 85%, 90%, or 95% sequence identity thereto); or
q) the amino acid sequence of SEQ ID NO: 177 (without the signal peptide SEQ ID NO: 74) (or a sequence having at least 70%, 75%, 80%, 85%, 90%, or 95% sequence identity thereto), and the amino acid sequence of SEQ ID NO: 178 (without the signal peptide SEQ ID NO: 74) (or a sequence having at least 70%, 75%, 80%, 85%, 90%, or 95% sequence identity thereto).

45-186. (canceled)

187. An isolated nucleic acid molecule encoding the multifunctional molecule of claim 1.

188. A vector comprising the nucleic acid molecule of claim 187.

189. A cell comprising the nucleic acid molecule of claim 187.

190. A method of making the multifunctional molecule of claim 1, comprising culturing the cell of claim 189, under suitable conditions.

191. A pharmaceutical composition comprising the multifunctional molecule of claim 1 and a pharmaceutically acceptable carrier, excipient, or stabilizer.

192. A method of treating a cancer, comprising administering to a subject in need thereof the multifunctional molecule of claim 1, wherein the multifunctional antibody is administered in an amount effective to treat the cancer.

193-198. (canceled)


SEQUENCE LISTING (293 sequences)

SEQ ID NO: 1 - protein, 144 aa, Homo sapiens
KPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 2 - protein, 152 aa, Homo sapiens
RTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 3 - protein, 158 aa, Homo sapiens
LPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 4 - protein, 126 aa, Homo sapiens
RIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 5 - protein, 146 aa, Homo sapiens
GDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 6 - protein, 139 aa, Homo sapiens
LRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 7 - protein, 155 aa, Homo sapiens
QRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 8 - protein, 172 aa, Homo sapiens
QRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 9 - protein, 167 aa, Homo sapiens
DPAGLLDLRQGMFAQLVAQNVLLIDGPLSWYSDPGLAGVSLTGGLSYKEDTKELVVAKAGVYYVFFQMELRRVVAGEGSGSVSLALHLMPLRSAAGAAALALTVDLPPASSEARNSAFGFQGRLLHLSAGQRLGVHLHTEARARHAWQLTQGATVLGLFRVTPEIPA

SEQ ID NO: 10 - protein, 163 aa, Homo sapiens
PQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 11 - protein, 156 aa, Homo sapiens
AQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 12 - protein, 145 aa, Homo sapiens
RAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 13 - protein, 136 aa, Homo sapiens
HSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 14 - protein, 146 aa, Homo sapiens
ETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 15 - protein, 148 aa, Homo sapiens
NPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 16 - protein, 161 aa, Homo sapiens
DGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 17 - protein, 128 aa, Homo sapiens
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 18 - protein, 559 aa, Artificial Sequence
  source: /note=“Description of Artificial Sequence: Synthetic polypeptide”
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGDQNPQIAAHVISEASSKTTSVLQWAEKGYYTMSNNLVTLENGKQLTVKRQGLYYIYAQVTFCSNREASSQAPFIASLALKSPGRFERILLRAANTHSSAKPCGQQSIHLGGVFELQPGASVFVNVTDPSQVSHGTGFTSFGLLKLGGGGSGDQNPQIAAHVISEASSKTTSVLQWAEKGYYTMSNNLVTLENGKQLTVKRQGLYYIYAQVTFCSNREASSQAPFIASLALKSPGRFERILLRAANTHSSAKPCGQQSIHLGGVFELQPGASVFVNVTDPSQVSHGTGFTSFGLLKL

SEQ ID NO: 19 - protein, 408 aa, Artificial Sequence
  source: /note=“Description of Artificial Sequence: Synthetic polypeptide”
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGDQNPQIAAHVISEASSKTTSVLQWAEKGYYTMSNNLVTLENGKQLTVKRQGLYYIYAQVTFCSNREASSQAPFIASLALKSPGRFERILLRAANTHSSAKPCGQQSIHLGGVFELQPGASVFVNVTDPSQVSHGTGFTSFGLLKL

SEQ ID NO: 20 - protein, 247 aa, Artificial Sequence
  source: /note=“Description of Artificial Sequence: Synthetic polypeptide”
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 662 aa, Artificial Sequence
  source: /note=“Description of Artificial Sequence: Synthetic polypeptide”
METDTLLLWVLLLWVPGSTGGDQNPQIAAHVISEASSKTTSVLQWAEKGYYTMSNNLVTLENGKQLTVKRQGLYYIYAQVTFCSNREASSQAPFIASLALKSPGRFERILLRAANTHSSAKPCGQQSIHLGGVFELQPGASVFVNVTDPSQVSHGTGFTSFGLLKLGGGGSGDQNPQIAAHVISEASSKTTSVLQWAEKGYYTMSNNLVTLENGKQLTVKRQGLYYIYAQVTFCSNREASSQAPFIASLALKSPGRFERILLRAANTHSSAKPCGQQSIHLGGVFELQPGASVFVNVTDPSQVSHGTGFTSFGLLKLGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 288 aa, Artificial Sequence
  source: /note=“Description of Artificial Sequence: Synthetic polypeptide”
METDTLLLWVLLLWVPGSTGGDQNPQIAAHVISEASSKTTSVLQWAEKGYYTMSNNLVTLENGKQLTVKRQGLYYIYAQVTFCSNREASSQAPFIASLALKSPGRFERILLRAANTHSSAKPCGQQSIHLGGVFELQPGASVFVNVTDPSQVSHGTGFTSFGLLKLGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 519 aa, Artificial Sequence
  source: /note=“Description of Artificial Sequence: Synthetic polypeptide”
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANGQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 24 - protein, 395 aa, Artificial Sequence
  source: /note=“Description of Artificial Sequence: Synthetic polypeptide”
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 25 - protein, 617 aa, Artificial Sequence
  source: /note=“Description of Artificial Sequence: Synthetic polypeptide”
METDTLLLWVLLLWVPGSTGQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANGQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 26 - protein, 270 aa, Artificial Sequence
  source: /note=“Description of Artificial Sequence: Synthetic polypeptide”
METDTLLLWVLLLWVPGSTGQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 573 aa, Artificial Sequence
  source: /note=“Description of Artificial Sequence: Synthetic polypeptide”
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGSGGSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 28 - protein, 419 aa, Artificial Sequence
  source: /note=“Description of Artificial Sequence: Synthetic polypeptide”
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 29 - protein, 671 aa, Artificial Sequence
  source: /note=“Description of Artificial Sequence: Synthetic polypeptide”
METDTLLLWVLLLWVPGSTGRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGSGGSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 30 - protein, 294 aa, Artificial Sequence
  source: /note=“Description of Artificial Sequence: Synthetic polypeptide”
METDTLLLWVLLLWVPGSTGRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 548 aa, Artificial Sequence
  source: /note=“Description of Artificial Sequence: Synthetic polypeptide”
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSLRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKLGGSGGKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 32 - protein, 406 aa, Artificial Sequence
  source: /note=“Description of Artificial Sequence: Synthetic polypeptide”
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSLRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 33 - protein, 646 aa, Artificial Sequence
  source: /note=“Description of Artificial Sequence: Synthetic polypeptide”
METDTLLLWVLLLWVPGSTGLRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKLGGSGGKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKLGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 34 - protein, 281 aa, Artificial Sequence
  source: /note=“Description of Artificial Sequence: Synthetic polypeptide”
METDTLLLWVLLLWVPGSTGLRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKLGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 568 aa, Artificial Sequence
  source: /note=“Description of Artificial Sequence: Synthetic polypeptide”
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMVGGSGGNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 36 - protein, 415 aa, Artificial Sequence
  source: /note=“Description of Artificial Sequence: Synthetic polypeptide”
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 37 - protein, 666 aa, Artificial Sequence
  source: /note=“Description of Artificial Sequence: Synthetic polypeptide”
METDTLLLWVLLLWVPGSTGNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMVGGSGGNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMVGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 38 - protein, 290 aa, Artificial Sequence
  source: /note=“Description of Artificial Sequence: Synthetic polypeptide”
METDTLLLWVLLLWVPGSTGNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMVGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 60 bp, Artificial Sequence
  source: /note=“Description of Artificial Sequence: Synthetic oligonucleotide”
atggaaaccgatacactgctgctgtgggtgctgctcctctgggtgccaggcagcaccggc

SEQ ID NO: 40 - DNA, 321 bp, Artificial Sequence
  source: /note=“Description of Artificial Sequence: Synthetic polynucleotide”
agaacagtggctgccccttccgtgttcatcttcccaccttccgacgagcagctgaagtctggcacagcctctgtcgtgtgtctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggactgtctagccccgtgaccaagtctttcaaccggggcgagtgc

SEQ ID NO: 41 - DNA, 309 bp, Artificial Sequence
  source: /note=“Description of Artificial Sequence: Synthetic polynucleotide”
gcttctaccaagggacctagcgtgttccctctggctccttccagcaagtctacctctggtggcacagctgctctgggctgcctggtcaaggattacttccctgagcctgtgaccgtgtcctggaattctggcgctctgacatccggcgtgcacacctttccagctgtcctgcaatcctccggcctgtacagcctgtcctctgtcgtgaccgtgccttctagctctctgggcacccagacctacatctgcaatgtgaaccacaagccttccaacaccaaggtggacaagagagtggaacccaagtcctgc

SEQ ID NO: 42 - DNA, 681 bp, Artificial Sequence
  source: /note=“Description of Artificial Sequence: Synthetic polynucleotide”
gataagacccacacctgtcctccatgtcctgcccctgagctgctgggcggacctagcgtgttcctgttccctccaaagcccaaggacaccctgatgatcagccggacccctgaagtgacctgcgtggtggtggatgtgtcccacgaggatcccgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccctatcgagaaaaccatcagcaaggccaagggccagccccgcgaacctcaggtgtacacactgcctccctgccgggaagagatgaccaagaaccaggtgtccctgtggtgcctggtcaagggcttctacccctccgatatcgccgtggaatgggagagcaacggccagcccgagaacaactacaagaccacccctcccgtgctggacagcgacggcagcttcttcctgtactccaaactgaccgtggacaagagccggtggcagcagggcaatgtgttcagctgtagcgtgatgcacgaggccctgcacaaccactacacccagaagtctctgagcctgagccccggcaag

SEQ ID NO: 43 - DNA, 681 bp, Artificial Sequence
  source: /note=“Description of Artificial Sequence: Synthetic polynucleotide”
gataagacccacacctgtcctccatgtcctgcccctgagctgctgggcggacctagcgtgttcctgttccctccaaagcccaaggacaccctgatgatcagccggacccctgaagtgacctgcgtggtggtggatgtgtcccacgaggatcccgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccctatcgagaaaaccatcagcaaggccaagggccagcctagagagcctcaggtctgcaccctgcctcccagccgggaagagatgaccaagaaccaggtgtccctgtcctgcgccgtgaagggcttctacccctccgatatcgccgtggaatgggagagcaacggccagcccgagaacaactacaagaccacccctcccgtgctggacagcgacggcagcttcttcctggtgtccaaactgaccgtggacaagagccggtggcagcagggcaatgtgttcagctgtagcgtgatgcacgaggccctgcacaaccactacacccagaagtctctgagcctgagccccggcaag

SEQ ID NO: 44 - DNA, 3 bp, Artificial Sequence
  source: /note=“Description of Artificial Sequence: Synthetic oligonucleotide”
ggc

SEQ ID NO: 45 - DNA, 15 bp, Artificial Sequence
  source: /note=“Description of Artificial Sequence: Synthetic oligonucleotide”
ggaggcggtggctct

SEQ ID NO: 46 - DNA, 15 bp, Artificial Sequence
  source: /note=“Description of Artificial Sequence: Synthetic oligonucleotide”
ggaggctccggcgga

SEQ ID NO: 47 - DNA, 18 bp, Artificial Sequence
  source: /note=“Description of Artificial Sequence: Synthetic oligonucleotide”
ggtggctccggcggctct

SEQ ID NO: 48 - DNA, 21 bp, Artificial Sequence
  source: /note=“Description of Artificial Sequence: Synthetic oligonucleotide”
ggtggtggtagtggcggaggc

SEQ ID NO: 49 - DNA, 45 bp, Artificial Sequence
  source: /note=“Description of Artificial Sequence: Synthetic oligonucleotide”
ggcggcggaggatctggcggaggcggttctggtggtggcggatcc

SEQ ID NO: 50 - DNA, 438 bp, Artificial Sequence
  source: /note=“Description of Artificial Sequence: Synthetic polynucleotide”
ggcgatcagaaccctcagatcgccgcccacgtgatctccgaggccagctctaagaccaccagcgtgctgcagtgggccgagaagggctactataccatgagcaacaacctcgtgaccctggaaaacggcaagcagctgactgtgaagagacagggcctgtactacatctacgcccaagtgaccttctgctccaacagagaggcctcctcccaggccccctttatcgcctctctggccctgaagtcccccggcagattcgagcggatcctgctgagagccgccaacacccactcctccgctaagccttgtggccagcagtccatccatctgggcggcgtgttcgaactgcagcctggcgcctctgtgttcgtgaacgtgaccgacccctctcaggtgtcacacggcaccggcttcaccagcttcggcctgctgaagctg

SEQ ID NO: 51 - DNA, 378 bp, Artificial Sequence
  source: /note=“Description of Artificial Sequence: Synthetic polynucleotide”
agaatccagtccatcaaggtgcagttcaccgagtacaagaaagagaagggcttcatcctgaccagccagaaagaggacgagatcatgaaggtgcagaacaactccgtgatcatcaactgcgacggcttttacctgatctccctgaagggctacttctcccaagaagtgaacatctccctgcactaccagaaggacgaggaacccctgttccagctgaagaaagtgcggagcgtgaactccctgatggtggcctctctgacctacaaggacaaggtgtacctgaacgtgaccaccgacaacaccagcctggacgacttccatgtgaatggcggcgagctgatcctgatccatcagaaccctggcgagttctgcgttctc

SEQ ID NO: 52 - DNA, 456 bp, Artificial Sequence
  source: /note=“Description of Artificial Sequence: Synthetic polynucleotide”
agaacaccttctgacaagcctgtggctcacgtggtggccaatcctcaggctgaaggacagctgcagtggctgaacagaagggccaacgctctgctggccaatggcgtggaactgagagacaatcagctggtggtgccttccgagggcctgtacctgatctatagccaggtgctgttcaaaggccagggctgcccttctactcacgtgctgctgacccacaccatcagcagaatcgccgtgtcctaccagaccaaagtgaacctgctgtccgccatcaagagcccctgccagagagaaacacctgagggcgctgaagccaagccttggtacgagcctatctatctcggcggcgtgttccagctcgagaagggcgatagactgagcgccgagatcaaccggcctgactacctggattttgccgagtccggccaggtgtacttcggcattattgccctt

SEQ ID NO: 53 - DNA, 444 bp, Artificial Sequence
  source: /note=“Description of Artificial Sequence: Synthetic polynucleotide”
gataagccagtggcacatgtcgtggctaacccacaggcagagggtcaactccagtggctcaatcggagagctaatgcactgctggctaacggcgttgagctgcgggataaccagctcgtggtcccaagcgagggactctatctcatctacagtcaggtcttgtttaaaggacaggggtgtcccagcacacatgttctgctgacacatacaatctcccggatcgccgtcagctatcagacaaaagtcaatctgctgagcgctatcaagtctccctgccagcgcgaaactccagaaggcgcagaggctaaaccttggtatgaacccatctaccttggcggagtgtttcagttggaaaaaggcgaccggctgtccgctgagatcaacagacccgattatctggatttcgctgagagcggacaggtctactttgggatcattgctctg

SEQ ID NO: 54 - DNA, 369 bp, Artificial Sequence
  source: /note=“Description of Artificial Sequence: Synthetic polynucleotide”
caactggaaaccgccaaagaaccctgcatggccaagttcggccctctgccttctaagtggcagatggcctcttccgagcctccttgcgtgaacaaagtgtccgactggaagctggaaatcctgcagaacggcctgtacctgatctacggacaggtggcccctaacgccaactacaacgatgtggcccctttcgaagtgcggctgtacaagaacaaggacatgatccagacactgaccaacaagtccaagatccagaacgtcggcggcacctacgagctgcatgtgggcgataccatcgacctgatcttcaactccgagcaccaggtgctgaagaacaacacctactggggcatcatcctgctggctaac

SEQ ID NO: 55 - DNA, 384 bp, Artificial Sequence
  source: /note=“Description of Artificial Sequence: Synthetic polynucleotide”
cagctggaaacagctaaagaaccttgtatggctaaatttgggcccctgcctagcaaatggcaaatggctagcagcgagccaccatgtgtgaacaaggtttcagattggaaactcgagatcctccagaatgggctctatctgatctatggccaggtcgcacccaatgctaactacaatgacgtcgcaccatttgaagtccgcctctataagaacaaagatatgattcaaaccctcacaaacaagagcaagattcagaatgttggcgggacatatgaactgcacgtgggagacacaatcgatctcatcttcaatagcgagcatcaggtcctcaaaaacaatacttattggggaatcattctgctcgccaatcctcagttcatctcc

SEQ ID NO: 56 - DNA, 444 bp, Artificial Sequence
  source: /note=“Description of Artificial Sequence: Synthetic polynucleotide”
aatcctgcagctcatctgaccggcgccaactcctctctgactggttctggtggacccctgctctgggaaactcagctgggactcgctttcctgcggggcctgtcttatcatgatggcgctctggtggtcaccaaggccggctactactacatctactccaaggtgcagctcggcggcgtgggatgtcctcttggactggcttctacaatcacccacggcctgtacaagcggacccctagataccccgaggaactggaactgctggtgtcccagcagtctccttgtggcagagccacctcctccagcagagtttggtgggactcttctttccttggtggcgtggtgcacctggaagctggcgagaaagtggtcgtcagagtgctggacgaaagactcgtgcggctgagagatggcacccggtcttacttcggcgcctttatggtt

SEQ ID NO: 57 - DNA, 417 bp, Artificial Sequence
  source: /note=“Description of Artificial Sequence: Synthetic polynucleotide”
ttgagaaaagtggctcacctgaccggcaagtccaacagcagatccatgcctctcgagtgggaagatacctacggcatcgtgctgctgtccggcgtgaagtacaagaaaggcggcctggtcatcaacgagactggcctgtacttcgtgtacagcaaggtgtacttccggggccagtcctgcaacaacctgcctctgagccataaggtgtacatgcggaacagcaagtaccctcaggacctggtcatgatggaaggcaagatgatgtcctactgcaccaccggccagatgtgggccagatcttcttatctgggcgccgtgttcaacctgaccagcgccgatcacctgtacgtgaacgtgtccgagctgtccctggtcaacttcgaggaatcccagaccttcttcggcctgtacaagctt

SEQ ID NO: 58 - DNA, 411 bp, Artificial Sequence
  source: /note=“Description of Artificial Sequence: Synthetic polynucleotide”
aaggtggcccatctgactggaaagtctaactcccggtctatgccccttgaatgggaagatacttatgggattgtcctgctgagcggggtcaagtataagaaaggtggactcgtgattaacgaaaccgggctctacttcgtctactctaaggtctacttcagagggcagagctgtaacaatctgcccctgtctcacaaggtctacatgagaaactccaagtatccccaggatctcgtgatgatggaagggaaaatgatgagctactgtacaacagggcaaatgtgggctagaagctcctacctgggagctgtgtttaatctgacctctgctgaccacctctatgtgaatgtgtctgaactgagcctcgtgaacttcgaagagagccagaccttttttgggctctataagctg

SEQ ID NO: 59 - DNA, 1677 bp, Artificial Sequence
  source: /note=“Description of Artificial Sequence: Synthetic polynucleotide”
atggaaaccgacaccctgctgctgtgggtgctgctgctgtgggtgccaggctctaccggcgacaagacccacacctgtcctccctgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggattggctgaacggcaaagagtacaagtgcaaggtgtccaacaaggccctgccagcccccatcgaaaagaccatctccaaggccaagggccagccccgggaaccccaggtgtacacactgcccccttgccgggaagagatgaccaagaaccaggtgtccctgtggtgcctcgtgaagggcttctacccctccgatatcgccgtggaatgggagtccaacggccagcctgagaacaactacaagaccaccccccctgtgctggactccgacggctcattcttcctgtactccaagctgacagtggacaagtcccggtggcagcagggcaacgtgttctcctgctccgtgatgcacgaggccctgcacaaccactacacccagaagtccctgtccctgagccctggaaaaggcggcggaggatctggcggaggcggttctggtggtggcggatccggcgatcagaaccctcagatcgccgcccacgtgatctccgaggccagctctaagaccaccagcgtgctgcagtgggccgagaagggctactacaccatgagcaacaacctcgtgacactggaaaacggcaagcagctgactgtgaagagacagggcctgtactacatctacgcccaagtgaccttctgctccaacagagaggcctcctcccaggccccctttatcgcctctctggccctgaagtcccccggcagattcgagcggatcctgctgagagccgccaacacccactcctccgctaagccttgtggccagcagtccatccatctgggcggcgtgttcgaactgcagcctggcgcctctgtgttcgtgaacgtgaccgacccctctcaggtgtcacacggcaccggcttcaccagcttcggcctgctgaaactgggaggcggtggctctggcgaccagaatccacagattgctgctcatgtgatcagcgaggcctccagcaagaccacttctgtgctgcagtgggctgaaaaagggtattataccatgtctaacaatctcgtgaccctggaaaatgggaaacagctgaccgtgaaacggcaggggctgtattatatctatgctcaagtgacattttgttctaaccgcgaggctagcagccaggcccctttcattgctagcctggctctgaaaagccctggccgcttcgagagaattctgctgcgggctgccaatacccacagctctgccaagccatgcggacagcagagcatccacctggggggagtgtttgagctgcagccaggtgccagtgtgtttgtgaatgtgacagatcccagccaggtgtcccatggcacaggctttacatcctttgggctgctgaagctg

SEQ ID NO: 60 - DNA, 1224 bp, Artificial Sequence
  source: /note=“Description of Artificial Sequence: Synthetic polynucleotide”
atggaaaccgacaccctgctgctgtgggtgctgctgctgtgggtgccaggctctaccggcgacaagacccacacctgtcctccctgtcctgcccctgaactgctgggcggaccttccgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggattggctgaacggcaaagagtacaagtgcaaggtgtccaacaaggccctgccagcccccatcgaaaagaccatctccaaggccaagggccagccccgggaacctcaagtgtgcaccctgccccctagccgggaagagatgaccaagaaccaggtgtccctgagctgcgccgtgaagggcttctacccctccgatatcgccgtggaatgggagtccaacggccagcctgagaacaactacaagaccaccccccctgtgctggactccgacggctcattcttcctggtgtccaagctgacagtggacaagtcccggtggcagcagggcaacgtgttctcctgctccgtgatgcacgaggccctgcacaaccactacacccagaagtccctgtccctgtctcccggaaaaggcggcggaggatctggcggaggcggttctggtggtggcggatccggcgatcagaaccctcagatcgccgcccacgtgatctccgaggccagctctaagaccaccagcgtgctgcagtgggccgagaagggctactataccatgagcaacaacctcgtgaccctggaaaacggcaagcagctgactgtgaagagacagggcctgtactacatctacgcccaagtgaccttctgctccaacagagaggcctcctcccaggccccctttatcgcctctctggccctgaagtcccccggcagattcgagcggatcctgctgagagccgccaacacccactcctccgctaagccttgtggccagcagtccatccatctgggcggcgtgttcgaactgcagcctggcgcctctgtgttcgtgaacgtgaccgacccctctcaggtgtcacacggcaccggcttcaccagcttcggcctgctgaagctg

SEQ ID NO: 61 - DNA, 741 bp, Artificial Sequence
  source: /note=“Description of Artificial Sequence: Synthetic polynucleotide”
atggaaaccgatacactgctgctgtgggtgctgctcctctgggtgccaggcagcaccggcgataagacccacacctgtcctccatgtcctgcccctgagctgctgggcggacctagcgtgttcctgttccctccaaagcccaaggacaccctgatgatcagccggacccctgaagtgacctgcgtggtggtggatgtgtcccacgaggatcccgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccctatcgagaaaaccatcagcaaggccaagggccagccccgcgaacctcaggtgtacacactgcctccctgccgggaagagatgaccaagaaccaggtgtccctgtggtgcctggtcaagggcttctacccctccgatatcgccgtggaatgggagagcaacggccagcccgagaacaactacaagaccacccctcccgtgctggacagcgacggcagcttcttcctgtactccaaactgaccgtggacaagagccggtggcagcagggcaatgtgttcagctgtagcgtgatgcacgaggccctgcacaaccactacacccagaagtctctgagcctgagccccggcaag

SEQ ID NO: 62 - DNA, 1986 bp, Artificial Sequence
  source: /note=“Description of Artificial Sequence: Synthetic polynucleotide”
atggaaaccgacacactgctgctgtgggtgctgctcttgtgggtgccaggatctacaggcggcgatcagaaccctcagatcgccgctcatgtgatctccgaggcctcttccaagaccacctctgtgctgcagtgggccgagaagggctactacaccatgtccaacaacctggtcaccctggaaaacggcaagcagctgaccgtgaagagacagggcctgtactacatctacgcccaagtgaccttctgctccaacagagaggcctcctctcaggccccttttatcgcctctctggccctgaagtcccctggcagattcgagagaatcctgctgagagccgctaacacccactcctctgctaagccttgtggccagcagtctatccatctcggcggagtgtttgagctgcagcctggcgcttctgtgttcgtgaacgtgaccgatcctagccaggtgtcccacggcaccggcttcacatcttttggcctgctgaaactcggaggcggcggaagcggagatcagaatccacaaattgctgcccacgtgatcagcgaggccagctctaagacaaccagcgtcctccagtgggctgaaaaggggtattatacaatgagcaacaatctcgtgaccctcgagaatgggaaacagctgacagtcaagcggcaggggctctattacatctatgctcaagtcacattttgctctaaccgcgaggcttccagccaggctcctttcattgcttccctggctctgaaaagccccggcagatttgagcggattctgctgcgggctgccaacacacactctagcgccaaaccatgcggccagcagagcattcatcttggcggcgttttcgaattgcagccaggcgcaagcgtgttcgtcaatgtgacagacccctctcaggtttcccatggcacagggtttaccagcttcggactgctcaaacttggcggtggtggatctggtggcggaggatctggcggtggcggttctgcttctaccaagggacctagcgtgttccctctggctccttccagcaagtctacctctggtggcacagctgctctgggctgcctggtcaaggattacttccctgagcctgtgaccgtgtcctggaattctggcgctctgacatccggcgtgcacacctttccagctgtcctgcaatcctccggcctgtacagcctgtcctctgtcgtgaccgtgccttctagctctctgggcacccagacctacatctgcaatgtgaaccacaagccttccaacaccaaggtggacaagagagtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtcgatgtgtctcacgaggatcccgaagtgaagttcaattggtacgtggacggcgtggaagtgcacaacgccaagaccaagcctagagaggaacagtacaactccacctacagagtggtgtccgtgctgacagtgctgcaccaggattggctgaacggcaaagagtacaagtgcaaggtttccaacaaggccctgcctgctcctatcgaaaagaccatctccaaggccaagggccagcctcgggaacctcaagtctgtaccctgcctcctagccgggaagagatgaccaagaatcaagtgtccctgtcctgcgccgtgaagggcttttacccttccgatatcgccgtggaatgggagagcaatggccagcctgagaacaactacaagacaacccctcctgtgctggactccgacggctcattcttcctggtgtccaagctgactgtggacaagtccagatggcagcagggcaatgtgttctcctgctccgtgatgcacgaggccctgcacaatcactacacccagaagtctctgtctctgagccctggcaag

SEQ ID NO: 63 - DNA, 864 bp, Artificial Sequence
  source: /note=“Description of Artificial Sequence: Synthetic polynucleotide”
atggaaaccgacacactgctgctgtgggtgctgctcttgtgggtgccaggatctacaggcggcgatcagaaccctcagatcgccgctcatgtgatctccgaggcctcttccaagaccacctctgtgctgcagtgggccgagaagggctactacaccatgtccaacaacctggtcaccctggaaaacggcaagcagctgaccgtgaagagacagggcctgtactacatctacgcccaagtgaccttctgctccaacagagaggcctcctctcaggccccttttatcgcctctctggccctgaagtcccctggcagattcgagagaatcctgctgagagccgctaacacccactcctctgctaagccttgtggccagcagtctatccatctcggcggagtgtttgagctgcagcctggcgcttctgtgttcgtgaacgtgaccgatcctagccaggtgtcccacggcaccggcttcacatcttttggcctgctgaaactcggaggcggcggatctggtggcggaggttctggcggaggcggatctagaacagtggctgccccttccgtgttcatcttcccaccttccgacgagcagctgaagtctggcacagcctctgtcgtgtgtctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggactgtctagccccgtgaccaagtctttcaaccggggcgagtgc

SEQ ID NO: 64 - DNA, 1578 bp, Artificial Sequence
  source: /note=“Description of Artificial Sequence: Synthetic polynucleotide”
atggaaaccgacacactgctgctgtgggtgctgctcttgtgggtgccaggatctaccggcgacaagacccatacctgtcctccatgtcctgctccagagctgctcggaggcccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccatgccgggaagagatgaccaagaaccaggtgtccctgtggtgcctggtcaagggcttctacccttccgatatcgccgtggaatgggagagcaatggccagcctgagaacaactacaagaccacacctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaaggcggcggaggatctggcggaggtggaagcggaggcggaggaagcggtggcggcggatctagaatccagtccatcaaggtgcagttcaccgagtacaagaaagagaagggcttcatcctgaccagccagaaagaggacgagatcatgaaggtgcagaacaactccgtgatcatcaactgcgacggcttttacctgatctccctgaagggctacttctcccaagaagtgaacatctccctgcactaccagaaggacgaggaacccctgttccagctgaagaaagtgcggagcgtgaactccctgatggtggcctctctgacctacaaggacaaggtgtacctgaacgtgaccaccgacaacaccagcctggacgacttccatgtgaatggcggcgagctgatcctgatccatcagaaccctggcgagttctgcgttctcggtggtggtagtggcggaggcagaatccagagtattaaggtccagtttacagagtataagaaagaaaaaggttttatcctcacctctcagaaagaagatgagattatgaaggtccaaaacaatagcgtcatcattaactgtgatgggttctacctcatcagcctcaagggctatttcagtcaagaagtcaatatcagcctccattatcagaaggatgaagaaccactctttcaactcaaaaaagttcgctccgtgaacagcctcatggtggctagcctgacttataaggacaaagtctatctcaatgtcaccacagataacacatccctcgacgatttccacgtcaacggcggagaactgattctcatccaccaaaatccaggcgagttttgtgtgctg

SEQ ID NO: 65 - DNA, 1179 bp, Artificial Sequence
  source: /note=“Description of Artificial Sequence: Synthetic polynucleotide”
atggaaaccgacacactgctgctgtgggtgctgctcttgtgggtgccaggatctaccggcgacaagacccatacctgtcctccatgtcctgctccagagctgctcggaggcccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccctgcctcctagccgggaagagatgaccaagaaccaggtgtccctgtcctgtgccgtgaagggcttctacccttccgatatcgccgtggaatgggagagcaatggccagcctgagaacaactacaagaccacacctcctgtgctggactccgacggctcattcttcctggtgtccaagctgacagtggacaagtccagatggcagcagggcaacgtgttctcctgctccgtgatgcacgaggccctgcacaatcactacacccagaagtccctgagcctgtctcctggaaaaggcggcggaggatctggcggaggtggaagcggaggcggaggaagcggtggcggcggatctagaatccagtccatcaaggtgcagttcaccgagtacaagaaagagaagggctttatcctgaccagccagaaagaggacgagatcatgaaggtgcagaacaactccgtgatcatcaactgcgacggcttttacctgatctccctgaagggctacttctcccaagaagtgaacatctccctgcactaccagaaggacgaggaacccctgttccagctgaagaaagtgcggagcgtgaactccctgatggtggcctctctgacctacaaggacaaggtgtacctgaacgtgaccaccgacaacaccagcctggacgacttccatgtgaatggcggcgagctgatcctgatccatcagaaccctggcgagttctgcgtgctg

SEQ ID NO: 66 - DNA, 1719 bp, Artificial Sequence
  source: /note=“Description of Artificial Sequence: Synthetic polynucleotide”
atggaaaccgacacactgctgctgtgggtgctgctcttgtgggtgccaggatctaccggcgacaagacccatacctgtcctccatgtcctgctccagagctgctcggaggcccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccatgccgggaagagatgaccaagaaccaggtgtccctgtggtgcctggtcaagggcttctacccttccgatatcgccgtggaatgggagagcaatggccagcctgagaacaactacaagaccacacctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaaggcggcggaggatctggcggaggtggaagcggaggcggaggaagcggtggcggcggatctagaacaccttctgacaagcctgtggctcacgtggtggccaatcctcaggctgaaggacagctgcagtggctgaacagaagggccaacgctctgctggccaatggcgtggaactgagagacaatcagctggtggtgccttccgagggcctgtacctgatctatagccaggtgctgttcaaaggccagggctgcccttctactcacgtgctgctgacccacaccatcagcagaatcgccgtgtcctaccagaccaaagtgaacctgctgtccgccatcaagagcccctgccagagagaaacacctgagggcgctgaagccaagccttggtacgagcctatctatctcggcggcgtgttccagctcgagaagggcgatagactgagcgccgagatcaaccggcctgactacctggattttgccgagtccggccaggtgtacttcggcattattgcccttggtggctccggcggctctgataagccagtggcacatgtcgtggctaacccacaggcagagggtcaactccagtggctcaatcggagagctaatgcactgctggctaacggcgttgagctgcgggataaccagctcgtggtcccaagcgagggactctatctcatctacagtcaggtcttgtttaaaggacaggggtgtcccagcacacatgttctgctgacacatacaatctcccggatcgccgtcagctatcagacaaaagtcaatctgctgagcgctatcaagtctccctgccagcgcgaaactccagaaggcgcagaggctaaaccttggtatgaacccatctaccttggcggagtgtttcagttggaaaaaggcgaccggctgtccgctgagatcaacagacccgattatctggatttcgctgagagcggacaggtctactttgggatcattgctctg

SEQ ID NO: 67 - DNA, 1257 bp, Artificial Sequence
  source: /note=“Description of Artificial Sequence: Synthetic polynucleotide”
atggaaaccgacacactgctgctgtgggtgctgctcttgtgggtgccaggatctaccggcgacaagacccatacctgtcctccatgtcctgctccagagctgctcggaggcccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccctgcctcctagccgggaagagatgaccaagaaccaggtgtccctgtcctgtgccgtgaagggcttctacccttccgatatcgccgtggaatgggagagcaatggccagcctgagaacaactacaagaccacacctcctgtgctggactccgacggctcattcttcctggtgtccaagctgacagtggacaagtccagatggcagcagggcaacgtgttctcctgctccgtgatgcacgaggccctgcacaatcactacacccagaagtccctgagcctgtctcctggaaaaggcggcggaggatctggcggaggtggaagcggaggcggaggaagcggtggcggcggatctagaacaccttctgacaagcctgtggctcacgtggtggccaatcctcaggctgaaggacagctgcagtggctgaacagaagggccaacgctctgctggccaatggcgtggaactgagagacaatcagctggtggtgccttccgagggcctgtacctgatctatagccaggtgctgttcaaaggccagggctgcccttctactcacgtgctgctgacccacaccatcagcagaatcgccgtgtcctaccagaccaaagtgaacctgctgtccgccatcaagagcccctgccagagagaaacacctgagggcgctgaagccaagccttggtacgagcctatctatctcggcggcgtgttccagctcgagaagggcgatagactgagcgccgagatcaaccggcctgactacctggattttgccgagtccggccaggtgtacttcggcatcattgccctg

SEQ ID NO: 68 - DNA, 1644 bp, Artificial Sequence
  source: /note=“Description of Artificial Sequence: Synthetic polynucleotide”
atggaaaccgacacactgctgctgtgggtgctgctcttgtgggtgccaggatctaccggcgacaagacccatacctgtcctccatgtcctgctccagagctgctcggaggcccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccatgccgggaagagatgaccaagaaccaggtgtccctgtggtgcctggtcaagggcttctacccttccgatatcgccgtggaatgggagagcaatggccagcctgagaacaactacaagaccacacctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaaggcggcggaggatctggcggaggtggaagcggaggcggaggaagcggtggcggcggatctttgagaaaagtggctcacctgaccggcaagtccaacagcagatccatgcctctcgagtgggaagatacctacggcatcgtgctgctgtccggcgtgaagtacaagaaaggcggcctggtcatcaacgagactggcctgtacttcgtgtacagcaaggtgtacttccggggccagtcctgcaacaacctgcctctgagccataaggtgtacatgcggaacagcaagtaccctcaggacctggtcatgatggaaggcaagatgatgtcctactgcaccaccggccagatgtgggccagatcttcttatctgggcgccgtgttcaacctgaccagcgccgatcacctgtacgtgaacgtgtccgagctgtccctggtcaacttcgaggaatcccagaccttcttcggcctgtacaagcttggtggctctggcggaaaggtggcccatctgactggaaagtctaactcccggtctatgccccttgaatgggaagatacttatgggattgtcctgctgagcggggtcaagtataagaaaggtggactcgtgattaacgaaaccgggctctacttcgtctactctaaggtctacttcagagggcagagctgtaacaatctgcccctgtctcacaaggtctacatgagaaactccaagtatccccaggatctcgtgatgatggaagggaaaatgatgagctactgtacaacagggcaaatgtgggctagaagctcctacctgggagctgtgtttaatctgacctctgctgaccacctctatgtgaatgtgtctgaactgagcctcgtgaacttcgaagagagccagaccttttttgggctctataagctg

SEQ ID NO: 69 - DNA, 1218 bp, Artificial Sequence
  source: /note=“Description of Artificial Sequence: Synthetic polynucleotide”
atggaaaccgacacactgctgctgtgggtgctgctcttgtgggtgccaggatctaccggcgacaagacccatacctgtcctccatgtcctgctccagagctgctcggaggcccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccctgcctcctagccgggaagagatgaccaagaaccaggtgtccctgtcctgtgccgtgaagggcttctacccttccgatatcgccgtggaatgggagagcaatggccagcctgagaacaactacaagaccacacctcctgtgctggactccgacggctcattcttcctggtgtccaagctgacagtggacaagtccagatggcagcagggcaacgtgttctcctgctccgtgatgcacgaggccctgcacaatcactacacccagaagtccctgagcctgtctcctggaaaaggcggcggaggatctggcggaggtggaagcggaggcggaggaagcggtggcggcggatctttgagaaaagtggctcacctgaccggcaagtccaacagcagatccatgcctctcgagtgggaagatacctacggcatcgtgctgctgtccggcgtgaagtacaagaaaggcggcctggtcatcaacgagactggcctgtacttcgtgtactccaaggtgtacttccggggccagtcctgcaacaacctgcctctgagccataaggtgtacatgcggaacagcaagtaccctcaggacctggtcatgatggaaggcaagatgatgtcctactgcaccaccggccagatgtgggccagatcttcttatctgggcgccgtgttcaacctgaccagcgccgatcacctgtacgtgaacgtgtccgagctgtccctggtcaacttcgaggaatcccagaccttcttcggcctgtacaagctg

SEQ ID NO: 70 - DNA, 1704 bp, Artificial Sequence
  source: /note=“Description of Artificial Sequence: Synthetic polynucleotide”
atggaaaccgacacactgctgctgtgggtgctgctcttgtgggtgccaggatctaccggcgacaagacccatacctgtcctccatgtcctgctccagagctgctcggaggcccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccatgccgggaagagatgaccaagaaccaggtgtccctgtggtgcctggtcaagggcttctacccttccgatatcgccgtggaatgggagagcaatggccagcctgagaacaactacaagaccacacctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaaggcggcggaggatctggcggaggtggaagcggaggcggaggaagcggtggcggcggatctaatcctgcagctcatctgaccggcgccaactcctctctgactggttctggtggacccctgctctgggaaactcagctgggactcgctttcctgcggggcctgtcttatcatgatggcgctctggtggtcaccaaggccggctactactacatctactccaaggtgcagctcggcggcgtgggatgtcctcttggactggcttctacaatcacccacggcctgtacaagcggacccctagataccccgaggaactggaactgctggtgtcccagcagtctccttgtggcagagccacctcctccagcagagtttggtgggactcttctttccttggtggcgtggtgcacctggaagctggcgagaaagtggtcgtcagagtgctggacgaaagactcgtgcggctgagagatggcacccggtcttacttcggcgcctttatggttggaggctccggcggaaatccagccgctcatttgacaggggccaattccagcttgaccggctctggcggacctctcctgtgggagactcaactcggactggcctttctgagaggcctgtcctaccacgacggtgctctggtcgttacaaaggctgggtactattatatctacagcaaagtccaactcggcggagtcggctgtccactcggactcgctagcaccatcacacatggcctgtataagagaaccccacgctatcctgaggaactcgagcttctcgtcagccagcagagcccttgtggacgggctacctccagctctcgagtgtggtgggatagctcttttcttggcggggtcgtgcatttggaggccggcgaaaaggtcgtcgttcgcgtgttggatgagaggctcgtcagactcagagatggaaccagaagctactttggcgctttcatggtc

SEQ ID NO: 71 - DNA, 1245 bp, Artificial Sequence
  source: /note=“Description of Artificial Sequence: Synthetic polynucleotide”
atggaaaccgacacactgctgctgtgggtgctgctcttgtgggtgccaggatctaccggcgacaagacccatacctgtcctccatgtcctgctccagagctgctcggaggcccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccctgcctcctagccgggaagagatgaccaagaaccaggtgtccctgtcctgtgccgtgaagggcttctacccttccgatatcgccgtggaatgggagagcaatggccagcctgagaacaactacaagaccacacctcctgtgctggactccgacggctcattcttcctggtgtccaagctgacagtggacaagtccagatggcagcagggcaacgtgttctcctgctccgtgatgcacgaggccctgcacaatcactacacccagaagtccctgagcctgtctcctggaaaaggcggcggaggatctggcggaggtggaagcggaggcggaggaagcggtggcggcggatctaatcctgcagctcatctgaccggcgccaactcctctctgactggttctggtggacccctgctctgggaaactcagctgggactcgctttcctgcggggcctgtcttatcatgatggcgctctggtggtcaccaaggccggctactactacatctactccaaggtgcagctcggcggcgtgggatgtcctcttggactggcttctacaatcacccacggcctgtacaagcggacccctagataccccgaggaactggaactgctggtgtcccagcagtctccttgtggcagagccacctcctccagcagagtttggtgggactcttctttccttggtggcgtggtgcacctggaagctggcgagaaagtggtcgtcagagtgctggacgaaagactcgtgcggctgagagatggcacccggtcttacttcggcgccttcatggtg

SEQ ID NO: 72 - DNA, 1557 bp, Artificial Sequence
  source: /note=“Description of Artificial Sequence: Synthetic polynucleotide”
atggaaaccgacacactgctgctgtgggtgctgctcttgtgggtgccaggatctaccggcgacaagacccatacctgtcctccatgtcctgctccagagctgctcggaggcccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccatgccgggaagagatgaccaagaaccaggtgtccctgtggtgcctggtcaagggcttctacccttccgatatcgccgtggaatgggagagcaatggccagcctgagaacaactacaagaccacacctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaaggcggcggaggatctggcggaggtggaagcggaggcggaggaagcggtggcggcggatctcaactggaaaccgccaaagaaccctgcatggccaagttcggccctctgccttctaagtggcagatggcctcttccgagcctccttgcgtgaacaaagtgtccgactggaagctggaaatcctgcagaacggcctgtacctgatctacggacaggtggcccctaacgccaactacaacgatgtggcccctttcgaagtgcggctgtacaagaacaaggacatgatccagacactgaccaacaagtccaagatccagaacgtcggcggcacctacgagctgcatgtgggcgataccatcgacctgatcttcaactccgagcaccaggtgctgaagaacaacacctactggggcatcatcctgctggctaacggccagctggaaacagctaaagaaccttgtatggctaaatttgggcccctgcctagcaaatggcaaatggctagcagcgagccaccatgtgtgaacaaggtttcagattggaaactcgagatcctccagaatgggctctatctgatctatggccaggtcgcacccaatgctaactacaatgacgtcgcaccatttgaagtccgcctctataagaacaaagatatgattcaaaccctcacaaacaagagcaagattcagaatgttggcgggacatatgaactgcacgtgggagacacaatcgatctcatcttcaatagcgagcatcaggtcctcaaaaacaatacttattggggaatcattctgctcgccaatcctcagttcatctcc

SEQ ID NO: 73 - DNA, 1185 bp, Artificial Sequence
  source: /note=“Description of Artificial Sequence: Synthetic polynucleotide”
atggaaaccgacacactgctgctgtgggtgctgctcttgtgggtgccaggatctaccggcgacaagacccatacctgtcctccatgtcctgctccagagctgctcggaggcccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctcgggaacctcaagtctgtaccctgcctcctagccgggaagagatgaccaagaaccaggtgtccctgtcctgtgccgtgaagggcttctacccttccgatatcgccgtggaatgggagagcaatggccagcctgagaacaactacaagaccacacctcctgtgctggactccgacggctcattcttcctggtgtccaagctgacagtggacaagtccagatggcagcagggcaacgtgttctcctgctccgtgatgcacgaggccctgcacaatcactacacccagaagtccctgagcctgtctcctggaaaaggcggcggaggatctggcggaggtggaagcggaggcggaggaagcggtggcggcggatctcaactggaaaccgccaaagaaccctgcatggccaagttcggccctctgccttctaagtggcagatggcctcttccgagcctccttgcgtgaacaaagtgtccgactggaagctggaaatcctgcagaacggcctgtacctgatctacggacaggtggcccctaacgccaactacaacgatgtggcccctttcgaagtgcggctgtacaagaacaaggacatgatccagacactgaccaacaagtccaagatccagaacgtcggcggcacctacgagctgcatgtgggcgataccatcgacctgatcttcaactccgagcaccaggtgctgaagaacaacacctactggggcatcatcctgctggccaatcctcagttcatctcc

SEQ ID NO: 74 - protein, 20 aa, Artificial Sequence
  source: /note=“Description of Artificial Sequence: Synthetic peptide”
METDTLLLWVLLLWVPGSTG

SEQ ID NO: 75 - protein, 107 aa, Artificial Sequence [sequence omitted]

SEQ ID NO: 76 - protein, 103 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 77 - protein, 227 aa, Artificial Sequence
  source: /note=“Description of Artificial Sequence: Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 227 aa, Artificial Sequence
  source: /note=“Description of Artificial Sequence: Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 79 - protein, 1 aa, Artificial Sequence
  source: /note=“Description of Artificial Sequence: Synthetic peptide”
G

SEQ ID NO: 80 - protein, 5 aa, Artificial Sequence
  source: /note=“Description of Artificial Sequence: Synthetic peptide”
GGGGS

SEQ ID NO: 81 - protein, 5 aa, Artificial Sequence
  source: /note=“Description of Artificial Sequence: Synthetic peptide”
GGSGG

SEQ ID NO: 82 - protein, 6 aa, Artificial Sequence
  source: /note=“Description of Artificial Sequence: Synthetic peptide”
GGSGGS

SEQ ID NO: 83 - protein, 7 aa, Artificial Sequence
  source: /note=“Description of Artificial Sequence: Synthetic peptide”
GGGSGGG

SEQ ID NO: 84 - protein, 15 aa, Artificial Sequence
  source: /note=“Description of Artificial Sequence: Synthetic peptide”
GGGGSGGGGSGGGGS

SEQ ID NO: 85 - protein, 146 aa, Artificial Sequence
  source: /note=“Description of Artificial Sequence: Synthetic polypeptide”
GDQNPQIAAHVISEASSKTTSVLQWAEKGYYTMSNNLVTLENGKQLTVKRQGLYYIYAQVTFCSNREASSQAPFIASLALKSPGRFERILLRAANTHSSAKPCGQQSIHLGGVFELQPGASVFVNVTDPSQVSHGTGFTSFGLLKL

SEQ ID NO: 86 - protein, 126 aa, Artificial Sequence; identical to SEQ ID NO: 4
  source: /note=“Description of Artificial Sequence: Synthetic polypeptide”

SEQ ID NO: 87 - protein, 152 aa, Artificial Sequence; identical to SEQ ID NO: 2
  source: /note=“Description of Artificial Sequence: Synthetic polypeptide”

SEQ ID NO: 88 - protein, 148 aa, Artificial Sequence
  source: /note=“Description of Artificial Sequence: Synthetic polypeptide”
DKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 89 - protein, 123 aa, Artificial Sequence
  source: /note=“Description of Artificial Sequence: Synthetic polypeptide”
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLAN

SEQ ID NO: 90 - protein, 128 aa, Artificial Sequence; identical to SEQ ID NO: 17
  source: /note=“Description of Artificial Sequence: Synthetic polypeptide”

SEQ ID NO: 91 - protein, 148 aa, Artificial Sequence; identical to SEQ ID NO: 15
  source: /note=“Description of Artificial Sequence: Synthetic polypeptide”

SEQ ID NO: 92 - protein, 139 aa, Artificial Sequence; identical to SEQ ID NO: 6
  source: /note=“Description of Artificial Sequence: Synthetic polypeptide”

SEQ ID NO: 93 - protein, 137 aa, Artificial Sequence
  source: /note=“Description of Artificial Sequence: Synthetic polypeptide”
KVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 94 - protein, 559 aa, Artificial Sequence; identical to SEQ ID NO: 18
  source: /note=“Description of Artificial Sequence: Synthetic polypeptide”

SEQ ID NO: 95 - protein, 408 aa, Artificial Sequence; identical to SEQ ID NO: 19
  source: /note=“Description of Artificial Sequence: Synthetic polypeptide”

SEQ ID NO: 96 - protein, 247 aa, Artificial Sequence; identical to SEQ ID NO: 20
  source: /note=“Description of Artificial Sequence: Synthetic polypeptide”

SEQ ID NO: 97 - protein, 662 aa, Artificial Sequence; identical to SEQ ID NO: 21
  source: /note=“Description of Artificial Sequence: Synthetic polypeptide”

SEQ ID NO: 98 - protein, 288 aa, Artificial Sequence; identical to SEQ ID NO: 22
  source: /note=“Description of Artificial Sequence: Synthetic polypeptide”

SEQ ID NO: 99 - protein, 526 aa, Artificial Sequence
  source: /note=“Description of Artificial Sequence: Synthetic polypeptide”
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGGSGGGRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 100 - protein, 393 aa, Artificial Sequence
  source: /note=“Description of Artificial Sequence: Synthetic polypeptide”
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 101 - protein, 573 aa, Artificial Sequence; identical to SEQ ID NO: 27
  source: /note=“Description of Artificial Sequence: Synthetic polypeptide”

SEQ ID NO: 102 - protein, 419 aa, Artificial Sequence; identical to SEQ ID NO: 28
  source: /note=“Description of Artificial Sequence: Synthetic polypeptide”

SEQ ID NO: 103 - protein, 548 aa, Artificial Sequence; identical to SEQ ID NO: 31
  source: /note=“Description of Artificial Sequence: Synthetic polypeptide”

SEQ ID NO: 104 - protein, 406 aa, Artificial Sequence; identical to SEQ ID NO: 32
  source: /note=“Description of Artificial Sequence: Synthetic polypeptide”

SEQ ID NO: 105 - protein, 568 aa, Artificial Sequence; identical to SEQ ID NO: 35
  source: /note=“Description of Artificial Sequence: Synthetic polypeptide”

SEQ ID NO: 106 - protein, 415 aa, Artificial Sequence; identical to SEQ ID NO: 36
  source: /note=“Description of Artificial Sequence: Synthetic polypeptide”

SEQ ID NO: 107 - protein, 519 aa, Artificial Sequence; identical to SEQ ID NO: 23
  source: /note=“Description of Artificial Sequence: Synthetic polypeptide”

SEQ ID NO: 108 - protein, 395 aa, Artificial Sequence; identical to SEQ ID NO: 24
  source: /note=“Description of Artificial Sequence: Synthetic polypeptide”

SEQ ID NO: 109-115 - skipped (7 entries)

SEQ ID NO: 116 - protein, 122 aa, Artificial Sequence
  source: /note=“Description of Artificial Sequence: Synthetic polypeptide”
EVQLQQSGPDLVKPGASVKISCKTSGYTFTEYIMHWVKQSHGKSLEWIGGIIPNNGGTSYNQKFKDKATMTVDKSSSTGYMELRSLTSEDSAVYYCTRREVYGRNYYALDYWGQGTLVTVSS

SEQ ID NO: 117 - protein, 107 aa, Artificial Sequence
  source: /note=“Description of Artificial Sequence: Synthetic polypeptide”
DIQMTQTTSSLSASLGDRVTITCSASQGINNYLNWYQQKPDGTVKLLIYYTSSLHSGVPSRFSGSGSGTDYSLTISNLEPEDIATYYCQQYSNLPYTFGGGTKLEIK

SEQ ID NO: 118 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 119 - protein, 93 aa, Unknown
  source: /note=“Description of Unknown: TCR-alpha domain sequence”
PDIQNPDPAVYQLRDSKSSDKSVCLFTDFDSQTNVSQSKDSDVYITDKCVLDMRSMDFKSNSAVAWSNKSDFACANAFNNSIIPEDTFFPSPE

SEQ ID NO: 120 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 121 - protein, 130 aa, Unknown
  source: /note=“Description of Unknown: TCR-beta constant domain sequence”
EDLNKVFPPEVALFEPSEAEISHTQKATLVCLATGFYPDHVELSWWVNGKEVHSGVCTDPQPLKEQPALNDSRYALSSRLRVSATFWQDPRNHFRCQVQFYGLSEADEWTQARAKPVTQIVSAEAWGRAD

SEQ ID NO: 122 - protein, 559 aa, Artificial Sequence
  source: /note=“Description of Artificial Sequence: Synthetic polypeptide”
METDTLLLWVLLLWVPGSTGDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGDQNPQIAAHVISEASSKTTSVLQWAEKGYYTMSNNLVTLENGKQLTVKRQGLYYIYAQVTFCSNREASSQAPFIASLALKSPGRFERILLRAANTHSSAKPCGQQSIHLGGVFELQPGASVFVNVTDPSQVSHGTGFTSFGLLKLGGGGSGDQNPQIAAHVISEASSKTTSVLQWAEKGYYTMSNNLVTLENGKQLTVKRQGLYYIYAQVTFCSNREASSQAPFIASLALKSPGRFERILLRAANTHSSAKPCGQQSIHLGGVFELQPGASVFVNVTDPSQVSHGTGFTSFGLLKL

SEQ ID NO: 123 - protein, 408 aa, Artificial Sequence
  source: /note=“Description of Artificial Sequence: Synthetic polypeptide”
METDTLLLWVLLLWVPGSTGDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGDQNPQIAAHVISEASSKTTSVLQWAEKGYYTMSNNLVTLENGKQLTVKRQGLYYIYAQVTFCSNREASSQAPFIASLALKSPGRFERILLRAANTHSSAKPCGQQSIHLGGVFELQPGASVFVNVTDPSQVSHGTGFTSFGLLKL

SEQ ID NO: 124 - protein, 616 aa, Artificial Sequence
  source: /note=“Description of Artificial Sequence: Synthetic polypeptide”
METDTLLLWVLLLWVPGSTGQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANGQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 125 - protein, 269 aa, Artificial Sequence
  source: /note=“Description of Artificial Sequence: Synthetic polypeptide”
METDTLLLWVLLLWVPGSTGQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 126 - protein, 470 aa, Artificial Sequence
  source: /note=“Description of Artificial Sequence: Synthetic polypeptide”
METDTLLLWVLLLWVPGSTGEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 236 aa, Artificial Sequence
  source: /note=“Description of Artificial Sequence: Synthetic polypeptide”
METDTLLLWVLLLWVPGSTGQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 128 - protein, 502 aa, Artificial Sequence
  source: /note=“Description of Artificial Sequence: Synthetic polypeptide”
METDTLLLWVLLLWVPGSTGEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSGGGGSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 736 aa, Artificial Sequence
  source: /note=“Description of Artificial Sequence: Synthetic polypeptide”
METDTLLLWVLLLWVPGSTGEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGSGGKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 130 - protein, 608 aa, Artificial Sequence
  source: /note=“Description of Artificial Sequence: Synthetic polypeptide”
METDTLLLWVLLLWVPGSTGEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 131 - protein, 768 aa, Artificial Sequence
  source: /note=“Description of Artificial Sequence: Synthetic polypeptide”
METDTLLLWVLLLWVPGSTGEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSGGGGSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGGGGSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGSGGKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 132 - protein, 640 aa, Artificial Sequence
  source: /note=“Description of Artificial Sequence: Synthetic polypeptide”
METDTLLLWVLLLWVPGSTGEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSGGGGSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGGGGSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 133 - protein, 499 aa, Artificial Sequence
  source: /note=“Description of Artificial Sequence: Synthetic polypeptide”
METDTLLLWVLLLWVPGSTGDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPEGGGGSGGGGSGGGGSKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGSGGKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 134 - protein, 408 aa, Artificial Sequence
  source: /note=“Description of Artificial Sequence: Synthetic polypeptide”
METDTLLLWVLLLWVPGSTGDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKEDLNKVFPPEVALFEPSEAEISHTQKATLVCLATGFYPDHVELSWWVNGKEVHSGVSTDPQPLKEQPALNDSRYALSSRLRVSATFWQDPRNHFRCQVQFYGLSEADEWTQARAKPVTQIVSAEAWGRADGGGGSGGGGSGGGGSKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 135 - protein, 722 aa, Artificial Sequence
  source: /note=“Description of Artificial Sequence: Synthetic polypeptide”
METDTLLLWVLLLWVPGSTGEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPEGGGGSGGGGSGGGGSKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGSGGKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 136 - protein, 631 aa, Artificial Sequence
  source: /note=“Description of Artificial Sequence: Synthetic polypeptide”
METDTLLLWVLLLWVPGSTGEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKEDLNKVFPPEVALFEPSEAEISHTQKATLVCLATGFYPDHVELSWWVNGKEVHSGVSTDPQPLKEQPALNDSRYALSSRLRVSATFWQDPRNHFRCQVQFYGLSEADEWTQARAKPVTQIVSAEAWGRADGGGGSGGGGSGGGGSKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 137 - protein, 754 aa, Artificial Sequence
  source: /note=“Description of Artificial Sequence: Synthetic polypeptide”
METDTLLLWVLLLWVPGSTGEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSGGGGSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGGGGSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPEGGGGSGGGGSGGGGSKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGSGGKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 138 - protein, 663 aa, Artificial Sequence
  source: /note=“Description of Artificial Sequence: Synthetic polypeptide”
METDTLLLWVLLLWVPGSTGEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSGGGGSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGGGGSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKEDLNKVFPPEVALFEPSEAEISHTQKATLVCLATGFYPDHVELSWWVNGKEVHSGVSTDPQPLKEQPALNDSRYALSSRLRVSATFWQDPRNHFRCQVQFYGLSEADEWTQARAKPVTQIVSAEAWGRADGGGGSGGGGSGGGGSKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 139 - protein, 638 aa, Artificial Sequence
  source: /note=“Description of Artificial Sequence: Synthetic polypeptide”
METDTLLLWVLLLWVPGSTGKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGSGGKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKEDLNKVFPPEVALFEPSEAEISHTQKATLVCLATGFYPDHVELSWWVNGKEVHSGVSTDPQPLKEQPALNDSRYALSSRLRVSATFWQDPRNHFRCQVQFYGLSEADEWTQARAKPVTQIVSAEAWGRAD

SEQ ID NO: 140 - protein, 456 aa, Artificial Sequence
  source: /note=“Description of Artificial Sequence: Synthetic polypeptide”
METDTLLLWVLLLWVPGSTGEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPE

SEQ ID NO: 141 - protein, 488 aa, Artificial Sequence
  source: /note=“Description of Artificial Sequence: Synthetic polypeptide”
METDTLLLWVLLLWVPGSTGEVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSGGGGSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPE

SEQ ID NO: 142-157 - skipped (16 entries)

SEQ ID NO: 158 - protein, 507 aa, Artificial Sequence
  source: /note=“Description of Artificial Sequence: Synthetic polypeptide”
DKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANGGGGSGGGGQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 159 - protein, 375 aa, Artificial Sequence
  source: /note=“Description of Artificial Sequence: Synthetic polypeptide”
DKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 160 - protein, 507 aa, Artificial Sequence
  source: /note=“Description of Artificial Sequence: Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANGGGGSGGGGQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 161 - protein, 375 aa, Artificial Sequence
  source: /note=“Description of Artificial Sequence: Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 162 - protein, 605 aa, Artificial Sequence
  source: /note=“Description of Artificial Sequence: Synthetic polypeptide”
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANGGGGSGGGGQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 250 aa, Artificial Sequence
  source: /note=“Description of Artificial Sequence: Synthetic polypeptide”
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 164 - protein, 227 aa, Artificial Sequence; identical to SEQ ID NO: 78
  source: /note=“Description of Artificial Sequence: Synthetic polypeptide”

SEQ ID NO: 165 - protein, 605 aa, Artificial Sequence
  source: /note=“Description of Artificial Sequence: Synthetic polypeptide”
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANGGGGSGGGGQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 250 aa, Artificial Sequence
  source: /note=“Description of Artificial Sequence: Synthetic polypeptide”
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - protein, 528 aa, Artificial Sequence
  source: /note=“Description of Artificial Sequence: Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSLRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKLGGSGGKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 168 - protein, 386 aa, Artificial Sequence
  source: /note=“Description of Artificial Sequence: Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSLRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 169 - protein, 528 aa, Artificial Sequence
  source: /note=“Description of Artificial Sequence: Synthetic polypeptide”
DKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSLRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKLGGSGGKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 170 - protein, 386 aa, Artificial Sequence
  source: /note=“Description of Artificial Sequence: Synthetic polypeptide”
DKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSLRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 171 - protein, 623 aa, Artificial Sequence
  source: /note=“Description of Artificial Sequence: Synthetic polypeptide”
DKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRS

SEQ ID NO: 172 - protein, 430 aa, Artificial Sequence
  source: /note=“Description of Artificial Sequence: Synthetic polypeptide”
DKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRS

SEQ ID NO: 173 - protein, 623 aa, Artificial Sequence
  source: /note=“Description of Artificial Sequence: Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRS

SEQ ID NO: 174 - protein, 430 aa, Artificial Sequence
  source: /note=“Description of Artificial Sequence: Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRS

SEQ ID NO: 175 - protein, 544 aa, Artificial Sequence
  source: /note=“Description of Artificial Sequence: Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLLGGGGSETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 176 - protein, 383 aa, Artificial Sequence
  source: /note=“Description of Artificial Sequence: Synthetic polypeptide”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSHSVLHLVPINAASKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 177 - protein, 544 aa, Artificial Sequence
  source: /note=“Description of Artificial Sequence: Synthetic polypeptide”
DKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLLGGGGSETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 178 - protein, 383 aa, Artificial Sequence
  source: /note=“Description of Artificial Sequence: Synthetic polypeptide”
DKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSHSVLHLVPINAASKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 179 - skipped

SEQ ID NO: 180 - protein, 136 aa, Artificial Sequence
  source: /note=“Description of Artificial Sequence: Synthetic polypeptide”
HSVLHLVPINAASKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 181 - protein, 11 aa, Artificial Sequence
  source: /note=“Description of Artificial Sequence: Synthetic peptide”
EPKSDKTHTCP

SEQ ID NO: 182 - protein, 12 aa, Artificial Sequence
  source: /note=“Description of Artificial Sequence: Synthetic peptide”
EPKSCDKTHTCP

SEQ ID NO: 183 - protein, 106 aa, Artificial Sequence
  source: /note=“Description of Artificial Sequence: Synthetic polypeptide”
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 184 - protein, 227 aa, Artificial Sequence
  source: /note=“Description of Artificial Sequence: Synthetic polypeptide”
DKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 227 aa, Artificial Sequence
  source: /note=“Description of Artificial Sequence: Synthetic polypeptide”
DKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 186 - protein, 102 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKS

SEQ ID NO: 187 - protein, 329 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 188 - protein, 329 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 9 aa, Artificial Sequence
  source: /note=“Description of Artificial Sequence: Synthetic peptide”
GGGGSGGGG

SEQ ID NO: 190 - protein, 20 aa, Artificial Sequence
  source: /note=“Description of Artificial Sequence: Synthetic peptide”
GGGGSGGGGSGGGGSGGGGS

SEQ ID NO: 191 - protein, 107 aa, Artificial Sequence
  source: /note=“Description of Artificial Sequence: Synthetic polypeptide”
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 192 - protein, 103 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSS

SEQ ID NO: 193 - protein, 330 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 194 - protein, 330 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 184 aa, Artificial Sequence
  source: /note=“Description of Artificial Sequence: Synthetic polypeptide”
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 196 - protein, 183 aa, Artificial Sequence
  source: /note=“Description of Artificial Sequence: Synthetic polypeptide”
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRS

SEQ ID NO: 197 - protein, 12 aa, Artificial Sequence
  source: /note=“Description of Artificial Sequence: Synthetic peptide”
EPKSSDKTHTCP

SEQ ID NO: 198 - protein, 146 aa, Artificial Sequence; identical to SEQ ID NO: 85
  source: /note=“Description of Artificial Sequence: Synthetic polypeptide”

SEQ ID NO: 199-217 - skipped (19 entries)

SEQ ID NO: 218 - protein, 144 aa, Homo sapiens
QPAVVHLQGQGSAIQVKNDLSGGVLNDWSRITMNPKVFKLHPRSGELEVLVDGTYFIYSQVEVYYINFTDFASYEVVVDEKPFLQCTRSIETGKTNYNTCYTAGVCLLKARQKIAVKMVHADISINMSKHTTFFGAIRLGEAPA

SEQ ID NO: 219-238 - skipped (20 entries)

SEQ ID NO: 239 - protein, 133 aa, Homo sapiens
QPRPAFSAIRRNPPMGGNVVIFDTVITNQEEPYQNHSGRFVCTVPGYYYFTFQVLSQWEICLSIVSSSRGQVRRSLGFCDTTNKGLFQVVSGGMVLQLQQGDQVWVEKDPKKGHIYQGSEADSVFSGFLIFPS

SEQ ID NO: 240 - protein, 132 aa, Homo sapiens
TQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNSLLGMEGANSIFSGFLLFPD

SEQ ID NO: 241 - protein, 129 aa, Homo sapiens
KFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPD

SEQ ID NO: 242 - protein, 131 aa, Homo sapiens
PRVAFYAGLKNPHEGYEVLKFDDVVTNLGNNYDAASGKFTCNIPGTYFFTYHVLMRGGDGTSMWADLCKNGQVRASAIAQDADQNYDYASNSVILHLDAGDEVFIKLDGGKAHGGNSNKYSTFSGFIIYSD

SEQ ID NO: 243 - protein, 131 aa, Homo sapiens
PKIAFYVGLKSPHEGYEVLKFDDVVTNLGNHYDPTTGKFSCQVRGIYFFTYHILMRGGDGTSMWADLCKNGQVRASAIAQDADQNYDYASNSVVLHLDSGDEVYVKLDGGKAHGGNNNKYSTFSGFLLYPD

SEQ ID NO: 244 - protein, 131 aa, Homo sapiens
PKIAFYAGLKRQHEGYEVLKFDDVVTNLGNHYDPTTGKFTCSIPGIYFFTYHVLMRGGDGTSMWADLCKNNQVRASAIAQDADQNYDYASNSVVLHLEPGDEVYIKLDGGKAHGGNNNKYSTFSGFIIYAD

SEQ ID NO: 245 - protein, 132 aa, Homo sapiens
MRVAFSAARTSNLAPGTLDQPIVFDLLLNNLGETFDLQLGRFNCPVNGTYVFIFHMLKLAVNVPLYVNLMKNEEVLVSAYANDGAPDHETASNHAILQLFQGDQIWLRLHRGAIYGSSWKYSTFSGYLLYQD

SEQ ID NO: 246 - protein, 136 aa, Homo sapiens
GSAKVAFSAIRSTNHEPSEMSNRTMIIYFDQVLVNIGNNFDSERSTFIAPRKGIYSFNFHVVKVYNRQTIQVSLMLNGWPVISAFAGDQDVTREAASNGVLIQMEKGDRAYLKLERGNLMGGWKYSTFSGFLVFPL

SEQ ID NO: 247 - protein, 141 aa, Homo sapiens
PGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 248 - protein, 119 aa, Artificial Sequence
  source: /note=“Description of Artificial Sequence: Synthetic polypeptide”
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSS

SEQ ID NO: 249 - protein, 10 aa, Artificial Sequence
  source: /note=“Description of Artificial Sequence: Synthetic peptide”
GYSFTGYTMN

SEQ ID NO: 250 - protein, 17 aa, Artificial Sequence
  source: /note=“Description of Artificial Sequence: Synthetic peptide”
LITPYNGASSYNQKFRG

SEQ ID NO: 251 - protein, 10 aa, Artificial Sequence
  source: /note=“Description of Artificial Sequence: Synthetic peptide”
GGYDGRGFDY

SEQ ID NO: 252 - protein, 108 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHT

SEQ ID NO: 253 - protein, 19 aa, Artificial Sequence
  source: /note=“Description of Artificial Sequence: Synthetic peptide”
MEFGLSWVFLVALFRGVQC

SEQ ID NO: 254 - protein, 106 aa, Artificial Sequence
  source: /note=“Description of Artificial Sequence: Synthetic polypeptide”
DIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIK

SEQ ID NO: 255 - protein, 10 aa, Artificial Sequence
  source: /note=“Description of Artificial Sequence: Synthetic peptide”
SASSSVSYMH

SEQ ID NO: 256 - protein, 7 aa, Artificial Sequence
  source: /note=“Description of Artificial Sequence: Synthetic peptide”
DTSKLAS

SEQ ID NO: 257 - protein, 9 aa, Artificial Sequence
  source: /note=“Description of Artificial Sequence: Synthetic peptide”
QQWSGYPLT

SEQ ID NO: 258 - protein, 106 aa, Artificial Sequence; identical to SEQ ID NO: 254
  source: /note=“Description of Artificial Sequence: Synthetic polypeptide”

SEQ ID NO: 259 - protein, 107 aa, Artificial Sequence [sequence omitted]

SEQ ID NO: 260 - protein, 22 aa, Artificial Sequence
  source: /note=“Description of Artificial Sequence: Synthetic peptide”
MKYLLPTAAAGLLLLAAQPAMA

SEQ ID NO: 261 - protein, 124 aa, Artificial Sequence
  source: /note=“Description of Artificial Sequence: Synthetic polypeptide”
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 262 - protein, 103 aa, Artificial Sequence; identical to SEQ ID NO: 76
  source: /note=“Description of Artificial Sequence: Synthetic polypeptide”

SEQ ID NO: 263 - protein, 21 aa, Artificial Sequence
  source: /note=“Description of Artificial Sequence: Synthetic peptide”
MEFGLSWVFLVALFRGVQCEV

SEQ ID NO: 264 - protein, 113 aa, Artificial Sequence
  source: /note=“Description of Artificial Sequence: Synthetic polypeptide”
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 265 - protein, 107 aa, Artificial Sequence [sequence omitted]

SEQ ID NO: 266 - protein, 22 aa, Artificial Sequence
  source: /note=“Description of Artificial Sequence: Synthetic peptide”
MKYLLPTAAAGLLLLAAQPAMA

SEQ ID NO: 267 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 268 - protein, 77 aa, Homo sapiens
MAPRRARGCRTLGLPALLLLLLLRPPATRGITCPPPMSVEHADIWVKSYSLYSRERYICNSGFKRKAGTSSLTECVL

SEQ ID NO: 269 - protein, 20 aa, Artificial Sequence
  source: /note=“Description of Artificial Sequence: Synthetic peptide”
SGGSGGGGSGGGSGGGGSLQ

SEQ ID NO: 270 - protein, 133 aa, Homo sapiens [sequence omitted]

SEQ ID NO: 271 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 272 - protein, 133 aa, Homo sapiens [sequence omitted]

SEQ ID NO: 273 - protein, 138 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 274 - protein, 238 aa, Artificial Sequence
  source: /note=“Description of Artificial Sequence: Synthetic polypeptide”
DLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFS

SEQ ID NO: 275 - protein, 284 aa, Artificial Sequence
  source: /note=“Description of Artificial Sequence: Synthetic polypeptide”
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHW

SEQ ID NO: 276 - protein, 287 aa, Artificial Sequence
  source: /note=“Description of Artificial Sequence: Synthetic polypeptide”
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKVLVLQSQRTD

SEQ ID NO: 277 - protein, 191 aa, Artificial Sequence
  source: /note=“Description of Artificial Sequence: Synthetic polypeptide”
GWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPG

SEQ ID NO: 278 - protein, 329 aa, Artificial Sequence
  source: /note=“Description of Artificial Sequence: Synthetic polypeptide”
QDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGG

SEQ ID NO: 279 - protein, 323 aa, Artificial Sequence
  source: /note=“Description of Artificial Sequence: Synthetic polypeptide”
WPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRN

SEQ ID NO: 280 - protein, 330 aa, Artificial Sequence
  source: /note=“Description of Artificial Sequence: Synthetic polypeptide”
QNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDSRAGEEGSIRAVDH

SEQ ID NO: 281 - protein, 155 aa, Artificial Sequence; identical to SEQ ID NO: 7
  source: /note=“Description of Artificial Sequence: Synthetic polypeptide”

SEQ ID NO: 282 - protein, 294 aa, Artificial Sequence
  source: /note=“Description of Artificial Sequence: Synthetic polypeptide”
WTYHYSEKPMNWQRARRFCRDNYTDLVAIQNKAEIEYLEKTLPFSRSYYWIGIRKIGGIWTWVGTNKSLTEEAENWGDGEPNNKKNKEDCVEIYIKRNKDAGKWNDDACHKLKAALCYTASCQPWSCSGHGECVEIINNYTCNCDVGYYGPQCQFVIQCEPLEAPELGTMDCTHPLGNFSFSSQCAFSCSEGTNLTGIEETTCGPFGNWSSPEPTCQVIQCEPLSAPDLGIMNCSHPLASFSFTSACTFICSEGTELIGKKKTICESSGIWSNPSPICQKLDKSFSMIKEGDYN

SEQ ID NO: 283 - protein, 323 aa, Artificial Sequence; identical to SEQ ID NO: 279
  source: /note=“Description of Artificial Sequence: Synthetic polypeptide”

SEQ ID NO: 284 - protein, 243 aa, Artificial Sequence
  source: /note=“Description of Artificial Sequence: Synthetic polypeptide”
RYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 285 - protein, 124 aa, Artificial Sequence
  source: /note=“Description of Artificial Sequence: Synthetic polypeptide”
KLTRDSQSLCPYDWIGFQNKCYYFSKEEGDWNSSKYNCSTQHADLTIIDNIEEMNFLRRYKCSSDHWIGLKMAKNRTGQWVDGATFTKSFGMRGSEGCAYLSDDGAATARCYTERKWICRKRIH

SEQ ID NO: 286 - protein, 194 aa, Artificial Sequence
  source: /note=“Description of Artificial Sequence: Synthetic polypeptide”
QGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARS

SEQ ID NO: 287 - protein, 133 aa, Artificial Sequence
  source: /note=“Description of Artificial Sequence: Synthetic polypeptide”
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 288 - protein, 149 aa, Artificial Sequence
  source: /note=“Description of Artificial Sequence: Synthetic polypeptide”
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 289 - protein, 205 aa, Artificial Sequence
  source: /note=“Description of Artificial Sequence: Synthetic polypeptide”
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 290 - protein, 5 aa, Artificial Sequence
  source: /note=“Description of Artificial Sequence: Synthetic peptide”
GGGGS

SEQ ID NO: 291 - protein, 10 aa, Artificial Sequence
  source: /note=“Description of Artificial Sequence: Synthetic peptide”
GGGGSGGGGS

SEQ ID NO: 292 - protein, 15 aa, Artificial Sequence
  source: /note=“Description of Artificial Sequence: Synthetic peptide”
GGGGSGGGGSGGGGS

SEQ ID NO: 293 - protein, 17 aa, Artificial Sequence
  source: /note=“Description of Artificial Sequence: Synthetic peptide”
DVPSGPGGGGGSGGGGS